(19) United States
(12) Patent Application Publication
(10) Pub. No.: US 2026/0242798 A1
(43) Pub. Date: Aug. 20, 2026

(54) RNAi FORMULATION FOR INHIBITING PCSK9 GENE EXPRESSION AND USE THEREOF

(71) Applicant: MABWELL (SHANGHAI) BIOSCIENCE CO., LTD., Shanghai (CN)

(72) Inventors: Fuzheng Yang, Shanghai (CN); Hao Chen, Shanghai (CN); Bing Jin, Shanghai (CN); Denghui Hu, Shanghai (CN); Chengxin Ma, Shanghai (CN); Xun Gui, Shanghai (CN)

(21) Appl. No.: 19/461,345

(22) Filed: Jan. 27, 2026

Related U.S. Application Data
(63) Continuation of application No. PCT/CN2025/099696, filed on Jun. 6, 2025.

(30) Foreign Application Priority Data
Jul. 6, 2024 (CN) 202410743381.6

(51) Int. Cl. C12N 15/113; A61K 31/7125; A61K 31/713; A61P 3/06

(52) U.S. Cl. CPC C12N 15/1137; A61K 31/7125; A61K 31/713; A61P 3/06; C12N 2310/11; C12N 2310/14; C12N 2310/316; C12N 2310/351; C12Y 304/21061

(57) ABSTRACT

The present application provides an RNA inhibitor for inhibiting PCSK9 gene expression, which comprises an antisense strand, wherein the antisense strand comprises a complementary region that is complementary to at least a part of an mRNA encoding PCSK9, and the complementary region is 17-23 nucleotides in length. The antisense strand comprises the following nucleotide sequence: any one of SEQ ID NOs: 328-654, 1045-1107, 1125-1146, or 1149-1170, or a sequence differing by no more than 3 nucleotides therefrom. In addition, the present application also provides a pharmaceutical composition, which comprises the RNA inhibitor for inhibiting PCSK9 gene expression, and further comprises a delivery vehicle, and/or a physiologically acceptable excipient and/or carrier and/or diluent.

### TECHNICAL FIELD

The present disclosure pertains to the field of molecular biology, relates to a modified double-stranded RNAi formulation and use thereof, and particularly relates to a double-stranded RNAi agent for inhibiting PCSK9 gene expression and a pharmaceutical composition thereof, and use of the double-stranded RNAi agent or the pharmaceutical composition thereof in treating a disease mediated by PCSK9 expression.

### BACKGROUND

Hypercholesterolemia itself is asymptomatic, but long-term serum cholesterol elevation can lead to atherosclerosis. It has been pointed out in the prior art that the causal relationship between low-density lipoprotein cholesterol (LDL-C) and the risk of cardiovascular disease depends on LDL-C level and cumulative duration of exposure (J am Coll Cardiol, 2018, 72:1141-1156), and the formation of atherosclerotic plaques is caused by the accumulation of LDL particles in the artery wall over time. Moreover, studies have shown that there is a remarkably consistent dose-dependent log-linear association between the absolute magnitude of exposure of the vasculature to LDL-C and the risk of ASCVD; and this effect appears to increase with increasing duration of exposure to LDL-C(European Heart Journal (2017) 38, 2459-2472). Therefore, lowering the blood cholesterol level can effectively reduce the risk of atherosclerotic cardiovascular disease (ASCVD).

PCSK9 is a member of the proprotein convertase family, and the proprotein convertase family is a family of serine proteases. PCSK9 is expressed as a 692-residue, 74.3-kDa zymogen with three distinguishable domains. From the N-terminus, it contains a prodomain, a catalytic domain, and a C-terminal cysteine- and histidine-rich (CHR) domain. The domains play important roles in the biological function and intracellular transport of PCSK9, as well as in proteolytic activation, modification, and degradation of secreted proteins. PCSK9 is a main regulator for the level of low-density lipoprotein receptor (LDLR) on the surface of a hepatocyte, and PCSK9 binds to the LDLR receptor, causing it to be degraded by lysosomes, and thus inhibiting the circulating pathway of LDLR. The function of LDLR is critical for cholesterol homeostasis as it is responsible for both cellular uptake and subsequent degradation of low-density lipoproteins. Circulating LDL binds to the N-terminal ligand-binding domain of LDLR via apolipoprotein B100, and the LDL/LDLR complex is internalized through receptor-mediated endocytosis. Upon migration to the endosome, in the low-pH environment, LDLR releases LDL and then is recirculated back to the cell surface. Free low-density lipoproteins are transported to lysosomes and degraded. PCSK9 binds to LDLR on the surface of the hepatocyte, so as to interfere with the circulating ability of LDLR.

Gain-of-function variations of PCSK9 are associated with extremely high LDL-C levels in plasma, and patients carrying these mutations are at an increased risk of suffering from early cardiovascular events, indicating that PCSK9 is a pharmacologically acceptable target. In addition, loss-of-function mutations in PCSK9 have been shown to significantly reduce LDL-C in patients without causing adverse health events, demonstrating that PCSK9 is not only an effective target, but also can be safely inhibited.

Currently, PCSK9 inhibitors have three main mechanisms of action: (1) blocking the binding of PCSK9 protein to LDLR; (2) interfering with the secretion of PCSK9 protein; and (3) inhibiting the expression of PCSK9 protein. Among them, the third approach utilizes the RNAi mechanism widely existing in nature to directly regulate the mRNA encoding PCSK9, and according to the Central Dogma, the expression of PCSK9 protein can be more effectively inhibited, thereby achieving stronger efficacy. Mainstream small-molecule statins for lowering LDL-C require a high frequency of administration and exhibit poor clinical compliance, and doubling the dose can only increase the LDL-C-lowering effect by 6%. Moreover, the long-term use of statins may lead to a number of potential side effects, such as liver, kidney, and muscle damage, new-onset diabetes, and the like. Moreover, due to racial differences, East Asian populations often cannot tolerate high levels of statins as European and American populations do in clinical practice. Compared with statins, PCSK9 monoclonal antibodies have fewer side effects after long-term administration, and the effect of combined administration on lowering LDL-C is better. However, subcutaneous injections are still required every 2 weeks, which are costly. Therefore, there is an urgent need to develop an efficient PCSK9 siRNA drug in the therapeutic field of regulating PCSK9 to lower LDL-C.

### SUMMARY

The present application provides an RNA inhibitor for inhibiting PCSK9 gene expression, which comprises an antisense strand, wherein the antisense strand comprises a complementary region that is complementary to at least a part of an mRNA encoding PCSK9, and the complementary region is 17-23 nucleotides in length, wherein the antisense strand comprises the following nucleotide sequence: any one of SEQ ID NOs: 328-654, 1045-1107, 1125-1146, or 1149-1170, or a sequence differing by no more than 3 nucleotides therefrom.

In some embodiments, the RNA inhibitor further comprises a sense strand, wherein at least 80% of bases between the sense strand and the antisense strand are complementary.

In some embodiments, in the RNA inhibitor, the sense nucleic acid strand and the antisense nucleic acid strand are present on two different nucleic acid strands.

In some embodiments, in the RNA inhibitor, the sense nucleic acid fragment and the antisense nucleic acid fragment are present on the same nucleic acid strand, wherein complementary regions of the sense nucleic acid fragment and the antisense nucleic acid fragment form a double-stranded nucleic acid structure.

In some embodiments, in the RNA inhibitor, at least one strand has a 3′ overhang of 0 to 6 nucleotides in length.

In some embodiments, in the RNA inhibitor, both strands have 3′ overhangs of 2-3 nucleotides in length, or the sense strand has a 3′ overhang of 2-3 nucleotides in length, or the antisense strand has a 3′ overhang of 2-3 nucleotides in length.

In some embodiments, in the RNA inhibitor, the sense nucleic acid strand and the antisense nucleic acid strand are each 16 to 35 nucleotides in length.

In some embodiments, in the RNA inhibitor, one strand of the RNA inhibitor for inhibiting PCSK9 gene expression has at least 75% homology or complementarity to any one of the nucleotide sequences selected from SEQ ID NOs: 655-981 and 1108-1111.

In some embodiments, in the RNA inhibitor, the sense strand is selected from any one of SEQ ID NOs: 1-327, 982-1044, 1123-1124, and 1147-1148, or a sequence differing by no more than 3 nucleotides therefrom.

In some embodiments, at least one nucleotide is a nucleotide with a chemical modification.

In some embodiments, the chemical modification is at least one of the following modifications:
(1) a modification of a phosphodiester bond linking nucleotides in the nucleotide sequence of the RNA inhibitor for inhibiting PCSK9 gene expression;
(2) a modification of 2′-OH of a ribose in the nucleotide sequence of the RNA inhibitor for inhibiting PCSK9 gene expression; and
(3) a modification of a base in the nucleotide sequence of the RNA inhibitor for inhibiting PCSK9 gene expression.

In some embodiments, at least two consecutive phosphorothioate bonds are present between nucleotides of the sense strand and/or the antisense strand.

In some embodiments, at least two consecutive phosphorothioate bonds are present between three consecutive nucleotides at the end of the sense strand and/or the end of the antisense strand. In some embodiments, the —OH groups at the 2′-positions of the glycosyl groups in the nucleotides at positions 7, 9, 10, and 11 starting from the 5′-end of the sense strand are replaced by fluorine, and the —OH groups at the 2′-positions of the glycosyl groups in the remaining nucleotides of the sense strand are replaced by methoxy groups.

In some embodiments, the —OH groups at the 2′-positions of the glycosyl groups in the nucleotides at positions 2, 14, and 16 starting from the 5′-end of the antisense strand are replaced by fluorine, and the —OH groups at the 2′-positions of the glycosyl groups in the remaining nucleotides of the antisense strand are replaced by methoxy groups.

In some embodiments, the RNA inhibitor includes: Ps3464, Ps3543, Ps3554, SP340, SP341, SP342, SP345, SP351, Pt2888, Pt3493, or Pt3553.

In some embodiments, the RNA inhibitor includes: P3119MW02, P3196MW02, P3464MW02, P3543MW02, P3554MW02, SP340MW02, SP341MW02, SP342MW02, SP344MW02, SP345MW02, SP349MW02, SP351MW02, WP3239MW02, WP3262MW02, WP3315MW02, WP3315-AMW02, WP3323MW02, Pt609MW02, Pt890MW02, Pt2530MW02, Pt2532MW02, Pt2533MW02, Pt2534MW02, Pt2536MW02, Pt2538MW02, Pt2541MW02, Pt2612MW02, Pt2675MW02, Pt2697MW02, Pt2698MW02, Pt2699MW02, Pt2700MW02, Pt2717MW02, Pt2727MW02, Pt2748MW02, Pt2750MW02, Pt2752MW02, Pt2818MW02, Pt2837MW02, Pt2838MW02, Pt2882MW02, Pt2883MW02, Pt2888MW02, Pt2889MW02, Pt2980MW02, Pt2991MW02, Pt3138MW02, Pt3139MW02, Pt3141MW02, Pt3185MW02, Pt3186MW02, Pt3191MW02, Pt3193MW02, Pt3194MW02, Pt3199MW02, Pt3236MW02, Pt3276MW02, Pt3304MW02, Pt3315MW02, Pt3436MW02, Pt3438MW02, Pt3493MW02, or Pt3553MW02.

In some embodiments, the RNA inhibitor comprises a ligand, wherein the ligand is conjugated to the sense strand and/or the antisense strand.

In some embodiments, the ligand is conjugated to the 5′-end and/or the 3′-end of the antisense strand.

In some embodiments, the ligand is conjugated to the 5′-end and/or the 3′-end of the sense strand.

In some embodiments, the ligand is conjugated to the 5′-end of the antisense strand, and the ligand is conjugated to the 3′-end of the sense strand.

In some embodiments, the ligand is conjugated to the 3′-end of the antisense strand, and the ligand is conjugated to the 5′-end of the sense strand.

In some embodiments, the ligand is conjugated to the 5′-end and the 3′-end of the sense strand.

In some embodiments, the ligand is conjugated to the 3′-end of the sense strand.

In some embodiments, in the RNA inhibitor for inhibiting PCSK9 gene expression, the ligand further comprises a targeting unit, i.e. a structure for enhancing uptake of the RNA inhibitor by a hepatocyte.

In some embodiments, the targeting unit is selected from a monosaccharide and a derivative thereof.

In some embodiments, the monosaccharide is selected from one or more of the following structures: mannose, galactose, D-arabinose, glucose, fructose, xylose, glucosamine, and ribose. In some embodiments, the monosaccharide derivative is selected from a mannose derivative, a galactose derivative, a glucose derivative, a ribose derivative, and other derivatives.

In some embodiments, the targeting unit is selected from galactose, galactosamine, N-acetylgalactosamine, and derivatives thereof.

In some embodiments, the targeting unit is N-acetylgalactosamine and a derivative thereof.

In another aspect, the present application provides a pharmaceutical composition, which comprises the RNA inhibitor for inhibiting PCSK9 gene expression, and further comprises a delivery vehicle, and/or a physiologically acceptable excipient and/or carrier and/or diluent.

In some embodiments, the delivery vehicle comprises a liposome.

In some embodiments, the delivery vehicle comprises a nanolipid.

In another aspect, the present application provides use of the RNA inhibitor for inhibiting PCSK9 gene expression and the pharmaceutical composition in the manufacture of a medicament for preventing or treating a disease or pathology, or reducing the risk of a disease or pathology.

In some embodiments, the disease or pathology comprises a disease or pathology associated with elevated levels of PCSK9.

In some embodiments, the disease or pathology comprises hypercholesterolemia.

In some embodiments, the disease or pathology comprises inflammatory, cardiovascular and cerebrovascular, or metabolic diseases.

In some embodiments, the cardiovascular and cerebrovascular disease comprises hyperlipidemia, stroke, atherosclerosis, thrombosis, coronary heart disease, or aortic stenosis.

In some embodiments, the method comprises administering to a subject in need thereof an effective amount of the RNA inhibitor for inhibiting PCSK9 gene expression according to any one of claims 1-29, a pharmaceutically acceptable salt thereof, or the pharmaceutical composition according to any one of claims 30-32.

In some embodiments, the RNA inhibitor for inhibiting PCSK9 gene expression, the pharmaceutically acceptable salt thereof, or the pharmaceutical composition is administered to the subject by a subcutaneous, intravenous, oral, rectal, or intraperitoneal administration route.

In another aspect, the present application provides a method for inhibiting PCSK9 expression in a cell, tissue, or subject, which comprises administering to the cell, tissue, or subject an effective amount of the RNA inhibitor for inhibiting PCSK9 gene expression, a pharmaceutically acceptable salt thereof, or the pharmaceutical composition according to any one of claims 30-32.

In some embodiments, the cell is a hepatocyte.

In some embodiments, the tissue is a liver tissue.

In some embodiments, the cell and tissue are ex vivo.

Other aspects and advantages of the present application will be readily apparent to those skilled in the art from the following detailed description. Only exemplary embodiments of the present application have been shown and described in the following detailed description. As those skilled in the art will recognize, the content of the present application enables those skilled in the art to make changes in the specific embodiments disclosed without departing from the spirit and scope of the invention to which the present application relates. Accordingly, the drawings and description of the present application are provided only for the purpose of illustration rather than limitation.

### BRIEF DESCRIPTION OF THE DRAWINGS

The specific features of the invention to which the present application relates are set forth in the appended claims. The features and advantages of the invention to which the present application relates may be more fully understood with reference to the exemplary embodiments and drawings described in detail below. The drawings are briefly described below:

FIG. 1 shows IC50 values of different compounds described in the present application;

FIG. 2 shows a schematic diagram of the molecular structure of L96 described in the present application;

FIG. 3 shows a schematic diagram of the conjugation mode of L96 and siRNA described in the present application;

FIG. 4 shows a schematic diagram of the psi-CHECK2 plasmid according to the present application;

FIG. 5 shows the inhibitory effects of SP341.1MW02-SP341.11MW02;

FIG. 6 shows the inhibitory effects of Ps3543.1MW02-Ps3543.11;

FIG. 7 shows the off-target analysis of SP341MW02, Ps3543MW02, and SP345MW02 in HepG2;

FIG. 8 shows the off-target analysis of SP341MW02, Ps3543MW02, and SP345MW02 in primary human hepatocytes;

FIG. 9 shows the toxicity assessment of Ps3543MW02, SP341MW02, and SP345MW02 in rats;

FIGS. 10-11 show the efficacy assay of SP345MW02 in non-human primates;

FIG. 12 shows the structures of linkers L1 and L2;

FIG. 13 shows the efficacy assay of bivalent molecules in a PCSK9&LPA HDI model;

FIG. 14 shows the assessment results of the activity of bivalent molecules in a PCSK9 and LPA double humanized mouse model;

FIGS. 15A-15C show the detection results of the in vivo inhibitory effects of dual-target molecules on PCSK9 and LPA in NHP;

FIG. 16 shows the detection results of the in vitro plasma stability;

FIG. 17 shows the detection results of lysosome stability;

FIGS. 18A-18B show the mRNA detection results of the inhibition of hAPOC3 by bivalent compounds;

FIG. 19 shows a schematic diagram of the structure of InvdA;

FIGS. 20A-20B show IC50 values of a total of 5 compounds, SP345MW02-L3b-L4230MW02, under free uptake conditions in primary human hepatocytes.

### DETAILED DESCRIPTION

The implementation of the invention of the present application is described below with reference to specific embodiments, and other advantages and effects of the invention of the present application will be readily apparent to those skilled in the art from the disclosure of the specification.

### Definitions of Terms

In the present application, the terms “iRNA”, “RNAi agent”, “iRNA agent”, “RNA interfering agent”, and “RNA inhibitor” are used interchangeably and generally refer to an agent comprising RNA as defined by the terms herein, which can mediate targeted cleavage of RNA transcripts by the RNA-induced silencing complex (RISC) pathway. The iRNA directs sequence-specific degradation of mRNA via a process known as RNA interference (RNAi). The iRNA regulates (e.g., inhibits) the expression of the PCSK9 gene (e.g., NCBI Reference Sequence: NM_001407240.1, Homo sapiens PCSK9 mRNA, as set forth in SEQ ID NO: 1112) in a cell (e.g., a cell in a subject, such as a mammalian subject).

In certain embodiments, the RNAi agent may be a single-stranded siRNA (ssRNAi) that is introduced into a cell or organism to inhibit a target mRNA. The single-stranded RNAi agent binds to the RISC endonuclease, Argonaute 2, which then cleaves the target mRNA. The single-stranded siRNA is generally 15 to 30 nucleotides and is chemically modified. The design and testing of single-stranded siRNAs are described in U.S. Pat. No. 8,101,348 and Lima et al. (2012) Cell, 150:883-894, the entire contents of each of which are incorporated herein by reference. Any of the antisense nucleotide sequences described herein may be used as a single-stranded siRNA as described herein or as chemically modified by the methods described in Lima et al. (2012) Cell, 150:883-894.

In certain embodiments, the “iRNA” used herein is a double-stranded RNA and is referred to herein as a “double-stranded RNAi agent”, “double-stranded RNA (dsRNA) molecule”, “dsRNA agent”, or “dsRNA”. The term “dsRNA” refers to a complex of ribonucleic acid molecules, which has a duplex structure comprising two anti-parallel and substantially complementary nucleic acid strands, referred to as having “sense” and “antisense” orientations with respect to a target RNA (i.e., the PCSK9 gene). In some embodiments of the present application, the double-stranded RNA (dsRNA) triggers the degradation of a target RNA (e.g., an mRNA) through a post-transcriptional gene silencing mechanism (referred to herein as RNA interference or RNAi).

The duplex structure may be of any length that permits specific degradation of a desired target RNA through the RISC pathway, and may range from about 19 to 36 base pairs in length, e.g., about 19-30 base pairs in length, such as about 19, 20, 21, 22, 23, 24, 25, 26, 27, 28, 29, 30, 31, 32, 33, 34, 35, or 36 base pairs in length. Ranges and lengths intermediate to the ranges and lengths described above are also included as part of the present application. In certain embodiments, the iRNA agent of the present application is a dsRNA comprising 15-23 nucleotides in each strand, which interacts with a target RNA sequence (e.g., the PCSK9 gene) to direct the cleavage of the target RNA. In certain embodiments, the iRNA of the present application is a dsRNA comprising 24-30 nucleotides, which interacts with a target RNA sequence (e.g., a PCSK9 target mRNA sequence) to direct the cleavage of the target RNA.

In the present application, the terms “nucleic acid” and “polynucleotide” are used interchangeably and refer to a polymeric form of nucleotides (deoxyribonucleotides or ribonucleotides or analogs thereof) of any length. The polynucleotide may have any three-dimensional structure and may perform any function. The following are non-limiting examples of polynucleotides: genes or gene fragments (e.g., probes, primers, EST or SAGE tags), exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, siRNA, miRNA, shRNA, RNAi agents, and primers. The polynucleotide may be modified or substituted at one or more bases, sugars, and/or phosphates with any of the various modifications or substitutions described in the present application or known in the art. The polynucleotides may comprise modified nucleotides, such as methylated nucleotides and nucleotide analogs. If present, the nucleotide structure may be modified before or after the assembly of the polymer. The sequence of nucleotides may be blocked by non-nucleotide components. The polynucleotide may be modified after polymerization, for example, by coupling to a labeling component. The term may be double-stranded and single-stranded molecules. Unless otherwise stated or required, any embodiment of the present application as a polynucleotide includes a double-stranded form and each of two complementary single-stranded forms known or predicted to constitute the double-stranded form.

In the present application, the term “target nucleic acid” or “target sequence” generally refers to a contiguous portion of the nucleotide sequence of an mRNA molecule formed during the transcription of the PCSK9 gene, including mRNA that is a product of RNA processing of a primary transcription product. The target portion of the sequence should be at least long enough to serve as a substrate for iRNA-directed cleavage at or near this portion of the nucleotide sequence of an mRNA molecule formed during the transcription of the PCSK9 gene. In one embodiment, the target sequence is within the protein coding region of PCSK9. The target sequence may be about 19-36 nucleotides in length, e.g., preferably about 19-30 nucleotides in length. Ranges and lengths intermediate to the ranges and lengths described above are also included as part of the present application.

In the present application, the term “nucleotide sequence” generally refers to a series or order of nucleobases, nucleotides, and/or nucleosides, whether modified or unmodified, which are described by a series of letters using the standard nucleotide nomenclature and a list of symbols for modified nucleotides described in the present application.

In the present application, the term “oligonucleotide” generally refers to a polymer consisting of a plurality of nucleotide residues (deoxyribonucleotides or ribonucleotides, or related structural variants or synthetic analogs thereof) linked by phosphodiester bonds (or related structural variants or synthetic analogs thereof). Therefore, although the term “oligonucleotide” generally refers to a polymer of nucleotides in which the nucleotide residues and linkages therebetween are naturally occurring, it should be understood that the scope of the term also includes various analogs, including but not limited to: peptide nucleic acids (PNAs), phosphoramidates, phosphorothioates, methylphosphonates, 2-O-methyl ribonucleic acids, and the like. The exact size of the molecule may depend on the particular application. Oligonucleotides are generally of relatively short length, usually about 10-30 nucleotide residues, but the term may also refer to molecules of any length, although the term “polynucleotide” or “nucleic acid” is generally used for relatively large oligonucleotides.

In certain embodiments, the oligonucleotide comprises one or more unmodified ribonucleosides (RNA) and/or unmodified deoxyribonucleosides (DNA) and/or one or more modified nucleosides. The term “modified oligonucleotide” generally refers to an oligonucleotide comprising at least one modified nucleoside and/or at least one modified internucleoside linkage.

In the present application, the term “modified nucleoside” generally refers to a nucleoside comprising at least one chemical modification as compared with a naturally occurring RNA or DNA nucleoside. A modified nucleoside comprises a modified sugar moiety and/or a modified nucleobase.

In the present application, the term “nucleobase” generally refers to a heterocyclic pyrimidine or purine compound, which is a component of all nucleic acids and includes adenine (a), guanine (g), cytosine (c), thymine (t), and uracil (u). The nucleotide may include a modified nucleotide or nucleotide mimic, an abasic site (Ab or X), or a surrogate replacement moiety. As used herein, “nucleobase sequence” generally refers to the order of consecutive nucleobases independent of any sugar, linkage, or nucleobase modification. The term “unmodified nucleobase” or “naturally occurring nucleobase” generally refers to naturally occurring heterocyclic nucleobases of RNA or DNA, including purine bases adenine (a) and guanine (g); and pyrimidine bases thymine (t), cytosine (c) (including 5-methyl c), and uracil (u). “Modified nucleobase” generally refers to any nucleobase that is not a naturally occurring nucleobase.

In the present application, the term “sugar moiety” generally refers to a naturally occurring sugar moiety or a modified sugar moiety of a nucleoside. The term “naturally occurring sugar moiety” generally refers to a ribofuranosyl group as found in naturally occurring RNA or a deoxyribofuranosyl group as found in naturally occurring DNA. “Modified sugar moiety” refers to a substituted sugar moiety or a sugar surrogate.

In the present application, the term “internucleoside linkage” generally refers to a covalent linkage between adjacent nucleosides in an oligonucleotide. “Naturally occurring internucleoside linkage” refers to a 3′ to 5′ phosphodiester linkage. “Modified internucleoside linkage” refers to any internucleoside linkage other than a naturally occurring internucleoside linkage.

As used herein, the label “d” preceding a monomer (e.g., nucleotides A, U, C, G, T, etc.) indicates that the monomer is modified by 2′-deoxy. As used herein, the label “f” following a monomer (e.g., nucleotides A, U, C, G, T, etc.) indicates that the monomer is modified by 2′-fluoro (2′-F modification). The label “m” following a monomer (e.g., nucleotides A, U, C, G, T, etc.) indicates that the monomer is modified by 2′-O-methyl(2′-OMe). In the present disclosure, the label “s” between monomers (e.g., nucleotides A, U, C, G, T, etc.) indicates that the two monomers are linked by a phosphorothioate bond (i.e., a phosphorothioate diester bond), i.e., the monomers are modified by phosphorothioate (PS). In the present disclosure, the absence of the label “s” between monomers (e.g., nucleotides A, U, C, G, T, etc.) indicates that the two nucleotides are linked by a phosphate bond (i.e., a phosphodiester bond).

In the present application, the term “antisense oligonucleotide” refers to a single-stranded oligonucleotide molecule having a nucleobase sequence that is complementary to a corresponding fragment of a target nucleic acid (e.g., a target genomic sequence, an mRNA precursor, or an mRNA molecule). In certain embodiments, the antisense oligonucleotide is 12 to 30 nucleobases in length. In certain embodiments, the antisense oligonucleotide is an unmodified or modified nucleic acid having a nucleotide sequence that is complementary to the sequence of a target nucleic acid (e.g., a PCSK9 polynucleotide).

In the present application, the term “antisense strand” generally refers to a strand of an RNA inhibitor (e.g., dsRNA), which includes a region that is substantially complementary to a target sequence. As used herein, the term “complementary region” generally refers to a region on the antisense strand that is substantially complementary to a sequence defined in the present application (e.g., a target sequence). Where the complementary region is not fully complementary to the target sequence, the mismatches may be in the internal or terminal regions of the molecule. Generally, the most tolerated mismatches are present in the terminal regions, e.g., within 5, 4, 3, or 2 nucleotides of the 5′-end and/or the 3′-end.

In the present application, the term “sense strand” (S) generally refers to a strand of an RNA inhibitor, which includes a region that is substantially complementary to a region of the antisense strand as that term is defined herein. The “sense” strand is sometimes referred to as a “passenger” strand or an “anti-guide” strand. With their sequence, the antisense strand targets a desired mRNA, while the sense strand targets a different target. Therefore, if the antisense strand is incorporated into RISC, the correct target is targeted. Incorporation of the sense strand may result in off-target effects. These off-target effects may be limited by the use of modifications or 5′-end caps on the sense strand.

In the present application, the term “complementary”, when used to describe a first nucleotide sequence (e.g., RNAi agent sense strand or PCSK9 mRNA) with respect to a second nucleotide sequence (e.g., RNAi agent antisense strand), refers to the ability of an oligonucleotide or polynucleotide comprising the first nucleotide sequence to hybridize (form base pair hydrogen bonds) with an oligonucleotide or polynucleotide comprising the second nucleotide sequence and form a duplex or double helix structure under certain conditions. The complementary sequences include Watson-Crick base pairs or non-Watson-Crick base pairs, and include natural or modified nucleotides or nucleotide mimics, so long as the above requirements with respect to their hybridization ability are achieved. “Complementary” does not require nucleobase complementarity on each nucleoside. On the contrary, some mismatches can be tolerated.

LPA is the name of the gene encoding apolipoprotein(a) (apo (a)), which is mainly expressed in the liver, and its expression is restricted to humans and non-primates. Apolipoprotein(a) is attached to apo (B)-100 by a disulfide bond and combined with the lipid core to form lipoprotein(a) (Lp(a)) particles. Lp(a) particles are special cholesterol-rich macromolecular lipoproteins, the surface of which is wrapped by cholesterol and phospholipids, and are embedded with hydrophilic apolipoprotein components apolipoprotein(a) and apo (B)-100. Lp(a) can enter and deposit on the wall of blood vessels, and has the effect of promoting atherosclerosis. Lp(a) is structurally homologous to plasminogen (PLG), and can compete with fibrinogen for binding to fibrin sites, thereby inhibiting fibrinogen hydrolysis and promoting thrombosis. Therefore, LP (a) is closely associated with atherosclerosis and thrombosis. Studies have shown that the level of Lp(a) in blood is an independent risk factor for cardiovascular diseases, stroke, and atherosclerotic stenosis.

In the present application, the term “fully complementary” generally means that all (100%) bases in the contiguous sequence of the first polynucleotide will hybridize with the same number of bases in the contiguous sequence of the second polynucleotide. The contiguous sequence may comprise all or part of the first or second nucleotide sequence. As used herein, “partially complementary” generally means that in a hybridized nucleobase sequence pair, at least about 70% of the bases in the contiguous sequence of the first polynucleotide will hybridize with the same number of bases in the contiguous sequence of the second polynucleotide. As used herein, “substantially complementary” generally means that in a hybridized nucleobase sequence pair, at least about 90% of the bases in the contiguous sequence of the first polynucleotide will hybridize with the same number of bases in the contiguous sequence of the second polynucleotide. The terms “complementary”, “fully complementary”, and “substantially complementary” as used herein may be used in terms of base matching between the sense strand and the antisense strand of the RNA inhibitor or between the antisense strand of the RNA inhibitor and the sequence of PCSK9 mRNA. Sequence identity or complementarity is independent of modification. For the purpose of determining identity or complementarity, for example, a and Af are complementary to U (or T) and identical to A.

In the present application, the term “homologous” or “homology” generally refers to the number of nucleotides of a subject nucleic acid sequence that have matched the same nucleotide of a reference nucleic acid sequence, typically as determined by a sequence analysis program (e.g., Karlin and Altschul, 1990, PNAS, 87:2264-2268; Karlin and Altschul, 1993, PNAS, 90:5873-5877), or by visual inspection. As used herein, the term “complete homology” or “completely homologous” generally refers to complete (100%) homology or “identity” between a reference sequence and a subject nucleic acid sequence. As used herein, the term “substantially homologous” or “substantial homology” generally means that nucleotides at the same nucleotide positions in a subject sequence and a reference sequence are at least 50% (e.g., at least 55%, 60%, 65%, 70%, 75%, 80%, 85%, 90%, 95%, 96%, 97%, 98%, or 99%) homologous.

In the present application, the term “ligand” generally refers to any compound or molecule capable of binding to a bioactive substance (e.g., an oligonucleotide) covalently or otherwise chemically. In certain embodiments, the ligand is capable of interacting directly or indirectly with another compound, such as a receptor. The receptor interacting with the ligand may be present on the surface of a cell, or alternatively may be an intracellular and/or intercellular receptor. The interaction of the ligand with the receptor may result in a biochemical reaction, or may simply be a physical interaction or binding.

In the present application, the terms “induce”, “inhibit”, “strengthen”, “elevate”, “increase”, “reduce”, “decrease”, and the like generally indicate a quantitative difference between two states.

For example, “an amount effective to inhibit the activity or expression of PCSK9” means that the level of the activity or expression of PCSK9 in a treated sample will be lower than the level of the activity or expression of PCSK9 in an untreated sample. The terms apply, for example, to expression levels and activity levels. The terms “reduce” and “decrease” are used interchangeably and generally refer to any change less than the original. “Reduce” and “decrease” are relative terms that need to be compared before and after measurement. “Reduce” and “decrease” include complete depletion.

In certain embodiments, the term “decrease” may refer to an overall decrease of about 5%, 10%, 20%, 25%, 30%, 40%, 50%, 60%, 70%, 80%, 85%, 90%, 95%, or 100% in the expression level/amount of a gene or gene product such as protein or biomarker in a first sample as compared with the expression level/amount of the corresponding gene or gene product such as protein or biomarker in a second sample, as detected by standard methods known in the art (such as those described in the present application). In certain embodiments, the term “decrease” refers to a decrease in the expression level/amount of a gene or biomarker in the first sample, wherein the decrease is at least about 0.9 times, 0.8 times, 0.7 times, 0.6 times, 0.5 times, 0.4 times, 0.3 times, 0.2 times, 0.1 times, 0.05 times, or 0.01 times the expression level/amount of the corresponding gene or biomarker in the second sample. In certain embodiments, the first sample is a sample obtained from a subject, and the second sample is a reference sample.

In the present application, the term “expression” generally refers to the process by which a gene ultimately produces a protein. The expression includes, but is not limited to, transcription, post-transcriptional modification (e.g., splicing, polyadenylation, and addition of a 5′-cap), and translation.

In the present application, the term “pharmaceutically acceptable” generally refers to one or more non-toxic substances that do not interfere with the effectiveness of the biological activity of an active ingredient. Such formulations may typically comprise a salt, an excipient, a buffer, a preservative, a compatible carrier, and optionally other therapeutic agents. Such pharmaceutically acceptable formulations may also typically comprise a compatible solid or liquid filler, a diluent, or an encapsulating material, suitable for administration to humans. When used in medicine, the salt should be a pharmaceutically acceptable salt. However, non-pharmaceutically acceptable salts can be conveniently used to prepare pharmaceutically acceptable salts and cannot be excluded from the scope of the present application. Such pharmacologically and pharmaceutically acceptable salts include, but are not limited to, salts prepared from the following acids: hydrochloric acid, hydrobromic acid, sulfuric acid, nitric acid, phosphoric acid, maleic acid, acetic acid, salicylic acid, citric acid, boric acid, formic acid, malonic acid, succinic acid, and the like. The pharmaceutically acceptable salts can also be prepared into alkali metal salts or alkaline earth metal salts, such as sodium salts, potassium salts, or calcium salts.

In the present application, the term “lipid nanoparticle” or “LNP” generally refers to a vesicle comprising a lipid layer encapsulating a pharmacologically active molecule (e.g., a nucleic acid molecule, such as an iRNA or a plasmid from which an iRNA is transcribed). LNPs are described in, e.g., Chinese Patent No. CN103189057B, the entire contents of which are incorporated herein by reference.

In the present application, the term “preventing and/or treating” includes not only preventing and/or treating a disease, but also generally includes preventing the onset of a disease, slowing or reversing the progression of a disease, preventing or slowing the onset of one or more symptoms associated with a disease, reducing and/or alleviating one or more symptoms associated with a disease, reducing the severity and/or duration of a disease and/or any symptom associated therewith, and/or preventing further increase in the severity of a disease and/or any symptom associated therewith, preventing, reducing, or reversing any physiological damage caused by a disease, and any pharmacological effect that would normally be beneficial to the patient being treated. The viable therapeutic agent formed from the RNAi agent or pharmaceutical composition of the present application is not required to achieve a complete cure or eradication of any symptom or manifestation of a disease. As recognized in the relevant art, a drug used as a therapeutic agent may reduce the severity of a given disease state, and does not need to eliminate every manifestation of the disease to be considered a useful therapeutic agent. Similarly, a viable prophylactic agent constituted by applying prophylactic treatment is not required to be entirely effective in preventing the onset of a condition. It is enough to simply reduce the impact of a disease (e.g., by reducing the number or severity of its symptoms, or by increasing the effectiveness of another treatment, or by producing another beneficial effect), or reduce the likelihood of the disease occurrence or worsening, in a subject.

In the present application, the terms “disease” and “condition” are used interchangeably and generally refer to any deviation of a subject from a normal state, e.g., any change in the state of the body or certain organs that prevents or disrupts the implementation of a function, and/or causes symptoms such as discomfort, dysfunction, distress, or even death in a person suffering from or in contact with the disease. The disease or condition may also be referred to as distemper, ailing, ailment, malady, disorder, sickness, illness, or complaint.

In the present application, the term “administering” generally refers to the introduction of the pharmaceutical formulation of the present application into the body of a subject by any route of introduction or delivery. Any method known to those skilled in the art for contacting a cell, organ, or tissue with the drug can be employed. The administration may include, but is not limited to, intravenous, intra-arterial, intranasal, intraperitoneal, intramuscular, subcutaneous transdermal, or oral administration. The daily dose may be divided into one, two, or more suitable forms of dose to be administered at one, two, or more times during a certain period of time.

In the present application, the term “contact” generally means that two or more different types of substances come into contact in any order, in any manner, and for any duration. The “contact” may occur in vivo, ex vivo, or in vitro. In certain embodiments, it may mean that the RNAi agent or composition of the present application is in direct contact with a cell or tissue. In other embodiments, the term means that the RNA inhibitor or composition of the present application is in indirect contact with a cell or tissue. For example, the method of the present application includes a method in which the subject is contacted with the RNA inhibitor or composition of the present application, and then the RNA inhibitor or composition is contacted with the cell or tissue by diffusion or any other active or passive transport process known in the art through which the compound circulates in vivo.

In the present application, the term “effective amount” or “effective dose” generally refers to an amount sufficient to achieve, or at least partially achieve, a desired effect. The “therapeutically effective amount” or “therapeutically effective dose” of a drug or therapeutic agent is generally any amount of the drug that promotes the regression of a disease (as evidenced by a decrease in the severity of disease symptoms, an increase in the frequency and duration of the asymptomatic phase of the disease, or the prevention of damage or disability due to the development of the disease) when being used alone or in combination with another therapeutic agent. The “prophylactically effective amount” or “prophylactically effective dose” of a drug generally refers to an amount of the drug that inhibits the progression or recurrence of a disease when being administered, alone or in combination with another therapeutic agent, to a subject at risk of disease progression or disease recurrence. The capacity of a therapeutic agent or prophylactic agent to promote disease regression or inhibit disease progression or recurrence can be assessed using a variety of methods known to those skilled in the art, such as by predicting efficacy in humans in human subjects during clinical trials and in animal model systems, or by measuring the activity of the agent in an in vitro assay. In certain embodiments, “effective amount” refers to an amount of an RNA inhibitor that produces an intended pharmacological, therapeutic, or prophylactic result.

In the present application, the term “subject” generally refers to a human or a non-human animal (including mammals) in need of diagnosis, prognosis, amelioration, prevention, and/or treatment of a disease, such as human, non-human primates (apes, gibbons, gorillas, chimpanzees, orangutans, and macaques), livestock (dogs and cats), farm animals (poultry such as chickens and ducks, horses, cattle, goats, sheep, and pigs), and laboratory animals (mice, rats, rabbits, and guinea pigs). Human subjects include fetal, neonatal, infant, juvenile, and adult subjects. The subject includes an animal disease model.

In the present application, the terms “comprising”, “including”, “having”, “may”, “containing”, and variations thereof are generally intended to be open-ended transitional phrases, terms, or words, which do not exclude the possibility of additional acts or structures. The term “consisting of . . . ” generally means that no other components (or likewise, features, integers, steps, etc.) can be present. The singular forms “a”, “an”, and “the” generally include plural forms of the referent unless otherwise specified clearly in the context.

In the present application, the term “about” generally means approximately, roughly, or around. When the term “about” is used to refer to the range of values, the cut-off or specified value is used to indicate that the stated value may differ from the listed value by up to 10%. Therefore, the term “about” may be used to encompass variations of ±10% or less, variations of ±5% or less, variations of ±1% or less, variations of ±0.5% or less, or variations of ±0.1% or less from a specified value.

It should be understood that the term “at least” preceding a number or a series of numbers includes numbers that are adjacent to the term “at least” and all subsequent numbers or integers that are logically included, as is clear from the context. For example, the number of nucleotides in a nucleic acid molecule must be an integer. For example, “at least 19 nucleotides in a nucleic acid molecule of 21 nucleotides” means that 19, 20, or 21 nucleotides have the indicated properties. When “at least” appears preceding a series of numbers or a range, it should be understood that “at least” may modify each number in the series or range.

It should be understood that “no more than” or “less than” used herein refers to a value or an integer that is adjacent to the phrase and is logically lower, e.g., logically from the context, to zero. For example, a duplex having an overhang of “no more than 3 nucleotides” has an overhang of 3, 2, 1, or 0 nucleotides. For example, a duplex having an overhang of “no more than 3 nucleotides” has an overhang of 3, 2, 1, or 0 nucleotides. When “no more than” appears preceding a series of numbers or a range, it should be understood that “no more than” may modify each number in the series or range. Ranges used herein include both upper and lower limits.

### DETAILED DESCRIPTION OF INVENTION

### Antisense Strand and Sense Strand

In one aspect, the present application provides an RNA inhibitor for inhibiting PCSK9 gene expression.

In certain embodiments, the RNA inhibitor includes a single-stranded oligonucleotide or a double-stranded ribonucleic acid (dsRNA) molecule for inhibiting PCSK9 gene expression in a cell, such as a cell of a subject (e.g., a mammal). The dsRNA comprises an antisense strand having a complementary region that is complementary to at least a part of an mRNA formed during the expression of the PCSK9 gene. The complementary region is about 12-30 nucleotides in length (e.g., about 30, 29, 28, 27, 26, 25, 24, 23, 22, 21, 20, 19, 18, 17, 16, 15, 14, 13, or 12 nucleotides in length).

The dsRNA comprises two RNA strands that can be complementary and hybridized to form a duplex structure (complementary region) under the conditions in which the dsRNA is used. One strand (the antisense strand) of the dsRNA comprises a complementary region that is substantially complementary, and generally fully complementary, to a target sequence. The target sequence may be derived from a sequence that forms an mRNA during the expression of the PCSK9 gene. The other strand (the sense strand) comprises a region that is complementary to the antisense strand, such that the two strands can be hybridized and form a duplex structure when combined under suitable conditions. Generally, the duplex structure is 12 to 30 base pairs in length. Similarly, the complementary region to the target sequence is 12 to 30 nucleotides in length.

In certain embodiments, the dsRNA is about 19 to about 23 nucleotides in length, or about 24 to about 30 nucleotides in length. Generally, the dsRNA is of sufficient length to serve as a substrate for the Dicer enzyme. For example, it is well known in the art that dsRNAs longer than about 21-23 nucleotides in length can serve as substrates for Dicer. It is also understood by those skilled in the art that the region of an RNA targeted for cleavage is generally part of a larger RNA molecule (generally an mRNA molecule). “Part” of a target is consecutive nucleotides of an mRNA target of sufficient length to allow it to be a substrate for RNAi-directed cleavage (i.e., cleavage via the RISC pathway).

It should also be understood by those skilled in the art that the duplex region is a major functional portion of the dsRNA, such as a duplex region of about 19 to about 30 base pairs, e.g., about 19-30, 19-29, 19-28, 19-27, 19-26, 19-25, 19-24, 19-23, 19-22, 19-21, or 19-20 base pairs. Therefore, in one embodiment, to the extent that it becomes a functional duplex (e.g., of 15-30 base pairs) that targets the desired RNA for cleavage, an RNA molecule or a complex of RNA molecules having a duplex region of more than 30 base pairs is a dsRNA.

In certain embodiments, at least about 80% of bases between the sense strand and the antisense strand are complementary.

In certain embodiments, the sense strand and the antisense strand are each independently of 15-30 nucleotides.

In certain embodiments, the sense strand and the antisense strand are each independently of 17-25 nucleotides.

In certain embodiments, the sense strand and the antisense strand are each independently of 19-23 nucleotides.

In some embodiments, the sense strand is selected from any one of SEQ ID NOs: 1-327, 982-1044, 1123-1124, and 1147-1148, or a sequence differing by no more than 3 nucleotides therefrom. In some embodiments, the sense strand of the RNA inhibitor is selected from the sequences in

Table 1 or sequences that differ by one, two, or three nucleotides from each of the sequences in Table 1.

In some embodiments, the antisense strand is selected from any one of SEQ ID NOs: 328-654, 1045-1107, 1125-1146, or 1149-1170, or a sequence differing by no more than 3 nucleotides therefrom.

In some embodiments, both strands of the RNA inhibitor have 3′ overhangs of 2-3 nucleotides in length, or the sense strand has a 3′ overhang of 2-3 nucleotides in length, or the antisense strand has a 3′ overhang of 2-3 nucleotides in length.

In some embodiments, the RNA inhibitor has a 3′ overhang of 2 nucleotides in length only in 5 the antisense strand.

In some embodiments, the antisense strand of the RNA inhibitor is selected from the sequences in Table 1 or sequences that differ by one, two, or three nucleotides from each of the sequences in Table 1.

In some embodiments, the sense strand and the antisense strand of the RNA inhibitor are selected from the sequences in Table 1 or differ by one, two, or three nucleotides from each of the sequences in Table 1.

TABLE 1

| | | Sense | | | | |
|---|---|---|---|---|---|---|
| | Sense | strand | Antisense | Antisense | Target | Target |
| RNA | strand | SEQ | strand | strand SEQ | sequence | sequence |
| inhibitor | (5′-3′) | ID NO | (5′-3′) | ID NO | (5′-3′) | SEQ ID NO |
| Ps178 | CUUCAG | 1 | AGGACUG | 328 | AACUUCA | 655 |
| | CUCCUGC | | UGCAGGA | | GCUCCUG | |
| | ACAGUC | | GCUGAAG | | CACAGUC | |
| | CU | | UU | | CU | |
| Ps2428 | GGUUCC | 2 | AGAGAGG | 329 | AUGGUUC | 656 |
| | GACUUG | | GACAAGU | | CGACUUG | |
| | UCCCUCU | | CGGAACC | | UCCCUCU | |
| | CU | | AU | | CU | |
| Ps2537 | GCCUGG | 3 | CAGAGUG | 330 | UUGCCUG | 657 |
| | AACUCA | | AGUGAGU | | GAACUCA | |
| | CUCACUC | | UCCAGGC | | CUCACUC | |
| | UG | | AA | | UG | |
| Ps2610 | GCAUUU | 4 | CCUGUUU | 331 | GGGCAUU | 658 |
| | CACCAU | | GAAUGGU | | UCACCAU | |
| | UCAAAC | | GAAAUGC | | UCAAACA | |
| | AGG | | CC | | GG | |
| Ps2617 | ACCAUU | 5 | AGCUCGA | 332 | UCACCAU | 659 |
| | CAAACA | | CCUGUUU | | UCAAACA | |
| | GGUCGA | | GAAUGGU | | GGUCGAG | |
| | GCU | | GA | | CU | |
| Ps2674 | UGUCCG | 6 | AAGUCAU | 333 | GAUGUCC | 660 |
| | UGGGCA | | UCUGCCC | | GUGGGCA | |
| | GAAUGA | | ACGGACA | | GAAUGAC | |
| | CUU | | UC | | UU | |
| Ps2683 | GCAGAA | 7 | GCUCAAU | 334 | GGGCAGA | 661 |
| | UGACUU | | AAAAGUC | | AUGACUU | |
| | UUAUUG | | AUUCUGC | | UUAUUGA | |
| | AGC | | CC | | GC | |
| Ps2719 | GGCAUU | 8 | GAGACCU | 335 | CAGGCAU | 662 |
| | CAAUCC | | GAGGAUU | | UCAAUCC | |
| | UCAGGU | | GAAUGCC | | UCAGGUC | |
| | CUC | | UG | | UC | |
| Ps2721 | CAUUCA | 9 | UGGAGAC | 336 | GGCAUUC | 663 |
| | | | CUGAGGA | | AAUCCUC | |
| | AUCCUC | | UUGAAUG | | AGGUCUC | |
| | AGGUCU | | CC | | CA | |
| | CCA | | | | | |
| Ps2723 | UUCAAU | 10 | GGUGGAG | 337 | CAUUCAA | 664 |
| | CCUCAG | | ACCUGAG | | UCCUCAG | |
| | GUCUCC | | GAUUGAA | | GUCUCCA | |
| | ACC | | UG | | CC | |
| Ps2725 | CAAUCC | 11 | UUGGUGG | 338 | UUCAAUC | 665 |
| | UCAGGU | | AGACCUG | | CUCAGGU | |
| | CUCCACC | | AGGAUUG | | CUCCACC | |
| | AA | | AA | | AA | |
| Ps2740 | CACCAA | 12 | AAGAAUC | 339 | UCCACCA | 666 |
| | GGAGGC | | CUGCCUC | | AGGAGGC | |
| | AGGAUU | | CUUGGUG | | AGGAUUC | |
| | CUU | | GA | | UU | |
| Ps2749 | GGCAGG | 13 | AUCCAUG | 340 | GAGGCAG | 667 |
| | AUUCUU | | GGAAGAA | | GAUUCUU | |
| | CCCAUG | | UCCUGCC | | CCCAUGG | |
| | GAU | | UC | | AU | |
| Ps2751 | CAGGAU | 14 | CUAUCCA | 341 | GGCAGGA | 668 |
| | UCUUCCC | | UGGGAAG | | UUCUUCC | |
| | AUGGAU | | AAUCCUG | | CAUGGAU | |
| | AG | | CC | | AG | |
| Ps2817 | GAGUGU | 15 | CCAUCAG | 342 | GUGAGUG | 669 |
| | GAAAGG | | CACCUUU | | UGAAAGG | |
| | UGCUGA | | CACACUC | | UGCUGAU | |
| | UGG | | AC | | GG | |
| Ps2824 | AAAGGU | 16 | AUGAGGG | 343 | UGAAAGG | 670 |
| | GCUGAU | | CCAUCAG | | UGCUGAU | |
| | GGCCCUC | | CACCUUU | | GGCCCUC | |
| | AU | | CA | | AU | |
| Ps2836 | GGCCCUC | 17 | AGUUAGC | 344 | AUGGCCC | 671 |
| | AUCUCC | | UGGAGAU | | UCAUCUC | |
| | AGCUAA | | GAGGGCC | | CAGCUAA | |
| | CU | | AU | | CU | |
| Ps2880 | CUGAUU | 18 | AGCUAAG | 345 | CCCUGAU | 672 |
| | AAUGGA | | CCUCCAU | | UAAUGGA | |
| | GGCUUA | | UAAUCAG | | GGCUUAG | |
| | GCU | | GG | | CU | |
| Ps2884 | UUAAUG | 19 | AGAAAGC | 346 | GAUUAAU | 673 |
| | GAGGCU | | UAAGCCU | | GGAGGCU | |
| | UAGCUU | | CCAUUAA | | UAGCUUU | |
| | UCU | | UC | | CU | |
| Ps2892 | GGCUUA | 20 | UGCCAUC | 347 | GAGGCUU | 674 |
| | GCUUUC | | CAGAAAG | | AGCUUUC | |
| | UGGAUG | | CUAAGCC | | UGGAUGG | |
| | GCA | | UC | | CA | |
| Ps2971 | CCUGAG | 21 | GCAGAGU | 348 | UUCCUGA | 675 |
| | CCACCUU | | AAAGGUG | | GCCACCU | |
| | UACUCU | | GCUCAGG | | UUACUCU | |
| | GC | | AA | | GC | |
| Ps2978 | CACCUU | 22 | GCAUAGA | 349 | GCCACCU | 676 |
| | UACUCU | | GCAGAGU | | UUACUCU | |
| | GCUCUA | | AAAGGUG | | GCUCUAU | |
| | UGC | | GC | | GC | |
| Ps2984 | UACUCU | 23 | AGCCUGG | 350 | UUUACUC | 677 |
| | GCUCUA | | CAUAGAG | | UGCUCUA | |
| | UGCCAG | | CAGAGUA | | UGCCAGG | |
| | GCU | | AA | | CU | |
| Ps2986 | CUCUGC | 24 | ACAGCCU | 351 | UACUCUG | 678 |
| | UCUAUG | | GGCAUAG | | CUCUAUG | |
| | CCAGGC | | AGCAGAG | | CCAGGCU | |
| | UGU | | UA | | GU | |
| Ps3057 | UCGGCA | 25 | UGCACCA | 352 | ACUCGGC | 679 |
| | GUGUGC | | CUGCACA | | AGUGUGC | |
| | AGUGGU | | CUGCCGA | | AGUGGUG | |
| | GCA | | GU | | CA | |
| Ps3117 | UACACA | 26 | AAGUAGG | 353 | GGUACAC | 680 |
| | UUCGCA | | GGUGCGA | | AUUCGCA | |
| | CCCCUAC | | AUGUGUA | | CCCCUAC | |
| | UU | | CC | | UU | |
| Ps3119 | CACAUU | 27 | UGAAGUA | 354 | UACACAU | 681 |
| | CGCACCC | | GGGGUGC | | UCGCACC | |
| | CUACUU | | GAAUGUG | | CCUACUU | |
| | CA | | UA | | CA | |
| Ps3122 | AUUCGC | 28 | CUGUGAA | 355 | ACAUUCG | 682 |
| | ACCCCUA | | GUAGGGG | | CACCCCU | |
| | CUUCAC | | UGCGAAU | | ACUUCAC | |
| | AG | | GU | | AG | |
| Ps3124 | UCGCACC | 29 | CUCUGUG | 356 | AUUCGCA | 683 |
| | CCUACU | | AAGUAGG | | CCCCUAC | |
| | UCACAG | | GGUGCGA | | UUCACAG | |
| | AG | | AU | | AG | |
| Ps3129 | CCCCUAC | 30 | UCUUCCU | 357 | CACCCCU | 684 |
| | UUCACA | | CUGUGAA | | ACUUCAC | |
| | GAGGAA | | GUAGGGG | | AGAGGAA | |
| | GA | | UG | | GA | |
| Ps3140 | CAGAGG | 31 | GGUUCCA | 358 | CACAGAG | 685 |
| | AAGAAA | | GGUUUCU | | GAAGAAA | |
| | CCUGGA | | UCCUCUG | | CCUGGAA | |
| | ACC | | UG | | CC | |
| Ps3196 | ACUGAG | 32 | AAUCUGC | 359 | GAACUGA | 686 |
| | CCAGAA | | GUUUCUG | | GCCAGAA | |
| | ACGCAG | | GCUCAGU | | ACGCAGA | |
| | AUU | | UC | | UU | |
| Ps3198 | UGAGCC | 33 | CCAAUCU | 360 | ACUGAGC | 687 |
| | AGAAAC | | GCGUUUC | | CAGAAAC | |
| | GCAGAU | | UGGCUCA | | GCAGAUU | |
| | UGG | | GU | | GG | |
| Ps3233 | CAAGCC | 34 | GGUGAAG | 361 | GCCAAGC | 688 |
| | UCUUCU | | UAAGAAG | | CUCUUCU | |
| | UACUUC | | AGGCUUG | | UACUUCA | |
| | ACC | | GC | | CC | |
| Ps3316 | GCUCGG | 35 | UUCUGCC | 362 | AAGCUCG | 689 |
| | | | AUCACUC | | GUGAGUG | |
| | UGAGUG | | ACCGAGC | | AUGGCAG | |
| | AUGGCA | | UU | | AA | |
| | GAA | | | | | |
| Ps3320 | GGUGAG | 36 | AUCGUUC | 363 | UCGGUGA | 690 |
| | UGAUGG | | UGCCAUC | | GUGAUGG | |
| | CAGAAC | | ACUCACC | | CAGAACG | |
| | GAU | | GA | | AU | |
| Ps3464 | CCCAAGC | 37 | AUAAAUG | 364 | CACCCAA | 691 |
| | AAGCAG | | UCUGCUU | | GCAAGCA | |
| | ACAUUU | | GCUUGGG | | GACAUUU | |
| | AU | | UG | | AU | |
| Ps3466 | CAAGCA | 38 | AGAUAAA | 365 | CCCAAGC | 692 |
| | AGCAGA | | UGUCUGC | | AAGCAGA | |
| | CAUUUA | | UUGCUUG | | CAUUUAU | |
| | UCU | | GG | | CU | |
| Ps3489 | UGGGUC | 39 | GCAACAG | 366 | UUUGGGU | 693 |
| | UGUCCU | | AGAGGAC | | CUGUCCU | |
| | CUCUGU | | AGACCCA | | CUCUGUU | |
| | UGC | | AA | | GC | |
| Ps3543 | CUUUUG | 40 | AAUAUCU | 367 | UGCUUUU | 694 |
| | UAACUU | | UCAAGUU | | GUAACUU | |
| | GAAGAU | | ACAAAAG | | GAAGAUA | |
| | AUU | | CA | | UU | |
| Ps3548 | GUAACU | 41 | GAAUAAA | 368 | UUGUAAC | 695 |
| | UGAAGA | | UAUCUUC | | UUGAAGA | |
| | UAUUUA | | AAGUUAC | | UAUUUAU | |
| | UUC | | AA | | UC | |
| Ps3554 | UGAAGA | 42 | AACCCAG | 369 | CUUGAAG | 696 |
| | UAUUUA | | AAUAAAU | | AUAUUUA | |
| | UUCUGG | | AUCUUCA | | UUCUGGG | |
| | GUU | | AG | | UU | |
| P446 | CCGAAG | 43 | UUCCGUG | 370 | GGCCGAA | 697 |
| | CACCCGA | | CUCGGGU | | GCACCCG | |
| | GCACGG | | GCUUCGG | | AGCACGG | |
| | AA | | CC | | AA | |
| P516 | CACCUAC | 44 | UUCAGCA | 371 | GGCACCU | 698 |
| | GUGGUG | | CCACCAC | | ACGUGGU | |
| | GUGCUG | | GUAGGUG | | GGUGCUG | |
| | AA | | CC | | AA | |
| P607 | UACCUC | 45 | AUGCAGG | 372 | GAUACCU | 699 |
| | ACCAAG | | AUCUUGG | | CACCAAG | |
| | AUCCUG | | UGAGGUA | | AUCCUGC | |
| | CAU | | UC | | AU | |
| P612 | CACCAA | 46 | AAGACAU | 373 | CUCACCA | 700 |
| | GAUCCU | | GCAGGAU | | AGAUCCU | |
| | GCAUGU | | CUUGGUG | | GCAUGUC | |
| | CUU | | AG | | UU | |
| P629 | UCUUCC | 47 | CAGGAAG | 374 | UGUCUUC | 701 |
| | AUGGCC | | AAGGCCA | | CAUGGCC | |
| | UUCUUC | | UGGAAGA | | UUCUUCC | |
| | CUG | | CA | | UG | |
| P633 | CCAUGG | 48 | AAGCCAG | 375 | UUCCAUG | 702 |
| | CCUUCU | | GAAGAAG | | GCCUUCU | |
| | UCCUGG | | GCCAUGG | | UCCUGGC | |
| | CUU | | AA | | UU | |
| P693 | GAAGUU | 49 | UAGUCGA | 376 | UUGAAGU | 703 |
| | GCCCCAU | | CAUGGGG | | UGCCCCA | |
| | GUCGAC | | CAACUUC | | UGUCGAC | |
| | UA | | AA | | UA | |
| P823 | CUGGUG | 50 | UAGGAGA | 377 | GCCUGGU | 704 |
| | GAGGUG | | UACACCU | | GGAGGUG | |
| | UAUCUC | | CCACCAG | | UAUCUCC | |
| | CUA | | GC | | UA | |
| P835 | UAUCUC | 51 | UAUGCUG | 378 | UGUAUCU | 705 |
| | CUAGAC | | GUGUCUA | | CCUAGAC | |
| | ACCAGC | | GGAGAUA | | ACCAGCA | |
| | AUA | | CA | | UA | |
| P839 | UCCUAG | 52 | UCUGUAU | 379 | UCUCCUA | 706 |
| | ACACCA | | GCUGGUG | | GACACCA | |
| | GCAUAC | | UCUAGGA | | GCAUACA | |
| | AGA | | GA | | GA | |
| P842 | UAGACA | 53 | CACUCUG | 380 | CCUAGAC | 707 |
| | CCAGCA | | UAUGCUG | | ACCAGCA | |
| | UACAGA | | GUGUCUA | | UACAGAG | |
| | GUG | | GG | | UG | |
| P845 | ACACCA | 54 | GGUCACU | 381 | AGACACC | 708 |
| | GCAUAC | | CUGUAUG | | AGCAUAC | |
| | AGAGUG | | CUGGUGU | | AGAGUGA | |
| | ACC | | CU | | CC | |
| P854 | UACAGA | 55 | UUUCCCG | 382 | CAUACAG | 709 |
| | GUGACC | | GUGGUCA | | AGUGACC | |
| | ACCGGG | | CUCUGUA | | ACCGGGA | |
| | AAA | | UG | | AA | |
| P882 | CAGGGU | 56 | AAGUCGG | 383 | GGCAGGG | 710 |
| | CAUGGU | | UGACCAU | | UCAUGGU | |
| | CACCGAC | | GACCCUG | | CACCGAC | |
| | UU | | CC | | UU | |
| P888 | CAUGGU | 57 | UUCUCGA | 384 | GUCAUGG | 711 |
| | CACCGAC | | AGUCGGU | | UCACCGA | |
| | UUCGAG | | GACCAUG | | CUUCGAG | |
| | AA | | AC | | AA | |
| P891 | GGUCAC | 58 | ACAUUCU | 385 | AUGGUCA | 712 |
| | CGACUU | | CGAAGUC | | CCGACUU | |
| | CGAGAA | | GGUGACC | | CGAGAAU | |
| | UGU | | AU | | GU | |
| P1066 | ACGGUU | 59 | UAUGAGG | 386 | GCACGGU | 713 |
| | AGCGGC | | GUGCCGC | | UAGCGGC | |
| | ACCCUC | | UAACCGU | | ACCCUCA | |
| | AUA | | GC | | UA | |
| P1079 | CCCUCAU | 60 | UAAACUC | 387 | CACCCUC | 714 |
| | AGGCCU | | CAGGCCU | | AUAGGCC | |
| | GGAGUU | | AUGAGGG | | UGGAGUU | |
| | UA | | UG | | UA | |
| P1081 | CUCAUA | 61 | AAUAAAC | 388 | CCCUCAU | 715 |
| | GGCCUG | | UCCAGGC | | AGGCCUG | |
| | GAGUUU | | CUAUGAG | | GAGUUUA | |
| | AUU | | GG | | UU | |
| P1085 | UAGGCC | 62 | UCCGAAU | 389 | CAUAGGC | 716 |
| | UGGAGU | | AAACUCC | | CUGGAGU | |
| | UUAUUC | | AGGCCUA | | UUAUUCG | |
| | GGA | | UG | | GA | |
| P1161 | UGGGUA | 63 | UUGAGGA | 390 | GGUGGGU | 717 |
| | CAGCCGC | | CGCGGCU | | ACAGCCG | |
| | GUCCUC | | GUACCCA | | CGUCCUC | |
| | AA | | CC | | AA | |
| P1242 | CCGGGA | 64 | UAGAGGC | 391 | UUCCGGG | 718 |
| | CGAUGC | | AGGCAUC | | ACGAUGC | |
| | CUGCCUC | | GUCCCGG | | CUGCCUC | |
| | UA | | AA | | UA | |
| P1413 | CAGCACC | 65 | UGUGACA | 392 | UGCAGCA | 719 |
| | UGCUUU | | CAAAGCA | | CCUGCUU | |
| | GUGUCA | | GGUGCUG | | UGUGUCA | |
| | CA | | CA | | CA | |
| P1514 | CCGAGU | 66 | UCAGUCU | 393 | GGCCGAG | 720 |
| | UGAGGC | | CUGCCUC | | UUGAGGC | |
| | AGAGAC | | AACUCGG | | AGAGACU | |
| | UGA | | CC | | GA | |
| P1518 | GUUGAG | 67 | UGGAUCA | 394 | GAGUUGA | 721 |
| | GCAGAG | | GUCUCUG | | GGCAGAG | |
| | ACUGAU | | CCUCAAC | | ACUGAUC | |
| | CCA | | UC | | CA | |
| P1521 | GAGGCA | 68 | AAGUGGA | 395 | UUGAGGC | 722 |
| | GAGACU | | UCAGUCU | | AGAGACU | |
| | GAUCCA | | CUGCCUC | | GAUCCAC | |
| | CUU | | AA | | UU | |
| P1523 | GGCAGA | 69 | AGAAGUG | 396 | GAGGCAG | 723 |
| | GACUGA | | GAUCAGU | | AGACUGA | |
| | UCCACU | | CUCUGCC | | UCCACUU | |
| | UCU | | UC | | CU | |
| P1531 | CUGAUC | 70 | UUUGGCA | 397 | GACUGAU | 724 |
| | CACUUC | | GAGAAGU | | CCACUUC | |
| | UCUGCC | | GGAUCAG | | UCUGCCA | |
| | AAA | | UC | | AA | |
| P1533 | GAUCCA | 71 | UCUUUGG | 398 | CUGAUCC | 725 |
| | CUUCUC | | CAGAGAA | | ACUUCUC | |
| | UGCCAA | | GUGGAUC | | UGCCAAA | |
| | AGA | | AG | | GA | |
| P1536 | CCACUUC | 72 | ACAUCUU | 399 | AUCCACU | 726 |
| | UCUGCC | | UGGCAGA | | UCUCUGC | |
| | AAAGAU | | GAAGUGG | | CAAAGAU | |
| | GU | | AU | | GU | |
| P1539 | CUUCUC | 73 | AUGACAU | 400 | CACUUCU | 727 |
| | UGCCAA | | CUUUGGC | | CUGCCAA | |
| | AGAUGU | | AGAGAAG | | AGAUGUC | |
| | CAU | | UG | | AU | |
| P1547 | CCAAAG | 74 | CCUCAUU | 401 | UGCCAAA | 728 |
| | AUGUCA | | GAUGACA | | GAUGUCA | |
| | UCAAUG | | UCUUUGG | | UCAAUGA | |
| | AGG | | CA | | GG | |
| P1659 | CAGGAC | 75 | UGUGCUG | 402 | UGCAGGA | 729 |
| | UGUAUG | | ACCAUAC | | CUGUAUG | |
| | GUCAGC | | AGUCCUG | | GUCAGCA | |
| | ACA | | CA | | CA | |
| P1661 | GGACUG | 76 | AGUGUGC | 403 | CAGGACU | 730 |
| | UAUGGU | | UGACCAU | | GUAUGGU | |
| | CAGCAC | | ACAGUCC | | CAGCACA | |
| | ACU | | UG | | CU | |
| P2101 | GGUCUG | 77 | CUUGACU | 404 | CAGGUCU | 731 |
| | GAAUGC | | UUGCAUU | | GGAAUGC | |
| | AAAGUC | | CCAGACC | | AAAGUCA | |
| | AAG | | UG | | AG | |
| P2112 | CAAAGU | 78 | AUUCCAU | 405 | UGCAAAG | 732 |
| | CAAGGA | | GCUCCUU | | UCAAGGA | |
| | GCAUGG | | GACUUUG | | GCAUGGA | |
| | AAU | | CA | | AU | |
| P2114 | AAGUCA | 79 | GGAUUCC | 406 | CAAAGUC | 733 |
| | AGGAGC | | AUGCUCC | | AAGGAGC | |
| | AUGGAA | | UUGACUU | | AUGGAAU | |
| | UCC | | UG | | CC | |
| P2116 | GUCAAG | 80 | CGGGAUU | 407 | AAGUCAA | 734 |
| | GAGCAU | | CCAUGCU | | GGAGCAU | |
| | GGAAUC | | CCUUGAC | | GGAAUCC | |
| | CCG | | UU | | CG | |
| SP71 | ACACAU | 81 | GAAGUAG | 408 | GUACACA | 735 |
| | UCGCACC | | GGGUGCG | | UUCGCAC | |
| | CCUACU | | AAUGUGU | | CCCUACU | |
| | UC | | AC | | UC | |
| SP73 | ACAUUC | 82 | GUGAAGU | 409 | ACACAUU | 736 |
| | GCACCCC | | AGGGGUG | | CGCACCC | |
| | UACUUC | | CGAAUGU | | CUACUUC | |
| | AC | | GU | | AC | |
| SP74 | CAUUCG | 83 | UGUGAAG | 410 | CACAUUC | 737 |
| | CACCCCU | | UAGGGGU | | GCACCCC | |
| | ACUUCA | | GCGAAUG | | UACUUCA | |
| | CA | | UG | | CA | |
| SP76 | UUCGCA | 84 | UCUGUGA | 411 | CAUUCGC | 738 |
| | CCCCUAC | | AGUAGGG | | ACCCCUA | |
| | UUCACA | | GUGCGAA | | CUUCACA | |
| | GA | | UG | | GA | |
| SP107 | AACUGA | 85 | AUCUGCG | 412 | GGAACUG | 739 |
| | GCCAGA | | UUUCUGG | | AGCCAGA | |
| | AACGCA | | CUCAGUU | | AACGCAG | |
| | GAU | | CC | | AU | |
| SP109 | CUGAGC | 86 | CAAUCUG | 413 | AACUGAG | 740 |
| | CAGAAA | | CGUUUCU | | CCAGAAA | |
| | CGCAGA | | GGCUCAG | | CGCAGAU | |
| | UUG | | UU | | UG | |
| SP133 | GCCAAG | 87 | UGAAGUA | 414 | AAGCCAA | 741 |
| | CCUCUUC | | AGAAGAG | | GCCUCUU | |
| | UUACUU | | GCUUGGC | | CUUACUU | |
| | CA | | UU | | CA | |
| SP134 | CCAAGCC | 88 | GUGAAGU | 415 | AGCCAAG | 742 |
| | UCUUCU | | AAGAAGA | | CCUCUUC | |
| | UACUUC | | GGCUUGG | | UUACUUC | |
| | AC | | CU | | AC | |
| SP227 | CUUUUU | 89 | CCUGGGU | 416 | AACUUUU | 743 |
| | CCGUUA | | GAUAACG | | UCCGUUA | |
| | UCACCCA | | GAAAAAG | | UCACCCA | |
| | GG | | UU | | GG | |
| SP300 | CACCCAA | 90 | AAAUGUC | 417 | CCCACCC | 744 |
| | GCAAGC | | UGCUUGC | | AAGCAAG | |
| | AGACAU | | UUGGGUG | | CAGACAU | |
| | UU | | GG | | UU | |
| SP301 | ACCCAA | 91 | UAAAUGU | 418 | CCACCCA | 745 |
| | GCAAGC | | CUGCUUG | | AGCAAGC | |
| | AGACAU | | CUUGGGU | | AGACAUU | |
| | UUA | | GG | | UA | |
| SP304 | AAGCAA | 92 | AAGAUAA | 419 | CCAAGCA | 746 |
| | GCAGAC | | AUGUCUG | | AGCAGAC | |
| | AUUUAU | | CUUGCUU | | AUUUAUC | |
| | CUU | | GG | | UU | |
| SP305 | AAGCAG | 93 | CCAAAAG | 420 | GCAAGCA | 747 |
| | ACAUUU | | AUAAAUG | | GACAUUU | |
| | AUCUUU | | UCUGCUU | | AUCUUUU | |
| | UGG | | GC | | GG | |
| SP306 | AGCAGA | 94 | CCCAAAA | 421 | CAAGCAG | 748 |
| | CAUUUA | | GAUAAAU | | ACAUUUA | |
| | UCUUUU | | GUCUGCU | | UCUUUUG | |
| | GGG | | UG | | GG | |
| SP326 | UGUCCU | 95 | AAAAAGG | 422 | UCUGUCC | 749 |
| | CUCUGU | | CAACAGA | | UCUCUGU | |
| | UGCCUU | | GAGGACA | | UGCCUUU | |
| | UUU | | GA | | UU | |
| SP327 | UCCUCUC | 96 | GUAAAAA | 423 | UGUCCUC | 750 |
| | UGUUGC | | GGCAACA | | UCUGUUG | |
| | CUUUUU | | GAGAGGA | | CCUUUUU | |
| | AC | | CA | | AC | |
| SP332 | GCCUUU | 97 | AAAAGUU | 424 | UUGCCUU | 751 |
| | UUACAG | | GGCUGUA | | UUUACAG | |
| | CCAACU | | AAAAGGC | | CCAACUU | |
| | UUU | | AA | | UU | |
| SP334 | UUUACA | 98 | UCUAGAA | 425 | UUUUUAC | 752 |
| | GCCAAC | | AAGUUGG | | AGCCAAC | |
| | UUUUCU | | CUGUAAA | | UUUUCUA | |
| | AGA | | AA | | GA | |
| SP340 | GCCAAC | 99 | AACAGGU | 426 | CAGCCAA | 753 |
| | UUUUCU | | CUAGAAA | | CUUUUCU | |
| | AGACCU | | AGUUGGC | | AGACCUG | |
| | GUU | | UG | | UU | |
| SP341 | CCAACU | 100 | AAACAGG | 427 | AGCCAAC | 754 |
| | UUUCUA | | UCUAGAA | | UUUUCUA | |
| | GACCUG | | AAGUUGG | | GACCUGU | |
| | UUU | | CU | | UU | |
| SP342 | CAACUU | 101 | AAAACAG | 428 | GCCAACU | 755 |
| | UUCUAG | | GUCUAGA | | UUUCUAG | |
| | ACCUGU | | AAAGUUG | | ACCUGUU | |
| | UUU | | GC | | UU | |
| SP343 | AACUUU | 102 | CAAAACA | 429 | CCAACUU | 756 |
| | UCUAGA | | GGUCUAG | | UUCUAGA | |
| | CCUGUU | | AAAAGUU | | CCUGUUU | |
| | UUG | | GG | | UG | |
| SP344 | ACUUUU | 103 | GCAAAAC | 430 | CAACUUU | 757 |
| | CUAGAC | | AGGUCUA | | UCUAGAC | |
| | CUGUUU | | GAAAAGU | | CUGUUUU | |
| | UGC | | UG | | GC | |
| SP345 | CUUUUC | 104 | AGCAAAA | 431 | AACUUUU | 758 |
| | UAGACC | | CAGGUCU | | CUAGACC | |
| | UGUUUU | | AGAAAAG | | UGUUUUG | |
| | GCU | | UU | | CU | |
| SP348 | UUCUAG | 105 | AAAAGCA | 432 | UUUUCUA | 759 |
| | ACCUGU | | AAACAGG | | GACCUGU | |
| | UUUGCU | | UCUAGAA | | UUUGCUU | |
| | UUU | | AA | | UU | |
| SP349 | UCUAGA | 106 | CAAAAGC | 433 | UUUCUAG | 760 |
| | CCUGUU | | AAAACAG | | ACCUGUU | |
| | UUGCUU | | GUCUAGA | | UUGCUUU | |
| | UUG | | AA | | UG | |
| SP351 | AGACCU | 107 | UUACAAA | 434 | CUAGACC | 761 |
| | GUUUUG | | AGCAAAA | | UGUUUUG | |
| | CUUUUG | | CAGGUCU | | CUUUUGU | |
| | UAA | | AG | | AA | |
| SP357 | AUUUAU | 108 | GCUACAA | 435 | AUAUUUA | 762 |
| | UCUGGG | | AACCCAG | | UUCUGGG | |
| | UUUUGU | | AAUAAAU | | UUUUGUA | |
| | AGC | | AU | | GC | |
| Pt608 | ACCUCAC | 109 | CAUGCAG | 436 | AUACCUC | 763 |
| | CAAGAU | | GAUCUUG | | ACCAAGA | |
| | CCUGCA | | GUGAGGU | | UCCUGCA | |
| | UG | | AU | | UG | |
| Pt609 | CCUCACC | 110 | ACAUGCA | 437 | UACCUCA | 764 |
| | AAGAUC | | GGAUCUU | | CCAAGAU | |
| | CUGCAU | | GGUGAGG | | CCUGCAU | |
| | GU | | UA | | GU | |
| Pt692 | UGAAGU | 111 | AGUCGAC | 438 | CUUGAAG | 765 |
| | UGCCCCA | | AUGGGGC | | UUGCCCC | |
| | UGUCGA | | AACUUCA | | AUGUCGA | |
| | CU | | AG | | CU | |
| Pt694 | AAGUUG | 112 | GUAGUCG | 439 | UGAAGUU | 766 |
| | CCCCAUG | | ACAUGGG | | GCCCCAU | |
| | UCGACU | | GCAACUU | | GUCGACU | |
| | AC | | CA | | AC | |
| Pt695 | AGUUGC | 113 | UGUAGUC | 440 | GAAGUUG | 767 |
| | CCCAUG | | GACAUGG | | CCCCAUG | |
| | UCGACU | | GGCAACU | | UCGACUA | |
| | ACA | | UC | | CA | |
| Pt837 | UCUCCU | 114 | UGUAUGC | 441 | UAUCUCC | 768 |
| | AGACAC | | UGGUGUC | | UAGACAC | |
| | CAGCAU | | UAGGAGA | | CAGCAUA | |
| | ACA | | UA | | CA | |
| Pt838 | CUCCUA | 115 | CUGUAUG | 442 | AUCUCCU | 769 |
| | GACACC | | CUGGUGU | | AGACACC | |
| | AGCAUA | | CUAGGAG | | AGCAUAC | |
| | CAG | | AU | | AG | |
| Pt840 | CCUAGA | 116 | CUCUGUA | 443 | CUCCUAG | 770 |
| | CACCAGC | | UGCUGGU | | ACACCAG | |
| | AUACAG | | GUCUAGG | | CAUACAG | |
| | AG | | AG | | AG | |
| Pt841 | CUAGAC | 117 | ACUCUGU | 444 | UCCUAGA | 771 |
| | ACCAGC | | AUGCUGG | | CACCAGC | |
| | AUACAG | | UGUCUAG | | AUACAGA | |
| | AGU | | GA | | GU | |
| Pt844 | GACACC | 118 | GUCACUC | 445 | UAGACAC | 772 |
| | AGCAUA | | UGUAUGC | | CAGCAUA | |
| | CAGAGU | | UGGUGUC | | CAGAGUG | |
| | GAC | | UA | | AC | |
| Pt847 | ACCAGC | 119 | GUGGUCA | 446 | ACACCAG | 773 |
| | AUACAG | | CUCUGUA | | CAUACAG | |
| | AGUGAC | | UGCUGGU | | AGUGACC | |
| | CAC | | GU | | AC | |
| Pt848 | CCAGCA | 120 | GGUGGUC | 447 | CACCAGC | 774 |
| | UACAGA | | ACUCUGU | | AUACAGA | |
| | GUGACC | | AUGCUGG | | GUGACCA | |
| | ACC | | UG | | CC | |
| Pt855 | ACAGAG | 121 | AUUUCCC | 448 | AUACAGA | 775 |
| | UGACCA | | GGUGGUC | | GUGACCA | |
| | CCGGGA | | ACUCUGU | | CCGGGAA | |
| | AAU | | AU | | AU | |
| Pt856 | CAGAGU | 122 | GAUUUCC | 449 | UACAGAG | 776 |
| | GACCACC | | CGGUGGU | | UGACCAC | |
| | GGGAAA | | CACUCUG | | CGGGAAA | |
| | UC | | UA | | UC | |
| Pt889 | AUGGUC | 123 | AUUCUCG | 450 | UCAUGGU | 777 |
| | ACCGAC | | AAGUCGG | | CACCGAC | |
| | UUCGAG | | UGACCAU | | UUCGAGA | |
| | AAU | | GA | | AU | |
| Pt890 | UGGUCA | 124 | CAUUCUC | 451 | CAUGGUC | 778 |
| | CCGACU | | GAAGUCG | | ACCGACU | |
| | UCGAGA | | GUGACCA | | UCGAGAA | |
| | AUG | | UG | | UG | |
| Pt892 | GUCACC | 125 | CACAUUC | 452 | UGGUCAC | 779 |
| | GACUUC | | UCGAAGU | | CGACUUC | |
| | GAGAAU | | CGGUGAC | | GAGAAUG | |
| | GUG | | CA | | UG | |
| Pt941 | GACAGG | 126 | UGUCACA | 453 | CAGACAG | 780 |
| | CCAGCA | | CUUGCUG | | GCCAGCA | |
| | AGUGUG | | GCCUGUC | | AGUGUGA | |
| | ACA | | UG | | CA | |
| Pt1080 | CCUCAU | 127 | AUAAACU | 454 | ACCCUCA | 781 |
| | AGGCCU | | CCAGGCC | | UAGGCCU | |
| | GGAGUU | | UAUGAGG | | GGAGUUU | |
| | UAU | | GU | | AU | |
| Pt1082 | UCAUAG | 128 | GAAUAAA | 455 | CCUCAUA | 782 |
| | GCCUGG | | CUCCAGG | | GGCCUGG | |
| | AGUUUA | | CCUAUGA | | AGUUUAU | |
| | UUC | | GG | | UC | |
| Pt1083 | CAUAGG | 129 | CGAAUAA | 456 | CUCAUAG | 783 |
| | CCUGGA | | ACUCCAG | | GCCUGGA | |
| | GUUUAU | | GCCUAUG | | GUUUAUU | |
| | UCG | | AG | | CG | |
| Pt1084 | AUAGGC | 130 | CCGAAUA | 457 | UCAUAGG | 784 |
| | CUGGAG | | AACUCCA | | CCUGGAG | |
| | UUUAUU | | GGCCUAU | | UUUAUUC | |
| | CGG | | GA | | GG | |
| Pt1086 | AGGCCU | 131 | UUCCGAA | 458 | AUAGGCC | 785 |
| | GGAGUU | | UAAACUC | | UGGAGUU | |
| | UAUUCG | | CAGGCCU | | UAUUCGG | |
| | GAA | | AU | | AA | |
| Pt1087 | GGCCUG | 132 | UUUCCGA | 459 | UAGGCCU | 786 |
| | GAGUUU | | AUAAACU | | GGAGUUU | |
| | AUUCGG | | CCAGGCC | | AUUCGGA | |
| | AAA | | UA | | AA | |
| Pt1275 | AGCUCCC | 133 | ACUGUGA | 460 | UCAGCUC | 787 |
| | GAGGUC | | UGACCUC | | CCGAGGU | |
| | AUCACA | | GGGAGCU | | CAUCACA | |
| | GU | | GA | | GU | |
| Pt1516 | GAGUUG | 134 | GAUCAGU | 461 | CCGAGUU | 788 |
| | AGGCAG | | CUCUGCC | | GAGGCAG | |
| | AGACUG | | UCAACUC | | AGACUGA | |
| | AUC | | GG | | UC | |
| Pt1517 | AGUUGA | 135 | GGAUCAG | 462 | CGAGUUG | 789 |
| | GGCAGA | | UCUCUGC | | AGGCAGA | |
| | GACUGA | | CUCAACU | | GACUGAU | |
| | UCC | | CG | | CC | |
| Pt1519 | UUGAGG | 136 | GUGGAUC | 463 | AGUUGAG | 790 |
| | CAGAGA | | AGUCUCU | | GCAGAGA | |
| | CUGAUC | | GCCUCAA | | CUGAUCC | |
| | CAC | | CU | | AC | |
| Pt1520 | UGAGGC | 137 | AGUGGAU | 464 | GUUGAGG | 791 |
| | AGAGAC | | CAGUCUC | | CAGAGAC | |
| | UGAUCC | | UGCCUCA | | UGAUCCA | |
| | ACU | | AC | | CU | |
| Pt1522 | AGGCAG | 138 | GAAGUGG | 465 | UGAGGCA | 792 |
| | AGACUG | | AUCAGUC | | GAGACUG | |
| | AUCCAC | | UCUGCCU | | AUCCACU | |
| | UUC | | CA | | UC | |
| Pt1530 | ACUGAU | 139 | UUGGCAG | 466 | AGACUGA | 793 |
| | CCACUUC | | AGAAGUG | | UCCACUU | |
| | UCUGCC | | GAUCAGU | | CUCUGCC | |
| | AA | | CU | | AA | |
| Pt1532 | UGAUCC | 140 | CUUUGGC | 467 | ACUGAUC | 794 |
| | ACUUCU | | AGAGAAG | | CACUUCU | |
| | CUGCCA | | UGGAUCA | | CUGCCAA | |
| | AAG | | GU | | AG | |
| Pt1534 | AUCCAC | 141 | AUCUUUG | 468 | UGAUCCA | 795 |
| | UUCUCU | | GCAGAGA | | CUUCUCU | |
| | GCCAAA | | AGUGGAU | | GCCAAAG | |
| | GAU | | CA | | AU | |
| Pt1535 | UCCACU | 142 | CAUCUUU | 469 | GAUCCAC | 796 |
| | UCUCUG | | GGCAGAG | | UUCUCUG | |
| | CCAAAG | | AAGUGGA | | CCAAAGA | |
| | AUG | | UC | | UG | |
| Pt1537 | CACUUC | 143 | GACAUCU | 470 | UCCACUU | 797 |
| | UCUGCC | | UUGGCAG | | CUCUGCC | |
| | AAAGAU | | AGAAGUG | | AAAGAUG | |
| | GUC | | GA | | UC | |
| Pt1538 | ACUUCU | 144 | UGACAUC | 471 | CCACUUC | 798 |
| | CUGCCA | | UUUGGCA | | UCUGCCA | |
| | AAGAUG | | GAGAAGU | | AAGAUGU | |
| | UCA | | GG | | CA | |
| Pt2102 | GUCUGG | 145 | CCUUGAC | 472 | AGGUCUG | 799 |
| | AAUGCA | | UUUGCAU | | GAAUGCA | |
| | AAGUCA | | UCCAGAC | | AAGUCAA | |
| | AGG | | CU | | GG | |
| Pt2105 | UGGAAU | 146 | GCUCCUU | 473 | UCUGGAA | 800 |
| | GCAAAG | | GACUUUG | | UGCAAAG | |
| | UCAAGG | | CAUUCCA | | UCAAGGA | |
| | AGC | | GA | | GC | |
| Pt2106 | GGAAUG | 147 | UGCUCCU | 474 | CUGGAAU | 801 |
| | CAAAGU | | UGACUUU | | GCAAAGU | |
| | CAAGGA | | GCAUUCC | | CAAGGAG | |
| | GCA | | AG | | CA | |
| Pt2113 | AAAGUC | 148 | GAUUCCA | 475 | GCAAAGU | 802 |
| | AAGGAG | | UGCUCCU | | CAAGGAG | |
| | CAUGGA | | UGACUUU | | CAUGGAA | |
| | AUC | | GC | | UC | |
| Pt2115 | AGUCAA | 149 | GGGAUUC | 476 | AAAGUCA | 803 |
| | GGAGCA | | CAUGCUC | | AGGAGCA | |
| | UGGAAU | | CUUGACU | | UGGAAUC | |
| | CCC | | UU | | CC | |
| Pt2426 | AUGGUU | 150 | AGAGGGA | 477 | AAAUGGU | 804 |
| | CCGACU | | CAAGUCG | | UCCGACU | |
| | UGUCCC | | GAACCAU | | UGUCCCU | |
| | UCU | | UU | | CU | |
| Pt2427 | UGGUUC | 151 | GAGAGGG | 478 | AAUGGUU | 805 |
| | CGACUU | | ACAAGUC | | CCGACUU | |
| | GUCCCUC | | GGAACCA | | GUCCCUC | |
| | UC | | UU | | UC | |
| Pt2432 | CCGACU | 152 | GCUGAGA | 479 | UUCCGAC | 806 |
| | UGUCCC | | GAGGGAC | | UUGUCCC | |
| | UCUCUC | | AAGUCGG | | UCUCUCA | |
| | AGC | | AA | | GC | |
| Pt2530 | CCUCCUU | 153 | AGUGAGU | 480 | AGCCUCC | 807 |
| | GCCUGG | | UCCAGGC | | UUGCCUG | |
| | AACUCA | | AAGGAGG | | GAACUCA | |
| | CU | | CU | | CU | |
| Pt2532 | UCCUUG | 154 | UGAGUGA | 481 | CCUCCUU | 808 |
| | CCUGGA | | GUUCCAG | | GCCUGGA | |
| | ACUCAC | | GCAAGGA | | ACUCACU | |
| | UCA | | GG | | CA | |
| Pt2533 | CCUUGCC | 155 | GUGAGUG | 482 | CUCCUUG | 809 |
| | UGGAAC | | AGUUCCA | | CCUGGAA | |
| | UCACUC | | GGCAAGG | | CUCACUC | |
| | AC | | AG | | AC | |
| Pt2534 | CUUGCC | 156 | AGUGAGU | 483 | UCCUUGC | 810 |
| | UGGAAC | | GAGUUCC | | CUGGAAC | |
| | UCACUC | | AGGCAAG | | UCACUCA | |
| | ACU | | GA | | CU | |
| Pt2535 | UUGCCU | 157 | GAGUGAG | 484 | CCUUGCC | 811 |
| | GGAACU | | UGAGUUC | | UGGAACU | |
| | CACUCAC | | CAGGCAA | | CACUCAC | |
| | UC | | GG | | UC | |
| Pt2536 | UGCCUG | 158 | AGAGUGA | 485 | CUUGCCU | 812 |
| | GAACUC | | GUGAGUU | | GGAACUC | |
| | ACUCAC | | CCAGGCA | | ACUCACU | |
| | UCU | | AG | | CU | |
| Pt2538 | CCUGGA | 159 | CCAGAGU | 486 | UGCCUGG | 813 |
| | ACUCAC | | GAGUGAG | | AACUCAC | |
| | UCACUC | | UUCCAGG | | UCACUCU | |
| | UGG | | CA | | GG | |
| Pt2540 | UGGAAC | 160 | ACCCAGA | 487 | CCUGGAA | 814 |
| | UCACUC | | GUGAGUG | | CUCACUC | |
| | ACUCUG | | AGUUCCA | | ACUCUGG | |
| | GGU | | GG | | GU | |
| Pt2541 | GGAACU | 161 | CACCCAG | 488 | CUGGAAC | 815 |
| | CACUCAC | | AGUGAGU | | UCACUCA | |
| | UCUGGG | | GAGUUCC | | CUCUGGG | |
| | UG | | AG | | UG | |
| Pt2542 | GAACUC | 162 | GCACCCA | 489 | UGGAACU | 816 |
| | ACUCAC | | GAGUGAG | | CACUCAC | |
| | UCUGGG | | UGAGUUC | | UCUGGGU | |
| | UGC | | CA | | GC | |
| Pt2544 | ACUCAC | 163 | AGGCACC | 490 | GAACUCA | 817 |
| | UCACUC | | CAGAGUG | | CUCACUC | |
| | UGGGUG | | AGUGAGU | | UGGGUGC | |
| | CCU | | UC | | CU | |
| Pt2545 | CUCACUC | 164 | GAGGCAC | 491 | AACUCAC | 818 |
| | ACUCUG | | CCAGAGU | | UCACUCU | |
| | GGUGCC | | GAGUGAG | | GGGUGCC | |
| | UC | | UU | | UC | |
| Pt2611 | CAUUUC | 165 | ACCUGUU | 492 | GGCAUUU | 819 |
| | ACCAUU | | UGAAUGG | | CACCAUU | |
| | CAAACA | | UGAAAUG | | CAAACAG | |
| | GGU | | CC | | GU | |
| Pt2612 | AUUUCA | 166 | GACCUGU | 493 | GCAUUUC | 820 |
| | CCAUUC | | UUGAAUG | | ACCAUUC | |
| | AAACAG | | GUGAAAU | | AAACAGG | |
| | GUC | | GC | | UC | |
| Pt2613 | UUUCAC | 167 | CGACCUG | 494 | CAUUUCA | 821 |
| | CAUUCA | | UUUGAAU | | CCAUUCA | |
| | AACAGG | | GGUGAAA | | AACAGGU | |
| | UCG | | UG | | CG | |
| Pt2614 | UUCACC | 168 | UCGACCU | 495 | AUUUCAC | 822 |
| | AUUCAA | | GUUUGAA | | CAUUCAA | |
| | ACAGGU | | UGGUGAA | | ACAGGUC | |
| | CGA | | AU | | GA | |
| Pt2615 | UCACCA | 169 | CUCGACC | 496 | UUUCACC | 823 |
| | UUCAAA | | UGUUUGA | | AUUCAAA | |
| | CAGGUC | | AUGGUGA | | CAGGUCG | |
| | GAG | | AA | | AG | |
| Pt2616 | CACCAU | 170 | GCUCGAC | 497 | UUCACCA | 824 |
| | UCAAAC | | CUGUUUG | | UUCAAAC | |
| | AGGUCG | | AAUGGUG | | AGGUCGA | |
| | AGC | | AA | | GC | |
| Pt2618 | CCAUUC | 171 | CAGCUCG | 498 | CACCAUU | 825 |
| | AAACAG | | ACCUGUU | | CAAACAG | |
| | GUCGAG | | UGAAUGG | | GUCGAGC | |
| | CUG | | UG | | UG | |
| Pt2656 | CUGCUCC | 172 | ACAUCGG | 499 | AGCUGCU | 826 |
| | CAAUGU | | CACAUUG | | CCCAAUG | |
| | GCCGAU | | GGAGCAG | | UGCCGAU | |
| | GU | | CU | | GU | |
| Pt2673 | AUGUCC | 173 | AGUCAUU | 500 | CGAUGUC | 827 |
| | GUGGGC | | CUGCCCA | | CGUGGGC | |
| | AGAAUG | | CGGACAU | | AGAAUGA | |
| | ACU | | CG | | CU | |
| Pt2675 | GUCCGU | 174 | AAAGUCA | 501 | AUGUCCG | 828 |
| | GGGCAG | | UUCUGCC | | UGGGCAG | |
| | AAUGAC | | CACGGAC | | AAUGACU | |
| | UUU | | AU | | UU | |
| Pt2696 | UAUUGA | 175 | GCACGGA | 502 | UUUAUUG | 829 |
| | GCUCUU | | ACAAGAG | | AGCUCUU | |
| | GUUCCG | | CUCAAUA | | GUUCCGU | |
| | UGC | | AA | | GC | |
| Pt2697 | AUUGAG | 176 | GGCACGG | 503 | UUAUUGA | 830 |
| | CUCUUG | | AACAAGA | | GCUCUUG | |
| | UUCCGU | | GCUCAAU | | UUCCGUG | |
| | GCC | | AA | | CC | |
| Pt2698 | UUGAGC | 177 | UGGCACG | 504 | UAUUGAG | 831 |
| | UCUUGU | | GAACAAG | | CUCUUGU | |
| | UCCGUG | | AGCUCAA | | UCCGUGC | |
| | CCA | | UA | | CA | |
| Pt2699 | UGAGCU | 178 | CUGGCAC | 505 | AUUGAGC | 832 |
| | CUUGUU | | GGAACAA | | UCUUGUU | |
| | CCGUGCC | | GAGCUCA | | CCGUGCC | |
| | AG | | AU | | AG | |
| Pt2700 | GAGCUC | 179 | CCUGGCA | 506 | UUGAGCU | 833 |
| | UUGUUC | | CGGAACA | | CUUGUUC | |
| | CGUGCC | | AGAGCUC | | CGUGCCA | |
| | AGG | | AA | | GG | |
| Pt2717 | CAGGCA | 180 | GACCUGA | 507 | GCCAGGC | 834 |
| | UUCAAU | | GGAUUGA | | AUUCAAU | |
| | CCUCAG | | AUGCCUG | | CCUCAGG | |
| | GUC | | GC | | UC | |
| Pt2718 | AGGCAU | 181 | AGACCUG | 508 | CCAGGCA | 835 |
| | UCAAUC | | AGGAUUG | | UUCAAUC | |
| | CUCAGG | | AAUGCCU | | CUCAGGU | |
| | UCU | | GG | | CU | |
| Pt2720 | GCAUUC | 182 | GGAGACC | 509 | AGGCAUU | 836 |
| | AAUCCU | | UGAGGAU | | CAAUCCU | |
| | CAGGUC | | UGAAUGC | | CAGGUCU | |
| | UCC | | CU | | CC | |
| Pt2722 | AUUCAA | 183 | GUGGAGA | 510 | GCAUUCA | 837 |
| | UCCUCA | | CCUGAGG | | AUCCUCA | |
| | GGUCUC | | AUUGAAU | | GGUCUCC | |
| | CAC | | GC | | AC | |
| Pt2724 | UCAAUC | 184 | UGGUGGA | 511 | AUUCAAU | 838 |
| | CUCAGG | | GACCUGA | | CCUCAGG | |
| | UCUCCAC | | GGAUUGA | | UCUCCAC | |
| | CA | | AU | | CA | |
| Pt2726 | AAUCCU | 185 | CUUGGUG | 512 | UCAAUCC | 839 |
| | CAGGUC | | GAGACCU | | UCAGGUC | |
| | UCCACCA | | GAGGAUU | | UCCACCA | |
| | AG | | GA | | AG | |
| Pt2727 | AUCCUC | 186 | CCUUGGU | 513 | CAAUCCU | 840 |
| | AGGUCU | | GGAGACC | | CAGGUCU | |
| | CCACCAA | | UGAGGAU | | CCACCAA | |
| | GG | | UG | | GG | |
| Pt2741 | ACCAAG | 187 | GAAGAAU | 514 | CCACCAA | 841 |
| | GAGGCA | | CCUGCCU | | GGAGGCA | |
| | GGAUUC | | CCUUGGU | | GGAUUCU | |
| | UUC | | GG | | UC | |
| Pt2742 | CCAAGG | 188 | GGAAGAA | 515 | CACCAAG | 842 |
| | AGGCAG | | UCCUGCC | | GAGGCAG | |
| | GAUUCU | | UCCUUGG | | GAUUCUU | |
| | UCC | | UG | | CC | |
| Pt2743 | CAAGGA | 189 | GGGAAGA | 516 | ACCAAGG | 843 |
| | GGCAGG | | AUCCUGC | | AGGCAGG | |
| | AUUCUU | | CUCCUUG | | AUUCUUC | |
| | CCC | | GU | | CC | |
| Pt2744 | AAGGAG | 190 | UGGGAAG | 517 | CCAAGGA | 844 |
| | GCAGGA | | AAUCCUG | | GGCAGGA | |
| | UUCUUC | | CCUCCUU | | UUCUUCC | |
| | CCA | | GG | | CA | |
| Pt2745 | AGGAGG | 191 | AUGGGAA | 518 | CAAGGAG | 845 |
| | CAGGAU | | GAAUCCU | | GCAGGAU | |
| | UCUUCCC | | GCCUCCU | | UCUUCCC | |
| | AU | | UG | | AU | |
| Pt2746 | GGAGGC | 192 | CAUGGGA | 519 | AAGGAGG | 846 |
| | AGGAUU | | AGAAUCC | | CAGGAUU | |
| | CUUCCCA | | UGCCUCC | | CUUCCCA | |
| | UG | | UU | | UG | |
| Pt2747 | GAGGCA | 193 | CCAUGGG | 520 | AGGAGGC | 847 |
| | GGAUUC | | AAGAAUC | | AGGAUUC | |
| | UUCCCA | | CUGCCUC | | UUCCCAU | |
| | UGG | | CU | | GG | |
| Pt2748 | AGGCAG | 194 | UCCAUGG | 521 | GGAGGCA | 848 |
| | GAUUCU | | GAAGAAU | | GGAUUCU | |
| | UCCCAU | | CCUGCCU | | UCCCAUG | |
| | GGA | | CC | | GA | |
| Pt2750 | GCAGGA | 195 | UAUCCAU | 522 | AGGCAGG | 849 |
| | UUCUUC | | GGGAAGA | | AUUCUUC | |
| | CCAUGG | | AUCCUGC | | CCAUGGA | |
| | AUA | | CU | | UA | |
| Pt2752 | AGGAUU | 196 | CCUAUCC | 523 | GCAGGAU | 850 |
| | CUUCCCA | | AUGGGAA | | UCUUCCC | |
| | UGGAUA | | GAAUCCU | | AUGGAUA | |
| | GG | | GC | | GG | |
| Pt2753 | GGAUUC | 197 | CCCUAUC | 524 | CAGGAUU | 851 |
| | UUCCCA | | CAUGGGA | | CUUCCCA | |
| | UGGAUA | | AGAAUCC | | UGGAUAG | |
| | GGG | | UG | | GG | |
| Pt2789 | GCUGCA | 198 | AACGAUG | 525 | GGGCUGC | 852 |
| | GGGACA | | UUUGUCC | | AGGGACA | |
| | AACAUC | | CUGCAGC | | AACAUCG | |
| | GUU | | CC | | UU | |
| Pt2790 | CUGCAG | 199 | CAACGAU | 526 | GGCUGCA | 853 |
| | GGACAA | | GUUUGUC | | GGGACAA | |
| | ACAUCG | | CCUGCAG | | ACAUCGU | |
| | UUG | | CC | | UG | |
| Pt2791 | UGCAGG | 200 | CCAACGA | 527 | GCUGCAG | 854 |
| | GACAAA | | UGUUUGU | | GGACAAA | |
| | CAUCGU | | CCCUGCA | | CAUCGUU | |
| | UGG | | GC | | GG | |
| Pt2792 | GCAGGG | 201 | CCCAACG | 528 | CUGCAGG | 855 |
| | ACAAAC | | AUGUUUG | | GACAAAC | |
| | AUCGUU | | UCCCUGC | | AUCGUUG | |
| | GGG | | AG | | GG | |
| Pt2815 | GUGAGU | 202 | AUCAGCA | 529 | GGGUGAG | 856 |
| | GUGAAA | | CCUUUCA | | UGUGAAA | |
| | GGUGCU | | CACUCAC | | GGUGCUG | |
| | GAU | | CC | | AU | |
| Pt2816 | UGAGUG | 203 | CAUCAGC | 530 | GGUGAGU | 857 |
| | UGAAAG | | ACCUUUC | | GUGAAAG | |
| | GUGCUG | | ACACUCA | | GUGCUGA | |
| | AUG | | CC | | UG | |
| Pt2818 | AGUGUG | 204 | GCCAUCA | 531 | UGAGUGU | 858 |
| | AAAGGU | | GCACCUU | | GAAAGGU | |
| | GCUGAU | | UCACACU | | GCUGAUG | |
| | GGC | | CA | | GC | |
| Pt2819 | GUGUGA | 205 | GGCCAUC | 532 | GAGUGUG | 859 |
| | AAGGUG | | AGCACCU | | AAAGGUG | |
| | CUGAUG | | UUCACAC | | CUGAUGG | |
| | GCC | | UC | | CC | |
| Pt2820 | UGUGAA | 206 | GGGCCAU | 533 | AGUGUGA | 860 |
| | AGGUGC | | CAGCACC | | AAGGUGC | |
| | UGAUGG | | UUUCACA | | UGAUGGC | |
| | CCC | | CU | | CC | |
| Pt2821 | GUGAAA | 207 | AGGGCCA | 534 | GUGUGAA | 861 |
| | GGUGCU | | UCAGCAC | | AGGUGCU | |
| | GAUGGC | | CUUUCAC | | GAUGGCC | |
| | CCU | | AC | | CU | |
| Pt2822 | UGAAAG | 208 | GAGGGCC | 535 | UGUGAAA | 862 |
| | GUGCUG | | AUCAGCA | | GGUGCUG | |
| | AUGGCC | | CCUUUCA | | AUGGCCC | |
| | CUC | | CA | | UC | |
| Pt2823 | GAAAGG | 209 | UGAGGGC | 536 | GUGAAAG | 863 |
| | UGCUGA | | CAUCAGC | | GUGCUGA | |
| | UGGCCC | | ACCUUUC | | UGGCCCU | |
| | UCA | | AC | | CA | |
| Pt2837 | GCCCUCA | 210 | CAGUUAG | 537 | UGGCCCU | 864 |
| | UCUCCA | | CUGGAGA | | CAUCUCC | |
| | GCUAAC | | UGAGGGC | | AGCUAAC | |
| | UG | | CA | | UG | |
| Pt2838 | CCCUCAU | 211 | ACAGUUA | 538 | GGCCCUC | 865 |
| | CUCCAGC | | GCUGGAG | | AUCUCCA | |
| | UAACUG | | AUGAGGG | | GCUAACU | |
| | U | | CC | | GU | |
| Pt2839 | CCUCAUC | 212 | CACAGUU | 539 | GCCCUCA | 866 |
| | UCCAGC | | AGCUGGA | | UCUCCAG | |
| | UAACUG | | GAUGAGG | | CUAACUG | |
| | UG | | GC | | UG | |
| Pt2840 | CUCAUC | 213 | CCACAGU | 540 | CCCUCAU | 867 |
| | UCCAGC | | UAGCUGG | | CUCCAGC | |
| | UAACUG | | AGAUGAG | | UAACUGU | |
| | UGG | | GG | | GG | |
| Pt2850 | GCUAAC | 214 | AGGGGCU | 541 | CAGCUAA | 868 |
| | UGUGGA | | UCUCCAC | | CUGUGGA | |
| | GAAGCC | | AGUUAGC | | GAAGCCC | |
| | CCU | | UG | | CU | |
| Pt2851 | CUAACU | 215 | CAGGGGC | 542 | AGCUAAC | 869 |
| | GUGGAG | | UUCUCCA | | UGUGGAG | |
| | AAGCCCC | | CAGUUAG | | AAGCCCC | |
| | UG | | CU | | UG | |
| Pt2879 | CCUGAU | 216 | GCUAAGC | 543 | UCCCUGA | 870 |
| | UAAUGG | | CUCCAUU | | UUAAUGG | |
| | AGGCUU | | AAUCAGG | | AGGCUUA | |
| | AGC | | GA | | GC | |
| Pt2881 | UGAUUA | 217 | AAGCUAA | 544 | CCUGAUU | 871 |
| | AUGGAG | | GCCUCCA | | AAUGGAG | |
| | GCUUAG | | UUAAUCA | | GCUUAGC | |
| | CUU | | GG | | UU | |
| Pt2882 | GAUUAA | 218 | AAAGCUA | 545 | CUGAUUA | 872 |
| | UGGAGG | | AGCCUCC | | AUGGAGG | |
| | CUUAGC | | AUUAAUC | | CUUAGCU | |
| | UUU | | AG | | UU | |
| Pt2883 | AUUAAU | 219 | GAAAGCU | 546 | UGAUUAA | 873 |
| | GGAGGC | | AAGCCUC | | UGGAGGC | |
| | UUAGCU | | CAUUAAU | | UUAGCUU | |
| | UUC | | CA | | UC | |
| Pt2885 | UAAUGG | 220 | CAGAAAG | 547 | AUUAAUG | 874 |
| | AGGCUU | | CUAAGCC | | GAGGCUU | |
| | AGCUUU | | UCCAUUA | | AGCUUUC | |
| | CUG | | AU | | UG | |
| Pt2886 | AAUGGA | 221 | CCAGAAA | 548 | UUAAUGG | 875 |
| | GGCUUA | | GCUAAGC | | AGGCUUA | |
| | GCUUUC | | CUCCAUU | | GCUUUCU | |
| | UGG | | AA | | GG | |
| Pt2887 | AUGGAG | 222 | UCCAGAA | 549 | UAAUGGA | 876 |
| | GCUUAG | | AGCUAAG | | GGCUUAG | |
| | CUUUCU | | CCUCCAU | | CUUUCUG | |
| | GGA | | UA | | GA | |
| Pt2888 | UGGAGG | 223 | AUCCAGA | 550 | AAUGGAG | 877 |
| | CUUAGC | | AAGCUAA | | GCUUAGC | |
| | UUUCUG | | GCCUCCA | | UUUCUGG | |
| | GAU | | UU | | AU | |
| Pt2889 | GGAGGC | 224 | CAUCCAG | 551 | AUGGAGG | 878 |
| | UUAGCU | | AAAGCUA | | CUUAGCU | |
| | UUCUGG | | AGCCUCC | | UUCUGGA | |
| | AUG | | AU | | UG | |
| Pt2890 | GAGGCU | 225 | CCAUCCA | 552 | UGGAGGC | 879 |
| | UAGCUU | | GAAAGCU | | UUAGCUU | |
| | UCUGGA | | AAGCCUC | | UCUGGAU | |
| | UGG | | CA | | GG | |
| Pt2891 | AGGCUU | 226 | GCCAUCC | 553 | GGAGGCU | 880 |
| | AGCUUU | | AGAAAGC | | UAGCUUU | |
| | CUGGAU | | UAAGCCU | | CUGGAUG | |
| | GGC | | CC | | GC | |
| Pt2893 | GCUUAG | 227 | AUGCCAU | 554 | AGGCUUA | 881 |
| | CUUUCU | | CCAGAAA | | GCUUUCU | |
| | GGAUGG | | GCUAAGC | | GGAUGGC | |
| | CAU | | CU | | AU | |
| Pt2959 | GUGCCU | 228 | UGGCUCA | 555 | CUGUGCC | 882 |
| | UGGUUU | | GGAAACC | | UUGGUUU | |
| | CCUGAG | | AAGGCAC | | CCUGAGC | |
| | CCA | | AG | | CA | |
| Pt2960 | UGCCUU | 229 | GUGGCUC | 556 | UGUGCCU | 883 |
| | GGUUUC | | AGGAAAC | | UGGUUUC | |
| | CUGAGC | | CAAGGCA | | CUGAGCC | |
| | CAC | | CA | | AC | |
| Pt2962 | CCUUGG | 230 | AGGUGGC | 557 | UGCCUUG | 884 |
| | UUUCCU | | UCAGGAA | | GUUUCCU | |
| | GAGCCA | | ACCAAGG | | GAGCCAC | |
| | CCU | | CA | | CU | |
| Pt2963 | CUUGGU | 231 | AAGGUGG | 558 | GCCUUGG | 885 |
| | UUCCUG | | CUCAGGA | | UUUCCUG | |
| | AGCCACC | | AACCAAG | | AGCCACC | |
| | UU | | GC | | UU | |
| Pt2964 | UUGGUU | 232 | AAAGGUG | 559 | CCUUGGU | 886 |
| | UCCUGA | | GCUCAGG | | UUCCUGA | |
| | GCCACCU | | AAACCAA | | GCCACCU | |
| | UU | | GG | | UU | |
| Pt2965 | UGGUUU | 233 | UAAAGGU | 560 | CUUGGUU | 887 |
| | CCUGAG | | GGCUCAG | | UCCUGAG | |
| | CCACCUU | | GAAACCA | | CCACCUU | |
| | UA | | AG | | UA | |
| Pt2966 | GGUUUC | 234 | GUAAAGG | 561 | UUGGUUU | 888 |
| | CUGAGC | | UGGCUCA | | CCUGAGC | |
| | CACCUU | | GGAAACC | | CACCUUU | |
| | UAC | | AA | | AC | |
| Pt2967 | GUUUCC | 235 | AGUAAAG | 562 | UGGUUUC | 889 |
| | UGAGCC | | GUGGCUC | | CUGAGCC | |
| | ACCUUU | | AGGAAAC | | ACCUUUA | |
| | ACU | | CA | | CU | |
| Pt2968 | UUUCCU | 236 | GAGUAAA | 563 | GGUUUCC | 890 |
| | GAGCCA | | GGUGGCU | | UGAGCCA | |
| | CCUUUA | | CAGGAAA | | CCUUUAC | |
| | CUC | | CC | | UC | |
| Pt2969 | UUCCUG | 237 | AGAGUAA | 564 | GUUUCCU | 891 |
| | AGCCACC | | AGGUGGC | | GAGCCAC | |
| | UUUACU | | UCAGGAA | | CUUUACU | |
| | CU | | AC | | CU | |
| Pt2970 | UCCUGA | 238 | CAGAGUA | 565 | UUUCCUG | 892 |
| | GCCACCU | | AAGGUGG | | AGCCACC | |
| | UUACUC | | CUCAGGA | | UUUACUC | |
| | UG | | AA | | UG | |
| Pt2972 | CUGAGC | 239 | AGCAGAG | 566 | UCCUGAG | 893 |
| | CACCUU | | UAAAGGU | | CCACCUU | |
| | UACUCU | | GGCUCAG | | UACUCUG | |
| | GCU | | GA | | CU | |
| Pt2977 | CCACCUU | 240 | CAUAGAG | 567 | AGCCACC | 894 |
| | UACUCU | | CAGAGUA | | UUUACUC | |
| | GCUCUA | | AAGGUGG | | UGCUCUA | |
| | UG | | CU | | UG | |
| Pt2979 | ACCUUU | 241 | GGCAUAG | 568 | CCACCUU | 895 |
| | ACUCUG | | AGCAGAG | | UACUCUG | |
| | CUCUAU | | UAAAGGU | | CUCUAUG | |
| | GCC | | GG | | CC | |
| Pt2980 | CCUUUA | 242 | UGGCAUA | 569 | CACCUUU | 896 |
| | CUCUGC | | GAGCAGA | | ACUCUGC | |
| | UCUAUG | | GUAAAGG | | UCUAUGC | |
| | CCA | | UG | | CA | |
| Pt2981 | CUUUAC | 243 | CUGGCAU | 570 | ACCUUUA | 897 |
| | UCUGCU | | AGAGCAG | | CUCUGCU | |
| | CUAUGC | | AGUAAAG | | CUAUGCC | |
| | CAG | | GU | | AG | |
| Pt2982 | UUUACU | 244 | CCUGGCA | 571 | CCUUUAC | 898 |
| | CUGCUC | | UAGAGCA | | UCUGCUC | |
| | UAUGCC | | GAGUAAA | | UAUGCCA | |
| | AGG | | GG | | GG | |
| Pt2983 | UUACUC | 245 | GCCUGGC | 572 | CUUUACU | 899 |
| | UGCUCU | | AUAGAGC | | CUGCUCU | |
| | AUGCCA | | AGAGUAA | | AUGCCAG | |
| | GGC | | AG | | GC | |
| Pt2985 | ACUCUG | 246 | CAGCCUG | 573 | UUACUCU | 900 |
| | CUCUAU | | GCAUAGA | | GCUCUAU | |
| | GCCAGG | | GCAGAGU | | GCCAGGC | |
| | CUG | | AA | | UG | |
| Pt2987 | UCUGCU | 247 | CACAGCC | 574 | ACUCUGC | 901 |
| | CUAUGC | | UGGCAUA | | UCUAUGC | |
| | CAGGCU | | GAGCAGA | | CAGGCUG | |
| | GUG | | GU | | UG | |
| Pt2989 | UGCUCU | 248 | AGCACAG | 575 | UCUGCUC | 902 |
| | AUGCCA | | CCUGGCA | | UAUGCCA | |
| | GGCUGU | | UAGAGCA | | GGCUGUG | |
| | GCU | | GA | | CU | |
| Pt2990 | GCUCUA | 249 | UAGCACA | 576 | CUGCUCU | 903 |
| | UGCCAG | | GCCUGGC | | AUGCCAG | |
| | GCUGUG | | AUAGAGC | | GCUGUGC | |
| | CUA | | AG | | UA | |
| Pt2991 | CUCUAU | 250 | CUAGCAC | 577 | UGCUCUA | 904 |
| | GCCAGG | | AGCCUGG | | UGCCAGG | |
| | CUGUGC | | CAUAGAG | | CUGUGCU | |
| | UAG | | CA | | AG | |
| Pt3037 | AGCCAU | 251 | AGUCAGU | 578 | GGAGCCA | 905 |
| | CACCUA | | CCUAGGU | | UCACCUA | |
| | GGACUG | | GAUGGCU | | GGACUGA | |
| | ACU | | CC | | CU | |
| Pt3038 | GCCAUC | 252 | GAGUCAG | 579 | GAGCCAU | 906 |
| | ACCUAG | | UCCUAGG | | CACCUAG | |
| | GACUGA | | UGAUGGC | | GACUGAC | |
| | CUC | | UC | | UC | |
| Pt3039 | CCAUCAC | 253 | CGAGUCA | 580 | AGCCAUC | 907 |
| | CUAGGA | | GUCCUAG | | ACCUAGG | |
| | CUGACU | | GUGAUGG | | ACUGACU | |
| | CG | | CU | | CG | |
| Pt3042 | UCACCU | 254 | UGCCGAG | 581 | CAUCACC | 908 |
| | AGGACU | | UCAGUCC | | UAGGACU | |
| | GACUCG | | UAGGUGA | | GACUCGG | |
| | GCA | | UG | | CA | |
| Pt3043 | CACCUA | 255 | CUGCCGA | 582 | AUCACCU | 909 |
| | GGACUG | | GUCAGUC | | AGGACUG | |
| | ACUCGG | | CUAGGUG | | ACUCGGC | |
| | CAG | | AU | | AG | |
| Pt3125 | CGCACCC | 256 | CCUCUGU | 583 | UUCGCAC | 910 |
| | CUACUU | | GAAGUAG | | CCCUACU | |
| | CACAGA | | GGGUGCG | | UCACAGA | |
| | GG | | AA | | GG | |
| Pt3126 | GCACCCC | 257 | UCCUCUG | 584 | UCGCACC | 911 |
| | UACUUC | | UGAAGUA | | CCUACUU | |
| | ACAGAG | | GGGGUGC | | CACAGAG | |
| | GA | | GA | | GA | |
| Pt3127 | CACCCCU | 258 | UUCCUCU | 585 | CGCACCC | 912 |
| | ACUUCA | | GUGAAGU | | CUACUUC | |
| | CAGAGG | | AGGGGUG | | ACAGAGG | |
| | AA | | CG | | AA | |
| Pt3138 | CACAGA | 259 | UUCCAGG | 586 | UUCACAG | 913 |
| | GGAAGA | | UUUCUUC | | AGGAAGA | |
| | AACCUG | | CUCUGUG | | AACCUGG | |
| | GAA | | AA | | AA | |
| Pt3139 | ACAGAG | 260 | GUUCCAG | 587 | UCACAGA | 914 |
| | GAAGAA | | GUUUCUU | | GGAAGAA | |
| | ACCUGG | | CCUCUGU | | ACCUGGA | |
| | AAC | | GA | | AC | |
| Pt3141 | AGAGGA | 261 | UGGUUCC | 588 | ACAGAGG | 915 |
| | AGAAAC | | AGGUUUC | | AAGAAAC | |
| | CUGGAA | | UUCCUCU | | CUGGAAC | |
| | CCA | | GU | | CA | |
| Pt3177 | CCAAGC | 262 | GUUCCUG | 589 | UGCCAAG | 916 |
| | UCACAC | | CUGUGUG | | CUCACAC | |
| | AGCAGG | | AGCUUGG | | AGCAGGA | |
| | AAC | | CA | | AC | |
| Pt3178 | CAAGCU | 263 | AGUUCCU | 590 | GCCAAGC | 917 |
| | CACACA | | GCUGUGU | | UCACACA | |
| | GCAGGA | | GAGCUUG | | GCAGGAA | |
| | ACU | | GC | | CU | |
| Pt3179 | AAGCUC | 264 | CAGUUCC | 591 | CCAAGCU | 918 |
| | ACACAG | | UGCUGUG | | CACACAG | |
| | CAGGAA | | UGAGCUU | | CAGGAAC | |
| | CUG | | GG | | UG | |
| Pt3180 | AGCUCA | 265 | UCAGUUC | 592 | CAAGCUC | 919 |
| | CACAGC | | CUGCUGU | | ACACAGC | |
| | AGGAAC | | GUGAGCU | | AGGAACU | |
| | UGA | | UG | | GA | |
| Pt3185 | ACACAG | 266 | CUGGCUC | 593 | UCACACA | 920 |
| | CAGGAA | | AGUUCCU | | GCAGGAA | |
| | CUGAGC | | GCUGUGU | | CUGAGCC | |
| | CAG | | GA | | AG | |
| Pt3186 | CACAGC | 267 | UCUGGCU | 594 | CACACAG | 921 |
| | AGGAAC | | CAGUUCC | | CAGGAAC | |
| | UGAGCC | | UGCUGUG | | UGAGCCA | |
| | AGA | | UG | | GA | |
| Pt3187 | ACAGCA | 268 | UUCUGGC | 595 | ACACAGC | 922 |
| | GGAACU | | UCAGUUC | | AGGAACU | |
| | GAGCCA | | CUGCUGU | | GAGCCAG | |
| | GAA | | GU | | AA | |
| Pt3188 | CAGCAG | 269 | UUUCUGG | 596 | CACAGCA | 923 |
| | GAACUG | | CUCAGUU | | GGAACUG | |
| | AGCCAG | | CCUGCUG | | AGCCAGA | |
| | AAA | | UG | | AA | |
| Pt3189 | AGCAGG | 270 | GUUUCUG | 597 | ACAGCAG | 924 |
| | AACUGA | | GCUCAGU | | GAACUGA | |
| | GCCAGA | | UCCUGCU | | GCCAGAA | |
| | AAC | | GU | | AC | |
| Pt3190 | GCAGGA | 271 | CGUUUCU | 598 | CAGCAGG | 925 |
| | ACUGAG | | GGCUCAG | | AACUGAG | |
| | CCAGAA | | UUCCUGC | | CCAGAAA | |
| | ACG | | UG | | CG | |
| Pt3191 | CAGGAA | 272 | GCGUUUC | 599 | AGCAGGA | 926 |
| | CUGAGC | | UGGCUCA | | ACUGAGC | |
| | CAGAAA | | GUUCCUG | | CAGAAAC | |
| | CGC | | CU | | GC | |
| Pt3192 | AGGAAC | 273 | UGCGUUU | 600 | GCAGGAA | 927 |
| | UGAGCC | | CUGGCUC | | CUGAGCC | |
| | AGAAAC | | AGUUCCU | | AGAAACG | |
| | GCA | | GC | | CA | |
| Pt3193 | GGAACU | 274 | CUGCGUU | 601 | CAGGAAC | 928 |
| | GAGCCA | | UCUGGCU | | UGAGCCA | |
| | GAAACG | | CAGUUCC | | GAAACGC | |
| | CAG | | UG | | AG | |
| Pt3194 | GAACUG | 275 | UCUGCGU | 602 | AGGAACU | 929 |
| | AGCCAG | | UUCUGGC | | GAGCCAG | |
| | AAACGC | | UCAGUUC | | AAACGCA | |
| | AGA | | CU | | GA | |
| Pt3199 | GAGCCA | 276 | CCCAAUC | 603 | CUGAGCC | 930 |
| | GAAACG | | UGCGUUU | | AGAAACG | |
| | CAGAUU | | CUGGCUC | | CAGAUUG | |
| | GGG | | AG | | GG | |
| Pt3200 | AGCCAG | 277 | GCCCAAU | 604 | UGAGCCA | 931 |
| | AAACGC | | CUGCGUU | | GAAACGC | |
| | AGAUUG | | UCUGGCU | | AGAUUGG | |
| | GGC | | CA | | GC | |
| Pt3205 | GAAACG | 278 | AGCCAGC | 605 | CAGAAAC | 932 |
| | CAGAUU | | CCAAUCU | | GCAGAUU | |
| | GGGCUG | | GCGUUUC | | GGGCUGG | |
| | GCU | | UG | | CU | |
| Pt3206 | AAACGC | 279 | GAGCCAG | 606 | AGAAACG | 933 |
| | AGAUUG | | CCCAAUC | | CAGAUUG | |
| | GGCUGG | | UGCGUUU | | GGCUGGC | |
| | CUC | | CU | | UC | |
| Pt3207 | AACGCA | 280 | AGAGCCA | 607 | GAAACGC | 934 |
| | GAUUGG | | GCCCAAU | | AGAUUGG | |
| | GCUGGC | | CUGCGUU | | GCUGGCU | |
| | UCU | | UC | | CU | |
| Pt3234 | AAGCCU | 281 | GGGUGAA | 608 | CCAAGCC | 935 |
| | CUUCUU | | GUAAGAA | | UCUUCUU | |
| | ACUUCA | | GAGGCUU | | ACUUCAC | |
| | CCC | | GG | | CC | |
| Pt3235 | AGCCUC | 282 | CGGGUGA | 609 | CAAGCCU | 936 |
| | UUCUUA | | AGUAAGA | | CUUCUUA | |
| | CUUCACC | | AGAGGCU | | CUUCACC | |
| | CG | | UG | | CG | |
| Pt3236 | GCCUCU | 283 | CCGGGUG | 610 | AAGCCUC | 937 |
| | UCUUAC | | AAGUAAG | | UUCUUAC | |
| | UUCACCC | | AAGAGGC | | UUCACCC | |
| | GG | | UU | | GG | |
| Pt3237 | CCUCUUC | 284 | GCCGGGU | 611 | AGCCUCU | 938 |
| | UUACUU | | GAAGUAA | | UCUUACU | |
| | CACCCGG | | GAAGAGG | | UCACCCG | |
| | C | | CU | | GC | |
| Pt3238 | CUCUUC | 285 | AGCCGGG | 612 | GCCUCUU | 939 |
| | UUACUU | | UGAAGUA | | CUUACUU | |
| | CACCCGG | | AGAAGAG | | CACCCGG | |
| | CU | | GC | | CU | |
| Pt3239 | UCUUCU | 286 | CAGCCGG | 613 | CCUCUUC | 940 |
| | UACUUC | | GUGAAGU | | UUACUUC | |
| | ACCCGGC | | AAGAAGA | | ACCCGGC | |
| | UG | | GG | | UG | |
| Pt3240 | CUUCUU | 287 | CCAGCCG | 614 | CUCUUCU | 941 |
| | ACUUCA | | GGUGAAG | | UACUUCA | |
| | CCCGGCU | | UAAGAAG | | CCCGGCU | |
| | GG | | AG | | GG | |
| Pt3241 | UUCUUA | 288 | CCCAGCC | 615 | UCUUCUU | 942 |
| | CUUCACC | | GGGUGAA | | ACUUCAC | |
| | CGGCUG | | GUAAGAA | | CCGGCUG | |
| | GG | | GA | | GG | |
| Pt3243 | CUUACU | 289 | AGCCCAG | 616 | UUCUUAC | 943 |
| | UCACCCG | | CCGGGUG | | UUCACCC | |
| | GCUGGG | | AAGUAAG | | GGCUGGG | |
| | CU | | AA | | CU | |
| Pt3275 | CGGGUA | 290 | UCCCAGC | 617 | UACGGGU | 944 |
| | ACAGUG | | CUCACUG | | AACAGUG | |
| | AGGCUG | | UUACCCG | | AGGCUGG | |
| | GGA | | UA | | GA | |
| Pt3276 | GGGUAA | 291 | UUCCCAG | 618 | ACGGGUA | 945 |
| | CAGUGA | | CCUCACU | | ACAGUGA | |
| | GGCUGG | | GUUACCC | | GGCUGGG | |
| | GAA | | GU | | AA | |
| Pt3302 | ACACAG | 292 | ACCGAGC | 619 | GAACACA | 946 |
| | ACCAGG | | UUCCUGG | | GACCAGG | |
| | AAGCUC | | UCUGUGU | | AAGCUCG | |
| | GGU | | UC | | GU | |
| Pt3303 | CACAGA | 293 | CACCGAG | 620 | AACACAG | 947 |
| | CCAGGA | | CUUCCUG | | ACCAGGA | |
| | AGCUCG | | GUCUGUG | | AGCUCGG | |
| | GUG | | UU | | UG | |
| Pt3304 | ACAGAC | 294 | UCACCGA | 621 | ACACAGA | 948 |
| | CAGGAA | | GCUUCCU | | CCAGGAA | |
| | GCUCGG | | GGUCUGU | | GCUCGGU | |
| | UGA | | GU | | GA | |
| Pt3306 | AGACCA | 295 | ACUCACC | 622 | ACAGACC | 949 |
| | GGAAGC | | GAGCUUC | | AGGAAGC | |
| | UCGGUG | | CUGGUCU | | UCGGUGA | |
| | AGU | | GU | | GU | |
| Pt3308 | ACCAGG | 296 | UCACUCA | 623 | AGACCAG | 950 |
| | AAGCUC | | CCGAGCU | | GAAGCUC | |
| | GGUGAG | | UCCUGGU | | GGUGAGU | |
| | UGA | | CU | | GA | |
| Pt3309 | CCAGGA | 297 | AUCACUC | 624 | GACCAGG | 951 |
| | AGCUCG | | ACCGAGC | | AAGCUCG | |
| | GUGAGU | | UUCCUGG | | GUGAGUG | |
| | GAU | | UC | | AU | |
| Pt3310 | CAGGAA | 298 | CAUCACU | 625 | ACCAGGA | 952 |
| | GCUCGG | | CACCGAG | | AGCUCGG | |
| | UGAGUG | | CUUCCUG | | UGAGUGA | |
| | AUG | | GU | | UG | |
| Pt3313 | GAAGCU | 299 | UGCCAUC | 626 | AGGAAGC | 953 |
| | CGGUGA | | ACUCACC | | UCGGUGA | |
| | GUGAUG | | GAGCUUC | | GUGAUGG | |
| | GCA | | CU | | CA | |
| Pt3315 | AGCUCG | 300 | UCUGCCA | 627 | GAAGCUC | 954 |
| | GUGAGU | | UCACUCA | | GGUGAGU | |
| | GAUGGC | | CCGAGCU | | GAUGGCA | |
| | AGA | | UC | | GA | |
| Pt3317 | CUCGGU | 301 | GUUCUGC | 628 | AGCUCGG | 955 |
| | GAGUGA | | CAUCACU | | UGAGUGA | |
| | UGGCAG | | CACCGAG | | UGGCAGA | |
| | AAC | | CU | | AC | |
| Pt3319 | CGGUGA | 302 | UCGUUCU | 629 | CUCGGUG | 956 |
| | GUGAUG | | GCCAUCA | | AGUGAUG | |
| | GCAGAA | | CUCACCG | | GCAGAAC | |
| | CGA | | AG | | GA | |
| Pt3321 | GUGAGU | 303 | CAUCGUU | 630 | CGGUGAG | 957 |
| | GAUGGC | | CUGCCAU | | UGAUGGC | |
| | AGAACG | | CACUCAC | | AGAACGA | |
| | AUG | | CG | | UG | |
| Pt3322 | UGAGUG | 304 | GCAUCGU | 631 | GGUGAGU | 958 |
| | AUGGCA | | UCUGCCA | | GAUGGCA | |
| | GAACGA | | UCACUCA | | GAACGAU | |
| | UGC | | CC | | GC | |
| Pt3382 | AUUCAC | 305 | AUCUCCG | 632 | UGAUUCA | 959 |
| | UGGCCU | | CCAGGCC | | CUGGCCU | |
| | GGCGGA | | AGUGAAU | | GGCGGAG | |
| | GAU | | CA | | AU | |
| Pt3390 | GCCUGG | 306 | UUAGAAG | 633 | UGGCCUG | 960 |
| | CGGAGA | | CAUCUCC | | GCGGAGA | |
| | UGCUUC | | GCCAGGC | | UGCUUCU | |
| | UAA | | CA | | AA | |
| Pt3394 | GGCGGA | 307 | UGCCUUA | 634 | CUGGCGG | 961 |
| | GAUGCU | | GAAGCAU | | AGAUGCU | |
| | UCUAAG | | CUCCGCC | | UCUAAGG | |
| | GCA | | AG | | CA | |
| Pt3431 | CCAACA | 308 | UCAAGGA | 635 | GGCCAAC | 962 |
| | ACUGUC | | GGGACAG | | AACUGUC | |
| | CCUCCUU | | UUGUUGG | | CCUCCUU | |
| | GA | | CC | | GA | |
| Pt3432 | CAACAA | 309 | CUCAAGG | 636 | GCCAACA | 963 |
| | CUGUCCC | | AGGGACA | | ACUGUCC | |
| | UCCUUG | | GUUGUUG | | CUCCUUG | |
| | AG | | GC | | AG | |
| Pt3433 | AACAAC | 310 | GCUCAAG | 637 | CCAACAA | 964 |
| | UGUCCC | | GAGGGAC | | CUGUCCC | |
| | UCCUUG | | AGUUGUU | | UCCUUGA | |
| | AGC | | GG | | GC | |
| Pt3434 | ACAACU | 311 | UGCUCAA | 638 | CAACAAC | 965 |
| | GUCCCUC | | GGAGGGA | | UGUCCCU | |
| | CUUGAG | | CAGUUGU | | CCUUGAG | |
| | CA | | UG | | CA | |
| Pt3435 | CAACUG | 312 | GUGCUCA | 639 | AACAACU | 966 |
| | UCCCUCC | | AGGAGGG | | GUCCCUC | |
| | UUGAGC | | ACAGUUG | | CUUGAGC | |
| | AC | | UU | | AC | |
| Pt3436 | AACUGU | 313 | GGUGCUC | 640 | ACAACUG | 967 |
| | CCCUCCU | | AAGGAGG | | UCCCUCC | |
| | UGAGCA | | GACAGUU | | UUGAGCA | |
| | CC | | GU | | CC | |
| Pt3437 | ACUGUC | 314 | UGGUGCU | 641 | CAACUGU | 968 |
| | CCUCCUU | | CAAGGAG | | CCCUCCU | |
| | GAGCAC | | GGACAGU | | UGAGCAC | |
| | CA | | UG | | CA | |
| Pt3438 | CUGUCCC | 315 | CUGGUGC | 642 | AACUGUC | 969 |
| | UCCUUG | | UCAAGGA | | CCUCCUU | |
| | AGCACC | | GGGACAG | | GAGCACC | |
| | AG | | UU | | AG | |
| Pt3461 | CCACCCA | 316 | AAUGUCU | 643 | CCCCACC | 970 |
| | AGCAAG | | GCUUGCU | | CAAGCAA | |
| | CAGACA | | UGGGUGG | | GCAGACA | |
| | UU | | GG | | UU | |
| Pt3465 | CCAAGC | 317 | GAUAAAU | 644 | ACCCAAG | 971 |
| | AAGCAG | | GUCUGCU | | CAAGCAG | |
| | ACAUUU | | UGCUUGG | | ACAUUUA | |
| | AUC | | GU | | UC | |
| Pt3468 | AGCAAG | 318 | AAAGAUA | 645 | CAAGCAA | 972 |
| | CAGACA | | AAUGUCU | | GCAGACA | |
| | UUUAUC | | GCUUGCU | | UUUAUCU | |
| | UUU | | UG | | UU | |
| Pt3490 | GGGUCU | 319 | GGCAACA | 646 | UUGGGUC | 973 |
| | GUCCUC | | GAGAGGA | | UGUCCUC | |
| | UCUGUU | | CAGACCC | | UCUGUUG | |
| | GCC | | AA | | CC | |
| Pt3491 | GGUCUG | 320 | AGGCAAC | 647 | UGGGUCU | 974 |
| | UCCUCUC | | AGAGAGG | | GUCCUCU | |
| | UGUUGC | | ACAGACC | | CUGUUGC | |
| | CU | | CA | | CU | |
| Pt3492 | GUCUGU | 321 | AAGGCAA | 648 | GGGUCUG | 975 |
| | CCUCUCU | | CAGAGAG | | UCCUCUC | |
| | GUUGCC | | GACAGAC | | UGUUGCC | |
| | UU | | CC | | UU | |
| Pt3493 | UCUGUC | 322 | AAAGGCA | 649 | GGUCUGU | 976 |
| | CUCUCU | | ACAGAGA | | CCUCUCU | |
| | GUUGCC | | GGACAGA | | GUUGCCU | |
| | UUU | | CC | | UU | |
| Pt3552 | CUUGAA | 323 | CCCAGAA | 650 | AACUUGA | 977 |
| | GAUAUU | | UAAAUAU | | AGAUAUU | |
| | UAUUCU | | CUUCAAG | | UAUUCUG | |
| | GGG | | UU | | GG | |
| Pt3553 | UUGAAG | 324 | ACCCAGA | 651 | ACUUGAA | 978 |
| | AUAUUU | | AUAAAUA | | GAUAUUU | |
| | AUUCUG | | UCUUCAA | | AUUCUGG | |
| | GGU | | GU | | GU | |
| PPC1 | CUGUUU | 325 | CAAGUUA | 652 | ACCUGUU | 979 |
| | UGCUUU | | CAAAAGC | | UUGCUUU | |
| | UGUAAC | | AAAACAG | | UGUAACU | |
| | UUG | | GU | | UG | |
| PPC2 | CUAGAC | 326 | ACAAAAG | 653 | UUCUAGA | 980 |
| | CUGUTU | | CAAAACA | | CCUGUUU | |
| | UGCUUU | | GGUCUAG | | UGCUUUU | |
| | UGU | | AA | | GU | |
| P341-2 | AACUUU | 327 | AAACAGG | 654 | AACUUUU | 981 |
| | UCUAGA | | UCUAGAA | | CUAGACC | |
| | CCUGUU | | AAGUUTT | | UGUUU | |
| | UTT | | | | | |
| P3119M | CmsAmsC | 982 | UmsGfsAm | 1045 | UACACAU | 681 |
| W02 | mAmUmU | | AmGmUmA | | UCGCACC | |
| | mCfGmCf | | mGmGmGm | | CCUACUU | |
| | AfCfCmC | | GmUmGmC | | CA | |
| | mCmUmA | | fGmAfAmU | | | |
| | mCmUmU | | mGmUmGm | | | |
| | mCmAm | | sUmsAm | | | |
| P3196M | AmsCmsU | 983 | AmsAfsUm | 1046 | GAACUGA | 686 |
| W02 | mGmAmG | | CmUmGmC | | GCCAGAA | |
| | mCfCmAf | | mGmUmUm | | ACGCAGA | |
| | GfAfAmA | | UmCmUmG | | UU | |
| | mCmGmC | | fGmCfUmC | | | |
| | mAmGmA | | mAmGmUm | | | |
| | mUmUm | | sUmsCm | | | |
| P3464M | CmsCmsC | 984 | AmsUfsAm | 1047 | CACCCAA | 691 |
| W02 | mAmAmG | | AmAmUmG | | GCAAGCA | |
| | mCfAmAf | | mUmCmUm | | GACAUUU | |
| | GfCfAmG | | GmCmUmU | | AU | |
| | mAmCmA | | fGmCfUmU | | | |
| | mUmUmU | | mGmGmGm | | | |
| | mAmUm | | sUmsGm | | | |
| P3543M | CmsUmsU | 985 | AmsAfsUm | 1048 | UGCUUUU | 694 |
| W02 | mUmUmG | | AmUmCmU | | GUAACUU | |
| | mUfAmAf | | mUmCmAm | | GAAGAUA | |
| | CfUfUmG | | AmGmUmU | | UU | |
| | mAmAmG | | fAmCfAmA | | | |
| | mAmUmA | | mAmAmGm | | | |
| | mUmUm | | sCmsAm | | | |
| P3554M | UmsGmsA | 986 | AmsAfsCm | 1049 | CUUGAAG | 696 |
| W02 | mAmGmA | | CmCmAmG | | AUAUUUA | |
| | mUfAmUf | | mAmAmUm | | UUCUGGG | |
| | UfUfAmU | | AmAmAmU | | UU | |
| | mUmCmU | | fAmUfCmU | | | |
| | mGmGmG | | mUmCmAm | | | |
| | mUmUm | | sAmsGm | | | |
| SP340M | GmsCmsC | 987 | AmsAfsCm | 1050 | CAGCCAA | 753 |
| W02 | mAmAmC | | AmGmGmU | | CUUUUCU | |
| | mUfUmUf | | mCmUmAm | | AGACCUG | |
| | UfCfUmA | | GmAmAmA | | UU | |
| | mGmAmC | | fAmGfUmU | | | |
| | mCmUmG | | mGmGmCm | | | |
| | mUmUm | | sUmsGm | | | |
| SP341M | CmsCmsA | 988 | AmsAfsAm | 1051 | AGCCAAC | 754 |
| W02 | mAmCmU | | CmAmGmG | | UUUUCUA | |
| | mUfUmUf | | mUmCmUm | | GACCUGU | |
| | CfUfAmG | | AmGmAmA | | UU | |
| | mAmCmC | | fAmAfGmU | | | |
| | mUmGmU | | mUmGmGm | | | |
| | mUmUm | | sCmsUm | | | |
| SP342M | CmsAmsA | 989 | AmsAfsAm | 1052 | GCCAACU | 755 |
| W02 | mCmUmU | | AmCmAmG | | UUUCUAG | |
| | mUfUmCf | | mGmUmCm | | ACCUGUU | |
| | UfAfGmA | | UmAmGmA | | UU | |
| | mCmCmU | | fAmAfAmG | | | |
| | mGmUmU | | mUmUmGm | | | |
| | mUmUm | | sGmsCm | | | |
| SP344M | AmsCmsU | 990 | GmsCfsAm | 1053 | CAACUUU | 757 |
| W02 | mUmUmU | | AmAmAmC | | UCUAGAC | |
| | mCfUmAf | | mAmGmGm | | CUGUUUU | |
| | GfAfCmC | | UmCmUmA | | GC | |
| | mUmGmU | | fGmAfAmA | | | |
| | mUmUmU | | mAmGmUm | | | |
| | mGmCm | | sUmsGm | | | |
| SP345M | CmsUmsU | 991 | AmsGfsCm | 1054 | AACUUUU | 758 |
| W02 | mUmUmC | | AmAmAmA | | CUAGACC | |
| | mUfAmGf | | mCmAmGm | | UGUUUUG | |
| | AfCfCmU | | GmUmCmU | | CU | |
| | mGmUmU | | fAmGfAmA | | | |
| | mUmUmG | | mAmAmGm | | | |
| | mCmUm | | sUmsUm | | | |
| SP349M | UmsCmsU | 992 | CmsAfsAm | 1055 | UUUCUAG | 760 |
| W02 | mAmGmA | | AmAmGmC | | ACCUGUU | |
| | mCfCmUf | | mAmAmAm | | UUGCUUU | |
| | GfUfUmU | | AmCmAmG | | UG | |
| | mUmGmC | | fGmUfCmU | | | |
| | mUmUmU | | mAmGmAm | | | |
| | mUmGm | | sAmsAm | | | |
| SP351M | AmsGmsA | 993 | UmsUfsAm | 1056 | CUAGACC | 761 |
| W02 | mCmCmU | | CmAmAmA | | UGUUUUG | |
| | mGfUmUf | | mAmGmCm | | CUUUUGU | |
| | UfUfGmC | | AmAmAmA | | AA | |
| | mUmUmU | | fCmAfGmG | | | |
| | mUmGmU | | mUmCmUm | | | |
| | mAmAm | | sAmsGm | | | |
| WP3239 | GmsGmsG | 994 | UmsAfsGm | 1057 | CAGGGUA | 1108 |
| MW02 | mUmAmC | | GmGmGmU | | CACAUUC | |
| | mAfCmAf | | mGmCmGm | | GCACCCC | |
| | UfUfCmG | | AmAmUmG | | UA | |
| | mCmAmC | | fUmGfUmA | | | |
| | mCmCmC | | mCmCmCm | | | |
| | mUmAm | | sUmsGm | | | |
| WP3262 | UmsCmsA | 995 | UmsCfsCm | 1058 | CUUCACA | 1109 |
| MW02 | mCmAmG | | AmGmGmU | | GAGGAAG | |
| | mAfGmGf | | mUmUmCm | | AAACCUG | |
| | AfAfGmA | | UmUmCmC | | GA | |
| | mAmAmC | | fUmCfUmG | | | |
| | mCmUmG | | mUmGmAm | | | |
| | mGmAm | | sAmsGm | | | |
| WP3315 | GmsCmsA | 996 | CmsGfsUm | 1059 | CAGCAGG | 925 |
| MW02 | mGmGmA | | UmUmCmU | | AACUGAG | |
| | mAfCmUf | | mGmGmCm | | CCAGAAA | |
| | GfAfGmC | | UmCmAmG | | CG | |
| | mCmAmG | | fUmUfCmC | | | |
| | mAmAmA | | mUmGmCm | | | |
| | mCmGm | | sUmsGm | | | |
| WP3315- | GmsCmsA | 997 | UmsGfsUm | 1060 | CAGCAGG | 1110 |
| AMW02 | mGmGmA | | UmUmCmU | | AACUGAG | |
| | mAfCmUf | | mGmGmCm | | CCAGAAA | |
| | GfAfGmC | | UmCmAmG | | CA | |
| | mCmAmG | | fUmUfCmC | | | |
| | mAmAmA | | mUmGmCm | | | |
| | mCmAm | | sUmsGm | | | |
| WP3323 | UmsGmsA | 998 | AmsCfsUm | 1061 | CCAAUCU | 360 |
| MW02 | mGmCmC | | GmAmGmC | | GCGUUUC | |
| | mAfGmAf | | mCmAmGm | | UGGCUCA | |
| | AfAfCmG | | AmAmAmC | | GU | |
| | mCmAmG | | fGmCfAmG | | | |
| | mAmUmU | | mAmUmUm | | | |
| | mGmGm | | sGmsGm | | | |
| Pt609MW | CmsCmsU | 999 | AmsCfsAm | 1062 | UACCUCA | 764 |
| 02 | mCmAmC | | UmGmCmA | | CCAAGAU | |
| | mCfAmAf | | mGmGmAm | | CCUGCAU | |
| | GfAfUmC | | UmCmUmU | | GU | |
| | mCmUmG | | fGmGfUmG | | | |
| | mCmAmU | | mAmGmGm | | | |
| | mGmUm | | sUmsAm | | | |
| Pt890MW | UmsGmsG | 1000 | CmsAfsUm | 1063 | CAUGGUC | 778 |
| 02 | mUmCmA | | UmCmUmC | | ACCGACU | |
| | mCfCmGf | | mGmAmAm | | UCGAGAA | |
| | AfCfUmU | | GmUmCmG | | UG | |
| | mCmGmA | | fGmUfGmA | | | |
| | mGmAmA | | mCmCmAm | | | |
| | mUmGm | | sUmsGm | | | |
| Pt2530M | CmsCmsU | 1001 | AmsGfsUm | 1064 | AGCCUCC | 807 |
| W02 | mCmCmU | | GmAmGmU | | UUGCCUG | |
| | mUfGmCf | | mUmCmCm | | GAACUCA | |
| | CfUfGmG | | AmGmGmC | | CU | |
| | mAmAmC | | fAmAfGmG | | | |
| | mUmCmA | | mAmGmGm | | | |
| | mCmUm | | sCmsUm | | | |
| Pt2532M | UmsCmsC | 1002 | UmsGfsAm | 1065 | CCUCCUU | 808 |
| W02 | mUmUmG | | GmUmGmA | | GCCUGGA | |
| | mCfCmUf | | mGmUmUm | | ACUCACU | |
| | GfGfAmA | | CmCmAmG | | CA | |
| | mCmUmC | | fGmCfAmA | | | |
| | mAmCmU | | mGmGmAm | | | |
| | mCmAm | | sGmsGm | | | |
| Pt2533M | CmsCmsU | 1003 | GmsUfsGm | 1066 | CUCCUUG | 809 |
| W02 | mUmGmC | | AmGmUmG | | CCUGGAA | |
| | mCfUmGf | | mAmGmUm | | CUCACUC | |
| | GfAfAmC | | UmCmCmA | | AC | |
| | mUmCmA | | fGmGfCmA | | | |
| | mCmUmC | | mAmGmGm | | | |
| | mAmCm | | sAmsGm | | | |
| Pt2534M | CmsUmsU | 1004 | AmsGfsUm | 1067 | UCCUUGC | 810 |
| W02 | mGmCmC | | GmAmGmU | | CUGGAAC | |
| | mUfGmGf | | mGmAmGm | | UCACUCA | |
| | AfAfCmU | | UmUmCmC | | CU | |
| | mCmAmC | | fAmGfGmC | | | |
| | mUmCmA | | mAmAmGm | | | |
| | mCmUm | | sGmsAm | | | |
| Pt2536M | UmsGmsC | 1005 | AmsGfsAm | 1068 | CUUGCCU | 812 |
| W02 | mCmUmG | | GmUmGmA | | GGAACUC | |
| | mGfAmAf | | mGmUmGm | | ACUCACU | |
| | CfUfCmA | | AmGmUmU | | CU | |
| | mCmUmC | | fCmCfAmG | | | |
| | mAmCmU | | mGmCmAm | | | |
| | mCmUm | | sAmsGm | | | |
| Pt2538M | CmsCmsU | 1006 | CmsCfsAm | 1069 | UGCCUGG | 813 |
| W02 | mGmGmA | | GmAmGmU | | AACUCAC | |
| | mAfCmUf | | mGmAmGm | | UCACUCU | |
| | CfAfCmU | | UmGmAmG | | GG | |
| | mCmAmC | | fUmUfCmC | | | |
| | mUmCmU | | mAmGmGm | | | |
| | mGmGm | | sCmsAm | | | |
| Pt2541M | GmsGmsA | 1007 | CmsAfsCmC | 1070 | CUGGAAC | 815 |
| W02 | mAmCmU | | mCmAmGm | | UCACUCA | |
| | mCfAmCf | | AmGmUmG | | CUCUGGG | |
| | UfCfAmC | | mAmGmUf | | UG | |
| | mUmCmU | | GmAfGmU | | | |
| | mGmGmG | | mUmCmCm | | | |
| | mUmGm | | sAmsGm | | | |
| Pt2612M | AmsUmsU | 1008 | GmsAfsCm | 1071 | GCAUUUC | 820 |
| W02 | mUmCmA | | CmUmGmU | | ACCAUUC | |
| | mCfCmAf | | mUmUmGm | | AAACAGG | |
| | UfUfCmA | | AmAmUmG | | UC | |
| | mAmAmC | | fGmUfGmA | | | |
| | mAmGmG | | mAmAmUm | | | |
| | mUmCm | | sGmsCm | | | |
| Pt2675M | GmsUmsC | 1009 | AmsAfsAm | 1072 | AUGUCCG | 828 |
| W02 | mCmGmU | | GmUmCmA | | UGGGCAG | |
| | mGfGmGf | | mUmUmCm | | AAUGACU | |
| | CfAfGmA | | UmGmCmC | | UU | |
| | mAmUmG | | fCmAfCmG | | | |
| | mAmCmU | | mGmAmCm | | | |
| | mUmUm | | sAmsUm | | | |
| Pt2697M | AmsUmsU | 1010 | GmsGfsCm | 1073 | UUAUUGA | 830 |
| W02 | mGmAmG | | AmCmGmG | | GCUCUUG | |
| | mCfUmCf | | mAmAmCm | | UUCCGUG | |
| | UfUfGmU | | AmAmGmA | | CC | |
| | mUmCmC | | fGmCfUmC | | | |
| | mGmUmG | | mAmAmUm | | | |
| | mCmCm | | sAmsAm | | | |
| Pt2698M | UmsUmsG | 1011 | UmsGfsGm | 1074 | UAUUGAG | 831 |
| W02 | mAmGmC | | CmAmCmG | | CUCUUGU | |
| | mUfCmUf | | mGmAmAm | | UCCGUGC | |
| | UfGfUmU | | CmAmAmG | | CA | |
| | mCmCmG | | fAmGfCmU | | | |
| | mUmGmC | | mCmAmAm | | | |
| | mCmAm | | sUmsAm | | | |
| Pt2699M | UmsGmsA | 1012 | CmsUfsGm | 1075 | AUUGAGC | 832 |
| W02 | mGmCmU | | GmCmAmC | | UCUUGUU | |
| | mCfUmUf | | mGmGmAm | | CCGUGCC | |
| | GfUfUmC | | AmCmAmA | | AG | |
| | mCmGmU | | fGmAfGmC | | | |
| | mGmCmC | | mUmCmAm | | | |
| | mAmGm | | sAmsUm | | | |
| Pt2700M | GmsAmsG | 1013 | CmsCfsUm | 1076 | UUGAGCU | 833 |
| W02 | mCmUmC | | GmGmCmA | | CUUGUUC | |
| | mUfUmGf | | mCmGmGm | | CGUGCCA | |
| | UfUfCmC | | AmAmCmA | | GG | |
| | mGmUmG | | fAmGfAmG | | | |
| | mCmCmA | | mCmUmCm | | | |
| | mGmGm | | sAmsAm | | | |
| Pt2717M | CmsAmsG | 1014 | GmsAfsCm | 1077 | GCCAGGC | 834 |
| W02 | mGmCmA | | CmUmGmA | | AUUCAAU | |
| | mUfUmCf | | mGmGmAm | | CCUCAGG | |
| | AfAfUmC | | UmUmGmA | | UC | |
| | mCmUmC | | fAmUfGmC | | | |
| | mAmGmG | | mCmUmGm | | | |
| | mUmCm | | sGmsCm | | | |
| Pt2727M | AmsUmsC | 1015 | CmsCfsUm | 1078 | CAAUCCU | 840 |
| W02 | mCmUmC | | UmGmGmU | | CAGGUCU | |
| | mAfGmGf | | mGmGmAm | | CCACCAA | |
| | UfCfUmC | | GmAmCmC | | GG | |
| | mCmAmC | | fUmGfAmG | | | |
| | mCmAmA | | mGmAmUm | | | |
| | mGmGm | | sUmsGm | | | |
| Pt2748M | AmsGmsG | 1016 | UmsCfsCm | 1079 | GGAGGCA | 848 |
| W02 | mCmAmG | | AmUmGmG | | GGAUUCU | |
| | mGfAmUf | | mGmAmAm | | UCCCAUG | |
| | UfCfUmU | | GmAmAmU | | GA | |
| | mCmCmC | | fCmCfUmG | | | |
| | mAmUmG | | mCmCmUm | | | |
| | mGmAm | | sCmsCm | | | |
| Pt2750M | GmsCmsA | 1017 | UmsAfsUm | 1080 | AGGCAGG | 849 |
| W02 | mGmGmA | | CmCmAmU | | AUUCUUC | |
| | mUfUmCf | | mGmGmGm | | CCAUGGA | |
| | UfUfCmC | | AmAmGmA | | UA | |
| | mCmAmU | | fAmUfCmC | | | |
| | mGmGmA | | mUmGmCm | | | |
| | mUmAm | | sCmsUm | | | |
| Pt2752M | AmsGmsG | 1018 | CmsCfsUm | 1081 | GCAGGAU | 850 |
| W02 | mAmUmU | | AmUmCmC | | UCUUCCC | |
| | mCfUmUf | | mAmUmGm | | AUGGAUA | |
| | CfCfCmA | | GmGmAmA | | GG | |
| | mUmGmG | | fGmAfAmU | | | |
| | mAmUmA | | mCmCmUm | | | |
| | mGmGm | | sGmsCm | | | |
| Pt2818M | AmsGmsU | 1019 | GmsCfsCm | 1082 | UGAGUGU | 858 |
| W02 | mGmUmG | | AmUmCmA | | GAAAGGU | |
| | mAfAmAf | | mGmCmAm | | GCUGAUG | |
| | GfGfUmG | | CmCmUmU | | GC | |
| | mCmUmG | | fUmCfAmC | | | |
| | mAmUmG | | mAmCmUm | | | |
| | mGmCm | | sCmsAm | | | |
| Pt2837M | GmsCmsC | 1020 | CmsAfsGm | 1083 | UGGCCCU | 864 |
| W02 | mCmUmC | | UmUmAmG | | CAUCUCC | |
| | mAfUmCf | | mCmUmGm | | AGCUAAC | |
| | UfCfCmA | | GmAmGmA | | UG | |
| | mGmCmU | | fUmGfAmG | | | |
| | mAmAmC | | mGmGmCm | | | |
| | mUmGm | | sCmsAm | | | |
| Pt2838M | CmsCmsC | 1021 | AmsCfsAm | 1084 | GGCCCUC | 865 |
| W02 | mUmCmA | | GmUmUmA | | AUCUCCA | |
| | mUfCmUf | | mGmCmUm | | GCUAACU | |
| | CfCfAmG | | GmGmAmG | | GU | |
| | mCmUmA | | fAmUfGmA | | | |
| | mAmCmU | | mGmGmGm | | | |
| | mGmUm | | sCmsCm | | | |
| Pt2882M | GmsAmsU | 1022 | AmsAfsAm | 1085 | CUGAUUA | 872 |
| W02 | mUmAmA | | GmCmUmA | | AUGGAGG | |
| | mUfGmGf | | mAmGmCm | | CUUAGCU | |
| | AfGfGmC | | CmUmCmCf | | UU | |
| | mUmUmA | | AmUfUmA | | | |
| | mGmCmU | | mAmUmCm | | | |
| | mUmUm | | sAmsGm | | | |
| Pt2883M | AmsUmsU | 1023 | GmsAfsAm | 1086 | UGAUUAA | 873 |
| W02 | mAmAmU | | AmGmCmU | | UGGAGGC | |
| | mGfGmAf | | mAmAmGm | | UUAGCUU | |
| | GfGfCmU | | CmCmUmCf | | UC | |
| | mUmAmG | | CmAfUmU | | | |
| | mCmUmU | | mAmAmUm | | | |
| | mUmCm | | sCmsAm | | | |
| Pt2888M | UmsGmsG | 1024 | AmsUfsCm | 1087 | AAUGGAG | 877 |
| W02 | mAmGmG | | CmAmGmA | | GCUUAGC | |
| | mCfUmUf | | mAmAmGm | | UUUCUGG | |
| | AfGfCmU | | CmUmAmA | | AU | |
| | mUmUmC | | fGmCfCmU | | | |
| | mUmGmG | | mCmCmAm | | | |
| | mAmUm | | sUmsUm | | | |
| Pt2889M | GmsGmsA | 1025 | CmsAfsUm | 1088 | AUGGAGG | 878 |
| W02 | mGmGmC | | CmCmAmG | | CUUAGCU | |
| | mUfUmAf | | mAmAmAm | | UUCUGGA | |
| | GfCfUmU | | GmCmUmA | | UG | |
| | mUmCmU | | fAmGfCmC | | | |
| | mGmGmA | | mUmCmCm | | | |
| | mUmGm | | sAmsUm | | | |
| Pt2980M | CmsCmsU | 1026 | UmsGfsGm | 1089 | CACCUUU | 896 |
| W02 | mUmUmA | | CmAmUmA | | ACUCUGC | |
| | mCfUmCf | | mGmAmGm | | UCUAUGC | |
| | UfGfCmU | | CmAmGmA | | CA | |
| | mCmUmA | | fGmUfAmA | | | |
| | mUmGmC | | mAmGmGm | | | |
| | mCmAm | | sUmsGm | | | |
| Pt2991M | CmsUmsC | 1027 | CmsUfsAm | 1090 | UGCUCUA | 904 |
| W02 | mUmAmU | | GmCmAmC | | UGCCAGG | |
| | mGfCmCf | | mAmGmCm | | CUGUGCU | |
| | AfGfGmC | | CmUmGmG | | AG | |
| | mUmGmU | | fCmAfUmA | | | |
| | mGmCmU | | mGmAmGm | | | |
| | mAmGm | | sCmsAm | | | |
| Pt3138M | CmsAmsC | 1028 | UmsUfsCm | 1091 | UUCACAG | 913 |
| W02 | mAmGmA | | CmAmGmG | | AGGAAGA | |
| | mGfGmAf | | mUmUmUm | | AACCUGG | |
| | AfGfAmA | | CmUmUmC | | AA | |
| | mAmCmC | | fCmUfCmU | | | |
| | mUmGmG | | mGmUmGm | | | |
| | mAmAm | | sAmsAm | | | |
| Pt3139M | AmsCmsA | 1029 | GmsUfsUm | 1092 | UCACAGA | 914 |
| W02 | mGmAmG | | CmCmAmG | | GGAAGAA | |
| | mGfAmAf | | mGmUmUm | | ACCUGGA | |
| | GfAfAmA | | UmCmUmU | | AC | |
| | mCmCmU | | fCmCfUmC | | | |
| | mGmGmA | | mUmGmUm | | | |
| | mAmCm | | sGmsAm | | | |
| Pt3141M | AmsGmsA | 1030 | UmsGfsGm | 1093 | ACAGAGG | 915 |
| W02 | mGmGmA | | UmUmCmC | | AAGAAAC | |
| | mAfGmAf | | mAmGmGm | | CUGGAAC | |
| | AfAfCmC | | UmUmUmC | | CA | |
| | mUmGmG | | fUmUfCmC | | | |
| | mAmAmC | | mUmCmUm | | | |
| | mCmAm | | sGmsUm | | | |
| Pt3185M | AmsCmsA | 1031 | CmsUfsGm | 1094 | UCACACA | 920 |
| W02 | mCmAmG | | GmCmUmC | | GCAGGAA | |
| | mCfAmGf | | mAmGmUm | | CUGAGCC | |
| | GfAfAmC | | UmCmCmU | | AG | |
| | mUmGmA | | fGmCfUmG | | | |
| | mGmCmC | | mUmGmUm | | | |
| | mAmGm | | sGmsAm | | | |
| Pt3186M | CmsAmsC | 1032 | UmsCfsUm | 1095 | CACACAG | 921 |
| W02 | mAmGmC | | GmGmCmU | | CAGGAAC | |
| | mAfGmGf | | mCmAmGm | | UGAGCCA | |
| | AfAfCmU | | UmUmCmC | | GA | |
| | mGmAmG | | fUmGfCmU | | | |
| | mCmCmA | | mGmUmGm | | | |
| | mGmAm | | sUmsGm | | | |
| Pt3191M | CmsAmsG | 1033 | GmsCfsGm | 1096 | AGCAGGA | 926 |
| W02 | mGmAmA | | UmUmUmC | | ACUGAGC | |
| | mCfUmGf | | mUmGmGm | | CAGAAAC | |
| | AfGfCmC | | CmUmCmA | | GC | |
| | mAmGmA | | fGmUfUmC | | | |
| | mAmAmC | | mCmUmGm | | | |
| | mGmCm | | sCmsUm | | | |
| Pt3193M | GmsGmsA | 1034 | CmsUfsGm | 1097 | CAGGAAC | 928 |
| W02 | mAmCmU | | CmGmUmU | | UGAGCCA | |
| | mGfAmGf | | mUmCmUm | | GAAACGC | |
| | CfCfAmG | | GmGmCmU | | AG | |
| | mAmAmA | | fCmAfGmU | | | |
| | mCmGmC | | mUmCmCm | | | |
| | mAmGm | | sUmsGm | | | |
| Pt3194M | GmsAmsA | 1035 | UmsCfsUm | 1098 | AGGAACU | 929 |
| W02 | mCmUmG | | GmCmGmU | | GAGCCAG | |
| | mAfGmCf | | mUmUmCm | | AAACGCA | |
| | CfAfGmA | | UmGmGmC | | GA | |
| | mAmAmC | | fUmCfAmG | | | |
| | mGmCmA | | mUmUmCm | | | |
| | mGmAm | | sCmsUm | | | |
| Pt3199M | GmsAmsG | 1036 | CmsCfsCmA | 1099 | CUGAGCC | 930 |
| W02 | mCmCmA | | mAmUmCm | | AGAAACG | |
| | mGfAmAf | | UmGmCmG | | CAGAUUG | |
| | AfCfGmC | | mUmUmUf | | GG | |
| | mAmGmA | | CmUfGmG | | | |
| | mUmUmG | | mCmUmCm | | | |
| | mGmGm | | sAmsGm | | | |
| Pt3236M | GmsCmsC | 1037 | CmsCfsGm | 1100 | AAGCCUC | 937 |
| W02 | mUmCmU | | GmGmUmG | | UUCUUAC | |
| | mUfCmUf | | mAmAmGm | | UUCACCC | |
| | UfAfCmU | | UmAmAmG | | GG | |
| | mUmCmA | | fAmAfGmA | | | |
| | mCmCmC | | mGmGmCm | | | |
| | mGmGm | | sUmsUm | | | |
| Pt3276M | GmsGmsG | 1038 | UmsUfsCm | 1101 | ACGGGUA | 945 |
| W02 | mUmAmA | | CmCmAmG | | ACAGUGA | |
| | mCfAmGf | | mCmCmUm | | GGCUGGG | |
| | UfGfAmG | | CmAmCmU | | AA | |
| | mGmCmU | | fGmUfUmA | | | |
| | mGmGmG | | mCmCmCm | | | |
| | mAmAm | | sGmsUm | | | |
| Pt3304M | AmsCmsA | 1039 | UmsCfsAm | 1102 | ACACAGA | 948 |
| W02 | mGmAmC | | CmCmGmA | | CCAGGAA | |
| | mCfAmGf | | mGmCmUm | | GCUCGGU | |
| | GfAfAmG | | UmCmCmU | | GA | |
| | mCmUmC | | fGmGfUmC | | | |
| | mGmGmU | | mUmGmUm | | | |
| | mGmAm | | sGmsUm | | | |
| Pt3315M | AmsGmsC | 1040 | UmsCfsUm | 1103 | GAAGCUC | 954 |
| W02 | mUmCmG | | GmCmCmA | | GGUGAGU | |
| | mGfUmGf | | mUmCmAm | | GAUGGCA | |
| | AfGfUmG | | CmUmCmA | | GA | |
| | mAmUmG | | fCmCfGmA | | | |
| | mGmCmA | | mGmCmUm | | | |
| | mGmAm | | sUmsCm | | | |
| Pt3436M | AmsAmsC | 1041 | GmsGfsUm | 1104 | ACAACUG | 967 |
| W02 | mUmGmU | | GmCmUmC | | UCCCUCC | |
| | mCfCmCf | | mAmAmGm | | UUGAGCA | |
| | UfCfCmU | | GmAmGmG | | CC | |
| | mUmGmA | | fGmAfCmA | | | |
| | mGmCmA | | mGmUmUm | | | |
| | mCmCm | | sGmsUm | | | |
| Pt3438M | CmsUmsG | 1042 | CmsUfsGm | 1105 | AACUGUC | 969 |
| W02 | mUmCmC | | GmUmGmC | | CCUCCUU | |
| | mCfUmCf | | mUmCmAm | | GAGCACC | |
| | CfUfUmG | | AmGmGmA | | AG | |
| | mAmGmC | | fGmGfGmA | | | |
| | mAmCmC | | mCmAmGm | | | |
| | mAmGm | | sUmsUm | | | |
| Pt3493M | UmsCmsU | 1043 | AmsAfsAm | 1106 | GGUCUGU | 976 |
| W02 | mGmUmC | | GmGmCmA | | CCUCUCU | |
| | mCfUmCf | | mAmCmAm | | GUUGCCU | |
| | UfCfUmG | | GmAmGmA | | UU | |
| | mUmUmG | | fGmGfAmC | | | |
| | mCmCmU | | mAmGmAm | | | |
| | mUmUm | | sCmsCm | | | |
| Pt3553M | UmsUmsG | 1044 | AmsCfsCmC | 1107 | ACUUGAA | 978 |
| W02 | mAmAmG | | mAmGmAm | | GAUAUUU | |
| | mAfUmAf | | AmUmAmA | | AUUCUGG | |
| | UfUfUmA | | mAmUmAf | | GU | |
| | mUmUmC | | UmCfUmU | | | |
| | mUmGmG | | mCmAmAm | | | |
| | mGmUm | | sGmsUm | | | |
| SP341.1 | CCAACU | 100 | AAACAGG | 1125 | AGCCAAC | 754 |
| | UUUCUA | | UCUAGAA | | UUUUCUA | |
| | GACCUG | | AAGUUGG | | GACCUGU | |
| | UUU | | UU | | UU | |
| SP341.2 | CCAACU | 100 | AAACAGG | 1126 | AGCCAAC | 754 |
| | UUUCUA | | UCUAGAA | | UUUUCUA | |
| | GACCUG | | AAGUUGG | | GACCUGU | |
| | UUU | | UA | | UU | |
| SP341.3 | CCAACU | 100 | AAACAGG | 1127 | AGCCAAC | 754 |
| | UUUCUA | | UCUAGAA | | UUUUCUA | |
| | GACCUG | | AAGUUGG | | GACCUGU | |
| | UUU | | AU | | UU | |
| SP341.4 | CCAACU | 100 | AAACAGG | 1128 | AGCCAAC | 754 |
| | UUUCUA | | UCUAGAA | | UUUUCUA | |
| | GACCUG | | AAGUUGG | | GACCUGU | |
| | UUU | | AA | | UU | |
| SP341.5 | CCAACU | 100 | AAACAGG | 1129 | AGCCAAC | 754 |
| | UUUCUA | | UCUAGAA | | UUUUCUA | |
| | GACCUG | | AAGUUGG | | GACCUGU | |
| | UUU | | CA | | UU | |
| SP341.6 | CCAACU | 1123 | UAACAGG | 1130 | AGCCAAC | 754 |
| | UUUCUA | | UCUAGAA | | UUUUCUA | |
| | GACCUG | | AAGUUGG | | GACCUGU | |
| | UUA | | CU | | UU | |
| SP341.7 | CCAACU | 1123 | UAACAGG | 1131 | AGCCAAC | 754 |
| | UUUCUA | | UCUAGAA | | UUUUCUA | |
| | GACCUG | | AAGUUGG | | GACCUGU | |
| | UUA | | UU | | UU | |
| SP341.8 | CCAACU | 1123 | UAACAGG | 1132 | AGCCAAC | 754 |
| | UUUCUA | | UCUAGAA | | UUUUCUA | |
| | GACCUG | | AAGUUGG | | GACCUGU | |
| | UUA | | UA | | UU | |
| SP341.9 | CCAACU | 1123 | UAACAGG | 1133 | AGCCAAC | 754 |
| | UUUCUA | | UCUAGAA | | UUUUCUA | |
| | GACCUG | | AAGUUGG | | GACCUGU | |
| | UUA | | AU | | UU | |
| SP341.10 | CCAACU | 1123 | UAACAGG | 1134 | AGCCAAC | 754 |
| | UUUCUA | | UCUAGAA | | UUUUCUA | |
| | GACCUG | | AAGUUGG | | GACCUGU | |
| | UUA | | AA | | UU | |
| SP341.11 | CCAACU | 1123 | UAACAGG | 1135 | AGCCAAC | 754 |
| | UUUCUA | | UCUAGAA | | UUUUCUA | |
| | GACCUG | | AAGUUGG | | GACCUGU | |
| | UUA | | CA | | UU | |
| Ps3543.1 | CUUUUG | 40 | AAUAUCU | 1136 | UGCUUUU | 694 |
| | UAACUU | | UCAAGUU | | GUAACUU | |
| | GAAGAU | | ACAAAAG | | GAAGAUA | |
| | AUU | | UA | | UU | |
| Ps3543.2 | CUUUUG | 40 | AAUAUCU | 1137 | UGCUUUU | 694 |
| | UAACUU | | UCAAGUU | | GUAACUU | |
| | GAAGAU | | ACAAAAG | | GAAGAUA | |
| | AUU | | UU | | UU | |
| Ps3543.3 | CUUUUG | 40 | AAUAUCU | 1138 | UGCUUUU | 694 |
| | UAACUU | | UCAAGUU | | GUAACUU | |
| | GAAGAU | | ACAAAAG | | GAAGAUA | |
| | AUU | | AA | | UU | |
| Ps3543.4 | CUUUUG | 40 | AAUAUCU | 1139 | UGCUUUU | 694 |
| | UAACUU | | UCAAGUU | | GUAACUU | |
| | GAAGAU | | ACAAAAG | | GAAGAUA | |
| | AUU | | AU | | UU | |
| Ps3543.5 | CUUUUG | 40 | AAUAUCU | 1140 | UGCUUUU | 694 |
| | UAACUU | | UCAAGUU | | GUAACUU | |
| | GAAGAU | | ACAAAAG | | GAAGAUA | |
| | AUU | | CU | | UU | |
| Ps3543.6 | CUUUUG | 1124 | UAUAUCU | 1141 | UGCUUUU | 694 |
| | UAACUU | | UCAAGUU | | GUAACUU | |
| | GAAGAU | | ACAAAAG | | GAAGAUA | |
| | AUA | | CA | | UU | |
| Ps3543.7 | CUUUUG | 1124 | UAUAUCU | 1142 | UGCUUUU | 694 |
| | UAACUU | | UCAAGUU | | GUAACUU | |
| | GAAGAU | | ACAAAAG | | GAAGAUA | |
| | AUA | | UA | | UU | |
| Ps3543.8 | CUUUUG | 1124 | UAUAUCU | 1143 | UGCUUUU | 694 |
| | UAACUU | | UCAAGUU | | GUAACUU | |
| | GAAGAU | | ACAAAAG | | GAAGAUA | |
| | AUA | | UU | | UU | |
| Ps3543.9 | CUUUUG | 1124 | UAUAUCU | 1144 | UGCUUUU | 694 |
| | UAACUU | | UCAAGUU | | GUAACUU | |
| | GAAGAU | | ACAAAAG | | GAAGAUA | |
| | AUA | | AA | | UU | |
| Ps3543.10 | CUUUUG | 1124 | UAUAUCU | 1145 | UGCUUUU | 694 |
| | UAACUU | | UCAAGUU | | GUAACUU | |
| | GAAGAU | | ACAAAAG | | GAAGAUA | |
| | AUA | | AU | | UU | |
| Ps3543.11 | CUUUUG | 1124 | UAUAUCU | 1146 | UGCUUUU | 694 |
| | UAACUU | | UCAAGUU | | GUAACUU | |
| | GAAGAU | | ACAAAAG | | GAAGAUA | |
| | AUA | | CU | | UU | |
| SP341.1M | CmsCmsA | 988 | AmsAfsAm | 1149 | AGCCAAC | 754 |
| W02 | mAmCmU | | CmAmGmG | | UUUUCUA | |
| | mUfUmUf | | mUmCmUm | | GACCUGU | |
| | CfUfAmG | | AmGmAmA | | UU | |
| | mAmCmC | | fAmAfGmU | | | |
| | mUmGmU | | mUmGmGm | | | |
| | mUmUm | | sUmsUm | | | |
| SP341.2M | CmsCmsA | 988 | AmsAfsAm | 1150 | AGCCAAC | 754 |
| W02 | mAmCmUmUf | | CmAmGmG | | UUUUCUA | |
| | UmUfCfUf | | mUmCmUm | | GACCUGU | |
| | AmGmAmCmC | | AmGmAmA | | UU | |
| | mUmGmU | | fAmAfGmU | | | |
| | mUmUm | | mUmGmGm | | | |
| | | | sUmsAm | | | |
| SP341.3M | CmsCmsA | 988 | AmsAfsAm | 1151 | AGCCAAC | 754 |
| W02 | mAmCmUm | | CmAmGmG | | UUUUCUA | |
| | UfUmUf | | mUmCmUm | | GACCUGU | |
| | CfUfAmG | | AmGmAmA | | UU | |
| | mAmCmC | | fAmAfGmU | | | |
| | mUmGmU | | mUmGmGm | | | |
| | mUmUm | | sAmsUm | | | |
| SP341.4M | CmsCmsA | 988 | AmsAfsAm | 1152 | AGCCAAC | 754 |
| W02 | mAmCmU | | CmAmGmG | | UUUUCUA | |
| | mUfUmUf | | mUmCmUm | | GACCUGU | |
| | CfUfAmG | | AmGmAmA | | UU | |
| | mAmCmC | | fAmAfGmU | | | |
| | mUmGmU | | mUmGmGm | | | |
| | mUmUm | | sAmsAm | | | |
| SP341.5M | CmsCmsA | 988 | AmsAfsAm | 1153 | AGCCAAC | 754 |
| W02 | mAmCmUm | | CmAmGmG | | UUUUCUA | |
| | UfUmUf | | mUmCmUm | | GACCUGU | |
| | CfUfAmG | | AmGmAmA | | UU | |
| | mAmCmC | | fAmAfGmU | | | |
| | mUmGmU | | mUmGmGm | | | |
| | mUmUm | | sCmsAm | | | |
| SP341.6M | CmsCmsA | 1147 | UmsAfsAm | 1154 | AGCCAAC | 754 |
| W02 | mAmCmU | | CmAmGmG | | UUUUCUA | |
| | mUfUmUf | | mUmCmUm | | GACCUGU | |
| | CfUfAmG | | AmGmAmA | | UU | |
| | mAmCmC | | fAmAfGmU | | | |
| | mUmGmU | | mUmGmGm | | | |
| | mUmAm | | sCmsUm | | | |
| SP341.7M | CmsCmsA | 1147 | UmsAfsAm | 1155 | AGCCAAC | 754 |
| W02 | mAmCmUm | | CmAmGmG | | UUUUCUA | |
| | UfUmUf | | mUmCmUm | | GACCUGU | |
| | CfUfAmG | | AmGmAmA | | UU | |
| | mAmCmC | | fAmAfGmU | | | |
| | mUmGmU | | mUmGmGm | | | |
| | mUmAm | | sUmsUm | | | |
| SP341.8M | CmsCmsA | 1147 | UmsAfsAm | 1156 | AGCCAAC | 754 |
| W02 | mAmCmUm | | CmAmGmG | | UUUUCUA | |
| | UfUmUf | | mUmCmUm | | GACCUGU | |
| | CfUfAmG | | AmGmAmA | | UU | |
| | mAmCmC | | fAmAfGmU | | | |
| | mUmGmU | | mUmGmGm | | | |
| | mUmAm | | sUmsAm | | | |
| SP341.9M | CmsCmsA | 1147 | UmsAfsAm | 1157 | AGCCAAC | 754 |
| W02 | mAmCmUm | | CmAmGmG | | UUUUCUA | |
| | UfUmUf | | mUmCmUm | | GACCUGU | |
| | CfUfAmG | | AmGmAmA | | UU | |
| | mAmCmC | | fAmAfGmU | | | |
| | mUmGmU | | mUmGmGm | | | |
| | mUmAm | | sAmsUm | | | |
| SP341.10 | CmsCmsA | 1147 | UmsAfsAm | 1158 | AGCCAAC | 754 |
| MW02 | mAmCmU | | CmAmGmG | | UUUUCUA | |
| | mUfUmUf | | mUmCmUm | | GACCUGU | |
| | CfUfAmG | | AmGmAmA | | UU | |
| | mAmCmC | | fAmAfGmU | | | |
| | mUmGmU | | mUmGmGm | | | |
| | mUmAm | | sAmsAm | | | |
| SP341.11 | CmsCmsA | 1147 | UmsAfsAm | 1159 | AGCCAAC | 754 |
| MW02 | mAmCmU | | CmAmGmG | | UUUUCUA | |
| | mUfUmUf | | mUmCmUm | | GACCUGU | |
| | CfUfAmG | | AmGmAmA | | UU | |
| | mAmCmC | | fAmAfGmU | | | |
| | mUmGmU | | mUmGmGm | | | |
| | mUmAm | | sCmsAm | | | |
| Ps3543.1 | CmsUmsU | 985 | AmsAfsUm | 1160 | UGCUUUU | 694 |
| MW02 | mUmUmG | | AmUmCmU | | GUAACUU | |
| | mUfAmAf | | mUmCmAm | | GAAGAUA | |
| | CfUfUmG | | AmGmUmU | | UU | |
| | mAmAmG | | fAmCfAmA | | | |
| | mAmUmA | | mAmAmGm | | | |
| | mUmUm | | sUmsAm | | | |
| Ps3543.2 | CmsUmsU | 985 | AmsAfsUm | 1161 | UGCUUUU | 694 |
| MW02 | mUmUmG | | AmUmCmU | | GUAACUU | |
| | mUfAmAf | | mUmCmAm | | GAAGAUA | |
| | CfUfUmG | | AmGmUmU | | UU | |
| | mAmAmG | | fAmCfAmA | | | |
| | mAmUmA | | mAmAmGm | | | |
| | mUmUm | | sUmsUm | | | |
| Ps3543.3 | CmsUmsU | 985 | AmsAfsUm | 1162 | UGCUUUU | 694 |
| MW02 | mUmUmG | | AmUmCmU | | GUAACUU | |
| | mUfAmAf | | mUmCmAm | | GAAGAUA | |
| | CfUfUmG | | AmGmUmU | | UU | |
| | mAmAmG | | fAmCfAmA | | | |
| | mAmUmA | | mAmAmGm | | | |
| | mUmUm | | sAmsAm | | | |
| Ps3543.4 | CmsUmsU | 985 | AmsAfsUm | 1163 | UGCUUUU | 694 |
| MW02 | mUmUmG | | AmUmCmU | | GUAACUU | |
| | mUfAmAf | | mUmCmAm | | GAAGAUA | |
| | CfUfUmG | | AmGmUmU | | UU | |
| | mAmAmG | | fAmCfAmA | | | |
| | mAmUmA | | mAmAmGm | | | |
| | mUmUm | | sAmsUm | | | |
| Ps3543.5 | CmsUmsU | 985 | AmsAfsUm | 1164 | UGCUUUU | 694 |
| MW02 | mUmUmG | | AmUmCmU | | GUAACUU | |
| | mUfAmAf | | mUmCmAm | | GAAGAUA | |
| | CfUfUmG | | AmGmUmU | | UU | |
| | mAmAmG | | fAmCfAmA | | | |
| | mAmUmA | | mAmAmGm | | | |
| | mUmUm | | sCmsUm | | | |
| Ps3543.6 | CmsUmsU | 1148 | UmsAfsUm | 1165 | UGCUUUU | 694 |
| MW02 | mUmUmG | | AmUmCmU | | GUAACUU | |
| | mUfAmAf | | mUmCmAm | | GAAGAUA | |
| | CfUfUmG | | AmGmUmU | | UU | |
| | mAmAmG | | fAmCfAmA | | | |
| | mAmUmA | | mAmAmGm | | | |
| | mUmAm | | sCmsAm | | | |
| Ps3543.7 | CmsUmsU | 1148 | UmsAfsUm | 1166 | UGCUUUU | 694 |
| MW02 | mUmUmG | | AmUmCmU | | GUAACUU | |
| | mUfAmAf | | mUmCmAm | | GAAGAUA | |
| | CfUfUmG | | AmGmUmU | | UU | |
| | mAmAmG | | fAmCfAmA | | | |
| | mAmUmA | | mAmAmGm | | | |
| | mUmAm | | sUmsAm | | | |
| Ps3543.8 | CmsUmsU | 1148 | UmsAfsUm | 1167 | UGCUUUU | 694 |
| MW02 | mUmUmG | | AmUmCmU | | GUAACUU | |
| | mUfAmAf | | mUmCmAm | | GAAGAUA | |
| | CfUfUmG | | AmGmUmU | | UU | |
| | mAmAmG | | fAmCfAmA | | | |
| | mAmUmA | | mAmAmGm | | | |
| | mUmAm | | sUmsUm | | | |
| Ps3543.9 | CmsUmsU | 1148 | UmsAfsUm | 1168 | UGCUUUU | 694 |
| MW02 | mUmUmG | | AmUmCmU | | GUAACUU | |
| | mUfAmAf | | mUmCmAm | | GAAGAUA | |
| | CfUfUmG | | AmGmUmU | | UU | |
| | mAmAmG | | fAmCfAmA | | | |
| | mAmUmA | | mAmAmGm | | | |
| | mUmAm | | sAmsAm | | | |
| Ps3543.10 | CmsUmsU | 1148 | UmsAfsUm | 1169 | UGCUUUU | 694 |
| MW02 | mUmUmG | | AmUmCmU | | GUAACUU | |
| | mUfAmAf | | mUmCmAm | | GAAGAUA | |
| | CfUfUmG | | AmGmUmU | | UU | |
| | mAmAmG | | fAmCfAmA | | | |
| | mAmUmA | | mAmAmGm | | | |
| | mUmAm | | sAmsUm | | | |
| Ps3543.11 | CmsUmsU | 1148 | UmsAfsUm | 1170 | UGCUUUU | 694 |
| MW02 | mUmUmG | | AmUmCmU | | GUAACUU | |
| | mUfAmAf | | mUmCmAm | | GAAGAUA | |
| | CfUfUmG | | AmGmUmU | | UU | |
| | mAmAmG | | fAmCfAmA | | | |
| | mAmUmA | | mAmAmGm | | | |
| | mUmAm | | sCmsUm | | | |

Among them, the uppercase letters “G”, “C”, “A”, “T”, and “U” generally represent nucleotides containing guanine, cytosine, adenine, thymine, and uracil as bases, respectively; Am, Gm, Cm, and Tm represent 2′-methoxy-modified nucleotides; Af, Gf, Cf, and Uf represent 2′-fluoro-modified nucleotides; the lowercase letter s indicates that the two adjacent nucleotides flanking the letter s are linked by a phosphorothioate group.

### Modified Nucleotides

In order to enhance the in vivo stability of the RNA inhibitor described above, the sense strand and the antisense strand of the RNA inhibitor described above may be modified without affecting their activity or even with enhancing their activity, wherein the nucleotides may have a modification group, and the modification may be made in the entire strand or part of the strand. In certain embodiments, one or more nucleotides on the sense strand and/or the antisense strand are modified to form modified nucleotides.

The sense strand and the antisense strand in the RNA inhibitor structure provided in the present disclosure have a length of 15-30, preferably 19-23, and at least 85% of bases therebetween are complementary. In order to enhance the in vivo stability of the sense strand and the antisense strand, the sense strand and the antisense strand of the RNA inhibitor may be modified without affecting the activity or even with enhancing the activity, wherein the nucleotides may have a modification group, and the modification may be made in the entire strand or part of the strand, preferably in the entire strand. The modification is a technique readily understood by researchers in the art, may be in the glycosyl group, and may be selected from any one or more of the following in the glycosyl moiety: a deoxyribonucleotide, a nucleotide mimic, an abasic nucleotide, a 2′-modified nucleotide, a 3′ to 3′-linked (inverted) nucleotide, a non-natural base-containing nucleotide, a bridged nucleotide, a peptide nucleic acid (PNA), an unlocked nucleobase analog, a locked nucleotide, a 3′-O-methoxy (2′ internucleoside linked) nucleotide, a 2′-F-arabinose nucleotide, a 5′-Me/2′-fluoro nucleotide, a morpholino nucleotide, a vinyl phosphate deoxyribonucleotide, a vinyl phosphate-containing nucleotide, and a cyclopropyl phosphate-containing nucleotide. Among them, the 2′-modified nucleotide includes, but is not limited to: a 2′-O-methyl nucleotide, a 2′-deoxy-2′-fluoro nucleotide, a 2′-deoxynucleotide, a 2′-methoxyethyl nucleotide, a 2′-amino nucleotide, and a 2′-alkyl nucleotide. In the RNA inhibitor provided in the present disclosure, neither the sense strand nor the antisense strand of the RNA inhibitor needs to be uniformly modified, and one or more modifications may be incorporated into a single nucleotide thereof. The modification may also be made at the base moiety. Modified nucleobases include synthetic and natural nucleobases, such as 5-substituted pyrimidines, 6-azapyrimidines, and N-2/N-6 and O-6 substituted purines, 5-methylcytosine, 5-hydroxymethylcytosine, xanthine, hypoxanthine, 2-aminoadenine, 6-alkyl, 2-alkyl and other alkyl derivatives of adenine and guanine, 2-thiouracil, 2-thiothymine, 2-thiocytosine, 5-halouracil, cytosine, 5-propynyluracil, 5-propynylcytosine, 6-azouracil, 6-azocytosine, 6-azothymine, 5-uracil, 4-thiouracil, 8-halo, 8-amino, 8-thiol, 8-thioalkyl, 8-hydroxyl, and other 8-substituted adenines and guanines, 5-halo, 5-trifluoromethyl, and other 5-substituted uracils and cytosines, 7-methylguanine and 7-methyladenine, 8-azaguanine and 8-azaadenine, 7-deazaguanine, 7-deazaadenine, 3-deazaguanine, and 3-deazaadenine.

Part or all of the sense strand and the antisense strand of the RNA inhibitor described in the present disclosure are 2′-O-methyl nucleotides and/or 2′-deoxy-2′-fluoro nucleotides, and at least two consecutive phosphorothioate bonds are present between the nucleotides at the 5′-end of the sense strand and the 3′-end of the antisense strand. Preferably, the phosphate bonds between the terminal 3 consecutive nucleotides are thioated.

In certain embodiments, the modified nucleotide is selected from: a deoxyribonucleotide, a nucleotide mimic, an abasic nucleotide, a 2′-modified nucleotide, a 3′ to 3′-linked (inverted) nucleotide, a non-natural base-containing nucleotide, a bridged nucleotide, a peptide nucleic acid (PNA), an unlocked nucleobase analog, a locked nucleotide, a 3′-O-methoxy (2′ internucleoside linked) nucleotide, a 2′-F-arabinose nucleotide, a 5′-Me/2′-fluoro nucleotide, a morpholino nucleotide, a vinyl phosphate deoxyribonucleotide, a vinyl phosphate-containing nucleotide, and a cyclopropyl phosphate-containing nucleotide.

In certain embodiments, the 2′-modified nucleotide includes: a 2′-O-methyl nucleotide, a 2′-deoxy-2′-fluoro nucleotide, a 2′-deoxynucleotide, a 2′-methoxyethyl nucleotide, a 2′-amino nucleotide, and/or a 2′-alkyl nucleotide.

In some screened embodiments, the sense strand and the antisense strand of the RNAi agent are selected from the sequences in Table 1 or differ by one, two, or three nucleotides from each of the sequences in Table 1.

In some embodiments, at least one of the —OH groups at the 2′-positions of glycosyl groups in the nucleotides at positions 2, 14, and 16 starting from the 5′-end of the antisense strand is fluorinated.

The —OH groups at the 2′-positions of the glycosyl groups in the nucleotides at positions 2, 14, and 16 starting from the 5′-end of the antisense strand are fluorinated.

In some embodiments, in the antisense strand, except for the nucleotides at positions 2, 14, and 16 starting from the 5′-end, at least one of the —OH groups at the 2′-positions of the glycosyl groups in the remaining nucleotides is replaced by a methoxy group.

In some embodiments, the —OH groups at the 2′-positions of the glycosyl groups in the nucleotides at positions 2, 14, and 16 starting from the 5′-end of the antisense strand are replaced by fluorine, and the —OH groups at the 2′-positions of the glycosyl groups in the remaining nucleotides of the antisense strand are replaced by methoxy groups.

At least one of the —OH groups at the 2′-positions of the glycosyl groups in the nucleotides at positions 7, 9, 10, and 11 starting from the 5′-end of the sense strand is fluorinated.

In some embodiments, in the sense strand, except for the nucleotides at positions 7, 9, 10, and 11 starting from the 5′-end, at least one of the —OH groups at the 2′-positions of the glycosyl groups in the remaining nucleotides is replaced by a methoxy group.

In some embodiments, the —OH groups at the 2′-positions of the glycosyl groups in the nucleotides at positions 7, 9, 10, and 11 starting from the 5′-end of the sense strand are replaced by fluorine, and the —OH groups at the 2′-positions of the glycosyl groups in the remaining nucleotides of the sense strand are replaced by methoxy groups.

At least two consecutive phosphorothioate bonds are present between nucleotides of the sense strand and/or the antisense strand.

The —OH groups at the 2′-positions of the glycosyl groups in the nucleotides at positions 2, 14, and 16 starting from the 5′-end of the antisense strand are fluorinated, the —OH groups at the 2′-positions of the glycosyl groups in the remaining nucleotides of the antisense strand are replaced by methoxy groups, and at least two consecutive phosphorothioate bonds are present between 3 consecutive nucleotides at the end of the antisense strand; the —OH groups at the 2′-positions of the glycosyl groups in the nucleotides at positions 7, 9, 10, and 11 starting from the 5′-end of the sense strand are fluorinated, and the —OH groups at the 2′-positions of the glycosyl groups in the remaining nucleotides of the sense strand are replaced by methoxy groups; and at least two consecutive phosphorothioate bonds are present between 3 consecutive nucleotides at the end of the sense strand.

The modified RNA inhibitors may include P3119MW02, P3196MW02, P3464MW02, P3543MW02, P3554MW02, SP340MW02, SP341MW02, SP342MW02, SP344MW02, SP345MW02, SP349MW02, SP351MW02, WP3239MW02, WP3262MW02, WP3315MW02, WP3315-AMW02, WP3323MW02, Pt609MW02, Pt890MW02, Pt2530MW02, Pt2532MW02, Pt2533MW02, Pt2534MW02, Pt2536MW02, Pt2538MW02, Pt2541MW02, Pt2612MW02, Pt2675MW02, Pt2697MW02, Pt2698MW02, Pt2699MW02, Pt2700MW02, Pt2717MW02, Pt2727MW02, Pt2748MW02, Pt2750MW02, Pt2752MW02, Pt2818MW02, Pt2837MW02, Pt2838MW02, Pt2882MW02, Pt2883MW02, Pt2888MW02, Pt2889MW02, Pt2980MW02, Pt2991MW02, Pt3138MW02, Pt3139MW02, Pt3141MW02, Pt3185MW02, Pt3186MW02, Pt3191MW02, Pt3193MW02, Pt3194MW02, Pt3199MW02, Pt3236MW02, Pt3276MW02, Pt3304MW02, Pt3315MW02, Pt3436MW02, Pt3438MW02, Pt3493MW02, and Pt3553MW02 as shown in Table 1.

### Dual-Targeting RNA Inhibitor

In another aspect, the present application provides a dual-targeting RNA inhibitor comprising a first RNA inhibitor targeting the PCSK9 gene and a second RNA inhibitor targeting LPA, wherein the first RNA inhibitor and the second RNA inhibitor are covalently linked by a linker.

In some embodiments, the first RNA inhibitor is a duplex comprising a sense strand and an antisense strand, wherein the sequence of the sense strand is CCAACUUUUCUAGACCUGUUU (SEQ ID NO: 100), and the sequence of the antisense strand is AAACAGGUCUAGAAAA GUUGGCU (SEQ ID NO: 427).

In some embodiments, the first RNA inhibitor is a duplex comprising a sense strand and an antisense strand, wherein the sequence of the sense strand is CUUUUCUAGACCUGUUUUGCU (SEQ ID NO: 104), and the sequence of the antisense strand is AGCAAAACAGGUCUAG AAAAGUU (SEQ ID NO: 431).

In some embodiments, the first RNA inhibitor is a duplex comprising a sense strand and an antisense strand, wherein the sequence of the sense strand is CmsCmsAmAmCmUmUfUm UfCfUfAmGmAmCmCmUmGmUmUmUm (SEQ ID NO: 988), and the sequence of the antisense strand is AmsAfsAmCmAmGmGmUmCmUmAmGmAmAfAmAfGmUmUmGmGmsCmsUm (SEQ ID NO: 1051).

In some embodiments, the first RNA inhibitor is a duplex comprising a sense strand and an antisense strand, wherein the sequence of the sense strand is CmsUmsUmUmUmCmUfAm GfAfCfCmUmGmUmUmUmUmGmCmUm (SEQ ID NO: 991), and the sequence of the antisense strand is AmsGfsCmAmAmAmAmCmAmGmGmUmCmUfAmGfAmAmAmAmGmsUmsUm (SEQ ID NO: 1054).

In some embodiments, the second RNA inhibitor is a duplex comprising a sense strand and an antisense strand, wherein the sequence of the sense strand is GGUGAUGGACAGAGUUAUCGA (SEQ ID NO: 1171), and the sequence of the antisense strand is UCGAUAACUCUGUCCAU CACCUC (SEQ ID NO: 1172).

In some embodiments, the second RNA inhibitor is a duplex comprising a sense strand and an antisense strand, wherein the sequence of the sense strand is GmsGmsUmGmAmUmGfGmAfCf AfGmAmGmUmUmAmUmCmGmAm (SEQ ID NO: 1173), and the sequence of the antisense strand is UmsCfsGmAmUmAmAmCmUmCmUmGmUmCfCmAfUmCmAmCmCmsUmsCm (SEQ ID NO: 1174).

In some embodiments, a sense strand of the second RNA inhibitor and a sense strand of the second inhibitor are covalently linked by a linker.

In some embodiments, the 3′-end of the sense strand of the first RNA inhibitor is linked to the linker, and the 5′-end of the sense strand of the second inhibitor is linked to the linker.

In some embodiments, the linker comprises a nucleotide linker and a non-nucleotide linker. The nucleotide linker refers to a linker having a nucleotide structure, and the non-nucleotide linker is a linker without a nucleotide structure.

In some embodiments, the nucleotide linker comprises a phosphorothioate group attached to the 5′- and/or 3′-end of the linker.

In some embodiments, the nucleotide linker comprises a plurality of methoxy-modified uridines U.

In some embodiments, the nucleotide linker is a sequence consisting of at least 3 Um.

In some embodiments, the nucleotide linker is UmUmUm or UmUmUmUmUmUm, wherein Um is a 2′-methoxy-modified uridine.

In some embodiments, the nucleotide linker comprises a plurality of deoxyadenosines (dA). In some embodiments, the nucleotide linker comprises a plurality of deoxyadenosines (dA) or a plurality of deoxythymidines (dT).

In some embodiments, the nucleotide linker is sdAdAdAs, sdAdAdAdAs, sdAdAdAdAdAs, sdAdAdAdAdAdAs, sdTdTdTs, sdTdTdTdTs, sdTdTdTdTdTs, or sdTdTdTdTdTdTs, wherein s is a phosphorothioate group.

In some embodiments, the non-nucleotide linker is

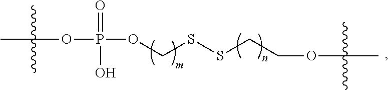

wherein m and n are each an integer of 3-10. For example, m is 5, and n is 5.

In some embodiments, the non-nucleotide linker is

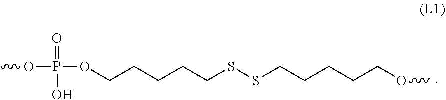

In some embodiments, the non-nucleotide linker comprises an ASGPR-binding group.

In some embodiments, the non-nucleotide linker comprises 2, 3, or 4 N-acetylgalactosamines.

In some embodiments, the non-nucleotide linker is

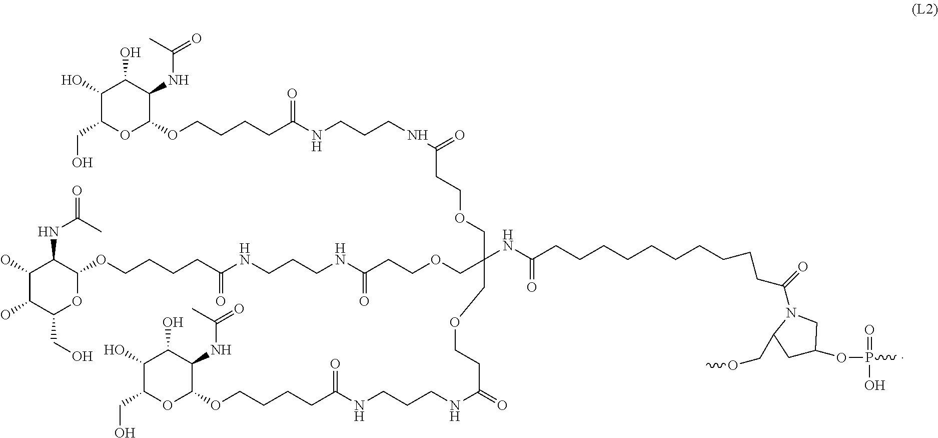

In some embodiments, the dual-targeting RNA inhibitor comprises a sense strand, a first antisense strand, and a second antisense strand, wherein the sense strand, the first antisense strand, and the second antisense strand comprise the following combinations:

(1) sense strand:
(SEQ ID NO: 988)
CmsCmsAmAmCmUmUfUmUfCfUfAmGmAmCmCmUmGmUmUmUm-
L1-
(SEQ ID NO: 1175)
GmsGmsUmGmAmUmGfGmAfCfAfGmAmGmUmUmAmUmCmsGmsAm;
first antisense strand:
(SEQ ID NO: 1051)
AmsAfsAmCmAmGmGmUmCmUmAmGmAmAfAmAfGmUmUmGmGmsCmsUm;
second antisense strand:
(SEQ ID NO: 1174)
UmsCfsGmAmUmAmAmCmUmCmUmGmUmCfCmAfUmCmAmCmCmsUmsCm
(2) sense strand:
(SEQ ID NO: 988)
CmsCmsAmAmCmUmUfUmUfCfUfAmGmAmCmCmUmGmUmUmUm-
L2-
(SEQ ID NO: 1173)
GmsGmsUmGmAmUmGfGmAfCfAfGmAmGmUmUmAmUmCmGmAm
first antisense strand:
(SEQ ID NO: 1051)
AmsAfsAmCmAmGmGmUmCmUmAmGmAmAfAmAfGmUmUmGmGmsCmsUm;
second antisense strand:
(SEQ ID NO: 1174)
UmsCfsGmAmUmAmAmCmUmCmUmGmUmCfCmAfUmCmAmCmCmsUmsCm
(3) sense strand:
(SEQ ID NO: 1176)
CmsCmsAmAmCmUmUfUmUfCfUfAmGmAmCmCmUmGmUmsUms
UmUmUmUmGmsGmsUmGmAmUmGfGmAfCfAfGmAmGmUmUmAmUmCmGmAm
first antisense strand:
(SEQ ID NO: 1051)
AmsAfsAmCmAmGmGmUmCmUmAmGmAmAfAmAfGmUmUmGmGmsCmsUm;
second antisense strand:
(SEQ ID NO: 1174)
UmsCfsGmAmUmAmAmCmUmCmUmGmUmCfCmAfUmCmAmCmCmsUmsCm
(4) sense strand:
(SEQ ID NO: 1177)
CmsCmsAmAmCmUmUfUmUfCfUfAmGmAmCmCmUmGmUmsUms
UmUmUmUmUmUmUmGmsGmsUmGmAmUmGfGmAfCfAfGmAmGmUmUmAmUmCmGmAm
first antisense strand:
(SEQ ID NO: 1051)
AmsAfsAmCmAmGmGmUmCmUmAmGmAmAfAmAfGmUmUmGmGmsCmsUm;
second antisense strand:
(SEQ ID NO: 1174)
UmsCfsGmAmUmAmAmCmUmCmUmGmUmCfCmAfUmCmAmCmCmsUmsCm
(5) sense strand:
(SEQ ID NO: 1178)
CmsUmsUmUmUmCmUfAmGfAfCfCmUmGmUmUmUmUmGmCmsUms-
L2-
(SEQ ID NO: 1173)
GmsGmsUmGmAmUmGfGmAfCfAfGmAmGmUmUmAmUmCmGmAm
first antisense strand:
(SEQ ID NO: 1054)
AmsGfsCmAmAmAmAmCmAmGmGmUmCmUfAmGfAmAmAmAmGmsUmsUm
second antisense strand:
(SEQ ID NO: 1174)
UmsCfsGmAmUmAmAmCmUmCmUmGmUmCfCmAfUmCmAmCmCmsUmsCm
(6) sense strand:
(SEQ ID NO: 1180)
CmsUmsUmUmUmCmUfAmGfAfCfCmUmGmUmUmUmUmGmCms
UmsdAdAdAdAdAdAsGmsGmUmGmAmUmGfGmAfCfAfGmAmGmUmUmAmUmCmGmAm;
first antisense strand:
(SEQ ID NO: 1054)
AmsGfsCmAmAmAmAmCmAmGmGmUmCmUfAmGfAmAmAmAmGmsUmsUm
second antisense strand:
(SEQ ID NO: 1174)
UmsCfsGmAmUmAmAmCmUmCmUmGmUmCfCmAfUmCmAmCmCmsUmsCm
(7) sense strand:
(SEQ ID NO: 1181)
CmsCmAmAmCmUmUfUmUfCfUfAmGmAmCmCmUmGmUmUms
AmsdAdAdAdAdAdAsGmsGmAmCmAmGmAfGmUfUfAfUmCmGmAmGmGmCmAmCmAm;
first antisense strand:
(SEQ ID NO: 1156)
UmsAfsAmCmAmGmGmUmCmUmAmGmAmAfAmAfGmUmUmGmGmsUmsAm
second antisense strand:
(SEQ ID NO: 1182)
UmsGfsUmGmCmCmUmCmGmAmUmAmAmCfUmCfUmGmUmCmCmsAmsUm
(8) sense strand:
(SEQ ID NO: 1183)
CmsCmsCmAmAmUmAfAmAfGfCfUmGmGmAmCmAmAmGmAms
AmsdAdAdAsGmsGmUmGmAmUmGfGmAfCfAfGmAmGmUmUmAmUmCmGmAm;
first antisense strand:
(SEQ ID NO: 1184)
UmsUfsCmUmUmGmUmCmCmAmGmCmUmUfUmAfUmUmGmGmGmsAmsGm
second antisense strand:
(SEQ ID NO: 1174)
UmsCfsGmAmUmAmAmCmUmCmUmGmUmCfCmAfUmCmAmCmCmsUmsCm
(9) sense strand:
(SEQ ID NO: 1185)
CmsCmsCmAmAmUmAfAmAfGfCfUmGmGmAmCmAmAmGmAms
AmsdAdAdAdAsGmsGmUmGmAmUmGfGmAfCfAfGmAmGmUmUmAmUmCmGmAm;
first antisense strand:
(SEQ ID NO: 1184)
UmsUfsCmUmUmGmUmCmCmAmGmCmUmUfUmAfUmUmGmGmGmsAmsGm
second antisense strand:
(SEQ ID NO: 1174)
UmsCfsGmAmUmAmAmCmUmCmUmGmUmCfCmAfUmCmAmCmCmsUmsCm
(10) sense strand:
(SEQ ID NO: 1186)
CmsCmsCmAmAmUmAfAmAfGfCfUmGmGmAmCmAmAmGmAms
AmsdAdAdAdAdAdAsGmsGmUmGmAmUmGfGmAfCfAfGmAmGmUmUmAmUmCmGmAm;
first antisense strand:
(SEQ ID NO: 1184)
UmsUfsCmUmUmGmUmCmCmAmGmCmUmUfUmAfUmUmGmGmGmsAmsGm
second antisense strand:
(SEQ ID NO: 1174)
UmsCfsGmAmUmAmAmCmUmCmUmGmUmCfCmAfUmCmAmCmCmsUmsCm
(11) sense strand:
(SEQ ID NO: 1187)
CmsCmsCmAmAmUmAfAmAfGfCfUmGmGmAmCmAmAmGmAms
AmsdTdTdTsGmsGmUmGmAmUmGfGmAfCfAfGmAmGmUmUmAmUmCmGmAm;
first antisense strand:
(SEQ ID NO: 1184)
UmsUfsCmUmUmGmUmCmCmAmGmCmUmUfUmAfUmUmGmGmGmsAmsGm
second antisense strand:
(SEQ ID NO: 1174)
UmsCfsGmAmUmAmAmCmUmCmUmGmUmCfCmAfUmCmAmCmCmsUmsCm
(12) sense strand:
(SEQ ID NO: 1188)
CmsCmsCmAmAmUmAfAmAfGfCfUmGmGmAmCmAmAmGm
AmsAmsdTdTdTdTsGmsGmsUmGmAmUmGfGmAfCfAfGmAmGmUmUmAmUmCmGmAm;
first antisense strand:
(SEQ ID NO: 1184)
UmsUfsCmUmUmGmUmCmCmAmGmCmUmUfUmAfUmUmGmGmGmsAmsGm
second antisense strand:
(SEQ ID NO: 1174)
UmsCfsGmAmUmAmAmCmUmCmUmGmUmCfCmAfUmCmAmCmCmsUmsCm
(13) sense strand:
(SEQ ID NO: 1189)
CmsCmsCmAmAmUmAfAmAfGfCfUmGmGmAmCmAmAmGmAms
AmsdTdTdTdTdTsGmsGmUmGmAmUmGfGmAfCfAfGmAmGmUmUmAmUmCmGmAm;
first antisense strand:
(SEQ ID NO: 1184)
UmsUfsCmUmUmGmUmCmCmAmGmCmUmUfUmAfUmUmGmGmGmsAmsGm
second antisense strand:
(SEQ ID NO: 1174)
UmsCfsGmAmUmAmAmCmUmCmUmGmUmCfCmAfUmCmAmCmCmsUmsCm
(14) sense strand:
(SEQ ID NO: 1190)
CmsCmsCmAmAmUmAfAmAfGfCfUmGmGmAmCmAmAmGmAms
AmsdTdTdTdTdTdTsGmsGmUmGmAmUmGfGmAfCfAfGmAmGmUmUmAmUmCmGmAm;
first antisense strand:
(SEQ ID NO: 1184)
UmsUfsCmUmUmGmUmCmCmAmGmCmUmUfUmAfUmUmGmGmGmsAmsGm
second antisense strand:
(SEQ ID NO: 1174)
UmsCfsGmAmUmAmAmCmUmCmUmGmUmCfCmAfUmCmAmCmCmsUmsCm
(15) sense strand:
(SEQ ID NO: 1191)
CmsCmsCmAmAmUmAfAmAfGfCfUmGmGmAmCmAmAmGm
AmsAmsdAdAdAdAdAsGmsGmUmGmAmUmGfGmAfCfAfGmAmGmUmUmAmUmCmGmAm;
first antisense strand:
(SEQ ID NO: 1184)
UmsUfsCmUmUmGmUmCmCmAmGmCmUmUfUmAfUmUmGmGmGmsAmsGm
second antisense strand:
(SEQ ID NO: 1174)
UmsCfsGmAmUmAmAmCmUmCmUmGmUmCfCmAfUmCmAmCmCmsUmsCm.

### RNA Inhibitor Coupled to Ligand

Another aspect of the RNA inhibitor of the present application relates to a way of coupling an interfering nucleic acid to a ligand to enhance the stability, activity, cellular distribution, or cellular uptake of the RNAi agent.

In certain embodiments, a ligand of a target tissue receptor is introduced to alter the distribution, targeting, or stability of the RNA inhibitor. For example, a specific ligand may provide an enhanced affinity for a selected target (e.g., a molecule, a cell or cell type, a compartment, (e.g., a cellular or organ compartment, a tissue, organ, or region of the body)) as compared with species where such a ligand is absent.

Ligands may include naturally occurring substances such as proteins (e.g., human serum albumin (HSA), low-density lipoprotein (LDL), or globulin); carbohydrates (e.g., dextran, pullulan, chitin, chitosan, inulin, cyclodextrin, N-acetylglucosamine, N-acetylgalactosamine, or hyaluronic acid); or lipids. Ligands may also be recombinant or synthetic molecules, such as synthetic polymers, e.g., a synthetic polyamino acid.

Ligands may also include targeting groups, such as a cell or tissue targeting agent, e.g., a lectin, glycoprotein, lipid, or protein, e.g., an antibody, that binds to a specified cell type, e.g., a kidney cell. The targeting groups may be thyrotropin, melanotropin, lectins, glycoproteins, surfactant protein A, mucin carbohydrates, multivalent lactose, multivalent galactose, N-acetyl-galactosamine, N-acetyl-glucosamine multivalent mannose, multivalent fucose, glycosylated polyamino acids, multivalent galactose, transferrin, bisphosphonates, polyglutamic acid, polyaspartic acid, lipids, cholesterol, steroids, bile acid, folic acid, vitamin B12, vitamin A, biotin, or RGD peptide or RGD peptide mimics. In certain embodiments, the ligand is multivalent galactose, e.g., N-acetyl-galactosamine.

The sense strand and the antisense strand contained in the RNA inhibitor of the present application may be conveniently and conventionally prepared by the well-known technique of solid-phase synthesis. Additionally or alternatively, any other method known in the art for such synthesis, such as liquid-phase synthesis or fermentation, may be used. It is also known to use similar techniques to prepare other oligonucleotides (e.g., phosphorothioates and alkylated derivatives).

In certain embodiments, in addition to standard nucleoside phosphoramidite monomers and non-standard nucleoside phosphoramidite monomers that are commercially available and conventionally used in oligonucleotide synthesis, the oligonucleotides or linked nucleotides of the present application may be synthesized by an automatic synthesizer using the phosphoramidite method derived from ligand-nucleoside phosphoramidite monomers.

In certain embodiments, the ligand conjugation method described in the present disclosure is to couple the ligand structure to the 5′-end and/or the 3′-end of the antisense strand, and/or the 5′-end and/or the 3′-end of the sense strand.

For example, the ligand structure may be coupled to the 5′-end and/or the 3′-end of the sense strand; or the ligand structure may be coupled to the 5′-end of the antisense strand, and the ligand structure is coupled to the 3′-end of the sense strand; or the ligand structure may be coupled to the 3′-end of the antisense strand, and the ligand is coupled to the 5′-end of the sense strand; or the ligand structure is coupled to the 5′-end and the 3′-end of the sense strand; or the ligand is coupled to the 3′-end of the sense strand.

In certain embodiments, the ligand described in the present application comprises an L96 structure, as shown in the following structural formula:

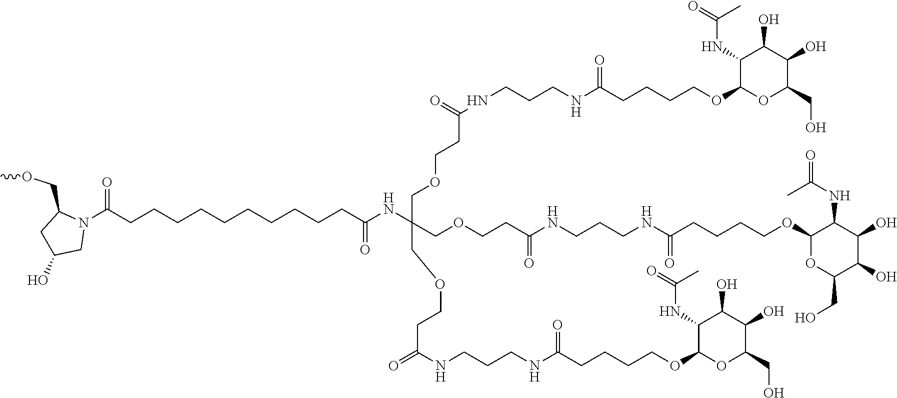

### Pharmaceutical Composition

The present application also includes a pharmaceutical composition comprising the RNA inhibitor in the present application or a pharmaceutically acceptable salt thereof.

In one embodiment, provided herein is a pharmaceutical composition comprising the RNA inhibitor described herein and a pharmaceutically acceptable pharmaceutical adjuvant. The pharmaceutical composition comprising the RNA inhibitor may be used for preventing and/or treating a PCSK9-associated disorder, e.g., hypercholesterolemia. Such a pharmaceutical composition is formulated according to the mode of delivery. An embodiment is to prepare a composition for systemic administration by parenteral delivery, e.g., subcutaneous (SC), intramuscular (IM), or intravenous (IV) delivery. The pharmaceutical composition of the present application may be administered at a dose sufficient to inhibit the expression of the PCSK9 gene.

Pharmaceutically acceptable “adjuvants” or “excipients” are pharmaceutically acceptable solvents, suspending agents, or any other pharmaceutically inert vehicles for delivering one or more nucleic acids to animals. The excipients may be liquid or solid and are selected taking into account of the intended mode of administration to provide a desired volume, consistency, etc. when combined with nucleic acids and other components in a given pharmaceutical composition. The RNA inhibitor may be delivered in a manner that targets specific tissues (e.g., hepatocytes).

In certain embodiments, the pharmaceutical composition further comprises a delivery vehicle (e.g., a nanoparticle, a dendrimer, a polymer, a liposome, or a cationic delivery system).

In certain embodiments, the delivery vehicle comprises a liposome.

In certain embodiments, the delivery vehicle comprises a nanolipid capable of forming a liposome-nucleic acid nanoparticle with a nucleic acid molecule.

### Use

In another aspect, the present application provides use of the aforementioned RNA inhibitor for inhibiting PCSK9 gene expression or the pharmaceutically acceptable salt thereof and the aforementioned pharmaceutical composition in the manufacture of a medicament for preventing or treating a disease or pathology, or reducing the risk of a disease or pathology.

In certain embodiments, the disease or pathology comprises a disease or pathology associated with elevated levels of PCSK9.

In certain embodiments, the disease or pathology comprises hypercholesterolemia.

In certain embodiments, the disease or pathology comprises inflammatory, cardiovascular and cerebrovascular, or metabolic diseases.

In certain embodiments, the cardiovascular and cerebrovascular disease comprises hyperlipidemia, stroke, atherosclerosis, thrombosis, coronary heart disease, or aortic stenosis.

In another aspect, the present application provides a method for preventing or treating a disease, condition, or syndrome, which comprises administering to a subject in need thereof an effective amount of the aforementioned RNA inhibitor for inhibiting PCSK9 gene expression, the pharmaceutically acceptable salt thereof, or the aforementioned pharmaceutical composition.

In certain embodiments, the RNA inhibitor for inhibiting PCSK9 gene expression, the pharmaceutically acceptable salt, or the pharmaceutical composition is administered to the subject by a subcutaneous, intravenous, oral, rectal, or intraperitoneal administration route.

In another aspect, the present application provides a method for inhibiting the expression of PCSK9 mRNA or protein in a cell, tissue, or subject, which comprises administering to the subject in need thereof an effective amount of the aforementioned RNA inhibitor for inhibiting PCSK9 gene expression, the pharmaceutically acceptable salt thereof, or the aforementioned pharmaceutical composition.

In certain embodiments, the cell is a hepatocyte.

In certain embodiments, the tissue is a liver tissue.

In certain embodiments, the cell and tissue are ex vivo.

Cells suitable for treatment using the method of the present application may be any cells expressing the PCSK9 gene, e.g., liver cells, brain cells, gallbladder cells, heart cells, or kidney cells, but preferably liver cells. Cells suitable for use in the method of the present application may be mammalian cells. When in contact with cells expressing the PCSK9 gene, the RNAi agent inhibits the expression of the PCSK9 gene (e.g., a human, primate, non-primate, or rat PCSK9 gene) by at least about 50%, as determined by, for example, PCR or branched DNA (bDNA)-based methods, or protein-based methods such as immunofluorescence assay, western blot, or flow cytometry.

As used herein, the term “inhibit” is used interchangeably with “reduce”, “decrease”, “silence”, “down-regulate”, “suppress”, and other similar terms, and includes any level of inhibition. The expression of the PCSK9 gene may be assessed according to the level or change in the level of any variable associated with PCSK9 gene expression, e.g., PCSK9 mRNA level or PCSK9 protein level. This level may be assayed in a single cell or in a cell population (including, for example, a sample derived from a subject). The inhibition may be assessed by a decrease in an absolute or relative level of one or more variables associated with PCSK9 expression compared with a control level. The control level may be any type of control level used in the art, such as a pre-dose baseline level or a level determined from a similar subject, cell, or sample that is untreated or treated with a control (e.g., a buffer-only control or an active agent-free control).

The inhibition of PCSK9 gene expression may be manifested by a decrease in the amount of an mRNA expressed by a first cell or cell population (such a cell may be, for example, present in a sample derived from a subject) in which the PCSK9 gene is transcribed and which has been treated (e.g., by contacting one or more cells with the RNA inhibitor of the present application, or by administering the RNA inhibitor of the present application to a subject in which the cell is present) such that the PCSK9 gene expression is inhibited, as compared with a second cell or cell population substantially identical to the first cell or cell population but not so treated (a control cell not treated with the RNA inhibitor or not treated with an RNA inhibitor targeting the target gene). In a preferred embodiment, the inhibition is assessed by the method provided in Example 2 using an appropriate concentration of siRNA in a cell line highly expressing PCSK9, and the mRNA level in the interfered cells is expressed as a percentage of the mRNA level in control cells without interference.

In other embodiments, the inhibition of PCSK9 gene expression may be assessed by a decrease in parameters functionally related to the PCSK9 gene expression, such as the level of the PCSK9 protein in the blood or serum of a subject. PCSK9 gene silencing may be determined in any cell expressing PCSK9 (endogenous or exogenously from an expression construct) and by any assay known in the art.

The inhibition of PCSK9 protein expression may be manifested by a decrease in the level of PCSK9 protein expressed by a cell or cell population or a subject sample (e.g., the level of protein in a blood sample derived from the subject). As described above, for the assessment of mRNA inhibition, the inhibition of the protein expression level of a treated cell or cell population may be similarly expressed as a percentage of the protein level of a control cell or cell population, or a change in the protein level in a subject sample (e.g., blood or serum derived therefrom).

Control group cells, cell populations, or subject samples that may be used for assessing the inhibition of the PCSK9 gene include cells, cell populations, or subject samples not in contact with the RNAi agent of the present application. For example, the control cells, cell populations, or subject samples may be derived from a single subject (e.g., a human or animal subject) prior to the treatment with the RNAi agent, or an appropriately matched population control.

The level of PCSK9 mRNA expressed by a cell or cell population may be determined using any method known in the art for assessing mRNA expression. For example, qRT-PCR is used to assess the decrease in gene expression. The decrease in protein production may be assessed by any method known in the art, e.g., ELISA. In certain embodiments, a puncture liver biopsy sample is used as a tissue material for monitoring the decrease in the expression of the PCSK9 gene or protein. In other embodiments, a blood sample is used as a subject sample for monitoring the decrease in PCSK9 protein expression.

The present application further comprises the following embodiments:
1. An RNA inhibitor for inhibiting PCSK9 gene expression, comprising an antisense strand, wherein the antisense strand comprises a complementary region that is complementary to at least a part of an mRNA encoding PCSK9, and the complementary region is 17-23 nucleotides in length, wherein the antisense strand comprises one of the following nucleotide sequences:
(1) SEQ ID NO: 431 or a sequence differing by no more than 3 nucleotides therefrom;
(2) any one of SEQ ID NOs: 328-430, 432-654, 1045-1107, 1125-1146, or 1149-1170, or a sequence differing by no more than 3 nucleotides therefrom.
2. The RNA inhibitor for inhibiting PCSK9 gene expression according to embodiment 1, further comprising a sense strand, wherein at least 80% of bases between the sense strand and the antisense strand are complementary.
3. The RNA inhibitor for inhibiting PCSK9 gene expression according to any one of embodiments 1-2, wherein the sense nucleic acid strand and the antisense nucleic acid strand are present on two different nucleic acid strands.
4. The RNA inhibitor for inhibiting PCSK9 gene expression according to any one of embodiments 1-2, wherein the sense nucleic acid fragment and the antisense nucleic acid fragment are present on the same nucleic acid strand, wherein complementary regions of the sense nucleic acid fragment and the antisense nucleic acid fragment form a double-stranded nucleic acid structure.
5. The RNA inhibitor for inhibiting PCSK9 gene expression according to any one of embodiments 1-4, wherein at least one strand has a 3′ overhang of 0 to 6 nucleotides in length.
6. The RNA inhibitor for inhibiting PCSK9 gene expression according to any one of embodiments 1-5, wherein both strands have 3′ overhangs of 2-3 nucleotides in length, or the sense strand has a 3′ overhang of 2-3 nucleotides in length, or the antisense strand has a 3′ overhang of 2-3 nucleotides in length.
7. The RNA inhibitor for inhibiting PCSK9 gene expression according to any one of embodiments 1-6, wherein the sense nucleic acid strand and the antisense nucleic acid strand are each 16 to 35 nucleotides in length.
8. The RNA inhibitor for inhibiting PCSK9 gene expression according to any one of embodiments 1-7, wherein one strand of the RNA inhibitor for inhibiting PCSK9 gene expression has at least 75% homology or complementarity to any one of the nucleotide sequences selected from SEQ ID NOs: 655-981 and 1108-1111.
9. The RNA inhibitor for inhibiting PCSK9 gene expression according to any one of embodiments 1-8, wherein the sense strand is selected from any one of SEQ ID NOs: 1-327, 982-1044, 1123-1124, and 1147-1148, and a sequence differing by no more than 3 nucleotides therefrom.
10. The RNA inhibitor for inhibiting PCSK9 gene expression according to any one of embodiments 1-9, wherein at least one nucleotide is a nucleotide with a chemical modification.
11. The RNA inhibitor for inhibiting PCSK9 gene expression according to any one of embodiments 1-10, wherein the chemical modification is at least one of the following modifications:
(1) a modification of a phosphodiester bond linking nucleotides in the nucleotide sequence of the RNA inhibitor for inhibiting PCSK9 gene expression;
(2) a modification of 2′-OH of a ribose in the nucleotide sequence of the RNA inhibitor for inhibiting PCSK9 gene expression; and
(3) a modification of a base in the nucleotide sequence of the RNA inhibitor for inhibiting PCSK9 gene expression.
12. The RNA inhibitor for inhibiting PCSK9 gene expression according to any one of embodiments 1-11, wherein at least two consecutive phosphorothioate bonds are present between nucleotides of the sense strand and/or the antisense strand.
13. The RNA inhibitor for inhibiting PCSK9 gene expression according to any one of embodiments 1-12, wherein at least two consecutive phosphorothioate bonds are present between three consecutive nucleotides at the end of the sense strand and/or the end of the antisense strand.
14. The RNA inhibitor for inhibiting PCSK9 gene expression according to any one of embodiments 1-13, wherein the —OH groups at the 2′ positions of the glycosyl groups in the nucleotides at positions 7, 9, 10, and 11 starting from the 5′-end of the sense strand are replaced by fluorine, and the —OH groups at the 2′-positions of the glycosyl groups in the remaining nucleotides of the sense strand are replaced by methoxy groups.
15. The RNA inhibitor for inhibiting PCSK9 gene expression according to any one of embodiments 1-14, wherein the —OH groups at the 2′-positions of the glycosyl groups in the nucleotides at positions 2, 14, and 16 starting from the 5′-end of the antisense strand are replaced by fluorine, and the —OH groups at the 2′-positions of the glycosyl groups in the remaining nucleotides of the antisense strand are replaced by methoxy groups.
16. The RNA inhibitor for inhibiting PCSK9 gene expression according to any one of embodiments 1-15, comprising: SP345, Ps3464, Ps3543, Ps3554, SP340, SP341, SP342, SP351, Pt2888, Pt3493, or Pt3553.
17. The RNA inhibitor for inhibiting PCSK9 gene expression according to any one of embodiments 1-16, comprising: SP345MW02, P3119MW02, P3196MW02, P3464MW02, P3543MW02, P3554MW02, SP340MW02, SP341MW02, SP342MW02, SP344MW02, SP349MW02, SP351MW02, WP3239MW02, WP3262MW02, WP3315MW02, WP3315-AMW02, WP3323MW02, Pt609MW02, Pt890MW02, Pt2530MW02, Pt2532MW02, Pt2533MW02, Pt2534MW02, Pt2536MW02, Pt2538MW02, Pt2541MW02, Pt2612MW02, Pt2675MW02, Pt2697MW02, Pt2698MW02, Pt2699MW02, Pt2700MW02, Pt2717MW02, Pt2727MW02, Pt2748MW02, Pt2750MW02, Pt2752MW02, Pt2818MW02, Pt2837MW02, Pt2838MW02, Pt2882MW02, Pt2883MW02, Pt2888MW02, Pt2889MW02, Pt2980MW02,

Pt2991MW02, Pt3138MW02, Pt3139MW02, Pt3141MW02, Pt3185MW02, Pt3186MW02, Pt3191MW02, Pt3193MW02, Pt3194MW02, Pt3199MW02, Pt3236MW02, Pt3276MW02, Pt3304MW02, Pt3315MW02, Pt3436MW02, Pt3438MW02, Pt3493MW02, or Pt3553MW02.
18. The RNA inhibitor for inhibiting PCSK9 gene expression according to any one of embodiments 1-17, further comprising a ligand, wherein the ligand is conjugated to the sense strand and/or the antisense strand.
19. The RNA inhibitor for inhibiting PCSK9 gene expression according to embodiment 18, wherein the ligand is conjugated to the 5′-end and/or the 3′-end of the antisense strand.
20. The RNA inhibitor for inhibiting PCSK9 gene expression according to any one of embodiments 18-19, wherein the ligand is conjugated to the 5′-end and/or the 3′-end of the sense strand.
21. The RNA inhibitor for inhibiting PCSK9 gene expression according to any one of embodiments 18-20 or a pharmaceutically acceptable salt thereof, wherein the ligand is conjugated to the 5′-end of the antisense strand, and the ligand is conjugated to the 3′-end of the sense strand.
22. The RNA inhibitor for inhibiting PCSK9 gene expression according to any one of embodiments 18-20, wherein the ligand is conjugated to the 3′-end of the antisense strand, and the ligand is conjugated to the 5′-end of the sense strand.
23. The RNA inhibitor for inhibiting PCSK9 gene expression according to any one of embodiments 18-20, wherein the ligand is conjugated to the 5′-end and the 3′-end of the sense strand.
24. The RNA inhibitor for inhibiting PCSK9 gene expression according to any one of embodiments 18-23, wherein the ligand further comprises a targeting unit, i.e. a structure for enhancing uptake of the RNA inhibitor by a hepatocyte.
25. The RNA inhibitor for inhibiting PCSK9 gene expression according to embodiment 24, wherein the targeting unit is selected from a monosaccharide and a derivative thereof.
26. The RNA inhibitor for inhibiting PCSK9 gene expression according to any one of embodiments 24-25, wherein the monosaccharide is selected from one or more of the following structures: mannose, galactose, D-arabinose, glucose, fructose, xylose, glucosamine, and ribose.
27. The RNA inhibitor for inhibiting PCSK9 gene expression according to any one of embodiments 24-26, wherein the monosaccharide derivative is selected from a mannose derivative, a galactose derivative, a glucose derivative, a ribose derivative, and other derivatives.
28. The RNA inhibitor for inhibiting PCSK9 gene expression according to any one of embodiments 24-27, wherein the targeting unit is selected from galactose, galactosamine, N-acetylgalactosamine, and derivatives thereof.
29. The RNA inhibitor for inhibiting PCSK9 gene expression according to any one of embodiments 24-28, wherein the targeting unit is N-acetylgalactosamine and a derivative thereof.
30. A dual-targeting RNA inhibitor, comprising a first RNA inhibitor targeting the PCSK9 gene and a second RNA inhibitor targeting LPA, wherein the first RNA inhibitor and the second RNA inhibitor are covalently linked by a linker.
31. The dual-targeting RNA inhibitor according to embodiment 30, wherein the first RNA inhibitor is a duplex comprising a sense strand and an antisense strand, wherein the sequence of the sense strand is CCAACUUUUCUAGACCUGUUU (SEQ ID NO: 100), and the sequence of the antisense strand is AAACAGGUCUAGAAAAGUUGGCU (SEQ ID NO: 427).
32. The dual-targeting RNA inhibitor according to embodiment 30, wherein the first RNA inhibitor is a duplex comprising a sense strand and an antisense strand, wherein the sequence of the sense strand is CUUUUCUAGACCUGUUUUGCU (SEQ ID NO: 104), and the sequence of the antisense strand is AGCAAAACAGGUCUAGAAAAGUU (SEQ ID NO: 431).
33. The dual-targeting RNA inhibitor according to embodiment 31, wherein the first RNA inhibitor is a duplex comprising a sense strand and an antisense strand, wherein the sequence of the sense strand is CmsCmsAmAmCmUmUfUmUfCfUfAmGmAmCmCmUmGmUmUmUm (SEQ ID NO: 988), and the sequence of the antisense strand is AmsAfsAmCmAmGmGmUm CmUmAmGmAmAfAmAfGmUmUmGmGmsCmsUm (SEQ ID NO: 1051).
34. The dual-targeting RNA inhibitor according to embodiment 32, wherein the first RNA inhibitor is a duplex comprising a sense strand and an antisense strand, wherein the sequence of the sense strand is CmsUmsUmUmUmCmUfAmGfAfCfCmUmGmUmUmUmUmGmCmUm (SEQ ID NO: 991), and the sequence of the antisense strand is AmsGfsCmAmAmAmAmCm AmGmGmUmCmUfAmGfAmAmAmAmGmsUmsUm (SEQ ID NO: 1054).
35. The dual-targeting RNA inhibitor according to embodiment 30, wherein the second RNA inhibitor is a duplex comprising a sense strand and an antisense strand, wherein the sequence of the sense strand is GGUGAUGGACAGAGUUAUCGA (SEQ ID NO: 1171), and the sequence of the antisense strand is UCGAUAACUCUGUCCAUCACCUC (SEQ ID NO: 1172).
36. The dual-targeting RNA inhibitor according to embodiment 35, wherein the second RNA inhibitor is a duplex comprising a sense strand and an antisense strand, wherein the sequence of the sense strand is GmsGmsUmGmAmUmGfGmAfCfAfGmAmGmUmUmAmUmCmGmAm (SEQ ID NO: 1173), and the sequence of the antisense strand is UmsCfsGmAmUmAmAmCm UmCmUmGmUmCfCmAfUmCmAmCmCmsUmsCm (SEQ ID NO: 1174).
37. The dual-targeting RNA inhibitor according to embodiment 30, wherein a sense strand of the second RNA inhibitor and a sense strand of the second inhibitor are covalently linked by a linker.
38. The dual-targeting RNA inhibitor according to embodiment 37, wherein the 3′-end of the sense strand of the first RNA inhibitor is linked to the linker, and the 5′-end of the sense strand of the second inhibitor is linked to the linker.
39. The dual-targeting RNA inhibitor according to embodiment 30, wherein the linker comprises a nucleotide linker and a non-nucleotide linker.
40. The dual-targeting RNA inhibitor according to embodiment 39, wherein the nucleotide linker comprises a phosphorothioate group attached to the 5′- and/or 3′-end of the linker.
41. The dual-targeting RNA inhibitor according to embodiment 39, wherein the nucleotide linker comprises a plurality of methoxy-modified uridines U.
42. The dual-targeting RNA inhibitor according to embodiment 40, wherein the nucleotide linker is a sequence consisting of at least 3 Um.
43. The dual-targeting RNA inhibitor according to embodiment 42, wherein the nucleotide linker is UmUmUm or UmUmUmUmUmUm, wherein Um is a 2′-methoxy-modified uridine.
44. The dual-targeting RNA inhibitor according to embodiment 39, wherein the nucleotide linker comprises a plurality of deoxyadenosines (dA) or a plurality of deoxythymidines (dT).
45. The dual-targeting RNA inhibitor according to embodiment 43, wherein the nucleotide linker is sdAdAdAs, sdAdAdAdAs, sdAdAdAdAdAs, sdAdAdAdAdAdAs, sdTdTdTs, sdTdTdTdTs, sdTdTdTdTdTs, or sdTdTdTdTdTdTs, wherein s is a phosphorothioate group.
46. The dual-targeting RNA inhibitor according to embodiment 39, wherein the non-nucleotide linker comprises

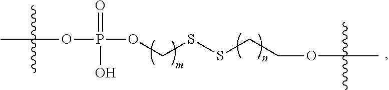

wherein m and n are each an integer of 3-10.
47. The dual-targeting RNA inhibitor according to embodiment 45, wherein the non-nucleotide linker is

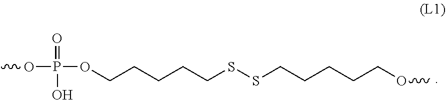

48. The dual-targeting RNA inhibitor according to embodiment 39, wherein the non-nucleotide linker comprises an ASGPR-binding group.
49. The dual-targeting RNA inhibitor according to embodiment 47, wherein the non-nucleotide linker comprises 2, 3, or 4 N-acetylgalactosamines.
50. The dual-targeting RNA inhibitor according to embodiment 48, wherein the non-nucleotide linker is

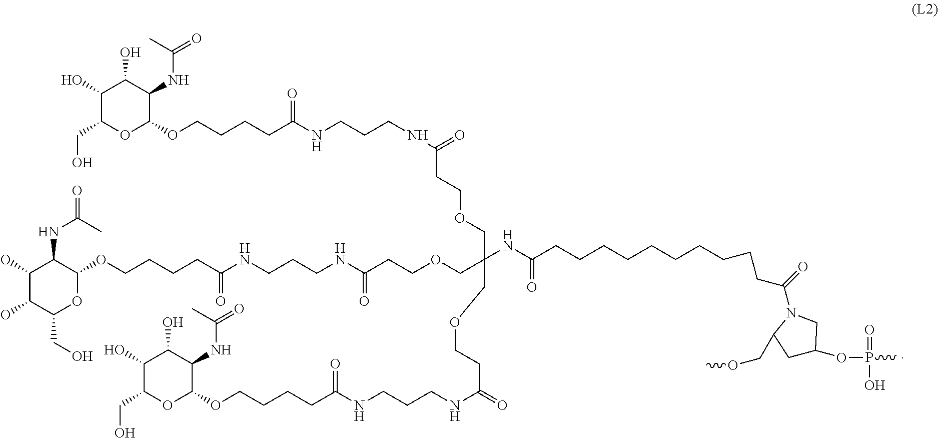

51. The dual-targeting RNA inhibitor according to embodiments 30-50, comprising a sense strand, a first antisense strand, and a second antisense strand, wherein the sense strand, the first antisense strand, and the second antisense strand comprise the following combinations:

(1) sense strand:
(SEQ ID NO: 988)
CmsCmsAmAmCmUmUfUmUfCfUfAmGmAmCmCmUmGmUmUmUm-
L1-
(SEQ ID NO: 1175)
GmsGmsUmGmAmUmGfGmAfCfAfGmAmGmUmUmAmUmCmsGmsAm;
first antisense strand:
(SEQ ID NO: 1051)
AmsAfsAmCmAmGmGmUmCmUmAmGmAmAfAmAfGmUmUmGmGmsCmsUm;
second antisense strand:
(SEQ ID NO: 1174)
UmsCfsGmAmUmAmAmCmUmCmUmGmUmCfCmAfUmCmAmCmCmsUmsCm
(2) sense strand:
(SEQ ID NO: 988)
CmsCmsAmAmCmUmUfUmUfCfUfAmGmAmCmCmUmGmUmUmUm-
L2-
(SEQ ID NO: 1173)
GmsGmsUmGmAmUmGfGmAfCfAfGmAmGmUmUmAmUmCmGmAm
1. first antisense strand:
(SEQ ID NO: 1051)
AmsAfsAmCmAmGmGmUmCmUmAmGmAmAfAmAfGmUmUmGmGmsCmsUm;
2. second antisense strand:
(SEQ ID NO: 1174)
UmsCfsGmAmUmAmAmCmUmCmUmGmUmCfCmAfUmCmAmCmCmsUmsCm
(3) sense strand:
(SEQ ID NO: 1176)
CmsCmsAmAmCmUmUfUmUfCfUfAmGmAmCmCmUmGmUmsUms
UmUmUmUmGmsGmsUmGmAmUmGfGmAfCfAfGmAmGmUmUmAmUmCmGmAm
first antisense strand:
(SEQ ID NO: 1051)
AmsAfsAmCmAmGmGmUmCmUmAmGmAmAfAmAfGmUmUmGmGmsCmsUm;
second antisense strand:
(SEQ ID NO: 1174)
UmsCfsGmAmUmAmAmCmUmCmUmGmUmCfCmAfUmCmAmCmCmsUmsCm
(4) sense strand:
(SEQ ID NO: 1177)
CmsCmsAmAmCmUmUfUmUfCfUfAmGmAmCmCmUmGmUmsUms
UmUmUmUmUmUmUmGmsGmsUmGmAmUmGfGmAfCfAfGmAmGmUmUmAmUmCmGmAm
first antisense strand:
(SEQ ID NO: 1051)
AmsAfsAmCmAmGmGmUmCmUmAmGmAmAfAmAfGmUmUmGmGmsCmsUm;
second antisense strand:
(SEQ ID NO: 1174)
UmsCfsGmAmUmAmAmCmUmCmUmGmUmCfCmAfUmCmAmCmCmsUmsCm
(5) sense strand:
(SEQ ID NO: 1178)
CmsUmsUmUmUmCmUfAmGfAfCfCmUmGmUmUmUmUmGmCmsUms-
L2-
(SEQ ID NO: 1173)
GmsGmsUmGmAmUmGfGmAfCfAfGmAmGmUmUmAmUmCmGmAm
first antisense strand:
(SEQ ID NO: 1054)
AmsGfsCmAmAmAmAmCmAmGmGmUmCmUfAmGfAmAmAmAmGmsUmsUm
second antisense strand:
(SEQ ID NO: 1174)
UmsCfsGmAmUmAmAmCmUmCmUmGmUmCfCmAfUmCmAmCmCmsUmsCm
(6) sense strand:
(SEQ ID NO: 1180)
CmsUmsUmUmUmCmUfAmGfAfCfCmUmGmUmUmUmUmGmCms
UmsdAdAdAdAdAdAsGmsGmUmGmAmUmGfGmAfCfAfGmAmGmUmUmAmUmCmGmAm;
3. first antisense strand:
(SEQ ID NO: 1054)
AmsGfsCmAmAmAmAmCmAmGmGmUmCmUfAmGfAmAmAmAmGmsUmsUm
4. second antisense strand:
(SEQ ID NO: 1174)
UmsCfsGmAmUmAmAmCmUmCmUmGmUmCfCmAfUmCmAmCmCmsUmsCm
(7) sense strand:
(SEQ ID NO: 1181)
CmsCmAmAmCmUmUfUmUfCfUfAmGmAmCmCmUmGmUmUms
AmsdAdAdAdAdAdAsGmsGmAmCmAmGmAfGmUfUfAfUmCmGmAmGmGmCmAmCmAm;
first antisense strand:
(SEQ ID NO: 1156)
UmsAfsAmCmAmGmGmUmCmUmAmGmAmAfAmAfGmUmUmGmGmsUmsAm
second antisense strand:
(SEQ ID NO: 1182)
UmsGfsUmGmCmCmUmCmGmAmUmAmAmCfUmCfUmGmUmCmCmsAmsUm
(8) sense strand:
(SEQ ID NO: 1183)
CmsCmsCmAmAmUmAfAmAfGfCfUmGmGmAmCmAmAmGmAms
AmsdAdAdAsGmsGmUmGmAmUmGfGmAfCfAfGmAmGmUmUmAmUmCmGmAm;
first antisense strand:
(SEQ ID NO: 1184)
UmsUfsCmUmUmGmUmCmCmAmGmCmUmUfUmAfUmUmGmGmGmsAmsGm
second antisense strand:
(SEQ ID NO: 1174)
UmsCfsGmAmUmAmAmCmUmCmUmGmUmCfCmAfUmCmAmCmCmsUmsCm
(9) sense strand:
(SEQ ID NO: 1185)
CmsCmsCmAmAmUmAfAmAfGfCfUmGmGmAmCmAmAmGmAms
AmsdAdAdAdAsGmsGmUmGmAmUmGfGmAfCfAfGmAmGmUmUmAmUmCmGmAm;
first antisense strand:
(SEQ ID NO: 1184)
UmsUfsCmUmUmGmUmCmCmAmGmCmUmUfUmAfUmUmGmGmGmsAmsGm
second antisense strand:
(SEQ ID NO: 1174)
UmsCfsGmAmUmAmAmCmUmCmUmGmUmCfCmAfUmCmAmCmCmsUmsCm
(10) sense strand:
(SEQ ID NO: 1186)
CmsCmsCmAmAmUmAfAmAfGfCfUmGmGmAmCmAmAmGmAms
AmsdAdAdAdAdAdAsGmsGmUmGmAmUmGfGmAfCfAfGmAmGmUmUmAmUmCmGmAm;
first antisense strand:
(SEQ ID NO: 1184)
UmsUfsCmUmUmGmUmCmCmAmGmCmUmUfUmAfUmUmGmGmGmsAmsGm
second antisense strand:
(SEQ ID NO: 1174)
UmsCfsGmAmUmAmAmCmUmCmUmGmUmCfCmAfUmCmAmCmCmsUmsCm
(11) sense strand:
(SEQ ID NO: 1187)
CmsCmsCmAmAmUmAfAmAfGfCfUmGmGmAmCmAmAmGmAms
AmsdTdTdTsGmsGmUmGmAmUmGfGmAfCfAfGmAmGmUmUmAmUmCmGmAm;
first antisense strand:
(SEQ ID NO: 1184)
UmsUfsCmUmUmGmUmCmCmAmGmCmUmUfUmAfUmUmGmGmGmsAmsGm
second antisense strand:
(SEQ ID NO: 1174)
UmsCfsGmAmUmAmAmCmUmCmUmGmUmCfCmAfUmCmAmCmCmsUmsCm
(12) sense strand:
(SEQ ID NO: 1188)
CmsCmsCmAmAmUmAfAmAfGfCfUmGmGmAmCmAmAmGmAms
AmsdTdTdTdTsGmsGmsUmGmAmUmGfGmAfCfAfGmAmGmUmUmAmUmCmGmAm;
first antisense strand:
(SEQ ID NO: 1184)
UmsUfsCmUmUmGmUmCmCmAmGmCmUmUfUmAfUmUmGmGmGmsAmsGm
second antisense strand:
(SEQ ID NO: 1174)
UmsCfsGmAmUmAmAmCmUmCmUmGmUmCfCmAfUmCmAmCmCmsUmsCm
(13) sense strand:
(SEQ ID NO: 1189)
CmsCmsCmAmAmUmAfAmAfGfCfUmGmGmAmCmAmAmGmAms
AmsdTdTdTdTdTsGmsGmUmGmAmUmGfGmAfCfAfGmAmGmUmUmAmUmCmGmAm;
first antisense strand:
(SEQ ID NO: 1184)
UmsUfsCmUmUmGmUmCmCmAmGmCmUmUfUmAfUmUmGmGmGmsAmsGm
second antisense strand:
(SEQ ID NO: 1174)
UmsCfsGmAmUmAmAmCmUmCmUmGmUmCfCmAfUmCmAmCmCmsUmsCm
(14) sense strand:
(SEQ ID NO: 1190)
CmsCmsCmAmAmUmAfAmAfGfCfUmGmGmAmCmAmAmGmAms
AmsdTdTdTdTdTdTsGmsGmUmGmAmUmGfGmAfCfAfGmAmGmUmUmAmUmCmGmAm;
first antisense strand:
(SEQ ID NO: 1184)
UmsUfsCmUmUmGmUmCmCmAmGmCmUmUfUmAfUmUmGmGmGmsAmsGm
second antisense strand:
(SEQ ID NO: 1174)
UmsCfsGmAmUmAmAmCmUmCmUmGmUmCfCmAfUmCmAmCmCmsUmsCm
(15) sense strand:
(SEQ ID NO: 1191)
CmsCmsCmAmAmUmAfAmAfGfCfUmGmGmAmCmAmAmGmAms
AmsdAdAdAdAdAsGmsGmUmGmAmUmGfGmAfCfAfGmAmGmUmUmAmUmCmGmAm;
first antisense strand:
(SEQ ID NO: 1184)
UmsUfsCmUmUmGmUmCmCmAmGmCmUmUfUmAfUmUmGmGmGmsAmsGm
second antisense strand:
(SEQ ID NO: 1174)
UmsCfsGmAmUmAmAmCmUmCmUmGmUmCfCmAfUmCmAmCmCmsUmsCm.

52. A pharmaceutical composition, comprising the RNA inhibitor for inhibiting PCSK9 gene expression according to any one of embodiments 1-51, and further comprising a delivery vehicle, and/or a physiologically acceptable excipient and/or carrier and/or diluent.
53. The pharmaceutical composition according to embodiment 52, wherein the delivery vehicle comprises a liposome.
54. The pharmaceutical composition according to embodiment 53, wherein the delivery vehicle comprises a nanolipid.
55. Use of the RNA inhibitor for inhibiting PCSK9 gene expression according to any one of embodiments 1-51 and the pharmaceutical composition according to any one of embodiments 52-54 in the manufacture of a medicament for preventing or treating a disease or pathology, or reducing the risk of a disease or pathology.
56. The use according to embodiment 55, wherein the disease or pathology comprises a disease or pathology associated with elevated levels of PCSK9.
57. The use according to any one of embodiments 55-56, wherein the disease or pathology comprises hypercholesterolemia.
58. The use according to any one of embodiments 55-57, wherein the disease or pathology comprises inflammatory, cardiovascular and cerebrovascular, or metabolic diseases.
59. The use according to any one of embodiments 55-58, wherein the cardiovascular and cerebrovascular disease comprises hyperlipidemia, stroke, atherosclerosis, thrombosis, coronary heart disease, or aortic stenosis.
60. A method for preventing or treating a disease, condition, or syndrome, comprising administering to a subject in need thereof an effective amount of the RNA inhibitor for inhibiting PCSK9 gene expression according to any one of embodiments 1-51, a pharmaceutically acceptable salt thereof, or the pharmaceutical composition according to any one of embodiments 52-54.
61. The method according to embodiment 60, wherein the RNA inhibitor for inhibiting PCSK9 gene expression, the pharmaceutically acceptable salt thereof, or the pharmaceutical composition is administered to the subject by a subcutaneous, intravenous, oral, rectal, or intraperitoneal administration route.
62. A method for inhibiting PCSK9 expression in a cell, tissue, or subject, comprising administering to the cell, tissue, or subject an effective amount of the RNA inhibitor for inhibiting PCSK9 gene expression according to any one of embodiments 1-51, a pharmaceutically acceptable salt thereof, or the pharmaceutical composition according to any one of embodiments 52-54.
63. The method according to embodiment 62, wherein the cell is a hepatocyte.
64. The method according to any one of embodiments 62-63, wherein the tissue is a liver tissue.
65. The method according to any one of embodiments 62-64, wherein the cell and tissue are ex vivo.

### EXAMPLES

### Example 1: Design and Synthesis of Unmodified Sequences of PCSK9 siRNA

According to a human transcript of the PCSK9 gene (derived from the NCBI website, transcript number: NM_001407240), a full-length region (comprising 5′-UTR, CDS, and 3′-UTR) of human PCSK9 mRNA was introduced into a customized R and Python script to design original sequences of siRNA targeting the PCSK9 gene, and artificial and rational screening was performed to improve the screening success rate.

The specific designed sequences are as follows:

TABLE 2
Duplex siRNA sequence information for DLR screening

| | | SEQ | | SEQ ID |
|---|---|---|---|---|
| Compound | Sense strand (5′-3′) | ID NO | Antisense strand (5′-3′) | NO |
| Ps178 | CUUCAGCUCCUGCACA | 1 | AGGACUGUGCAGGAGCUG | 328 |
| | GUCCU | | AAGUU | |
| Ps2428 | GGUUCCGACUUGUCCC | 2 | AGAGAGGGACAAGUCGG | 329 |
| | UCUCU | | AACCAU | |
| Ps2537 | GCCUGGAACUCACUCA | 3 | CAGAGUGAGUGAGUUCCA | 330 |
| | CUCUG | | GGCAA | |
| Ps2610 | GCAUUUCACCAUUCA | 4 | CCUGUUUGAAUGGUGAA | 331 |
| | AACAGG | | AUGCCC | |
| Ps2617 | ACCAUUCAAACAGGU | 5 | AGCUCGACCUGUUUGAAU | 332 |
| | CGAGCU | | GGUGA | |
| Ps2674 | UGUCCGUGGGCAGAA | 6 | AAGUCAUUCUGCCCACGG | 333 |
| | UGACUU | | ACAUC | |
| Ps2683 | GCAGAAUGACUUUUA | 7 | GCUCAAUAAAAGUCAUUC | 334 |
| | UUGAGC | | UGCCC | |
| Ps2719 | GGCAUUCAAUCCUCA | 8 | GAGACCUGAGGAUUGAA | 335 |
| | GGUCUC | | UGCCUG | |
| Ps2721 | CAUUCAAUCCUCAGG | 9 | UGGAGACCUGAGGAUUG | 336 |
| | UCUCCA | | AAUGCC | |
| Ps2723 | UUCAAUCCUCAGGUC | 10 | GGUGGAGACCUGAGGAU | 337 |
| | UCCACC | | UGAAUG | |
| Ps2725 | CAAUCCUCAGGUCUCC | 11 | UUGGUGGAGACCUGAGG | 338 |
| | ACCAA | | AUUGAA | |
| Ps2740 | CACCAAGGAGGCAGG | 12 | AAGAAUCCUGCCUCCUUG | 339 |
| | AUUCUU | | GUGGA | |
| Ps2749 | GGCAGGAUUCUUCCC | 13 | AUCCAUGGGAAGAAUCCU | 340 |
| | AUGGAU | | GCCUC | |
| Ps2751 | CAGGAUUCUUCCCAU | 14 | CUAUCCAUGGGAAGAAUC | 341 |
| | GGAUAG | | CUGCC | |
| Ps2817 | GAGUGUGAAAGGUGC | 15 | CCAUCAGCACCUUUCACA | 342 |
| | UGAUGG | | CUCAC | |
| Ps2824 | AAAGGUGCUGAUGGC | 16 | AUGAGGGCCAUCAGCACC | 343 |
| | CCUCAU | | UUUCA | |
| Ps2836 | GGCCCUCAUCUCCAGC | 17 | AGUUAGCUGGAGAUGAG | 344 |
| | UAACU | | GGCCAU | |
| Ps2880 | CUGAUUAAUGGAGGC | 18 | AGCUAAGCCUCCAUUAAU | 345 |
| | UUAGCU | | CAGGG | |
| Ps2884 | UUAAUGGAGGCUUAG | 19 | AGAAAGCUAAGCCUCCAU | 346 |
| | CUUUCU | | UAAUC | |
| Ps2892 | GGCUUAGCUUUCUGG | 20 | UGCCAUCCAGAAAGCUAA | 347 |
| | AUGGCA | | GCCUC | |
| Ps2971 | CCUGAGCCACCUUUAC | 21 | GCAGAGUAAAGGUGGCUC | 348 |
| | UCUGC | | AGGAA | |
| Ps2978 | CACCUUUACUCUGCUC | 22 | GCAUAGAGCAGAGUAAA | 349 |
| | UAUGC | | GGUGGC | |
| Ps2984 | UACUCUGCUCUAUGCC | 23 | AGCCUGGCAUAGAGCAGA | 350 |
| | AGGCU | | GUAAA | |
| Ps2986 | CUCUGCUCUAUGCCAG | 24 | ACAGCCUGGCAUAGAGCA | 351 |
| | GCUGU | | GAGUA | |
| Ps3057 | UCGGCAGUGUGCAGU | 25 | UGCACCACUGCACACUGC | 352 |
| | GGUGCA | | CGAGU | |
| Ps3117 | UACACAUUCGCACCCC | 26 | AAGUAGGGGUGCGAAUG | 353 |
| | UACUU | | UGUACC | |
| Ps3119 | CACAUUCGCACCCCUA | 27 | UGAAGUAGGGGUGCGAA | 354 |
| | CUUCA | | UGUGUA | |
| Ps3122 | AUUCGCACCCCUACUU | 28 | CUGUGAAGUAGGGGUGC | 355 |
| | CACAG | | GAAUGU | |
| Ps3124 | UCGCACCCCUACUUCA | 29 | CUCUGUGAAGUAGGGGU | 356 |
| | CAGAG | | GCGAAU | |
| Ps3129 | CCCCUACUUCACAGAG | 30 | UCUUCCUCUGUGAAGUAG | 357 |
| | GAAGA | | GGGUG | |
| Ps3140 | CAGAGGAAGAAACCU | 31 | GGUUCCAGGUUUCUUCCU | 358 |
| | GGAACC | | CUGUG | |
| Ps3196 | ACUGAGCCAGAAACG | 32 | AAUCUGCGUUUCUGGCUC | 359 |
| | CAGAUU | | AGUUC | |
| Ps3198 | UGAGCCAGAAACGCA | 33 | CCAAUCUGCGUUUCUGGC | 360 |
| | GAUUGG | | UCAGU | |
| Ps3233 | CAAGCCUCUUCUUACU | 34 | GGUGAAGUAAGAAGAGG | 361 |
| | UCACC | | CUUGGC | |
| Ps3316 | GCUCGGUGAGUGAUG | 35 | UUCUGCCAUCACUCACCG | 362 |
| | GCAGAA | | AGCUU | |
| Ps3320 | GGUGAGUGAUGGCAG | 36 | AUCGUUCUGCCAUCACUC | 363 |
| | AACGAU | | ACCGA | |
| Ps3464 | CCCAAGCAAGCAGACA | 37 | AUAAAUGUCUGCUUGCUU | 364 |
| | UUUAU | | GGGUG | |
| Ps3466 | CAAGCAAGCAGACAU | 38 | AGAUAAAUGUCUGCUUGC | 365 |
| | UUAUCU | | UUGGG | |
| Ps3489 | UGGGUCUGUCCUCUC | 39 | GCAACAGAGAGGACAGAC | 366 |
| | UGUUGC | | CCAAA | |
| Ps3543 | CUUUUGUAACUUGAA | 40 | AAUAUCUUCAAGUUACAA | 367 |
| | GAUAUU | | AAGCA | |
| Ps3548 | GUAACUUGAAGAUAU | 41 | GAAUAAAUAUCUUCAAG | 368 |
| | UUAUUC | | UUACAA | |
| Ps3554 | UGAAGAUAUUUAUUC | 42 | AACCCAGAAUAAAUAUCU | 369 |
| | UGGGUU | | UCAAG | |
| P446 | CCGAAGCACCCGAGCA | 43 | UUCCGUGCUCGGGUGCUU | 370 |
| | CGGAA | | CGGCC | |
| P516 | CACCUACGUGGUGGU | 44 | UUCAGCACCACCACGUAG | 371 |
| | GCUGAA | | GUGCC | |
| P607 | UACCUCACCAAGAUCC | 45 | AUGCAGGAUCUUGGUGA | 372 |
| | UGCAU | | GGUAUC | |
| P612 | CACCAAGAUCCUGCAU | 46 | AAGACAUGCAGGAUCUUG | 373 |
| | GUCUU | | GUGAG | |
| P629 | UCUUCCAUGGCCUUCU | 47 | CAGGAAGAAGGCCAUGGA | 374 |
| | UCCUG | | AGACA | |
| P633 | CCAUGGCCUUCUUCCU | 48 | AAGCCAGGAAGAAGGCCA | 375 |
| | GGCUU | | UGGAA | |
| P693 | GAAGUUGCCCCAUGU | 49 | UAGUCGACAUGGGGCAAC | 376 |
| | CGACUA | | UUCAA | |
| P823 | CUGGUGGAGGUGUAU | 50 | UAGGAGAUACACCUCCAC | 377 |
| | CUCCUA | | CAGGC | |
| P835 | UAUCUCCUAGACACCA | 51 | UAUGCUGGUGUCUAGGA | 378 |
| | GCAUA | | GAUACA | |
| P839 | UCCUAGACACCAGCAU | 52 | UCUGUAUGCUGGUGUCUA | 379 |
| | ACAGA | | GGAGA | |
| P842 | UAGACACCAGCAUAC | 53 | CACUCUGUAUGCUGGUGU | 380 |
| | AGAGUG | | CUAGG | |
| P845 | ACACCAGCAUACAGA | 54 | GGUCACUCUGUAUGCUGG | 381 |
| | GUGACC | | UGUCU | |
| P854 | UACAGAGUGACCACC | 55 | UUUCCCGGUGGUCACUCU | 382 |
| | GGGAAA | | GUAUG | |
| P882 | CAGGGUCAUGGUCAC | 56 | AAGUCGGUGACCAUGACC | 383 |
| | CGACUU | | CUGCC | |
| P888 | CAUGGUCACCGACUUC | 57 | UUCUCGAAGUCGGUGACC | 384 |
| | GAGAA | | AUGAC | |
| P891 | GGUCACCGACUUCGA | 58 | ACAUUCUCGAAGUCGGUG | 385 |
| | GAAUGU | | ACCAU | |
| P1066 | ACGGUUAGCGGCACCC | 59 | UAUGAGGGUGCCGCUAAC | 386 |
| | UCAUA | | CGUGC | |
| P1079 | CCCUCAUAGGCCUGGA | 60 | UAAACUCCAGGCCUAUGA | 387 |
| | GUUUA | | GGGUG | |
| P1081 | CUCAUAGGCCUGGAG | 61 | AAUAAACUCCAGGCCUAU | 388 |
| | UUUAUU | | GAGGG | |
| P1085 | UAGGCCUGGAGUUUA | 62 | UCCGAAUAAACUCCAGGC | 389 |
| | UUCGGA | | CUAUG | |
| P1161 | UGGGUACAGCCGCGU | 63 | UUGAGGACGCGGCUGUAC | 390 |
| | CCUCAA | | CCACC | |
| P1242 | CCGGGACGAUGCCUGC | 64 | UAGAGGCAGGCAUCGUCC | 391 |
| | CUCUA | | CGGAA | |
| P1413 | CAGCACCUGCUUUGU | 65 | UGUGACACAAAGCAGGUG | 392 |
| | GUCACA | | CUGCA | |
| P1514 | CCGAGUUGAGGCAGA | 66 | UCAGUCUCUGCCUCAACU | 393 |
| | GACUGA | | CGGCC | |
| P1518 | GUUGAGGCAGAGACU | 67 | UGGAUCAGUCUCUGCCUC | 394 |
| | GAUCCA | | AACUC | |
| P1521 | GAGGCAGAGACUGAU | 68 | AAGUGGAUCAGUCUCUGC | 395 |
| | CCACUU | | CUCAA | |
| P1523 | GGCAGAGACUGAUCC | 69 | AGAAGUGGAUCAGUCUCU | 396 |
| | ACUUCU | | GCCUC | |
| P1531 | CUGAUCCACUUCUCUG | 70 | UUUGGCAGAGAAGUGGA | 397 |
| | CCAAA | | UCAGUC | |
| P1533 | GAUCCACUUCUCUGCC | 71 | UCUUUGGCAGAGAAGUG | 398 |
| | AAAGA | | GAUCAG | |
| P1536 | CCACUUCUCUGCCAAA | 72 | ACAUCUUUGGCAGAGAAG | 399 |
| | GAUGU | | UGGAU | |
| P1539 | CUUCUCUGCCAAAGA | 73 | AUGACAUCUUUGGCAGAG | 400 |
| | UGUCAU | | AAGUG | |
| P1547 | CCAAAGAUGUCAUCA | 74 | CCUCAUUGAUGACAUCUU | 401 |
| | AUGAGG | | UGGCA | |
| P1659 | CAGGACUGUAUGGUC | 75 | UGUGCUGACCAUACAGUC | 402 |
| | AGCACA | | CUGCA | |
| P1661 | GGACUGUAUGGUCAG | 76 | AGUGUGCUGACCAUACAG | 403 |
| | CACACU | | UCCUG | |
| P2101 | GGUCUGGAAUGCAAA | 77 | CUUGACUUUGCAUUCCAG | 404 |
| | GUCAAG | | ACCUG | |
| P2112 | CAAAGUCAAGGAGCA | 78 | AUUCCAUGCUCCUUGACU | 405 |
| | UGGAAU | | UUGCA | |
| P2114 | AAGUCAAGGAGCAUG | 79 | GGAUUCCAUGCUCCUUGA | 406 |
| | GAAUCC | | CUUUG | |
| P2116 | GUCAAGGAGCAUGGA | 80 | CGGGAUUCCAUGCUCCUU | 407 |
| | AUCCCG | | GACUU | |
| PPC1 | CUGUUUUGCUUUUGU | 325 | CAAGUUACAAAAGCAAAA | 652 |
| | AACUUG | | CAGGU | |
| PPC2 | CUAGACCUGUTUUGCU | 326 | ACAAAAGCAAAACAGGUC | 653 |
| | UUUGU | | UAGAA | |
| SP70 | UACACAUUCGCACCCC | 26 | AAGUAGGGGUGCGAAUG | 353 |
| | UACUU | | UGUACC | |
| SP71 | ACACAUUCGCACCCCU | 81 | GAAGUAGGGGUGCGAAU | 408 |
| | ACUUC | | GUGUAC | |
| SP73 | ACAUUCGCACCCCUAC | 82 | GUGAAGUAGGGGUGCGA | 409 |
| | UUCAC | | AUGUGU | |
| SP74 | CAUUCGCACCCCUACU | 83 | UGUGAAGUAGGGGUGCG | 410 |
| | UCACA | | AAUGUG | |
| SP76 | UUCGCACCCCUACUUC | 84 | UCUGUGAAGUAGGGGUG | 411 |
| | ACAGA | | CGAAUG | |
| SP107 | AACUGAGCCAGAAAC | 85 | AUCUGCGUUUCUGGCUCA | 412 |
| | GCAGAU | | GUUCC | |
| SP109 | CUGAGCCAGAAACGC | 86 | CAAUCUGCGUUUCUGGCU | 413 |
| | AGAUUG | | CAGUU | |
| SP133 | GCCAAGCCUCUUCUUA | 87 | UGAAGUAAGAAGAGGCU | 414 |
| | CUUCA | | UGGCUU | |
| SP134 | CCAAGCCUCUUCUUAC | 88 | GUGAAGUAAGAAGAGGC | 415 |
| | UUCAC | | UUGGCU | |
| SP227 | CUUUUUCCGUUAUCA | 89 | CCUGGGUGAUAACGGAAA | 416 |
| | CCCAGG | | AAGUU | |
| SP300 | CACCCAAGCAAGCAGA | 90 | AAAUGUCUGCUUGCUUGG | 417 |
| | CAUUU | | GUGGG | |
| SP301 | ACCCAAGCAAGCAGAC | 91 | UAAAUGUCUGCUUGCUUG | 418 |
| | AUUUA | | GGUGG | |
| SP304 | AAGCAAGCAGACAUU | 92 | AAGAUAAAUGUCUGCUU | 419 |
| | UAUCUU | | GCUUGG | |
| SP305 | AAGCAGACAUUUAUC | 93 | CCAAAAGAUAAAUGUCUG | 420 |
| | UUUUGG | | CUUGC | |
| SP306 | AGCAGACAUUUAUCU | 94 | CCCAAAAGAUAAAUGUCU | 421 |
| | UUUGGG | | GCUUG | |
| SP326 | UGUCCUCUCUGUUGCC | 95 | AAAAAGGCAACAGAGAG | 422 |
| | UUUUU | | GACAGA | |
| SP327 | UCCUCUCUGUUGCCUU | 96 | GUAAAAAGGCAACAGAG | 423 |
| | UUUAC | | AGGACA | |
| SP332 | GCCUUUUUACAGCCA | 97 | AAAAGUUGGCUGUAAAA | 424 |
| | ACUUUU | | AGGCAA | |
| SP334 | UUUACAGCCAACUUU | 98 | UCUAGAAAAGUUGGCUG | 425 |
| | UCUAGA | | UAAAAA | |
| SP340 | GCCAACUUUUCUAGA | 99 | AACAGGUCUAGAAAAGU | 426 |
| | CCUGUU | | UGGCUG | |
| SP341 | CCAACUUUUCUAGACC | 100 | AAACAGGUCUAGAAAAG | 427 |
| | UGUUU | | UUGGCU | |
| SP342 | CAACUUUUCUAGACC | 101 | AAAACAGGUCUAGAAAA | 428 |
| | UGUUUU | | GUUGGC | |
| SP343 | AACUUUUCUAGACCU | 102 | CAAAACAGGUCUAGAAAA | 429 |
| | GUUUUG | | GUUGG | |
| SP344 | ACUUUUCUAGACCUG | 103 | GCAAAACAGGUCUAGAAA | 430 |
| | UUUUGC | | AGUUG | |
| SP345 | CUUUUCUAGACCUGU | 104 | AGCAAAACAGGUCUAGAA | 431 |
| | UUUGCU | | AAGUU | |
| SP348 | UUCUAGACCUGUUUU | 105 | AAAAGCAAAACAGGUCUA | 432 |
| | GCUUUU | | GAAAA | |
| SP349 | UCUAGACCUGUUUUG | 106 | CAAAAGCAAAACAGGUCU | 433 |
| | CUUUUG | | AGAAA | |
| SP351 | AGACCUGUUUUGCUU | 107 | UUACAAAAGCAAAACAGG | 434 |
| | UUGUAA | | UCUAG | |
| SP357 | AUUUAUUCUGGGUUU | 108 | GCUACAAAACCCAGAAUA | 435 |
| | UGUAGC | | AAUAU | |
| Pt608 | ACCUCACCAAGAUCCU | 109 | CAUGCAGGAUCUUGGUGA | 436 |
| | GCAUG | | GGUAU | |
| Pt609 | CCUCACCAAGAUCCUG | 110 | ACAUGCAGGAUCUUGGUG | 437 |
| | CAUGU | | AGGUA | |
| Pt692 | UGAAGUUGCCCCAUG | 111 | AGUCGACAUGGGGCAACU | 438 |
| | UCGACU | | UCAAG | |
| Pt694 | AAGUUGCCCCAUGUC | 112 | GUAGUCGACAUGGGGCAA | 439 |
| | GACUAC | | CUUCA | |
| Pt695 | AGUUGCCCCAUGUCG | 113 | UGUAGUCGACAUGGGGCA | 440 |
| | ACUACA | | ACUUC | |
| Pt837 | UCUCCUAGACACCAGC | 114 | UGUAUGCUGGUGUCUAG | 441 |
| | AUACA | | GAGAUA | |
| Pt838 | CUCCUAGACACCAGCA | 115 | CUGUAUGCUGGUGUCUAG | 442 |
| | UACAG | | GAGAU | |
| Pt840 | CCUAGACACCAGCAUA | 116 | CUCUGUAUGCUGGUGUCU | 443 |
| | CAGAG | | AGGAG | |
| Pt841 | CUAGACACCAGCAUAC | 117 | ACUCUGUAUGCUGGUGUC | 444 |
| | AGAGU | | UAGGA | |
| Pt844 | GACACCAGCAUACAG | 118 | GUCACUCUGUAUGCUGGU | 445 |
| | AGUGAC | | GUCUA | |
| Pt847 | ACCAGCAUACAGAGU | 119 | GUGGUCACUCUGUAUGCU | 446 |
| | GACCAC | | GGUGU | |
| Pt848 | CCAGCAUACAGAGUG | 120 | GGUGGUCACUCUGUAUGC | 447 |
| | ACCACC | | UGGUG | |
| Pt855 | ACAGAGUGACCACCG | 121 | AUUUCCCGGUGGUCACUC | 448 |
| | GGAAAU | | UGUAU | |
| Pt856 | CAGAGUGACCACCGG | 122 | GAUUUCCCGGUGGUCACU | 449 |
| | GAAAUC | | CUGUA | |
| Pt889 | AUGGUCACCGACUUC | 123 | AUUCUCGAAGUCGGUGAC | 450 |
| | GAGAAU | | CAUGA | |
| Pt890 | UGGUCACCGACUUCG | 124 | CAUUCUCGAAGUCGGUGA | 451 |
| | AGAAUG | | CCAUG | |
| Pt892 | GUCACCGACUUCGAG | 125 | CACAUUCUCGAAGUCGGU | 452 |
| | AAUGUG | | GACCA | |
| Pt941 | GACAGGCCAGCAAGU | 126 | UGUCACACUUGCUGGCCU | 453 |
| | GUGACA | | GUCUG | |
| Pt1080 | CCUCAUAGGCCUGGA | 127 | AUAAACUCCAGGCCUAUG | 454 |
| | GUUUAU | | AGGGU | |
| Pt1082 | UCAUAGGCCUGGAGU | 128 | GAAUAAACUCCAGGCCUA | 455 |
| | UUAUUC | | UGAGG | |
| Pt1083 | CAUAGGCCUGGAGUU | 129 | CGAAUAAACUCCAGGCCU | 456 |
| | UAUUCG | | AUGAG | |
| Pt1084 | AUAGGCCUGGAGUUU | 130 | CCGAAUAAACUCCAGGCC | 457 |
| | AUUCGG | | UAUGA | |
| Pt1086 | AGGCCUGGAGUUUAU | 131 | UUCCGAAUAAACUCCAGG | 458 |
| | UCGGAA | | CCUAU | |
| Pt1087 | GGCCUGGAGUUUAUU | 132 | UUUCCGAAUAAACUCCAG | 459 |
| | CGGAAA | | GCCUA | |
| Pt1275 | AGCUCCCGAGGUCAUC | 133 | ACUGUGAUGACCUCGGGA | 460 |
| | ACAGU | | GCUGA | |
| Pt1516 | GAGUUGAGGCAGAGA | 134 | GAUCAGUCUCUGCCUCAA | 461 |
| | CUGAUC | | CUCGG | |
| Pt1517 | AGUUGAGGCAGAGAC | 135 | GGAUCAGUCUCUGCCUCA | 462 |
| | UGAUCC | | ACUCG | |
| Pt1519 | UUGAGGCAGAGACUG | 136 | GUGGAUCAGUCUCUGCCU | 463 |
| | AUCCAC | | CAACU | |
| Pt1520 | UGAGGCAGAGACUGA | 137 | AGUGGAUCAGUCUCUGCC | 464 |
| | UCCACU | | UCAAC | |
| Pt1522 | AGGCAGAGACUGAUC | 138 | GAAGUGGAUCAGUCUCUG | 465 |
| | CACUUC | | CCUCA | |
| Pt1530 | ACUGAUCCACUUCUCU | 139 | UUGGCAGAGAAGUGGAU | 466 |
| | GCCAA | | CAGUCU | |
| Pt1532 | UGAUCCACUUCUCUGC | 140 | CUUUGGCAGAGAAGUGG | 467 |
| | CAAAG | | AUCAGU | |
| Pt1534 | AUCCACUUCUCUGCCA | 141 | AUCUUUGGCAGAGAAGU | 468 |
| | AAGAU | | GGAUCA | |
| Pt1535 | UCCACUUCUCUGCCAA | 142 | CAUCUUUGGCAGAGAAGU | 469 |
| | AGAUG | | GGAUC | |
| Pt1537 | CACUUCUCUGCCAAAG | 143 | GACAUCUUUGGCAGAGAA | 470 |
| | AUGUC | | GUGGA | |
| Pt1538 | ACUUCUCUGCCAAAG | 144 | UGACAUCUUUGGCAGAGA | 471 |
| | AUGUCA | | AGUGG | |
| Pt2102 | GUCUGGAAUGCAAAG | 145 | CCUUGACUUUGCAUUCCA | 472 |
| | UCAAGG | | GACCU | |
| Pt2105 | UGGAAUGCAAAGUCA | 146 | GCUCCUUGACUUUGCAUU | 473 |
| | AGGAGC | | CCAGA | |
| Pt2106 | GGAAUGCAAAGUCAA | 147 | UGCUCCUUGACUUUGCAU | 474 |
| | GGAGCA | | UCCAG | |
| Pt2113 | AAAGUCAAGGAGCAU | 148 | GAUUCCAUGCUCCUUGAC | 475 |
| | GGAAUC | | UUUGC | |
| Pt2115 | AGUCAAGGAGCAUGG | 149 | GGGAUUCCAUGCUCCUUG | 476 |
| | AAUCCC | | ACUUU | |
| Pt2426 | AUGGUUCCGACUUGU | 150 | AGAGGGACAAGUCGGAAC | 477 |
| | CCCUCU | | CAUUU | |
| Pt2427 | UGGUUCCGACUUGUC | 151 | GAGAGGGACAAGUCGGA | 478 |
| | CCUCUC | | ACCAUU | |
| Pt2432 | CCGACUUGUCCCUCUC | 152 | GCUGAGAGAGGGACAAG | 479 |
| | UCAGC | | UCGGAA | |
| Pt2530 | CCUCCUUGCCUGGAAC | 153 | AGUGAGUUCCAGGCAAGG | 480 |
| | UCACU | | AGGCU | |
| Pt2532 | UCCUUGCCUGGAACUC | 154 | UGAGUGAGUUCCAGGCAA | 481 |
| | ACUCA | | GGAGG | |
| Pt2533 | CCUUGCCUGGAACUCA | 155 | GUGAGUGAGUUCCAGGCA | 482 |
| | CUCAC | | AGGAG | |
| Pt2534 | CUUGCCUGGAACUCAC | 156 | AGUGAGUGAGUUCCAGGC | 483 |
| | UCACU | | AAGGA | |
| Pt2535 | UUGCCUGGAACUCAC | 157 | GAGUGAGUGAGUUCCAG | 484 |
| | UCACUC | | GCAAGG | |
| Pt2536 | UGCCUGGAACUCACUC | 158 | AGAGUGAGUGAGUUCCA | 485 |
| | ACUCU | | GGCAAG | |
| Pt2538 | CCUGGAACUCACUCAC | 159 | CCAGAGUGAGUGAGUUCC | 486 |
| | UCUGG | | AGGCA | |
| Pt2540 | UGGAACUCACUCACUC | 160 | ACCCAGAGUGAGUGAGUU | 487 |
| | UGGGU | | CCAGG | |
| Pt2541 | GGAACUCACUCACUCU | 161 | CACCCAGAGUGAGUGAGU | 488 |
| | GGGUG | | UCCAG | |
| Pt2542 | GAACUCACUCACUCUG | 162 | GCACCCAGAGUGAGUGAG | 489 |
| | GGUGC | | UUCCA | |
| Pt2544 | ACUCACUCACUCUGGG | 163 | AGGCACCCAGAGUGAGUG | 490 |
| | UGCCU | | AGUUC | |
| Pt2545 | CUCACUCACUCUGGGU | 164 | GAGGCACCCAGAGUGAGU | 491 |
| | GCCUC | | GAGUU | |
| Pt2611 | CAUUUCACCAUUCAA | 165 | ACCUGUUUGAAUGGUGA | 492 |
| | ACAGGU | | AAUGCC | |
| Pt2612 | AUUUCACCAUUCAAA | 166 | GACCUGUUUGAAUGGUG | 493 |
| | CAGGUC | | AAAUGC | |
| Pt2613 | UUUCACCAUUCAAAC | 167 | CGACCUGUUUGAAUGGUG | 494 |
| | AGGUCG | | AAAUG | |
| Pt2614 | UUCACCAUUCAAACA | 168 | UCGACCUGUUUGAAUGGU | 495 |
| | GGUCGA | | GAAAU | |
| Pt2615 | UCACCAUUCAAACAG | 169 | CUCGACCUGUUUGAAUGG | 496 |
| | GUCGAG | | UGAAA | |
| Pt2616 | CACCAUUCAAACAGG | 170 | GCUCGACCUGUUUGAAUG | 497 |
| | UCGAGC | | GUGAA | |
| Pt2618 | CCAUUCAAACAGGUC | 171 | CAGCUCGACCUGUUUGAA | 498 |
| | GAGCUG | | UGGUG | |
| Pt2656 | CUGCUCCCAAUGUGCC | 172 | ACAUCGGCACAUUGGGAG | 499 |
| | GAUGU | | CAGCU | |
| Pt2673 | AUGUCCGUGGGCAGA | 173 | AGUCAUUCUGCCCACGGA | 500 |
| | AUGACU | | CAUCG | |
| Pt2675 | GUCCGUGGGCAGAAU | 174 | AAAGUCAUUCUGCCCACG | 501 |
| | GACUUU | | GACAU | |
| Pt2696 | UAUUGAGCUCUUGUU | 175 | GCACGGAACAAGAGCUCA | 502 |
| | CCGUGC | | AUAAA | |
| Pt2697 | AUUGAGCUCUUGUUC | 176 | GGCACGGAACAAGAGCUC | 503 |
| | CGUGCC | | AAUAA | |
| Pt2698 | UUGAGCUCUUGUUCC | 177 | UGGCACGGAACAAGAGCU | 504 |
| | GUGCCA | | CAAUA | |
| Pt2699 | UGAGCUCUUGUUCCG | 178 | CUGGCACGGAACAAGAGC | 505 |
| | UGCCAG | | UCAAU | |
| Pt2700 | GAGCUCUUGUUCCGU | 179 | CCUGGCACGGAACAAGAG | 506 |
| | GCCAGG | | CUCAA | |
| Pt2717 | CAGGCAUUCAAUCCUC | 180 | GACCUGAGGAUUGAAUGC | 507 |
| | AGGUC | | CUGGC | |
| Pt2718 | AGGCAUUCAAUCCUC | 181 | AGACCUGAGGAUUGAAU | 508 |
| | AGGUCU | | GCCUGG | |
| Pt2720 | GCAUUCAAUCCUCAG | 182 | GGAGACCUGAGGAUUGA | 509 |
| | GUCUCC | | AUGCCU | |
| Pt2722 | AUUCAAUCCUCAGGU | 183 | GUGGAGACCUGAGGAUU | 510 |
| | CUCCAC | | GAAUGC | |
| Pt2724 | UCAAUCCUCAGGUCUC | 184 | UGGUGGAGACCUGAGGA | 511 |
| | CACCA | | UUGAAU | |
| Pt2726 | AAUCCUCAGGUCUCCA | 185 | CUUGGUGGAGACCUGAGG | 512 |
| | CCAAG | | AUUGA | |
| Pt2727 | AUCCUCAGGUCUCCAC | 186 | CCUUGGUGGAGACCUGAG | 513 |
| | CAAGG | | GAUUG | |
| Pt2741 | ACCAAGGAGGCAGGA | 187 | GAAGAAUCCUGCCUCCUU | 514 |
| | UUCUUC | | GGUGG | |
| Pt2742 | CCAAGGAGGCAGGAU | 188 | GGAAGAAUCCUGCCUCCU | 515 |
| | UCUUCC | | UGGUG | |
| Pt2743 | CAAGGAGGCAGGAUU | 189 | GGGAAGAAUCCUGCCUCC | 516 |
| | CUUCCC | | UUGGU | |
| Pt2744 | AAGGAGGCAGGAUUC | 190 | UGGGAAGAAUCCUGCCUC | 517 |
| | UUCCCA | | CUUGG | |
| Pt2745 | AGGAGGCAGGAUUCU | 191 | AUGGGAAGAAUCCUGCCU | 518 |
| | UCCCAU | | CCUUG | |
| Pt2746 | GGAGGCAGGAUUCUU | 192 | CAUGGGAAGAAUCCUGCC | 519 |
| | CCCAUG | | UCCUU | |
| Pt2747 | GAGGCAGGAUUCUUC | 193 | CCAUGGGAAGAAUCCUGC | 520 |
| | CCAUGG | | CUCCU | |
| Pt2748 | AGGCAGGAUUCUUCC | 194 | UCCAUGGGAAGAAUCCUG | 521 |
| | CAUGGA | | CCUCC | |
| Pt2750 | GCAGGAUUCUUCCCA | 195 | UAUCCAUGGGAAGAAUCC | 522 |
| | UGGAUA | | UGCCU | |
| Pt2752 | AGGAUUCUUCCCAUG | 196 | CCUAUCCAUGGGAAGAAU | 523 |
| | GAUAGG | | CCUGC | |
| Pt2753 | GGAUUCUUCCCAUGG | 197 | CCCUAUCCAUGGGAAGAA | 524 |
| | AUAGGG | | UCCUG | |
| Pt2789 | GCUGCAGGGACAAAC | 198 | AACGAUGUUUGUCCCUGC | 525 |
| | AUCGUU | | AGCCC | |
| Pt2790 | CUGCAGGGACAAACA | 199 | CAACGAUGUUUGUCCCUG | 526 |
| | UCGUUG | | CAGCC | |
| Pt2791 | UGCAGGGACAAACAU | 200 | CCAACGAUGUUUGUCCCU | 527 |
| | CGUUGG | | GCAGC | |
| Pt2792 | GCAGGGACAAACAUC | 201 | CCCAACGAUGUUUGUCCC | 528 |
| | GUUGGG | | UGCAG | |
| Pt2815 | GUGAGUGUGAAAGGU | 202 | AUCAGCACCUUUCACACU | 529 |
| | GCUGAU | | CACCC | |
| Pt2816 | UGAGUGUGAAAGGUG | 203 | CAUCAGCACCUUUCACAC | 530 |
| | CUGAUG | | UCACC | |
| Pt2818 | AGUGUGAAAGGUGCU | 204 | GCCAUCAGCACCUUUCAC | 531 |
| | GAUGGC | | ACUCA | |
| Pt2819 | GUGUGAAAGGUGCUG | 205 | GGCCAUCAGCACCUUUCA | 532 |
| | AUGGCC | | CACUC | |
| Pt2820 | UGUGAAAGGUGCUGA | 206 | GGGCCAUCAGCACCUUUC | 533 |
| | UGGCCC | | ACACU | |
| Pt2821 | GUGAAAGGUGCUGAU | 207 | AGGGCCAUCAGCACCUUU | 534 |
| | GGCCCU | | CACAC | |
| Pt2822 | UGAAAGGUGCUGAUG | 208 | GAGGGCCAUCAGCACCUU | 535 |
| | GCCCUC | | UCACA | |
| Pt2823 | GAAAGGUGCUGAUGG | 209 | UGAGGGCCAUCAGCACCU | 536 |
| | CCCUCA | | UUCAC | |
| Pt2837 | GCCCUCAUCUCCAGCU | 210 | CAGUUAGCUGGAGAUGA | 537 |
| | AACUG | | GGGCCA | |
| Pt2838 | CCCUCAUCUCCAGCUA | 211 | ACAGUUAGCUGGAGAUG | 538 |
| | ACUGU | | AGGGCC | |
| Pt2839 | CCUCAUCUCCAGCUAA | 212 | CACAGUUAGCUGGAGAUG | 539 |
| | CUGUG | | AGGGC | |
| Pt2840 | CUCAUCUCCAGCUAAC | 213 | CCACAGUUAGCUGGAGAU | 540 |
| | UGUGG | | GAGGG | |
| Pt2850 | GCUAACUGUGGAGAA | 214 | AGGGGCUUCUCCACAGUU | 541 |
| | GCCCCU | | AGCUG | |
| Pt2851 | CUAACUGUGGAGAAG | 215 | CAGGGGCUUCUCCACAGU | 542 |
| | CCCCUG | | UAGCU | |
| Pt2879 | CCUGAUUAAUGGAGG | 216 | GCUAAGCCUCCAUUAAUC | 543 |
| | CUUAGC | | AGGGA | |
| Pt2881 | UGAUUAAUGGAGGCU | 217 | AAGCUAAGCCUCCAUUAA | 544 |
| | UAGCUU | | UCAGG | |
| Pt2882 | GAUUAAUGGAGGCUU | 218 | AAAGCUAAGCCUCCAUUA | 545 |
| | AGCUUU | | AUCAG | |
| Pt2883 | AUUAAUGGAGGCUUA | 219 | GAAAGCUAAGCCUCCAUU | 546 |
| | GCUUUC | | AAUCA | |
| Pt2885 | UAAUGGAGGCUUAGC | 220 | CAGAAAGCUAAGCCUCCA | 547 |
| | UUUCUG | | UUAAU | |
| Pt2886 | AAUGGAGGCUUAGCU | 221 | CCAGAAAGCUAAGCCUCC | 548 |
| | UUCUGG | | AUUAA | |
| Pt2887 | AUGGAGGCUUAGCUU | 222 | UCCAGAAAGCUAAGCCUC | 549 |
| | UCUGGA | | CAUUA | |
| Pt2888 | UGGAGGCUUAGCUUU | 223 | AUCCAGAAAGCUAAGCCU | 550 |
| | CUGGAU | | CCAUU | |
| Pt2889 | GGAGGCUUAGCUUUC | 224 | CAUCCAGAAAGCUAAGCC | 551 |
| | UGGAUG | | UCCAU | |
| Pt2890 | GAGGCUUAGCUUUCU | 225 | CCAUCCAGAAAGCUAAGC | 552 |
| | GGAUGG | | CUCCA | |
| Pt2891 | AGGCUUAGCUUUCUG | 226 | GCCAUCCAGAAAGCUAAG | 553 |
| | GAUGGC | | CCUCC | |
| Pt2893 | GCUUAGCUUUCUGGA | 227 | AUGCCAUCCAGAAAGCUA | 554 |
| | UGGCAU | | AGCCU | |
| Pt2959 | GUGCCUUGGUUUCCU | 228 | UGGCUCAGGAAACCAAGG | 555 |
| | GAGCCA | | CACAG | |
| Pt2960 | UGCCUUGGUUUCCUG | 229 | GUGGCUCAGGAAACCAAG | 556 |
| | AGCCAC | | GCACA | |
| Pt2962 | CCUUGGUUUCCUGAG | 230 | AGGUGGCUCAGGAAACCA | 557 |
| | CCACCU | | AGGCA | |
| Pt2963 | CUUGGUUUCCUGAGC | 231 | AAGGUGGCUCAGGAAACC | 558 |
| | CACCUU | | AAGGC | |
| Pt2964 | UUGGUUUCCUGAGCC | 232 | AAAGGUGGCUCAGGAAAC | 559 |
| | ACCUUU | | CAAGG | |
| Pt2965 | UGGUUUCCUGAGCCA | 233 | UAAAGGUGGCUCAGGAA | 560 |
| | CCUUUA | | ACCAAG | |
| Pt2966 | GGUUUCCUGAGCCACC | 234 | GUAAAGGUGGCUCAGGA | 561 |
| | UUUAC | | AACCAA | |
| Pt2967 | GUUUCCUGAGCCACCU | 235 | AGUAAAGGUGGCUCAGG | 562 |
| | UUACU | | AAACCA | |
| Pt2968 | UUUCCUGAGCCACCUU | 236 | GAGUAAAGGUGGCUCAG | 563 |
| | UACUC | | GAAACC | |
| Pt2969 | UUCCUGAGCCACCUUU | 237 | AGAGUAAAGGUGGCUCA | 564 |
| | ACUCU | | GGAAAC | |
| Pt2970 | UCCUGAGCCACCUUUA | 238 | CAGAGUAAAGGUGGCUCA | 565 |
| | CUCUG | | GGAAA | |
| Pt2972 | CUGAGCCACCUUUACU | 239 | AGCAGAGUAAAGGUGGC | 566 |
| | CUGCU | | UCAGGA | |
| Pt2977 | CCACCUUUACUCUGCU | 240 | CAUAGAGCAGAGUAAAG | 567 |
| | CUAUG | | GUGGCU | |
| Pt2979 | ACCUUUACUCUGCUCU | 241 | GGCAUAGAGCAGAGUAA | 568 |
| | AUGCC | | AGGUGG | |
| Pt2980 | CCUUUACUCUGCUCUA | 242 | UGGCAUAGAGCAGAGUA | 569 |
| | UGCCA | | AAGGUG | |
| Pt2981 | CUUUACUCUGCUCUA | 243 | CUGGCAUAGAGCAGAGUA | 570 |
| | UGCCAG | | AAGGU | |
| Pt2982 | UUUACUCUGCUCUAU | 244 | CCUGGCAUAGAGCAGAGU | 571 |
| | GCCAGG | | AAAGG | |
| Pt2983 | UUACUCUGCUCUAUG | 245 | GCCUGGCAUAGAGCAGAG | 572 |
| | CCAGGC | | UAAAG | |
| Pt2985 | ACUCUGCUCUAUGCCA | 246 | CAGCCUGGCAUAGAGCAG | 573 |
| | GGCUG | | AGUAA | |
| Pt2987 | UCUGCUCUAUGCCAG | 247 | CACAGCCUGGCAUAGAGC | 574 |
| | GCUGUG | | AGAGU | |
| Pt2989 | UGCUCUAUGCCAGGC | 248 | AGCACAGCCUGGCAUAGA | 575 |
| | UGUGCU | | GCAGA | |
| Pt2990 | GCUCUAUGCCAGGCU | 249 | UAGCACAGCCUGGCAUAG | 576 |
| | GUGCUA | | AGCAG | |
| Pt2991 | CUCUAUGCCAGGCUG | 250 | CUAGCACAGCCUGGCAUA | 577 |
| | UGCUAG | | GAGCA | |
| Pt3037 | AGCCAUCACCUAGGAC | 251 | AGUCAGUCCUAGGUGAUG | 578 |
| | UGACU | | GCUCC | |
| Pt3038 | GCCAUCACCUAGGACU | 252 | GAGUCAGUCCUAGGUGAU | 579 |
| | GACUC | | GGCUC | |
| Pt3039 | CCAUCACCUAGGACUG | 253 | CGAGUCAGUCCUAGGUGA | 580 |
| | ACUCG | | UGGCU | |
| Pt3042 | UCACCUAGGACUGAC | 254 | UGCCGAGUCAGUCCUAGG | 581 |
| | UCGGCA | | UGAUG | |
| Pt3043 | CACCUAGGACUGACUC | 255 | CUGCCGAGUCAGUCCUAG | 582 |
| | GGCAG | | GUGAU | |
| Pt3125 | CGCACCCCUACUUCAC | 256 | CCUCUGUGAAGUAGGGGU | 583 |
| | AGAGG | | GCGAA | |
| Pt3126 | GCACCCCUACUUCACA | 257 | UCCUCUGUGAAGUAGGGG | 584 |
| | GAGGA | | UGCGA | |
| Pt3127 | CACCCCUACUUCACAG | 258 | UUCCUCUGUGAAGUAGGG | 585 |
| | AGGAA | | GUGCG | |
| Pt3138 | CACAGAGGAAGAAAC | 259 | UUCCAGGUUUCUUCCUCU | 586 |
| | CUGGAA | | GUGAA | |
| Pt3139 | ACAGAGGAAGAAACC | 260 | GUUCCAGGUUUCUUCCUC | 587 |
| | UGGAAC | | UGUGA | |
| Pt3141 | AGAGGAAGAAACCUG | 261 | UGGUUCCAGGUUUCUUCC | 588 |
| | GAACCA | | UCUGU | |
| Pt3177 | CCAAGCUCACACAGCA | 262 | GUUCCUGCUGUGUGAGCU | 589 |
| | GGAAC | | UGGCA | |
| Pt3178 | CAAGCUCACACAGCAG | 263 | AGUUCCUGCUGUGUGAGC | 590 |
| | GAACU | | UUGGC | |
| Pt3179 | AAGCUCACACAGCAG | 264 | CAGUUCCUGCUGUGUGAG | 591 |
| | GAACUG | | CUUGG | |
| Pt3180 | AGCUCACACAGCAGG | 265 | UCAGUUCCUGCUGUGUGA | 592 |
| | AACUGA | | GCUUG | |
| Pt3185 | ACACAGCAGGAACUG | 266 | CUGGCUCAGUUCCUGCUG | 593 |
| | AGCCAG | | UGUGA | |
| Pt3186 | CACAGCAGGAACUGA | 267 | UCUGGCUCAGUUCCUGCU | 594 |
| | GCCAGA | | GUGUG | |
| Pt3187 | ACAGCAGGAACUGAG | 268 | UUCUGGCUCAGUUCCUGC | 595 |
| | CCAGAA | | UGUGU | |
| Pt3188 | CAGCAGGAACUGAGC | 269 | UUUCUGGCUCAGUUCCUG | 596 |
| | CAGAAA | | CUGUG | |
| Pt3189 | AGCAGGAACUGAGCC | 270 | GUUUCUGGCUCAGUUCCU | 597 |
| | AGAAAC | | GCUGU | |
| Pt3190 | GCAGGAACUGAGCCA | 271 | CGUUUCUGGCUCAGUUCC | 598 |
| | GAAACG | | UGCUG | |
| Pt3191 | CAGGAACUGAGCCAG | 272 | GCGUUUCUGGCUCAGUUC | 599 |
| | AAACGC | | CUGCU | |
| Pt3192 | AGGAACUGAGCCAGA | 273 | UGCGUUUCUGGCUCAGUU | 600 |
| | AACGCA | | CCUGC | |
| Pt3193 | GGAACUGAGCCAGAA | 274 | CUGCGUUUCUGGCUCAGU | 601 |
| | ACGCAG | | UCCUG | |
| Pt3194 | GAACUGAGCCAGAAA | 275 | UCUGCGUUUCUGGCUCAG | 602 |
| | CGCAGA | | UUCCU | |
| Pt3199 | GAGCCAGAAACGCAG | 276 | CCCAAUCUGCGUUUCUGG | 603 |
| | AUUGGG | | CUCAG | |
| Pt3200 | AGCCAGAAACGCAGA | 277 | GCCCAAUCUGCGUUUCUG | 604 |
| | UUGGGC | | GCUCA | |
| Pt3205 | GAAACGCAGAUUGGG | 278 | AGCCAGCCCAAUCUGCGU | 605 |
| | CUGGCU | | UUCUG | |
| Pt3206 | AAACGCAGAUUGGGC | 279 | GAGCCAGCCCAAUCUGCG | 606 |
| | UGGCUC | | UUUCU | |
| Pt3207 | AACGCAGAUUGGGCU | 280 | AGAGCCAGCCCAAUCUGC | 607 |
| | GGCUCU | | GUUUC | |
| Pt3234 | AAGCCUCUUCUUACU | 281 | GGGUGAAGUAAGAAGAG | 608 |
| | UCACCC | | GCUUGG | |
| Pt3235 | AGCCUCUUCUUACUUC | 282 | CGGGUGAAGUAAGAAGA | 609 |
| | ACCCG | | GGCUUG | |
| Pt3236 | GCCUCUUCUUACUUCA | 283 | CCGGGUGAAGUAAGAAG | 610 |
| | CCCGG | | AGGCUU | |
| Pt3237 | CCUCUUCUUACUUCAC | 284 | GCCGGGUGAAGUAAGAA | 611 |
| | CCGGC | | GAGGCU | |
| Pt3238 | CUCUUCUUACUUCACC | 285 | AGCCGGGUGAAGUAAGA | 612 |
| | CGGCU | | AGAGGC | |
| Pt3239 | UCUUCUUACUUCACCC | 286 | CAGCCGGGUGAAGUAAGA | 613 |
| | GGCUG | | AGAGG | |
| Pt3240 | CUUCUUACUUCACCCG | 287 | CCAGCCGGGUGAAGUAAG | 614 |
| | GCUGG | | AAGAG | |
| Pt3241 | UUCUUACUUCACCCGG | 288 | CCCAGCCGGGUGAAGUAA | 615 |
| | CUGGG | | GAAGA | |
| Pt3243 | CUUACUUCACCCGGCU | 289 | AGCCCAGCCGGGUGAAGU | 616 |
| | GGGCU | | AAGAA | |
| Pt3275 | CGGGUAACAGUGAGG | 290 | UCCCAGCCUCACUGUUAC | 617 |
| | CUGGGA | | CCGUA | |
| Pt3276 | GGGUAACAGUGAGGC | 291 | UUCCCAGCCUCACUGUUA | 618 |
| | UGGGAA | | CCCGU | |
| Pt3302 | ACACAGACCAGGAAG | 292 | ACCGAGCUUCCUGGUCUG | 619 |
| | CUCGGU | | UGUUC | |
| Pt3303 | CACAGACCAGGAAGC | 293 | CACCGAGCUUCCUGGUCU | 620 |
| | UCGGUG | | GUGUU | |
| Pt3304 | ACAGACCAGGAAGCU | 294 | UCACCGAGCUUCCUGGUC | 621 |
| | CGGUGA | | UGUGU | |
| Pt3306 | AGACCAGGAAGCUCG | 295 | ACUCACCGAGCUUCCUGG | 622 |
| | GUGAGU | | UCUGU | |
| Pt3308 | ACCAGGAAGCUCGGU | 296 | UCACUCACCGAGCUUCCU | 623 |
| | GAGUGA | | GGUCU | |
| Pt3309 | CCAGGAAGCUCGGUG | 297 | AUCACUCACCGAGCUUCC | 624 |
| | AGUGAU | | UGGUC | |
| Pt3310 | CAGGAAGCUCGGUGA | 298 | CAUCACUCACCGAGCUUC | 625 |
| | GUGAUG | | CUGGU | |
| Pt3313 | GAAGCUCGGUGAGUG | 299 | UGCCAUCACUCACCGAGC | 626 |
| | AUGGCA | | UUCCU | |
| Pt3315 | AGCUCGGUGAGUGAU | 300 | UCUGCCAUCACUCACCGA | 627 |
| | GGCAGA | | GCUUC | |
| Pt3317 | CUCGGUGAGUGAUGG | 301 | GUUCUGCCAUCACUCACC | 628 |
| | CAGAAC | | GAGCU | |
| Pt3319 | CGGUGAGUGAUGGCA | 302 | UCGUUCUGCCAUCACUCA | 629 |
| | GAACGA | | CCGAG | |
| Pt3321 | GUGAGUGAUGGCAGA | 303 | CAUCGUUCUGCCAUCACU | 630 |
| | ACGAUG | | CACCG | |
| Pt3322 | UGAGUGAUGGCAGAA | 304 | GCAUCGUUCUGCCAUCAC | 631 |
| | CGAUGC | | UCACC | |
| Pt3382 | AUUCACUGGCCUGGC | 305 | AUCUCCGCCAGGCCAGUG | 632 |
| | GGAGAU | | AAUCA | |
| Pt3390 | GCCUGGCGGAGAUGC | 306 | UUAGAAGCAUCUCCGCCA | 633 |
| | UUCUAA | | GGCCA | |
| Pt3394 | GGCGGAGAUGCUUCU | 307 | UGCCUUAGAAGCAUCUCC | 634 |
| | AAGGCA | | GCCAG | |
| Pt3431 | CCAACAACUGUCCCUC | 308 | UCAAGGAGGGACAGUUG | 635 |
| | CUUGA | | UUGGCC | |
| Pt3432 | CAACAACUGUCCCUCC | 309 | CUCAAGGAGGGACAGUUG | 636 |
| | UUGAG | | UUGGC | |
| Pt3433 | AACAACUGUCCCUCCU | 310 | GCUCAAGGAGGGACAGUU | 637 |
| | UGAGC | | GUUGG | |
| Pt3434 | ACAACUGUCCCUCCUU | 311 | UGCUCAAGGAGGGACAGU | 638 |
| | GAGCA | | UGUUG | |
| Pt3435 | CAACUGUCCCUCCUUG | 312 | GUGCUCAAGGAGGGACAG | 639 |
| | AGCAC | | UUGUU | |
| Pt3436 | AACUGUCCCUCCUUGA | 313 | GGUGCUCAAGGAGGGACA | 640 |
| | GCACC | | GUUGU | |
| Pt3437 | ACUGUCCCUCCUUGAG | 314 | UGGUGCUCAAGGAGGGAC | 641 |
| | CACCA | | AGUUG | |
| Pt3438 | CUGUCCCUCCUUGAGC | 315 | CUGGUGCUCAAGGAGGGA | 642 |
| | ACCAG | | CAGUU | |
| Pt3461 | CCACCCAAGCAAGCAG | 316 | AAUGUCUGCUUGCUUGGG | 643 |
| | ACAUU | | UGGGG | |
| Pt3465 | CCAAGCAAGCAGACA | 317 | GAUAAAUGUCUGCUUGCU | 644 |
| | UUUAUC | | UGGGU | |
| Pt3468 | AGCAAGCAGACAUUU | 318 | AAAGAUAAAUGUCUGCU | 645 |
| | AUCUUU | | UGCUUG | |
| Pt3490 | GGGUCUGUCCUCUCU | 319 | GGCAACAGAGAGGACAGA | 646 |
| | GUUGCC | | CCCAA | |
| Pt3491 | GGUCUGUCCUCUCUG | 320 | AGGCAACAGAGAGGACAG | 647 |
| | UUGCCU | | ACCCA | |
| Pt3492 | GUCUGUCCUCUCUGU | 321 | AAGGCAACAGAGAGGACA | 648 |
| | UGCCUU | | GACCC | |
| Pt3493 | UCUGUCCUCUCUGUU | 322 | AAAGGCAACAGAGAGGAC | 649 |
| | GCCUUU | | AGACC | |
| Pt3552 | CUUGAAGAUAUUUAU | 323 | CCCAGAAUAAAUAUCUUC | 650 |
| | UCUGGG | | AAGUU | |
| Pt3553 | UUGAAGAUAUUUAUU | 324 | ACCCAGAAUAAAUAUCUU | 651 |
| | CUGGGU | | CAAGU | |
| PPC1 | CUGUUUUGCUUUUGU | 325 | CAAGUUACAAAAGCAAAA | 652 |
| | AACUUG | | CAGGU | |
| PPC2 | CUAGACCUGUTUUGCU | 326 | ACAAAAGCAAAACAGGUC | 653 |
| | UUUGU | | UAGAA | |
| SP341.1 | CCAACUUUUCUAGACC | 100 | AAACAGGUCUAGAAAAG | 1125 |
| | UGUUU | | UUGGUU | |
| SP341.2 | CCAACUUUUCUAGACC | 100 | AAACAGGUCUAGAAAAG | 1126 |
| | UGUUU | | UUGGUA | |
| SP341.3 | CCAACUUUUCUAGACC | 100 | AAACAGGUCUAGAAAAG | 1127 |
| | UGUUU | | UUGGAU | |
| SP341.4 | CCAACUUUUCUAGACC | 100 | AAACAGGUCUAGAAAAG | 1128 |
| | UGUUU | | UUGGAA | |
| SP341.5 | CCAACUUUUCUAGACC | 100 | AAACAGGUCUAGAAAAG | 1129 |
| | UGUUU | | UUGGCA | |
| SP341.6 | CCAACUUUUCUAGACC | 1123 | UAACAGGUCUAGAAAAG | 1130 |
| | UGUUA | | UUGGCU | |
| SP341.7 | CCAACUUUUCUAGACC | 1123 | UAACAGGUCUAGAAAAG | 1131 |
| | UGUUA | | UUGGUU | |
| SP341.8 | CCAACUUUUCUAGACC | 1123 | UAACAGGUCUAGAAAAG | 1132 |
| | UGUUA | | UUGGUA | |
| SP341.9 | CCAACUUUUCUAGACC | 1123 | UAACAGGUCUAGAAAAG | 1133 |
| | UGUUA | | UUGGAU | |
| SP341.10 | CCAACUUUUCUAGACC | 1123 | UAACAGGUCUAGAAAAG | 1134 |
| | UGUUA | | UUGGAA | |
| SP341.11 | CCAACUUUUCUAGACC | 1123 | UAACAGGUCUAGAAAAG | 1135 |
| | UGUUA | | UUGGCA | |
| Ps3543.1 | CUUUUGUAACUUGAA | 40 | AAUAUCUUCAAGUUACAA | 367 |
| | GAUAUU | | AAGCA | |
| Ps3543.2 | CUUUUGUAACUUGAA | 40 | AAUAUCUUCAAGUUACAA | 1136 |
| | GAUAUU | | AAGUA | |
| Ps3543.3 | CUUUUGUAACUUGAA | 40 | AAUAUCUUCAAGUUACAA | 1137 |
| | GAUAUU | | AAGUU | |
| Ps3543.4 | CUUUUGUAACUUGAA | 40 | AAUAUCUUCAAGUUACAA | 1138 |
| | GAUAUU | | AAGAA | |
| Ps3543.5 | CUUUUGUAACUUGAA | 40 | AAUAUCUUCAAGUUACAA | 1139 |
| | GAUAUU | | AAGAU | |
| Ps3543.6 | CUUUUGUAACUUGAA | 1124 | AAUAUCUUCAAGUUACAA | 1140 |
| | GAUAUA | | AAGCU | |
| Ps3543.7 | CUUUUGUAACUUGAA | 1124 | UAUAUCUUCAAGUUACAA | 1141 |
| | GAUAUA | | AAGCA | |
| Ps3543.8 | CUUUUGUAACUUGAA | 1124 | UAUAUCUUCAAGUUACAA | 1142 |
| | GAUAUA | | AAGUA | |
| Ps3543.9 | CUUUUGUAACUUGAA | 1124 | UAUAUCUUCAAGUUACAA | 1143 |
| | GAUAUA | | AAGUU | |
| Ps3543.10 | CUUUUGUAACUUGAA | 1124 | UAUAUCUUCAAGUUACAA | 1144 |
| | GAUAUA | | AAGAA | |
| Ps3543.11 | CUUUUGUAACUUGAA | 1124 | UAUAUCUUCAAGUUACAA | 1145 |
| | GAUAUA | | AAGAU | |

The sense strands and the antisense strands of the siRNA duplexes were synthesized using a classical oligonucleotide solid-phase synthesis method, in which a solid carrier was linked as an initial cycle, and nucleoside monomers were linked one by one in the 3′-5′ direction in the order in which nucleotides were arranged. Each time a nucleoside was linked, a four-step reaction of deprotection, coupling, capping, oxidation or sulfurization, or the like was involved, and finally, the sense or antisense strand molecules of the siRNA duplexes carrying the solid carrier were obtained. The conditions for each reaction step are as follows:
(1) Nucleoside monomer: The nucleoside monomers were dissolved in an acetonitrile solution at a concentration of 0.1 mol/L.
(2) Deprotection: A 3% dichloroacetic acid-dichloromethane solution was added.
(3) Coupling reaction: A 0.3 mol/L solution of ETT in acetonitrile was added.
(4) Oxidation reaction: A 0.05 mol/L solution of iodine in tetrahydrofuran/pyridine/water (70/20/10, v/v/v) was added.
(5) Sulfurization reaction: A 0.2 mol/L solution of xanthane hydride in pyridine was added.
(6) Capping reaction: 20% acetic anhydride-acetonitrile and pyridine/N-methylimidazole/acetonitrile (10/14/76, v/v/v) solutions were added.

The sense strands or the antisense strands of the synthesized siRNA duplexes carrying the solid carrier were added to 2-mL centrifuge tubes, and 25%-28% ammonia water was added. The mixtures were left to react at 55° C. for 16 h, filtered, and then washed 3 times with 1 mL of 50% aqueous ethanol solution to remove the solid carrier. The filtrates were concentrated to dryness to obtain crude products of the sense strands or the antisense strands of the siRNA duplexes, which were to be purified. The crude products of the sense strands or the antisense strands of the siRNA duplexes were dissolved in 1 mL of RNase-free water and then purified by ion-pair reversed-phase liquid chromatography or ion exchange chromatography. The collected samples were tested, and qualified samples were combined and desalted to obtain purified products of the sense strands or antisense strands of the siRNA duplexes containing amino modifications.

The purified products of the sense strands and antisense strands of the siRNA duplexes were mixed in a certain molar ratio, denatured at 90° C. for 3 min, then cooled to room temperature, and finally lyophilized to obtain purified products of the siRNA duplexes.

### Example 2: Screening of PCSK9 Protein siRNA Duplexes in Dual-Luciferase Reporter (DLR) Gene System

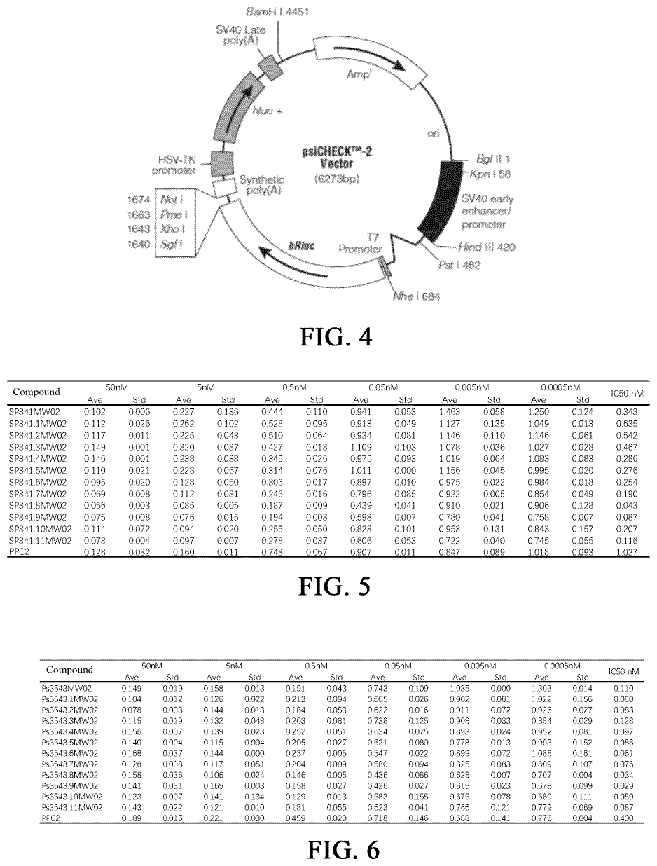

The method used in this example was to co-transfect a psi-CHECK2 plasmid (specifically shown in FIG. 4) containing the PCSK9 mRNA sequence and the PCSK9 protein siRNA duplex into a COS-7 cell line by liposome encapsulation. 24 h after transfection, the chemiluminescence values of the firefly fluorophore (Firefly) and renilla fluorophore (Renilla) on the plasmid were read. The fluorescence activity was determined by a microplate reader, the collected Renilla signals were normalized according to the Firefly signal standard, and the inhibitory effect of the siRNA was obtained by comparing the processed results (residual inhibitory activity). PPC1 was synthesized with reference to the patent WO2023208106A1, and PPC2 was synthesized with reference to the patent CN104854242B, both of which were positive controls.

The calculation process is as follows:

Normalized Ren/Fir ratio: Ratio=Renilla (renilla luciferase)/Firefly (firefly luciferase).

Residual inhibition rate: Average value of (Ratio siRNA/Ratio control)×100% from 2 replicate wells, where Ratio control is the average value of Ratio from 2 replicate control wells (without siRNA); Ratio siRNA/Ratio control is calculated for each of the 2 replicate wells, and then the average value is taken as the residual inhibition rate.

### 2.1 Construction of Psi-CHECK2 Plasmid

Since the human transcript of the PCSK9 gene was 3760 bp, when the psi-CHECK2 plasmid was constructed, the original transcript was inserted into the 3′-UTR region of psi-CHECK2. The sequence information of the vector containing the insert fragment is attached at the end of the text. After the plasmid construction was completed, the plasmid was diluted to 1000 ng/μL for later use. The psi-CHECK2 plasmid map is shown in FIG. 4.

2.2 Preparation of Working Solution Containing Double-Stranded siRNA

The synthesized double-stranded siRNA was a lyophilized powder. Each of the siRNA lyophilized powders described above was centrifuged, and an appropriate amount of sterile enzyme-free water was added according to the label amount of each tube for dissolution to prepare a 100 μM mother liquor. The mother liquor was further subjected to gradient dilution with sterile enzyme-free water to prepare a 10 μM, 1 μM, or 0.1 μM working solution.

### 2.3 Cell Culture, Plating, and Transfection

After the cells were washed once with PBS, the COS-7 cells that had grown to a nearly confluent state were digested with trypsin to prepare a cell suspension. The cell suspension was added to a 96-well plate at 100 μL/well, with each well containing 3×10⁴ cells. After the cells adhered to the wall for 24 h, the DMEM medium in the 96-well plate was discarded by pipetting, and 70 μL of Opti-MEM medium was added to each well. Then, the 96-well plate was placed in a carbon dioxide incubator for further culture. At this point, a plasmid mixture and a Lipo mixture were prepared. 1 μL of the plasmid was added to 14 μL of opti-MEM, and the mixture was uniformly mixed to prepare a plasmid mixture. 1 μL of Lipo2000 was added to 14 μL of opti-MEM, and the mixture was uniformly mixed to prepare a Lipo mixture. After the preparation of the plasmid mixture and the Lipo mixture was completed, the mixtures were left to stand at room temperature for 5 min, and then 15 μL of the plasmid mixture, 1 μL of the siRNA lyophilized powder, and 15 μL of the Lipo mixture were transferred to the same tube, mixed uniformly, and left to stand at room temperature for 15 min to form a transfection complex. The transfection complex was then sequentially transferred to a 96-well plate at 30 μL/well. After the plate was incubated in a carbon dioxide incubator for 4 h, 100 μL of a DMEM medium containing 20% FBS was added to each well, and the plate was incubated in the carbon dioxide incubator for another 24 h. Finally, the chemiluminescence value was measured.

### 2.4. Measurement of Chemiluminescence Value

The relevant reagents in the Dual-Luciferase® Reporter Assay System (Promega, E1960) kit were taken out and thawed to room temperature before the experiment.

After the cell culture was completed, the original medium in the 96-well plate was discarded by pipetting, and a certain amount of PBS was added to the 96-well plate for cell washing. After the cell washing was completed, 1× lysis buffer (the passive lysis buffer was prepared into 1× with water before use) was added to the 96-well cell culture plate at 30 μL/well, and the plate was incubated in a horizontal shaker at a certain rotation speed for 5 min to fully lyse the cells. After the lysis was completed, 20 μL of the lysed supernatant was transferred to a white impermeable microplate, LARII was added to the microplate at 50 μL/well, and the Firefly chemiluminescence values were read in a microplate reader. After the reading was completed, Stop&Glo was added to the microplate at 50 μL/well, and the Renilla chemiluminescence values were read in the microplate reader. After the reading was completed, the Firefly and Renilla chemiluminescence values were derived, the Renilla signals and the Firefly signal standard were normalized according to the formula described above, and the processed results were compared to obtain the inhibitory effects of the duplex siRNAs. The specific residual inhibitory activity is shown in Tables 3 and 4, wherein the PPC2 sequence was synthesized with reference to the patent CN104854242B and served as a positive control.

TABLE 3
Screening results of unmodified sequence
duplex siRNAs by 10 μM DLR

Residual activity

| Sequence | Ave (10 μM) | STDEV |
|---|---|---|
| NC | 100.00% | 17.40% |
| PPC1 | 45.72% | 1.81% |
| PPC2 | 36.64% | 1.10% |
| Ps178 | 53.69% | 11.10% |
| P446 | 63.73% | 11.32% |
| P516 | 68.45% | 11.67% |
| P607 | 58.55% | 13.74% |
| P612 | 38.88% | 6.65% |
| P629 | 90.51% | 2.20% |
| P633 | 48.37% | 12.21% |
| P693 | 44.87% | 5.25% |
| P823 | 66.58% | 3.21% |
| P835 | 47.74% | 6.42% |
| P839 | 49.11% | 14.08% |
| P842 | 62.14% | 9.46% |
| P845 | 64.92% | 17.34% |
| P854 | 57.77% | 10.03% |
| P882 | 58.42% | 16.57% |
| P888 | 37.19% | 2.50% |
| P891 | 40.78% | 9.64% |
| P1066 | 54.83% | 16.57% |
| P1079 | 34.02% | 6.21% |
| P1081 | 39.63% | 7.04% |
| P1085 | 79.33% | 16.10% |
| P1161 | 44.60% | 8.77% |
| P1242 | 62.57% | 16.52% |
| P1413 | 50.65% | 6.98% |
| P1514 | 78.74% | 12.02% |
| P1518 | 43.54% | 9.78% |
| P1521 | 59.13% | 9.20% |
| P1523 | 39.43% | 9.82% |
| P1531 | 43.61% | 7.82% |
| P1533 | 39.98% | 14.77% |
| P1536 | 47.12% | 10.51% |
| P1539 | 60.92% | 7.89% |
| P1547 | 43.19% | 4.21% |
| P1659 | 29.01% | 2.16% |
| P1661 | 39.47% | 18.05% |
| P2101 | 95.39% | 8.48% |
| P2112 | 37.51% | 9.19% |
| P2114 | 53.28% | 19.73% |
| P2116 | 28.24% | 2.58% |
| Ps2428 | 73.48% | 18.65% |
| Ps2537 | 54.92% | 9.09% |
| Ps2610 | 49.07% | 8.47% |
| Ps2617 | 47.31% | 16.27% |
| Ps2674 | 79.21% | 2.04% |
| Ps2683 | 64.83% | 13.58% |
| Ps2719 | 64.43% | 13.58% |
| Ps2721 | 63.94% | 11.03% |
| Ps2723 | 93.02% | 7.25% |
| Ps2725 | 74.95% | 13.69% |
| Ps2740 | 51.84% | 6.12% |
| Ps2749 | 34.12% | 3.49% |
| Ps2751 | 33.30% | 11.48% |
| Ps2817 | 29.71% | 6.96% |
| Ps2824 | 54.15% | 2.67% |
| Ps2836 | 40.34% | 5.55% |
| Ps2880 | 26.51% | 5.16% |
| Ps2884 | 27.31% | 6.23% |
| Ps2892 | 35.39% | 6.23% |
| Ps2971 | 40.34% | 4.28% |
| Ps2978 | 26.32% | 3.82% |
| Ps2984 | 46.93% | 1.28% |
| Ps2986 | 28.81% | 0.47% |
| Ps3057 | 26.61% | 5.96% |
| Ps3117 | 105.02% | 8.33% |
| Ps3119 | 14.18% | 1.82% |
| Ps3122 | 44.71% | 6.52% |
| Ps3124 | 59.00% | 7.75% |
| Ps3129 | 24.86% | 2.11% |
| Ps3140 | 18.32% | 2.80% |
| Ps3196 | 21.58% | 3.79% |
| Ps3198 | 34.84% | 8.07% |
| Ps3233 | 17.41% | 0.34% |
| Ps3316 | 37.75% | 2.98% |
| Ps3320 | 28.99% | 2.65% |
| Ps3464 | 19.24% | 4.99% |
| Ps3466 | 22.31% | 1.17% |
| Ps3489 | 24.85% | 2.00% |
| Ps3543 | 12.45% | 2.69% |
| Ps3548 | 23.27% | 7.35% |
| Ps3554 | 23.50% | 5.75% |

TABLE 4
Screening results of unmodified sequence duplex
siRNAs by 1 μM and 0.1 μM DLR

| | Residual activity | | | |
|---|---|---|---|---|
| Sequence | Ave (1 μM) | STDEV | Ave (0.1 μM) | STDEV |
| NC | 100.00% | 6.92% | 100.00% | 1.30% |
| PPC1 | 4.98% | 0.35% | 6.25% | 0.74% |
| PPC2 | 5.26% | 0.39% | 6.26% | 0.65% |
| SP70 | 57.80% | 0.53% | 92.89% | 5.75% |
| SP71 | 27.17% | 0.18% | 50.51% | 4.50% |
| SP73 | 25.20% | 0.02% | 39.74% | 1.34% |
| SP74 | 39.33% | 4.36% | 59.44% | 6.84% |
| SP76 | 49.46% | 2.74% | 68.85% | 3.67% |
| SP107 | 28.56% | 2.44% | 37.09% | 2.48% |
| SP109 | 18.32% | 2.93% | 21.28% | 1.74% |
| SP133 | 21.16% | 2.81% | 23.05% | 3.68% |
| SP134 | 23.63% | 0.37% | 32.40% | 1.42% |
| SP227 | 92.85% | 8.80% | 106.73% | 5.17% |
| SP300 | 32.25% | 0.20% | 42.19% | 0.13% |
| SP301 | 27.78% | 2.43% | 37.62% | 0.97% |
| SP304 | 24.49% | 0.86% | 31.72% | 4.06% |
| SP305 | 18.67% | 1.49% | 35.52% | 0.03% |
| SP306 | 16.21% | 0.20% | 39.80% | 3.88% |
| SP326 | 16.45% | 1.25% | 29.70% | 3.51% |
| SP327 | 17.11% | 1.18% | 31.75% | 1.97% |
| SP332 | 6.16% | 0.47% | 10.85% | 0.11% |
| SP334 | 28.20% | 2.00% | 35.97% | 2.49% |
| SP340 | 6.14% | 0.62% | 10.14% | 0.55% |
| SP341 | 9.56% | 0.98% | 12.82% | 0.69% |
| SP342 | 10.52% | 1.72% | 10.56% | 1.04% |
| SP343 | 7.85% | 0.41% | 9.86% | 1.07% |
| SP344 | 10.76% | 1.94% | 18.02% | 4.71% |
| SP345 | 6.68% | 0.51% | 10.14% | 0.94% |
| SP348 | 8.20% | 0.15% | 9.16% | 0.44% |
| SP349 | 11.94% | 0.15% | 17.79% | 0.39% |
| SP351 | 7.27% | 0.50% | 8.39% | 0.73% |
| SP357 | 24.09% | 0.28% | 63.60% | 7.32% |

It can be seen from this example that in the DLR screening system, some of the siRNA double strands exhibited more than 80% inhibitory activity against PCSK9 mRNA.

### Example 3: Assessment of Activity of Unmodified Sequences in HepG2 Cells

In this example, the inhibitory activity of 19 siRNA double-stranded unmodified sequences including Ps3119 and 216 siRNA double-stranded unmodified sequences including Pt609 against the target gene PCSK9 in HepG2 cells was assessed. The 19 siRNA double-stranded unmodified sequences including Ps3119 were transfected using 12-well cell plates, and the 216 siRNA double-stranded unmodified sequences including Pt609 were transfected using 96-well cell plates. PPC1 was synthesized with reference to the patent WO2023208106A1, and PPC2 was synthesized with reference to the patent CN104854242B, both of which were positive controls.

3.1 Preparation of Working Solution Containing Double-Stranded siRNA

The double-stranded siRNA was a lyophilized powder. Each of the siRNA lyophilized powders was centrifuged, and an appropriate amount of sterile enzyme-free water was added according to the label of each tube for dissolution to prepare a 100 μM mother liquor. The mother liquor was further subjected to gradient dilution with DEPC water to finally prepare a 10 μM working solution.

### 3.2 Cell Transfection and Detection

In this example, 12-well cell plates and 96-well cell plates were used for transfection. The transfection in the 12-well cell plate was performed as follows: after the cells were washed once with PBS, the HepG2 cells that had grown to a nearly confluent state were digested with trypsin to prepare a cell suspension, and the cells were plated in the 12-well plate. The 10 μM siRNA working solution was added to 90 μL of opti-MEM medium at 10 μL/well to prepare an siRNA mixture. 3 μL of RNAiMAX was then added to 97 μL of opti-MEM medium to prepare an RNAiMAX mixture. After the mixture was prepared, the siRNA mixture and the RNAiMAX mixture were mixed uniformly and incubated at room temperature for 15 min to prepare a transfection complex. After the incubation was completed, the transfection complex was transferred to the 12-well plate at 200 μL/well, and 800 μL of the cell suspension was added dropwise to each well. After the 12-well plate was placed in a cell incubator and cultured overnight for 48 h, the cells were collected and subjected to cellular RNA extraction using an RNA extraction kit. After the cellular extraction was completed, the concentration was determined using Nanodrop One. The procedures for the 96-well cell plate are as follows: after the cells were washed once with PBS, the HepG2 cells that had grown to a nearly confluent state were digested with trypsin to prepare a cell suspension, and the cells were plated in the 96-well plate. The siRNA test sample working solution was added to 9 μL of opti-MEM medium at 1 μL/well to prepare an siRNA test sample mixture. 0.3 μL of RNAiMAX was then added to 9.7 μL of opti-MEM medium to prepare an RNAiMAX mixture. After the mixture was prepared, the siRNA test sample mixture and the RNAiMAX mixture were mixed uniformly and incubated at room temperature for 15 min to prepare a transfection complex. After the incubation was completed, the transfection complex was transferred to the 96-well plate at 20 μL/well, and 80 μL of the cell suspension was added to each well. After the 96-well plate was placed in a cell incubator and cultured overnight for 24 h, the cells were collected and subjected to cellular RNA extraction using the RNA extraction kit.

After the RNA extraction in the 12-well cell plate was completed, the RNA was quantified to 500 ng according to the concentration determination result, and was reverse-transcribed using an RNA reverse transcription kit. The volume of RNA was specifically calculated as follows: V_{(RNA)}=500 ng/Conc._{(RNA)}. After the volume of RNA was determined, water was added to bring the volume to 8 μL. Subsequently, 2 μL of reverse transcriptase was added to each RNA, and reverse transcription was performed according to the procedures of 37° C. for 15 min and 85° C. for 5 min. After the reaction was completed, 40 μL of sterile enzyme-free water was added to each tube to obtain cDNA for subsequent qPCR. After the RNA extraction in the 96-well cell plate was completed, 8 μL of RNA was directly added to each well. Subsequently, 2 μL of reverse transcriptase was added to each RNA, and reverse transcription was performed according to the procedures of 37° C. for 15 min and 85° C. for 5 min. After the reaction was completed, cDNA was obtained for subsequent qPCR.

TABLE 5
Primer sequence information of the target gene and internal reference
gene used for qPCR in the 12-well plate

| Gene | | Primer type | Primer information |
|---|---|---|---|
| Target gene | PCSK9 | Upstream | AGACCCACCTCTCGCAGTC (SEQ ID |
| | | primer | NO 1113) |
| | | Downstream | GGAGTCCTCCTCGATGTAGTC (SEQ |
| | | primer | ID NO 1114) |
| Internal | GAPDH | Upstream | GGAGCGAGATCCCTCCAAAAT (SEQ |
| reference | | primer | ID NO 1115) |
| gene | | Downstream | GGCTGTTGTCATACTTCTCATGG (SEQ |
| | | primer | ID NO 1116) |

TABLE 6
Primer sequence information of the target gene and internal reference gene
used for qPCR in the 96-well plate

| Gene | | Primer type | Primer information |
|---|---|---|---|
| Target | PCSK9 | Probe primer | CCACCCAAGCAAGC (SEQ ID NO 1117) |
| gene | | Upstream | GGGAGAGGGCCAACAACTG (SEQ ID NO |
| | | primer | 1118) |
| | | Downstream | AGAGGACAGACCCAAAAGATAAATGT |
| | | primer | (SEQ ID NO 1119) |
| Internal | GAPDH | Probe primer | CAACAGCGACACCCACTC (SEQ ID NO |
| reference | | | 1120) |
| gene | | Upstream | AAGAAGGTGGTGAAGCAGG (SEQ ID NO |
| | | primer | 1121) |
| | | Downstream | CAAAGTGGTCGTTGAGGG (SEQ ID NO |
| | | primer | 1122) |

The qPCR method used in the 12-well plate was the SYBR GREEN method. The specific procedures were as follows: 20 μL of a Real-time PCR reaction system was prepared for each qPCR detection well according to the method described in the instructions of the real-time fluorescence quantitative PCR kit. Each reaction system contained 8 μL of the cDNA solution obtained from the reverse transcription reaction described above, 10 μL of TB Green®Premix Ex Taq (2×), 0.4 μL of 10 μM upstream primer, 0.4 μL of 10 μM downstream primer (the primer sequences are shown in Table 5), and 1.2 μL of RNase-Free H₂O. The prepared reaction system was placed on a real-time fluorescence quantitative PCR instrument, and Real-time PCR amplification was performed using a two-step method. The amplification procedure was as follows: pre-denaturation at 95° C. for 30 s, denaturation at 95° C. for 5 s, and annealing and extension at 60° C. for 30 s; and the process of denaturation, annealing, and extension was repeated for 40 cycles. The relative quantitative calculation of the expression level and inhibition rate of the target gene mRNA in each test group was performed by using the ΔΔCt method, and the specific results are shown in Table 7. The qPCR method used in the 96-well plate was the Taqman probe method. The specific procedures were as follows: 20 μL of a Real-time PCR reaction system was prepared for each qPCR detection well according to the method described in the instructions of the real-time fluorescence quantitative PCR kit. Each reaction system contained 5 μL of the cDNA solution obtained from the reverse transcription reaction described above, 10 μL of TaqMan™ Fast Advanced Mix (2×), 0.15 μL of 10 μM probe primer, 0.15 μL of 10 μM upstream primer, 0.15 μL of 10 μM downstream primer (the primer sequences are shown in Table 6), and 4.1 μL of RNase-Free H₂O. The prepared reaction system was placed on the real-time fluorescence quantitative PCR instrument, and Real-time PCR amplification was performed using a two-step method. The amplification procedure was as follows: pre-denaturation at 95° C. for 30 s, denaturation at 95° C. for 5 s, and annealing and extension at 60° C. for 30 s; and the process of denaturation, annealing, and extension was repeated for 40 cycles. The relative quantitative calculation of the expression level and inhibition rate of the target gene mRNA in each test group was performed by using the ΔΔCt method, and the specific results are shown in Table 8.

### 3.3 Data Calculation Method

$$Δ Ct ( test group ) = Ct ( target gene in test group ) - Ct ( internal reference gene in test group )$$

$$Δ Ct ( control group ) = Ct ( target gene in control group ) - Ct ( internal reference gene in control group )$$

$$ΔΔ Ct ( test group ) = Δ Ct ( test group ) - Δ Ct ( average of control group )$$

$$ΔΔ Ct ( control group ) = Δ Ct ( control group ) - Δ Ct ( average of control group )$$

Taking the control group as a baseline, the mRNA expression level of the target gene in the test group was normalized, and the residual expression level of the target gene mRNA in the control group was defined as 100%

Relative residual expression level of target gene mRNA in test group=2^{-ΔΔCt} (test group)×100%

Inhibition rate of target gene mRNA in test group=100%−relative expression level of target gene mRNA in test group

TABLE 7
Assessment results of the activity of 19 unmodified
siRNAs such as Ps3119 at 10 μM in HepG2 cells

Residual expression level (10 μM)

| Sequence | Ave (%) | STDEV |
|---|---|---|
| NC | 100.00% | 1.55% |
| PPC1 | 6.69% | 1.43% |
| PPC2 | 12.48% | 3.13% |
| Ps3119 | 6.40% | 0.12% |
| Ps3196 | 10.04% | 0.53% |
| Ps3233 | 4.70% | 0.46% |
| Ps3464 | 8.10% | 0.09% |
| Ps3466 | 9.08% | 0.44% |
| Ps3543 | 3.14% | 0.10% |
| Ps3548 | 7.60% | 0.15% |
| Ps3554 | 3.90% | 0.20% |
| SP332 | 1.75% | 0.07% |
| SP340 | 12.56% | 1.43% |
| SP341 | 21.02% | 0.00% |
| SP342 | 18.70% | 0.26% |
| SP343 | 25.85% | 0.12% |
| SP344 | 24.64% | 1.20% |
| SP345 | 17.40% | 0.01% |
| SP348 | 18.70% | 0.46% |
| SP349 | 11.24% | 0.22% |
| SP351 | 20.33% | 0.27% |

TABLE 8
Assessment results of the activity of 216 unmodified siRNAs
such as Pt609 at 1 μM and 0.1 μM in HepG2 cells

| | Residual expression level | | | |
|---|---|---|---|---|
| Sequence | Ave (1 μM) | STDEV | Ave (0.1 μM) | STDEV |
| Blank | 100.00% | 5.60% | — | — |
| PPC2 | 20.85% | 0.51% | 25.72% | 0.90% |
| Pt608 | 25.69% | 0.91% | 25.47% | 0.13% |
| Pt609 | 8.74% | 0.19% | 11.13% | 0.35% |
| Pt692 | 19.49% | 0.31% | 22.24% | 1.41% |
| Pt694 | 50.23% | 2.25% | 55.35% | 2.26% |
| Pt695 | 31.13% | 0.76% | 37.02% | 1.26% |
| Pt837 | 30.89% | 1.47% | 37.23% | 5.24% |
| Pt838 | 31.84% | 1.59% | 48.71% | 0.72% |
| Pt840 | 22.65% | 0.76% | 31.85% | 0.13% |
| Pt841 | 37.44% | 2.66% | 42.83% | 7.14% |
| Pt844 | 59.32% | 3.18% | 78.59% | 8.41% |
| Pt847 | 29.79% | 3.48% | 44.75% | 0.37% |
| Pt848 | 16.65% | 2.55% | 26.07% | 4.70% |
| Pt855 | 24.91% | 0.43% | 32.04% | 0.03% |
| Pt856 | 23.75% | 1.62% | 34.75% | 0.18% |
| Pt889 | 25.94% | 1.33% | 38.23% | 2.68% |
| Pt890 | 11.80% | 0.61% | 14.55% | 0.53% |
| Pt892 | 24.16% | 5.37% | 31.78% | 0.60% |
| Pt941 | 16.02% | 0.15% | 22.78% | 1.44% |
| Pt1080 | 16.40% | 1.68% | 21.93% | 0.78% |
| Pt1082 | 34.76% | 0.97% | 59.83% | 6.99% |
| Pt1083 | 38.19% | 4.27% | 57.86% | 2.83% |
| Pt1084 | 23.46% | 0.30% | 36.43% | 0.46% |
| Pt1086 | 10.68% | 0.45% | 14.04% | 0.65% |
| Pt1087 | 19.51% | 1.01% | 22.40% | 1.61% |
| Pt1275 | 20.33% | 0.75% | 24.42% | 1.30% |
| Pt1516 | 31.53% | 0.94% | 45.68% | 2.97% |
| Pt1517 | 27.83% | 0.14% | 31.37% | 1.45% |
| Pt1519 | 57.99% | 0.88% | 60.87% | 1.94% |
| Pt1520 | 52.66% | 1.21% | 60.67% | 3.45% |
| Pt1522 | 32.98% | 1.83% | 45.42% | 3.43% |
| Pt1530 | 30.79% | 0.79% | 39.07% | 2.51% |
| Pt1532 | 59.50% | 1.14% | 78.30% | 8.66% |
| Pt1534 | 35.36% | 0.40% | 53.28% | 10.42% |
| Pt1535 | 94.53% | 6.01% | 86.48% | 1.11% |
| Pt1537 | 40.83% | 3.68% | 54.66% | 1.30% |
| Pt1538 | 49.67% | 0.55% | 58.46% | 9.26% |
| Pt2102 | 10.36% | 0.35% | 18.60% | 0.54% |
| Pt2105 | 25.48% | 0.88% | 32.26% | 2.02% |
| Pt2106 | 18.03% | 0.24% | 20.63% | 0.09% |
| Pt2113 | 17.53% | 1.92% | 26.45% | 2.77% |
| Pt2115 | 30.26% | 0.44% | 60.37% | 1.02% |
| Pt2426 | 23.00% | 0.92% | 22.74% | 2.02% |
| Pt2427 | 21.64% | 1.72% | 21.57% | 2.26% |
| Pt2432 | 13.27% | 0.58% | 17.81% | 1.96% |
| Pt2530 | 11.76% | 0.41% | 14.78% | 0.24% |
| Pt2532 | 12.99% | 2.68% | 12.86% | 0.85% |
| Pt2533 | 8.93% | 1.02% | 12.93% | 0.16% |
| Pt2534 | 12.02% | 0.38% | 15.18% | 1.03% |
| Pt2535 | 15.52% | 1.23% | 17.47% | 0.29% |
| Pt2536 | 14.17% | 1.31% | 13.49% | 0.35% |
| Pt2538 | 16.05% | 0.21% | 14.21% | 0.96% |
| Pt2540 | 24.11% | 0.83% | 25.39% | 2.52% |
| Pt2541 | 12.96% | 1.93% | 13.33% | 0.35% |
| Pt2542 | 20.57% | 0.35% | 33.33% | 0.17% |
| Pt2544 | 62.54% | 1.41% | 67.48% | 2.05% |
| Pt2545 | 23.38% | 4.42% | 34.41% | 4.31% |
| Pt2611 | 19.19% | 3.55% | 25.57% | 2.44% |
| Pt2612 | 16.25% | 0.42% | 13.15% | 1.38% |
| Pt2613 | 36.58% | 0.62% | 32.47% | 1.92% |
| Pt2614 | 14.54% | 0.96% | 20.96% | 3.51% |
| Pt2615 | 20.73% | 0.92% | 24.84% | 1.62% |
| Pt2616 | 16.34% | 0.18% | 19.51% | 0.50% |
| Pt2618 | 24.57% | 0.70% | 26.00% | 0.96% |
| Pt2656 | 20.58% | 0.68% | 21.59% | 2.50% |
| Pt2673 | 17.60% | 1.51% | 20.10% | 1.94% |
| Pt2675 | 11.60% | 0.48% | 13.82% | 0.86% |
| Pt2696 | 14.07% | 0.16% | 18.06% | 1.11% |
| Pt2697 | 13.98% | 2.64% | 15.96% | 2.73% |
| Pt2698 | 9.33% | 0.49% | 9.50% | 0.21% |
| Pt2699 | 13.44% | 1.73% | 12.67% | 1.34% |
| Pt2700 | 6.89% | 0.10% | 9.00% | 1.12% |
| Pt2717 | 8.87% | 0.65% | 13.24% | 1.06% |
| Pt2718 | 15.19% | 0.35% | 16.67% | 0.04% |
| Pt2720 | 20.17% | 1.13% | 28.12% | 0.93% |
| Pt2722 | 31.09% | 2.11% | 38.68% | 0.24% |
| Pt2724 | 36.65% | 6.97% | 29.56% | 0.72% |
| Pt2726 | 27.53% | 1.97% | 32.83% | 0.20% |
| Pt2727 | 16.27% | 1.50% | 15.47% | 0.86% |
| Pt2741 | 15.51% | 0.30% | 18.26% | 1.54% |
| Pt2742 | 17.50% | 0.39% | 23.09% | 0.72% |
| Pt2743 | 25.60% | 0.42% | 23.15% | 0.59% |
| Pt2744 | 20.19% | 2.54% | 18.21% | 0.53% |
| Pt2745 | 20.19% | 1.60% | 27.63% | 1.62% |
| Pt2746 | 34.66% | 4.37% | 53.64% | 1.84% |
| Pt2747 | 15.38% | 0.58% | 17.75% | 1.76% |
| Pt2748 | 10.70% | 0.03% | 13.42% | 0.22% |
| Pt2750 | 10.61% | 2.01% | 14.53% | 0.32% |
| Pt2752 | 14.86% | 0.58% | 14.71% | 0.35% |
| Pt2753 | 18.77% | 0.75% | 22.22% | 1.04% |
| Pt2789 | 20.84% | 3.95% | 17.26% | 1.19% |
| Pt2790 | 14.07% | 0.09% | 17.03% | 0.75% |
| Pt2791 | 14.62% | 1.31% | 19.30% | 1.87% |
| Pt2792 | 10.56% | 1.76% | 20.20% | 0.65% |
| Pt2815 | 22.42% | 0.58% | 22.10% | 1.34% |
| Pt2816 | 26.26% | 2.34% | 33.34% | 8.70% |
| Pt2818 | 10.25% | 0.12% | 13.77% | 1.11% |
| Pt2819 | 19.75% | 0.03% | 21.97% | 1.08% |
| Pt2820 | 37.38% | 7.52% | 34.96% | 6.92% |
| Pt2821 | 54.65% | 4.01% | 38.84% | 6.25% |
| Pt2822 | 31.55% | 0.45% | 41.24% | 4.51% |
| Pt2823 | 13.38% | 1.03% | 18.30% | 1.09% |
| Pt2837 | 11.69% | 1.04% | 11.80% | 0.73% |
| Pt2838 | 11.10% | 0.94% | 14.33% | 0.12% |
| Pt2839 | 26.49% | 0.44% | 33.46% | 0.62% |
| Pt2840 | 19.19% | 0.16% | 29.17% | 3.94% |
| Pt2850 | 27.82% | 5.61% | 28.09% | 2.54% |
| Pt2851 | 41.18% | 7.13% | 41.20% | 3.13% |
| Pt2879 | 13.09% | 0.51% | 16.22% | — |
| Pt2881 | 17.51% | 0.34% | 16.79% | 2.53% |
| Pt2882 | 11.73% | 0.96% | 13.86% | 2.45% |
| Pt2883 | 12.19% | 0.03% | 12.70% | 1.72% |
| Pt2885 | 37.10% | 4.57% | 39.29% | 4.55% |
| Pt2886 | 27.18% | 0.28% | 33.06% | 4.62% |
| Pt2887 | 12.04% | 1.26% | 18.59% | 0.53% |
| Pt2888 | 9.81% | 0.15% | 12.59% | 0.62% |
| Pt2889 | 11.06% | 1.86% | 13.23% | 0.78% |
| Pt2890 | 14.60% | 2.00% | 18.44% | 0.68% |
| Pt2891 | 15.58% | 3.44% | 19.46% | 1.07% |
| Pt2893 | 13.21% | 0.78% | 21.41% | 0.34% |
| Pt2959 | 17.79% | 0.97% | 29.00% | 0.01% |
| Pt2960 | 33.72% | 1.80% | 32.92% | 1.02% |
| Pt2962 | 17.09% | 0.05% | 24.20% | 3.09% |
| Pt2963 | 13.34% | 1.06% | 16.02% | 0.65% |
| Pt2964 | 32.50% | 6.95% | 34.62% | 2.67% |
| Pt2965 | 23.47% | 1.38% | 26.19% | 1.04% |
| Pt2966 | 29.25% | 1.12% | 33.25% | 1.29% |
| Pt2967 | 15.45% | 0.10% | 19.89% | 0.52% |
| Pt2968 | 45.84% | 1.54% | 37.81% | 0.63% |
| Pt2969 | 51.34% | 4.20% | 35.34% | 0.70% |
| Pt2970 | 44.37% | 8.50% | 32.57% | 0.03% |
| Pt2972 | 16.75% | 0.40% | 24.31% | 1.93% |
| Pt2977 | 12.80% | 1.06% | 21.09% | 4.34% |
| Pt2979 | 18.42% | 1.26% | 24.99% | 0.28% |
| Pt2980 | 11.16% | 0.40% | 10.28% | 0.11% |
| Pt2981 | 24.43% | 1.63% | 35.07% | 4.45% |
| Pt2982 | 57.48% | 0.98% | 59.40% | 3.31% |
| Pt2983 | 87.45% | 24.02% | 101.87% | 5.93% |
| Pt2985 | 18.48% | 1.09% | 18.75% | 1.42% |
| Pt2987 | 27.66% | 0.05% | 30.82% | 2.67% |
| Pt2989 | 14.69% | 0.57% | 21.18% | 1.27% |
| Pt2990 | 16.34% | 0.98% | 17.85% | 0.82% |
| Pt2991 | 12.92% | 3.74% | 13.46% | 0.30% |
| Pt3037 | 18.46% | 1.38% | 22.42% | 0.56% |
| Pt3038 | 18.80% | 0.11% | 24.18% | 1.47% |
| Pt3039 | 27.92% | 0.66% | 25.57% | 0.18% |
| Pt3042 | 23.38% | 0.16% | 22.49% | 3.44% |
| Pt3043 | 14.94% | 0.06% | 19.94% | 2.31% |
| Pt3125 | 22.12% | 0.06% | 24.17% | 2.54% |
| Pt3126 | 19.01% | 2.01% | 23.23% | 0.46% |
| Pt3127 | 20.37% | 0.14% | 25.14% | 1.34% |
| Pt3138 | 14.14% | 0.41% | 15.67% | 0.54% |
| Pt3139 | 9.28% | 0.64% | 15.32% | 2.46% |
| Pt3141 | 11.92% | 0.46% | 14.97% | 0.68% |
| Pt3177 | 20.14% | 2.42% | 21.73% | 2.67% |
| Pt3178 | 18.17% | 0.01% | 22.85% | 0.68% |
| Pt3179 | 38.28% | 0.17% | 41.80% | 3.08% |
| Pt3180 | 20.02% | 0.58% | 20.31% | 1.83% |
| Pt3185 | 10.73% | 0.75% | 11.98% | 2.39% |
| Pt3186 | 13.96% | 0.88% | 12.91% | 1.30% |
| Pt3187 | 14.14% | 0.56% | 16.63% | 0.09% |
| Pt3188 | 17.34% | 3.02% | 18.95% | 2.01% |
| Pt3189 | 17.24% | 1.56% | 17.86% | 2.24% |
| Pt3190 | 13.96% | 1.84% | 17.24% | 1.51% |
| Pt3191 | 11.36% | 0.83% | 13.32% | 0.51% |
| Pt3192 | 13.69% | 0.44% | 17.91% | 1.23% |
| Pt3193 | 11.59% | 1.27% | 12.52% | 0.45% |
| Pt3194 | 6.98% | 0.02% | 13.31% | 1.51% |
| Pt3199 | 8.18% | 1.34% | 15.18% | 0.65% |
| Pt3200 | 18.09% | 2.81% | 20.97% | 0.07% |
| Pt3205 | 30.57% | 3.04% | 28.74% | 0.03% |
| Pt3206 | 33.78% | 0.72% | 46.97% | 2.36% |
| Pt3207 | 59.62% | 2.99% | 44.28% | 3.34% |
| Pt3234 | 15.59% | 2.33% | 16.68% | 0.22% |
| Pt3235 | 50.94% | 5.24% | 64.47% | 3.48% |
| Pt3236 | 8.47% | 1.04% | 12.15% | 0.18% |
| Pt3237 | 13.10% | 0.02% | 16.90% | 1.19% |
| Pt3238 | 28.89% | 0.17% | 35.61% | 0.89% |
| Pt3239 | 44.69% | 5.06% | 29.32% | 0.15% |
| Pt3240 | 18.65% | 0.02% | 17.74% | 0.52% |
| Pt3241 | 57.05% | 0.63% | 79.03% | 3.44% |
| Pt3243 | 28.01% | 0.95% | 26.96% | 0.94% |
| Pt3275 | 19.73% | 1.24% | 19.08% | 1.52% |
| Pt3276 | 6.91% | 1.24% | 11.34% | 1.08% |
| Pt3302 | 27.17% | 0.30% | 22.80% | 1.40% |
| Pt3303 | 21.41% | 0.31% | 21.25% | 1.47% |
| Pt3304 | 6.04% | 0.67% | 8.97% | 1.22% |
| Pt3306 | 14.13% | 1.15% | 17.76% | 1.28% |
| Pt3308 | 13.01% | 1.02% | 16.19% | 0.38% |
| Pt3309 | 11.32% | 0.75% | 17.60% | 2.60% |
| Pt3310 | 18.63% | 0.34% | 23.24% | 0.11% |
| Pt3313 | 22.43% | 1.72% | 21.52% | 1.25% |
| Pt3315 | 13.91% | 0.04% | 15.61% | 0.07% |
| Pt3317 | 38.32% | 3.07% | 37.09% | 1.86% |
| Pt3319 | 26.69% | 3.93% | 34.98% | 5.80% |
| Pt3321 | 22.34% | 5.56% | 20.46% | 1.38% |
| Pt3322 | 14.63% | 0.05% | 16.36% | 2.72% |
| Pt3382 | 43.64% | 1.92% | 49.16% | 3.71% |
| Pt3390 | 15.62% | 1.81% | 17.84% | 2.49% |
| Pt3394 | 18.43% | 1.26% | 22.08% | 2.73% |
| Pt3431 | 27.03% | 3.71% | 33.03% | 1.36% |
| Pt3432 | 20.94% | 0.12% | 28.37% | 1.04% |
| Pt3433 | 21.38% | 1.84% | 27.66% | 0.69% |
| Pt3434 | 12.18% | 0.10% | 19.97% | 0.04% |
| Pt3435 | 18.49% | 0.80% | 25.41% | 3.49% |
| Pt3436 | 10.07% | 0.99% | 13.19% | 2.03% |
| Pt3437 | 14.54% | 4.50% | 16.88% | 0.33% |
| Pt3438 | 18.19% | 4.44% | 13.72% | 0.38% |
| Pt3461 | 32.34% | 1.10% | 37.36% | 9.63% |
| Pt3465 | 24.19% | 0.71% | 35.02% | 1.63% |
| Pt3468 | 14.84% | 2.23% | 22.16% | 1.66% |
| Pt3490 | 23.26% | 0.46% | 24.47% | 0.72% |
| Pt3491 | 15.68% | 3.23% | 21.08% | 0.43% |
| Pt3492 | 23.05% | 0.70% | 37.59% | 1.04% |
| Pt3493 | 10.16% | 0.17% | 12.40% | 0.06% |
| Pt3552 | 14.18% | 1.24% | 19.50% | 0.35% |
| Pt3553 | 6.76% | 0.71% | 10.64% | 0.62% |

It can be seen from this example that in the HepG2 cells, some of the siRNA test samples exhibited more than 95% inhibitory activity against PCSK9 mRNA.

### Example 4: Preparation of Modified Sequences

In the sequences of this example, the base composition and modifications have the following meanings: The uppercase letters A, U, G, C, and T represent the base composition of a nucleotide; the lowercase letter m indicates that the nucleotide adjacent to the left of the letter m is a 2′-methoxy-modified nucleotide; the lowercase letter f indicates that the nucleotide adjacent to the left of the letter f is a 2′-fluoro-modified nucleotide; the lowercase letter s indicates that the two adjacent nucleotides flanking the letter s are linked by a phosphorothioate bond; L96 is an N-acetylgalactosamine conjugate. The specific modified sequences are shown in Table 9, wherein PPC1 was synthesized with reference to the patent WO2023208106A1, PPC2 was synthesized with reference to the patent CN104854242B, and the modifications of the positive control sequences were made according to the reference patents.

TABLE 9
Modified double-stranded siRNA sequences

| | | SEQ | | SEQ |
|---|---|---|---|---|
| Compound | Sense strand (5′-3′) | ID NO | Antisense strand (5′-3′) | ID NO |
| P3119MW02 | CmsAmsCmAmUmUmCf | 982 | UmsGfsAmAmGmUmAmGm | 1045 |
| | GmCfAfCfCmCmCmUm | | GmGmGmUmGmCfGmAfAm | |
| | AmCmUmUmCmAm | | UmGmUmGmsUmsAm | |
| P3196MW02 | AmsCmsUmGmAmGmCf | 983 | AmsAfsUmCmUmGmCmGm | 1046 |
| | CmAfGfAfAmAmCmGm | | UmUmUmCmUmGfGmCfUm | |
| | CmAmGmAmUmUm | | CmAmGmUmsUmsCm | |
| P3464MW02 | CmsCmsCmAmAmGmCf | 984 | AmsUfsAmAmAmUmGmUm | 1047 |
| | AmAfGfCfAmGmAmCm | | CmUmGmCmUmUfGmCfUm | |
| | AmUmUmUmAmUm | | UmGmGmGmsUmsGm | |
| P3543MW02 | CmsUmsUmUmUmGmU | 985 | AmsAfsUmAmUmCmUmUm | 1048 |
| | fAmAfCfUfUmGmAmA | | CmAmAmGmUmUfAmCfAm | |
| | mGmAmUmAmUmUm | | AmAmAmGmsCmsAm | |
| P3554MW02 | UmsGmsAmAmGmAmU | 986 | AmsAfsCmCmCmAmGmAmA | 1049 |
| | fAmUfUfUfAmUmUmC | | mUmAmAmAmUfAmUfCmU | |
| | mUmGmGmGmUmUm | | mUmCmAmsAmsGm | |
| SP340MW02 | GmsCmsCmAmAmCmUf | 987 | AmsAfsCmAmGmGmUmCm | 1050 |
| | UmUfUfCfUmAmGmAm | | UmAmGmAmAmAfAmGfUm | |
| | CmCmUmGmUmUm | | UmGmGmCmsUmsGm | |
| SP341MW02 | CmsCmsAmAmCmUmUf | 988 | AmsAfsAmCmAmGmGmUm | 1051 |
| | UmUfCfUfAmGmAmCm | | CmUmAmGmAmAfAmAfGm | |
| | CmUmGmUmUmUm | | UmUmGmGmsCmsUm | |
| SP342MW02 | CmsAmsAmCmUmUmUf | 989 | AmsAfsAmAmCmAmGmGm | 1052 |
| | UmCfUfAfGmAmCmCm | | UmCmUmAmGmAfAmAfAm | |
| | UmGmUmUmUmUm | | GmUmUmGmsGmsCm | |
| SP344MW02 | AmsCmsUmUmUmUmCf | 990 | GmsCfsAmAmAmAmCmAm | 1053 |
| | UmAfGfAfCmCmUmGm | | GmGmUmCmUmAfGmAfAm | |
| | UmUmUmUmGmCm | | AmAmGmUmsUmsGm | |
| SP345MW02 | CmsUmsUmUmUmCmUf | 991 | AmsGfsCmAmAmAmAmCm | 1054 |
| | AmGfAfCfCmUmGmUm | | AmGmGmUmCmUfAmGfAm | |
| | UmUmUmGmCmUm | | AmAmAmGmsUmsUm | |
| SP349MW02 | UmsCmsUmAmGmAmCf | 992 | CmsAfsAmAmAmGmCmAm | 1055 |
| | CmUfGfUfUmUmUmGm | | AmAmAmCmAmGfGmUfCm | |
| | CmUmUmUmUmGm | | UmAmGmAmsAmsAm | |
| SP351MW02 | AmsGmsAmCmCmUmGf | 993 | UmsUfsAmCmAmAmAmAm | 1056 |
| | UmUfUfUfGmCmUmUm | | GmCmAmAmAmAfCmAfGm | |
| | UmUmGmUmAmAm | | GmUmCmUmsAmsGm | |
| WP3239MW02 | GmsGmsGmUmAmCmA | 994 | UmsAfsGmGmGmGmUmGm | 1057 |
| | fCmAfUfUfCmGmCmA | | CmGmAmAmUmGfUmGfUm | |
| | mCmCmCmCmUmAm | | AmCmCmCmsUmsGm | |
| WP3262MW02 | UmsCmsAmCmAmGmAf | 995 | UmsCfsCmAmGmGmUmUm | 1058 |
| | GmGfAfAfGmAmAmAm | | UmCmUmUmCmCfUmCfUm | |
| | CmCmUmGmGmAm | | GmUmGmAmsAmsGm | |
| WP3315MW02 | GmsCmsAmGmGmAmA | 996 | CmsGfsUmUmUmCmUmGm | 1059 |
| | fCmUfGfAfGmCmCmA | | GmCmUmCmAmGfUmUfCm | |
| | mGmAmAmAmCmGm | | CmUmGmCmsUmsGm | |
| WP3315- | GmsCmsAmGmGmAmA | 997 | UmsGfsUmUmUmCmUmGm | 1060 |
| AMW02 | fCmUfGfAfGmCmCmA | | GmCmUmCmAmGfUmUfCm | |
| | mGmAmAmAmCmAm | | CmUmGmCmsUmsGm | |
| WP3323MW02 | UmsGmsAmGmCmCmAf | 998 | AmsCfsUmGmAmGmCmCmA | 1061 |
| | GmAfAfAfCmGmCmAm | | mGmAmAmAmCfGmCfAmG | |
| | GmAmUmUmGmGm | | mAmUmUmsGmsGm | |
| PPC1 | CmsUmsGmUmUmUmU | 1198 | CmsAfsAmGmUmUfAmCmA | 1199 |
| | fGmCfUfUfUmUmGmU | | mAmAmAmGmCfAmAfAmA | |
| | mAmAmCmUmUmGm | | mCmAmGmsGmsUm | |
| PPC2 | CmsUmsAmGmAmCmCf | 1200 | AmsCfsAmAfAfAfGmCfAmA | 1201 |
| | UmGfUmdTUmUmGmC | | fAmAfCmAfGmGfUmCfUmA | |
| | mUmUmUmUmGmUm | | mGmsAmsAm | |
| Pt609MW02 | CmsCmsUmCmAmCmCf | 999 | AmsCfsAmUmGmCmAmGm | 1062 |
| | AmAfGfAfUmCmCmUm | | GmAmUmCmUmUfGmGfUm | |
| | GmCmAmUmGmUm | | GmAmGmGmsUmsAm | |
| Pt890MW02 | UmsGmsGmUmCmAmCf | 1000 | CmsAfsUmUmCmUmCmGmA | 1063 |
| | CmGfAfCfUmUmCmGm | | mAmGmUmCmGfGmUfGmA | |
| | AmGmAmAmUmGm | | mCmCmAmsUmsGm | |
| Pt2530MW02 | CmsCmsUmCmCmUmUf | 1001 | AmsGfsUmGmAmGmUmUm | 1064 |
| | GmCfCfUfGmGmAmAm | | CmCmAmGmGmCfAmAfGm | |
| | CmUmCmAmCmUm | | GmAmGmGmsCmsUm | |
| Pt2532MW02 | UmsCmsCmUmUmGmCf | 1002 | UmsGfsAmGmUmGmAmGm | 1065 |
| | CmUfGfGfAmAmCmUm | | UmUmCmCmAmGfGmCfAm | |
| | CmAmCmUmCmAm | | AmGmGmAmsGmsGm | |
| Pt2533MW02 | CmsCmsUmUmGmCmCf | 1003 | GmsUfsGmAmGmUmGmAm | 1066 |
| | UmGfGfAfAmCmUmCm | | GmUmUmCmCmAfGmGfCm | |
| | AmCmUmCmAmCm | | AmAmGmGmsAmsGm | |
| Pt2534MW02 | CmsUmsUmGmCmCmUf | 1004 | AmsGfsUmGmAmGmUmGm | 1067 |
| | GmGfAfAfCmUmCmAm | | AmGmUmUmCmCfAmGfGm | |
| | CmUmCmAmCmUm | | CmAmAmGmsGmsAm | |
| Pt2536MW02 | UmsGmsCmCmUmGmGf | 1005 | AmsGfsAmGmUmGmAmGm | 1068 |
| | AmAfCfUfCmAmCmUm | | UmGmAmGmUmUfCmCfAm | |
| | CmAmCmUmCmUm | | GmGmCmAmsAmsGm | |
| Pt2538MW02 | CmsCmsUmGmGmAmAf | 1006 | CmsCfsAmGmAmGmUmGm | 1069 |
| | CmUfCfAfCmUmCmAm | | AmGmUmGmAmGfUmUfCm | |
| | CmUmCmUmGmGm | | CmAmGmGmsCmsAm | |
| Pt2541MW02 | GmsGmsAmAmCmUmCf | 1007 | CmsAfsCmCmCmAmGmAmG | 1070 |
| | AmCfUfCfAmCmUmCm | | mUmGmAmGmUfGmAfGmU | |
| | UmGmGmGmUmGm | | mUmCmCmsAmsGm | |
| Pt2612MW02 | AmsUmsUmUmCmAmCf | 1008 | GmsAfsCmCmUmGmUmUm | 1071 |
| | CmAfUfUfCmAmAmAm | | UmGmAmAmUmGfGmUfGm | |
| | CmAmGmGmUmCm | | AmAmAmUmsGmsCm | |
| Pt2675MW02 | GmsUmsCmCmGmUmGf | 1009 | AmsAfsAmGmUmCmAmUm | 1072 |
| | GmGfCfAfGmAmAmUm | | UmCmUmGmCmCfCmAfCm | |
| | GmAmCmUmUmUm | | GmGmAmCmsAmsUm | |
| Pt2697MW02 | AmsUmsUmGmAmGmC | 1010 | GmsGfsCmAmCmGmGmAm | 1073 |
| | fUmCfUfUfGmUmUmC | | AmCmAmAmGmAfGmCfUm | |
| | mCmGmUmGmCmCm | | CmAmAmUmsAmsAm | |
| Pt2698MW02 | UmsUmsGmAmGmCmU | 1011 | UmsGfsGmCmAmCmGmGm | 1074 |
| | fCmUfUfGfUmUmCmC | | AmAmCmAmAmGfAmGfCm | |
| | mGmUmGmCmCmAm | | UmCmAmAmsUmsAm | |
| Pt2699MW02 | UmsGmsAmGmCmUmCf | 1012 | CmsUfsGmGmCmAmCmGmG | 1075 |
| | UmUfGfUfUmCmCmGm | | mAmAmCmAmAfGmAfGmC | |
| | UmGmCmCmAmGm | | mUmCmAmsAmsUm | |
| Pt2700MW02 | GmsAmsGmCmUmCmUf | 1013 | CmsCfsUmGmGmCmAmCmG | 1076 |
| | UmGfUfUfCmCmGmUm | | mGmAmAmCmAfAmGfAmG | |
| | GmCmCmAmGmGm | | mCmUmCmsAmsAm | |
| Pt2717MW02 | CmsAmsGmGmCmAmUf | 1014 | GmsAfsCmCmUmGmAmGm | 1077 |
| | UmCfAfAfUmCmCmUm | | GmAmUmUmGmAfAmUfGm | |
| | CmAmGmGmUmCm | | CmCmUmGmsGmsCm | |
| Pt2727MW02 | AmsUmsCmCmUmCmAf | 1015 | CmsCfsUmUmGmGmUmGm | 1078 |
| | GmGfUfCfUmCmCmAm | | GmAmGmAmCmCfUmGfAm | |
| | CmCmAmAmGmGm | | GmGmAmUmsUmsGm | |
| Pt2748MW02 | AmsGmsGmCmAmGmG | 1016 | UmsCfsCmAmUmGmGmGm | 1079 |
| | fAmUfUfCfUmUmCmC | | AmAmGmAmAmUfCmCfUm | |
| | mCmAmUmGmGmAm | | GmCmCmUmsCmsCm | |
| Pt2750MW02 | GmsCmsAmGmGmAmU | 1017 | UmsAfsUmCmCmAmUmGm | 1080 |
| | fUmCfUfUfCmCmCmA | | GmGmAmAmGmAfAmUfCm | |
| | mUmGmGmAmUmAm | | CmUmGmCmsCmsUm | |
| Pt2752MW02 | AmsGmsGmAmUmUmC | 1018 | CmsCfsUmAmUmCmCmAmU | 1081 |
| | fUmUfCfCfCmAmUmG | | mGmGmGmAmAfGmAfAmU | |
| | mGmAmUmAmGmGm | | mCmCmUmsGmsCm | |
| Pt2818MW02 | AmsGmsUmGmUmGmA | 1019 | GmsCfsCmAmUmCmAmGmC | 1082 |
| | fAmAfGfGfUmGmCmU | | mAmCmCmUmUfUmCfAmC | |
| | mGmAmUmGmGmCm | | mAmCmUmsCmsAm | |
| Pt2837MW02 | GmsCmsCmCmUmCmAf | 1020 | CmsAfsGmUmUmAmGmCm | 1083 |
| | UmCfUfCfCmAmGmCm | | UmGmGmAmGmAfUmGfAm | |
| | UmAmAmCmUmGm | | GmGmGmCmsCmsAm | |
| Pt2838MW02 | CmsCmsCmUmCmAmUf | 1021 | AmsCfsAmGmUmUmAmGm | 1084 |
| | CmUfCfCfAmGmCmUm | | CmUmGmGmAmGfAmUfGm | |
| | AmAmCmUmGmUm | | AmGmGmGmsCmsCm | |
| Pt2882MW02 | GmsAmsUmUmAmAmU | 1022 | AmsAfsAmGmCmUmAmAm | 1085 |
| | fGmGfAfGfGmCmUmU | | GmCmCmUmCmCfAmUfUm | |
| | mAmGmCmUmUmUm | | AmAmUmCmsAmsGm | |
| Pt2883MW02 | AmsUmsUmAmAmUmG | 1023 | GmsAfsAmAmGmCmUmAm | 1086 |
| | fGmAfGfGfCmUmUmA | | AmGmCmCmUmCfCmAfUm | |
| | mGmCmUmUmUmCm | | UmAmAmUmsCmsAm | |
| Pt2888MW02 | UmsGmsGmAmGmGmC | 1024 | AmsUfsCmCmAmGmAmAm | 1087 |
| | fUmUfAfGfCmUmUmU | | AmGmCmUmAmAfGmCfCm | |
| | mCmUmGmGmAmUm | | UmCmCmAmsUmsUm | |
| Pt2889MW02 | GmsGmsAmGmGmCmU | 1025 | CmsAfsUmCmCmAmGmAmA | 1088 |
| | fUmAfGfCfUmUmUmC | | mAmGmCmUmAfAmGfCmC | |
| | mUmGmGmAmUmGm | | mUmCmCmsAmsUm | |
| Pt2980MW02 | CmsCmsUmUmUmAmCf | 1026 | UmsGfsGmCmAmUmAmGm | 1089 |
| | UmCfUfGfCmUmCmUm | | AmGmCmAmGmAfGmUfAm | |
| | AmUmGmCmCmAm | | AmAmGmGmsUmsGm | |
| Pt2991MW02 | CmsUmsCmUmAmUmGf | 1027 | CmsUfsAmGmCmAmCmAmG | 1090 |
| | CmCfAfGfGmCmUmGm | | mCmCmUmGmGfCmAfUmA | |
| | UmGmCmUmAmGm | | mGmAmGmsCmsAm | |
| Pt3138MW02 | CmsAmsCmAmGmAmGf | 1028 | UmsUfsCmCmAmGmGmUm | 1091 |
| | GmAfAfGfAmAmAmCm | | UmUmCmUmUmCfCmUfCm | |
| | CmUmGmGmAmAm | | UmGmUmGmsAmsAm | |
| Pt3139MW02 | AmsCmsAmGmAmGmG | 1029 | GmsUfsUmCmCmAmGmGm | 1092 |
| | fAmAfGfAfAmAmCmC | | UmUmUmCmUmUfCmCfUm | |
| | mUmGmGmAmAmCm | | CmUmGmUmsGmsAm | |
| Pt3141MW02 | AmsGmsAmGmGmAmA | 1030 | UmsGfsGmUmUmCmCmAm | 1093 |
| | fGmAfAfAfCmCmUmG | | GmGmUmUmUmCfUmUfCm | |
| | mGmAmAmCmCmAm | | CmUmCmUmsGmsUm | |
| Pt3185MW02 | AmsCmsAmCmAmGmCf | 1031 | CmsUfsGmGmCmUmCmAmG | 1094 |
| | AmGfGfAfAmCmUmGm | | mUmUmCmCmUfGmCfUmG | |
| | AmGmCmCmAmGm | | mUmGmUmsGmsAm | |
| Pt3186MW02 | CmsAmsCmAmGmCmAf | 1032 | UmsCfsUmGmGmCmUmCmA | 1095 |
| | GmGfAfAfCmUmGmAm | | mGmUmUmCmCfUmGfCmU | |
| | GmCmCmAmGmAm | | mGmUmGmsUmsGm | |
| Pt3191MW02 | CmsAmsGmGmAmAmCf | 1033 | GmsCfsGmUmUmUmCmUm | 1096 |
| | UmGfAfGfCmCmAmGm | | GmGmCmUmCmAfGmUfUm | |
| | AmAmAmCmGmCm | | CmCmUmGmsCmsUm | |
| Pt3193MW02 | GmsGmsAmAmCmUmG | 1034 | CmsUfsGmCmGmUmUmUmC | 1097 |
| | fAmGfCfCfAmGmAmA | | mUmGmGmCmUfCmAfGmU | |
| | mAmCmGmCmAmGm | | mUmCmCmsUmsGm | |
| Pt3194MW02 | GmsAmsAmCmUmGmA | 1035 | UmsCfsUmGmCmGmUmUm | 1098 |
| | fGmCfCfAfGmAmAmA | | UmCmUmGmGmCfUmCfAm | |
| | mCmGmCmAmGmAm | | GmUmUmCmsCmsUm | |
| Pt3199MW02 | GmsAmsGmCmCmAmGf | 1036 | CmsCfsCmAmAmUmCmUmG | 1099 |
| | AmAfAfCfGmCmAmGm | | mCmGmUmUmUfCmUfGmG | |
| | AmUmUmGmGmGm | | mCmUmCmsAmsGm | |
| Pt3236MW02 | GmsCmsCmUmCmUmUf | 1037 | CmsCfsGmGmGmUmGmAm | 1100 |
| | CmUfUfAfCmUmUmCm | | AmGmUmAmAmGfAmAfGm | |
| | AmCmCmCmGmGm | | AmGmGmCmsUmsUm | |
| Pt3276MW02 | GmsGmsGmUmAmAmC | 1038 | UmsUfsCmCmCmAmGmCmC | 1101 |
| | fAmGfUfGfAmGmGmC | | mUmCmAmCmUfGmUfUmA | |
| | mUmGmGmGmAmAm | | mCmCmCmsGmsUm | |
| Pt3304MW02 | AmsCmsAmGmAmCmCf | 1039 | UmsCfsAmCmCmGmAmGmC | 1102 |
| | AmGfGfAfAmGmCmUm | | mUmUmCmCmUfGmGfUmC | |
| | CmGmGmUmGmAm | | mUmGmUmsGmsUm | |
| Pt3315MW02 | AmsGmsCmUmCmGmGf | 1040 | UmsCfsUmGmCmCmAmUmC | 1103 |
| | UmGfAfGfUmGmAmUm | | mAmCmUmCmAfCmCfGmA | |
| | GmGmCmAmGmAm | | mGmCmUmsUmsCm | |
| Pt3436MW02 | AmsAmsCmUmGmUmCf | 1041 | GmsGfsUmGmCmUmCmAm | 1104 |
| | CmCfUfCfCmUmUmGm | | AmGmGmAmGmGfGmAfCm | |
| | AmGmCmAmCmCm | | AmGmUmUmsGmsUm | |
| Pt3438MW02 | CmsUmsGmUmCmCmCf | 1042 | CmsUfsGmGmUmGmCmUmC | 1105 |
| | UmCfCfUfUmGmAmGm | | mAmAmGmGmAfGmGfGmA | |
| | CmAmCmCmAmGm | | mCmAmGmsUmsUm | |
| Pt3493MW02 | UmsCmsUmGmUmCmCf | 1043 | AmsAfsAmGmGmCmAmAm | 1106 |
| | UmCfUfCfUmGmUmUm | | CmAmGmAmGmAfGmGfAm | |
| | GmCmCmUmUmUm | | CmAmGmAmsCmsCm | |
| Pt3553MW02 | UmsUmsGmAmAmGmA | 1044 | AmsCfsCmCmAmGmAmAmU | 1107 |
| | fUmAfUfUfUmAmUmU | | mAmAmAmUmAfUmCfUmU | |
| | mCmUmGmGmGmUm | | mCmAmAmsGmsUm | |
| SP341.1MW02 | CmsCmsAmAmCmUmUf | 988 | AmsAfsAmCmAmGmGmUm | 1149 |
| | UmUfCfUfAmGmAmCm | | CmUmAmGmAmAfAmAfGm | |
| | CmUmGmUmUmUm | | UmUmGmGmsUmsUm | |
| SP341.2MW02 | CmsCmsAmAmCmUmUf | 988 | AmsAfsAmCmAmGmGmUm | 1150 |
| | UmUfCfUfAmGmAmCm | | CmUmAmGmAmAfAmAfGm | |
| | CmUmGmUmUmUm | | UmUmGmGmsUmsAm | |
| SP341.3MW02 | CmsCmsAmAmCmUmUf | 988 | AmsAfsAmCmAmGmGmUm | 1151 |
| | UmUfCfUfAmGmAmCm | | CmUmAmGmAmAfAmAfGm | |
| | CmUmGmUmUmUm | | UmUmGmGmsAmsUm | |
| SP341.4MW02 | CmsCmsAmAmCmUmUf | 988 | AmsAfsAmCmAmGmGmUm | 1152 |
| | UmUfCfUfAmGmAmCm | | CmUmAmGmAmAfAmAfGm | |
| | CmUmGmUmUmUm | | UmUmGmGmsAmsAm | |
| SP341.5MW02 | CmsCmsAmAmCmUmUf | 988 | AmsAfsAmCmAmGmGmUm | 1153 |
| | UmUfCfUfAmGmAmCm | | CmUmAmGmAmAfAmAfGm | |
| | CmUmGmUmUmUm | | UmUmGmGmsCmsAm | |
| SP341.6MW02 | CmsCmsAmAmCmUmUf | 1147 | UmsAfsAmCmAmGmGmUm | 1154 |
| | UmUfCfUfAmGmAmCm | | CmUmAmGmAmAfAmAfGm | |
| | CmUmGmUmUmAm | | UmUmGmGmsCmsUm | |
| SP341.7MW02 | CmsCmsAmAmCmUmUf | 1147 | UmsAfsAmCmAmGmGmUm | 1155 |
| | UmUfCfUfAmGmAmCm | | CmUmAmGmAmAfAmAfGm | |
| | CmUmGmUmUmAm | | UmUmGmGmsUmsUm | |
| SP341.8MW02 | CmsCmsAmAmCmUmUf | 1147 | UmsAfsAmCmAmGmGmUm | 1156 |
| | UmUfCfUfAmGmAmCm | | CmUmAmGmAmAfAmAfGm | |
| | CmUmGmUmUmAm | | UmUmGmGmsUmsAm | |
| SP341.9MW02 | CmsCmsAmAmCmUmUf | 1147 | UmsAfsAmCmAmGmGmUm | 1157 |
| | UmUfCfUfAmGmAmCm | | CmUmAmGmAmAfAmAfGm | |
| | CmUmGmUmUmAm | | UmUmGmGmsAmsUm | |
| SP341. | CmsCmsAmAmCmUmUf | 1147 | UmsAfsAmCmAmGmGmUm | 1158 |
| 10MW02 | UmUfCfUfAmGmAmCm | | CmUmAmGmAmAfAmAfGm | |
| | CmUmGmUmUmAm | | UmUmGmGmsAmsAm | |
| SP341. | CmsCmsAmAmCmUmUf | 1147 | UmsAfsAmCmAmGmGmUm | 1159 |
| 11MW02 | UmUfCfUfAmGmAmCm | | CmUmAmGmAmAfAmAfGm | |
| | CmUmGmUmUmAm | | UmUmGmGmsCmsAm | |
| Ps3543. | CmsUmsUmUmUmGmU | 985 | AmsAfsUmAmUmCmUmUm | 1160 |
| 1MW02 | fAmAfCfUfUmGmAmA | | CmAmAmGmUmUfAmCfAm | |
| | mGmAmUmAmUmUm | | AmAmAmGmsUmsAm | |
| Ps3543. | CmsUmsUmUmUmGmU | 985 | AmsAfsUmAmUmCmUmUm | 1161 |
| 2MW02 | fAmAfCfUfUmGmAmA | | CmAmAmGmUmUfAmCfAm | |
| | mGmAmUmAmUmUm | | AmAmAmGmsUmsUm | |
| Ps3543. | CmsUmsUmUmUmGmU | 985 | AmsAfsUmAmUmCmUmUm | 1162 |
| 3MW02 | fAmAfCfUfUmGmAmA | | CmAmAmGmUmUfAmCfAm | |
| | mGmAmUmAmUmUm | | AmAmAmGmsAmsAm | |
| Ps3543. | CmsUmsUmUmUmGmU | 985 | AmsAfsUmAmUmCmUmUm | 1163 |
| 4MW02 | fAmAfCfUfUmGmAmA | | CmAmAmGmUmUfAmCfAm | |
| | mGmAmUmAmUmUm | | AmAmAmGmsAmsUm | |
| Ps3543. | CmsUmsUmUmUmGmU | 985 | AmsAfsUmAmUmCmUmUm | 1164 |
| 5MW02 | fAmAfCfUfUmGmAmA | | CmAmAmGmUmUfAmCfAm | |
| | mGmAmUmAmUmUm | | AmAmAmGmsCmsUm | |
| Ps3543. | CmsUmsUmUmUmGmU | 1148 | UmsAfsUmAmUmCmUmUm | 1165 |
| 6MW02 | fAmAfCfUfUmGmAmA | | CmAmAmGmUmUfAmCfAm | |
| | mGmAmUmAmUmAm | | AmAmAmGmsCmsAm | |
| Ps3543. | CmsUmsUmUmUmGmU | 1148 | UmsAfsUmAmUmCmUmUm | 1166 |
| 7MW02 | fAmAfCfUfUmGmAmA | | CmAmAmGmUmUfAmCfAm | |
| | mGmAmUmAmUmAm | | AmAmAmGmsUmsAm | |
| Ps3543. | CmsUmsUmUmUmGmU | 1148 | UmsAfsUmAmUmCmUmUm | 1167 |
| 8MW02 | fAmAfCfUfUmGmAmA | | CmAmAmGmUmUfAmCfAm | |
| | mGmAmUmAmUmAm | | AmAmAmGmsUmsUm | |
| Ps3543. | CmsUmsUmUmUmGmU | 1148 | UmsAfsUmAmUmCmUmUm | 1168 |
| 9MW02 | fAmAfCfUfUmGmAmA | | CmAmAmGmUmUfAmCfAm | |
| | mGmAmUmAmUmAm | | AmAmAmGmsAmsAm | |
| Ps3543. | CmsUmsUmUmUmGmU | 1148 | UmsAfsUmAmUmCmUmUm | 1169 |
| 10MW02 | fAmAfCfUfUmGmAmA | | CmAmAmGmUmUfAmCfAm | |
| | mGmAmUmAmUmAm | | AmAmAmGmsAmsUm | |
| Ps3543. | CmsUmsUmUmUmGmU | 1148 | UmsAfsUmAmUmCmUmUm | 1170 |
| 11MW02 | fAmAfCfUfUmGmAmA | | CmAmAmGmUmUfAmCfAm | |
| | mGmAmUmAmUmAm | | AmAmAmGmsCmsUm | |

### Example 5: Assessment of Activity of Modified Double-Stranded siRNA Sequences in HepG2 Cells

In this example, the inhibitory activity of 17 modified double-stranded siRNA sequences including Ps3119MW02 and 46 modified double-stranded siRNA sequences including Pt609MW02 against the target gene PCSK9 in HepG2 cells was assessed. The 17 siRNA double-stranded unmodified sequences including Ps3119MW02 were transfected using 12-well cell plates, and the 46 siRNA double-stranded unmodified sequences including Pt609MW02 were transfected using 96-well cell plates.

5.1 Preparation of Modified Double-Stranded siRNA Sequence Working Solution:

The modified double-stranded siRNA was a lyophilized powder. Each of the siRNA lyophilized powders was centrifuged, and an appropriate amount of sterile enzyme-free water was added according to the label of each tube for dissolution to prepare a 100 μM mother liquor. The mother liquor was further subjected to gradient dilution with DEPC water to finally prepare a 10 μM working solution.

### 5.2 Cell Transfection and Detection:

In this example, 12-well cell plates and 96-well cell plates were used for transfection. The transfection in the 12-well cell plate was performed as follows: after the cells were washed once with PBS, the HepG2 cells that had grown to a nearly confluent state were digested with trypsin to prepare a cell suspension, and the cells were plated in the 12-well plate. The 10 μM siRNA working solution was added to 90 μL of opti-MEM medium at 10 μL/well to prepare an siRNA mixture. 3 μL of RNAiMAX was then added to 97 μL of opti-MEM medium to prepare an RNAiMAX mixture. After the mixture was prepared, the siRNA mixture and the RNAiMAX mixture were mixed uniformly and incubated at room temperature for 15 min to prepare a transfection complex. After the incubation was completed, the transfection complex was transferred to the 12-well plate at 200 μL/well, and 800 μL of the cell suspension was added dropwise to each well. After the 12-well plate was placed in a cell incubator and cultured overnight for 48 h, the cells were collected and subjected to cellular RNA extraction using an RNA extraction kit. After the cellular extraction was completed, the concentration was determined using Nanodrop One. The procedures for the 96-well cell plate are as follows: after the cells were washed once with PBS, the HepG2 cells that had grown to a nearly confluent state were digested with trypsin to prepare a cell suspension, and the cells were plated in the 96-well plate. The siRNA test sample working solution was added to 9 μL of opti-MEM medium at 1 μL/well to prepare an siRNA test sample mixture. 0.3 μL of RNAiMAX was then added to 9.7 μL of opti-MEM medium to prepare an RNAiMAX mixture. After the mixture was prepared, the siRNA test sample mixture and the RNAiMAX mixture were mixed uniformly and incubated at room temperature for 15 min to prepare a transfection complex. After the incubation was completed, the transfection complex was transferred to the 96-well plate at 20 μL/well, and 80 μL of the cell suspension was added to each well. After the 96-well plate was placed in a cell incubator and cultured overnight for 24 h, the cells were collected and subjected to cellular RNA extraction using the RNA extraction kit.

After the RNA extraction in the 12-well cell plate was completed, the RNA was quantified to 500 ng according to the concentration determination result, and was reverse-transcribed using an RNA reverse transcription kit. The volume of RNA was specifically calculated as follows: V(RNA)=500 ng/Conc._{(RNA)}. After the volume of RNA was determined, water was added to bring the volume to 8 μL. Subsequently, 2 μL of reverse transcriptase was added to each RNA, and reverse transcription was performed according to the procedures of 37° C. for 15 min and 85° C. for 5 min. After the reaction was completed, 40 μL of sterile enzyme-free water was added to each tube to obtain cDNA for subsequent qPCR. After the RNA extraction in the 96-well cell plate was completed, 8 μL of RNA was directly added to each well. Subsequently, 2 μL of reverse transcriptase was added to each RNA, and reverse transcription was performed according to the procedures of 37° C. for 15 min and 85° C. for 5 min. After the reaction was completed, cDNA was obtained for subsequent qPCR.

TABLE 10
Primer sequence information of the target gene and internal reference gene used for
qPCR in the 12-well plate

| Gene | | Primer type | Primer information |
|---|---|---|---|
| Target gene | PCSK9 | Upstream | AGACCCACCTCTCGCAGTC (SEQ ID |
| | | primer | NO 1113) |
| | | Downstream | GGAGTCCTCCTCGATGTAGTC (SEQ |
| | | primer | ID NO 1114) |
| Internal | GAPDH | Upstream | GGAGCGAGATCCCTCCAAAAT (SEQ |
| reference | | primer | ID NO 1115) |
| gene | | Downstream | GGCTGTTGTCATACTTCTCATGG (SEQ |
| | | primer | ID NO 1116) |

TABLE 11
Primer sequence information of the target gene and internal reference gene used for
qPCR in the 96-well plate

| Gene | | Primer type | Primer information |
|---|---|---|---|
| Target | PCSK9 | Probe | CCACCCAAGCAAGC (SEQ ID NO 1117) |
| gene | | primer | |
| | | Upstream | GGGAGAGGGCCAACAACTG (SEQ ID NO |
| | | primer | 1118) |
| | | Downstream | AGAGGACAGACCCAAAAGATAAATGT (SEQ |
| | | primer | ID NO 1119) |
| Internal | GAPDH | Probe | CAACAGCGACACCCACTC (SEQ ID NO 1120) |
| reference | | primer | |
| gene | | Upstream | AAGAAGGTGGTGAAGCAGG (SEQ ID NO |
| | | primer | 1121) |
| | | Downstream | CAAAGTGGTCGTTGAGGG (SEQ ID NO 1122) |
| | | primer | |

The qPCR method used in the 12-well plate was the SYBR GREEN method. The specific procedures were as follows: 20 μL of a Real-time PCR reaction system was prepared for each qPCR detection well according to the method described in the instructions of the real-time fluorescence quantitative PCR kit. Each reaction system contained 8 μL of the cDNA solution obtained from the reverse transcription reaction described above, 10 μL of TB Green®Premix Ex Taq (2×), 0.4 μL of 10 μM upstream primer, 0.4 μL of 10 μM downstream primer (the primer sequences are shown in Table 10), and 1.2 μL of RNase-Free H₂O. The prepared reaction system was placed on a real-time fluorescence quantitative PCR instrument, and Real-time PCR amplification was performed using a two-step method. The amplification procedure was as follows: pre-denaturation at 95° C. for 30 s, denaturation at 95° C. for 5 s, and annealing and extension at 60° C. for 30 s; and the process of denaturation, annealing, and extension was repeated for 40 cycles. The relative quantitative calculation of the expression level and inhibition rate of the target gene mRNA in each test group was performed by using the ΔΔCt method, and the specific results are shown in Table 12. The qPCR method used in the 96-well plate was the Taqman probe method. The specific procedures were as follows: 20 μL of a Real-time PCR reaction system was prepared for each qPCR detection well according to the method described in the instructions of the real-time fluorescence quantitative PCR kit. Each reaction system contained 5 μL of the cDNA solution obtained from the reverse transcription reaction described above, 10 μL of TaqMan™ Fast Advanced Mix (2×), 0.15 μL of 10 μM probe primer, 0.15 μL of 10 μM upstream primer, 0.15 μL of 10 μM downstream primer (the primer sequences are shown in Table 11), and 4.1 μL of RNase-Free H₂O. The prepared reaction system was placed on the real-time fluorescence quantitative PCR instrument, and Real-time PCR amplification was performed using a two-step method. The amplification procedure was as follows: pre-denaturation at 95° C. for 30 s, denaturation at 95° C. for 5 s, and annealing and extension at 60° C. for 30 s; and the process of denaturation, annealing, and extension was repeated for 40 cycles. The relative quantitative calculation of the expression level and inhibition rate of the target gene mRNA in each test group was performed by using the ΔΔCt method, and the specific results are shown in Table 13.

### 5.3 Data Calculation Method

$$Δ Ct ( test group ) = Ct ( target gene in test group ) - Ct ( internal reference gene in test group )$$

$$Δ Ct ( control group ) = Ct ( target gene in control group ) - Ct ( internal reference gene in control group )$$

$$ΔΔ Ct ( test group ) = Δ Ct ( test group ) - Δ Ct ( average of control group )$$

$$ΔΔ Ct ( control group ) = Δ Ct ( control group ) - Δ Ct ( average of control group )$$

Taking the control group as a baseline, the mRNA expression level of the target gene in the test group was normalized, and the residual expression level of the target gene mRNA in the control group was defined as 100%

Relative residual expression level of target gene mRNA in test group=2^{-ΔΔCt} (test group)×100%

Inhibition rate of target gene mRNA in test group=100%−relative expression level of target gene mRNA in test group

TABLE 12
Assessment results of the activity of 17 modified siRNAs including
P3119MW02 at 1 μM and 0.1 μM in HepG2 cells

Residual expression level

| No. | Ave (1 μM) | Ave (0.1 μM) |
|---|---|---|
| Blank | 100.00% | 100.00% |
| PPC2 | 25.51% | 26.09% |
| P3119MW02 | 27.71% | 11.85% |
| P3196MW02 | 27.97% | 18.46% |
| P3464MW02 | 14.18% | 20.37% |
| P3543MW02 | 30.25% | 13.84% |
| P3554MW02 | 8.39% | 12.25% |
| SP340MW02 | 20.90% | 28.98% |
| SP341MW02 | 19.12% | 21.88% |
| SP342MW02 | 19.24% | 22.71% |
| SP344MW02 | 26.27% | 40.68% |
| SP345MW02 | 26.42% | 25.64% |
| SP349MW02 | 30.51% | 35.46% |
| SP351MW02 | 30.04% | 6.26% |
| WP3239MW02 | 57.26% | 68.44% |
| WP3262MW02 | 55.65% | 50.93% |
| WP3315MW02 | 70.02% | 70.79% |
| WP3315AMW02 | 56.05% | 61.11% |
| WP3323MW02 | 98.37% | 92.03% |

TABLE 13
Assessment results of the activity of 46 modified siRNAs including
Pt609MW02 at 1 μM and 0.1 μM in HepG2 cells

| | Residual expression level | | | |
|---|---|---|---|---|
| | Ave (10 nM) | STDEV | Ave (10 nM) | STDEV |
| Blank | 100.00% | 10.27% | | |
| PPC2 | 24.80% | 2.14% | 32.73% | 1.71% |
| Pt609MW02 | 18.38% | 0.53% | 39.95% | 4.66% |
| Pt890MW02 | 57.73% | 0.19% | 74.61% | 5.26% |
| Pt2530MW02 | 24.92% | 2.52% | 42.42% | 4.66% |
| Pt2532MW02 | 32.97% | 3.52% | 48.20% | 2.98% |
| Pt2533MW02 | 28.48% | 4.20% | 42.24% | 2.35% |
| Pt2534MW02 | 19.17% | 0.47% | 21.90% | 0.20% |
| Pt2536MW02 | 18.40% | 4.59% | 22.84% | 3.19% |
| Pt2538MW02 | 88.79% | 2.77% | 91.30% | 0.65% |
| Pt2541MW02 | 57.16% | 0.21% | 68.81% | 8.08% |
| Pt2612MW02 | 21.53% | 3.24% | 56.66% | 4.39% |
| Pt2675MW02 | 21.06% | 0.94% | 22.91% | 0.45% |
| Pt2697MW02 | 22.40% | 4.55% | 41.57% | 0.67% |
| Pt2698MW02 | 16.19% | 0.09% | 31.18% | 0.14% |
| Pt2699MW02 | 28.91% | 1.53% | 44.39% | 3.60% |
| Pt2700MW02 | 36.73% | 4.34% | 51.19% | 1.38% |
| Pt2717MW02 | 25.05% | 0.19% | 51.77% | 0.84% |
| Pt2727MW02 | 96.34% | 23.14% | 79.02% | 6.88% |
| Pt2748MW02 | 46.39% | 6.36% | 82.56% | 10.90% |
| Pt2750MW02 | 25.01% | 3.81% | 35.16% | 2.36% |
| Pt2752MW02 | 63.77% | 3.12% | 84.75% | 6.74% |
| Pt2818MW02 | 40.52% | 3.69% | 71.38% | 0.27% |
| Pt2837MW02 | 58.70% | 4.39% | 86.68% | 5.96% |
| Pt2883MW02 | 32.97% | 8.91% | 39.72% | 6.47% |
| Pt2888MW02 | 20.99% | 0.75% | 25.57% | 6.44% |
| Pt2889MW02 | 52.88% | 3.92% | 64.02% | 9.54% |
| Pt2980MW02 | 39.83% | 3.17% | 52.81% | 13.99% |
| Pt2991MW02 | 71.25% | 17.72% | 102.51% | 8.21% |
| Pt3138MW02 | 27.08% | 0.41% | 34.41% | 1.09% |
| Pt3139MW02 | 31.34% | 1.77% | 33.99% | 3.95% |
| Pt3141MW02 | 56.27% | 9.07% | 53.70% | 2.89% |
| Pt3185MW02 | 65.39% | 7.05% | 105.71% | 26.51% |
| Pt3186MW02 | 37.45% | 0.68% | 71.99% | 2.36% |
| Pt3191MW02 | 36.68% | 1.37% | 46.40% | 5.41% |
| Pt3193MW02 | 36.92% | 2.47% | 54.68% | 4.80% |
| Pt3194MW02 | 24.28% | 2.46% | 31.47% | 0.95% |
| Pt3199MW02 | 71.54% | 8.71% | 84.64% | 0.69% |
| Pt3236MW02 | 89.47% | 3.79% | 82.97% | 1.54% |
| Pt3276MW02 | 73.29% | 10.28% | 77.21% | 0.22% |
| Pt3304MW02 | 61.27% | 2.28% | 73.28% | 16.78% |
| Pt3315MW02 | 38.49% | 1.20% | 59.34% | 6.95% |
| Pt3436MW02 | 118.30% | 11.10% | 141.55% | 21.97% |
| Pt3438MW02 | 89.99% | 0.57% | 111.20% | 6.94% |
| Pt3493MW02 | 13.90% | 0.65% | 13.65% | 1.45% |
| Pt3553MW02 | 14.62% | 1.09% | 19.34% | 0.05% |
| Pt2838MW02 | 92.95% | 21.37% | 97.43% | 10.16% |
| Pt2882MW02 | 23.34% | 2.25% | 23.48% | 0.45% |

It can be seen from this example that in the HepG2 cells, some of the modified double-stranded siRNAs still exhibited relatively good inhibitory activity against PCSK9 mRNA.

### Example 6: Assessment of Activity of Modified Double-Stranded siRNAs in Primary Monkey Hepatocytes

In this example, the inhibitory activity of 11 modified double-stranded siRNAs, such as P3119MW02 on the same PCSK9 target in cells against the target gene PCSK9 was evaluated by a method for assessing the target gene inhibitory activity in primary monkey hepatocytes.

6.1 Preparation of Modified Double-Stranded siRNA Working Solution

The test sample was dissolved in an appropriate amount of sterile enzyme-free water to prepare a 100 μM mother liquor. The mother liquor was further subjected to gradient dilution with sterile enzyme-free water to prepare 1 μM and 0.1 μM working solutions.

### 6.2 Thawing, Transfection, and Detection of Primary Monkey Hepatocytes

After being thawed, the primary monkey hepatocytes were transferred to a recovery medium and centrifuged at 500 g for 5 min. The recovery medium was discarded by pipetting, and the cells were pipetted and mixed uniformly with a plating medium to prepare a cell suspension, and the cells were plated in a 12-well plate. 10 μL of 10 μM siRNA test sample working solution was added to 90 μL of opti-MEM medium to prepare an siRNA test sample mixture. 3 μL of RNAiMAX was then added to 97 μL of opti-MEM medium to prepare an RNAiMAX mixture. The siRNA test sample mixture and the RNAiMAX mixture were mixed uniformly and incubated at room temperature for 15 min to prepare a transfection complex. After the incubation was completed, the transfection complex was transferred to the 12-well plate at 200 μL/well, and then 800 μL of the cell suspension was added dropwise to each well, with the amount of cells at this point being 3×10⁵ cells/well. The 12-well plate was placed in a cell incubator and cultured overnight for 4-6 h. The plating medium in the 12-well plate was discarded by pipetting, and then 1 mL of a growth medium was added to the 12-well plate. After the plate was placed in the cell incubator and cultured for another 24 h, the cells were collected and subjected to cellular RNA extraction using an RNA extraction kit. After the cell extraction was completed, the concentration was determined using NanodropOne.

The RNA was quantified to 500 ng according to the concentration determination result, and the RNA reverse transcription was performed using an RNA reverse transcription kit. The volume of RNA was specifically calculated as follows: V_{(RNA)}=500 ng/Conc._{(RNA)}. After the volume of RNA was determined, water was added to bring the volume to 8 μL. Subsequently, 2 μL of reverse transcriptase was added to each RNA, and reverse transcription was performed according to the procedures of 37° C. for 15 min and 85° C. for 5 min. After the reaction was completed, 40 μL of sterile enzyme-free water was added to each tube to obtain cDNA.

TABLE 14
Primer sequence information of the target gene and internal reference gene

| Gene | | Primer type | Primer information |
|---|---|---|---|
| Target | PCSK9 | Upstream | AGGAGTGAGCCAGGCAGTGA (SEQ ID |
| gene | | primer | NO 1191) |
| | | Downstream | AGGACTGTGCAGGAGCTGGA (SEQ ID |
| | | primer | NO 1192) |
| Internal | GAPDH | Upstream | GCCGGACGTGTTCGC (SEQ ID NO 1193) |
| reference | | primer | |
| gene | | Downstream | TGGCAACAATATCCACTTTACCAG (SEQ |
| | | primer | ID NO 1194) |

20 μL of a Real-time PCR reaction system was prepared for each PCR detection well according to the method described in the instructions of the real-time fluorescence quantitative PCR kit. Each reaction system contained 8 μL of the cDNA solution obtained from the reverse transcription reaction described above, 10 μL of TB Green®Premix Ex Taq (2×), 0.4 μL of 10 μM upstream primer, 0.4 μL of 10 μM downstream primer (the primer information is shown in Table 10), and 1.2 μL of RNase-Free H₂O. The prepared reaction system was placed on a real-time fluorescence quantitative PCR instrument, and Real-time PCR amplification was performed using a two-step method. The amplification procedure was as follows: pre-denaturation at 95° C. for 30 s, denaturation at 95° C. for 5 s, and annealing and extension at 60° C. for 30 s; and the process of denaturation, annealing, and extension was repeated for 40 cycles. In the real-time fluorescence quantitative PCR method, the relative quantitative calculation of the expression level and inhibition rate of the target gene mRNA in each test group was performed by using the ΔΔCt method, and the specific results are shown in Table 15.

### 6.3 Data Calculation Method

$$Δ Ct ( test group ) = Ct ( target gene in test group ) - Ct ( internal reference gene in test group ) ;$$

$$Δ Ct ( control group ) = Ct ( target gene in control group ) - Ct ( internal reference gene in control group ) ;$$

$$ΔΔ Ct ( test group ) = Δ Ct ( test group ) - Δ Ct ( average of control group ) ;$$

$$ΔΔ Ct ( control group ) = Δ Ct ( control group ) - Δ Ct ( average of control group ) .$$

Taking the control group as a baseline, the mRNA expression level of the target gene in the test group was normalized, and the residual expression level of the target gene mRNA in the control group was defined as 100%. Relative residual expression level of target gene mRNA in test group=2^{-ΔΔCt}(test group)×100%. Inhibition rate of target gene mRNA in test group=100%−relative expression level of target gene mRNA in test group.

TABLE 15
Relative residual expression levels of modified double-
stranded siRNAs in primary monkey hepatocytes

| | Relative residual expression level | | | |
|---|---|---|---|---|
| Compound | Ave (10 nM) | STDEV | Ave (1 nM) | STDEV |
| Blank | 100.00% | 0.00% | 100.00% | 0.00% |
| PPC2 | 28.72% | 7.28% | 36.76% | 8.52% |
| P3119MW02 | 61.35% | 0.00% | 108.76% | 0.00% |
| P3196MW02 | 39.65% | 0.00% | 69.01% | 0.00% |
| P3464MW02 | 17.77% | 0.00% | 22.41% | 0.00% |
| P3543MW02 | 25.36% | 0.00% | 25.87% | 0.00% |
| P3554MW02 | 23.33% | 0.00% | 39.35% | 0.00% |
| SP340MW02 | 32.43% | 0.00% | 44.16% | 0.00% |
| SP341MW02 | 26.69% | 0.00% | 38.07% | 0.00% |
| SP342MW02 | 24.34% | 0.00% | 33.22% | 0.00% |
| SP345MW02 | 36.92% | 0.00% | 33.68% | 0.00% |
| SP349MW02 | 41.39% | 0.00% | 42.74% | 0.00% |
| SP351MW02 | 23.51% | 0.00% | 30.79% | 0.00% |

It can be seen from this example that some of the modified sequences still maintained relatively high knockout activity in cynomolgus monkey primary hepatocytes.

### Example 7: IC50 Assay of 9 Sequences Such as P3464MW02

In this example, the mRNA expression of 9 compounds (P3464MW02, P3543MW02, P3554MW02, SP340MW02, SP341MW02, SP342MW02, SP345MW02, SP349MW02, and SP351MW02) at different concentrations (starting from a final concentration of 100 M, 44-fold gradient dilution, 11 concentration points) in HepG2 cells was detected by lipofection.

After the cells were washed once with PBS, the HepG2 cells that had grown to a nearly confluent state were digested with trypsin to prepare a cell suspension, and the cells were plated in the 12-well plate. The 10 μM siRNA test sample working solution was added to 90 μL of opti-MEM medium at 10 μL/well to prepare an siRNA test sample mixture. 3 μL of RNAiMAX was then added to 97 μL of opti-MEM medium to prepare an RNAiMAX mixture. After the mixture was prepared, the siRNA test sample mixture and the RNAiMAX mixture were mixed uniformly and incubated at room temperature for 15 min to prepare a transfection complex. After the incubation was completed, the transfection complex was transferred to the 12-well plate at 200 μL/well, and 800 μL of the cell suspension was added dropwise to each well. After the 12-well plate was placed in a cell incubator and cultured overnight for 48 h, the cells were collected and subjected to cellular RNA extraction using an RNA extraction kit. After the cell extraction was completed, the concentration was determined in NanodropOne.

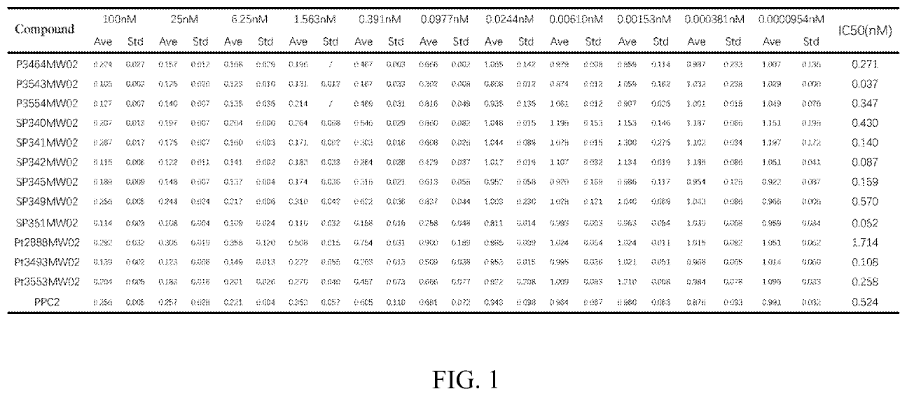

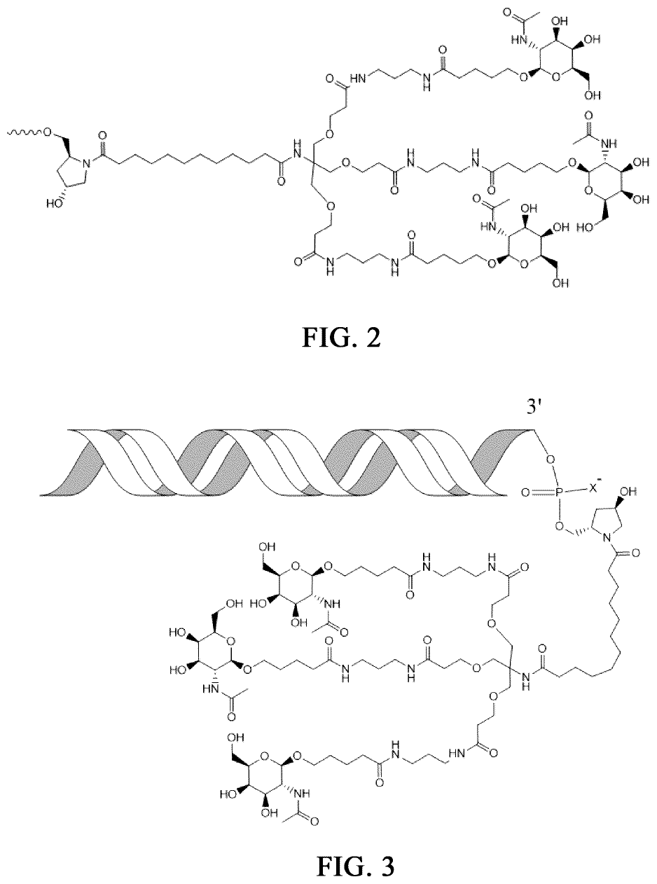

The RNA was quantified to 500 ng according to the concentration determination result, and the RNA reverse transcription was performed using an RNA reverse transcription kit. The volume of RNA was specifically calculated as follows: V_{(RNA)}=500 ng/Conc._{(RNA)}. After the volume of RNA was determined, water was added to bring the volume to 8 μL. Subsequently, 2n μL of reverse transcriptase was added to each RNA, and reverse transcription was performed according to the procedures of 37° C. for 15 min and 85° C. for 5 min. After the reaction was completed, 40 μL of sterile enzyme-free water was added to each tube to obtain cDNA, which was subjected to qPCR detection by the Taqman method. The inhibitory effect was calculated according to the obtained results, and the specific results are shown in FIG. 1.

It can be seen from this example that different compounds exhibited dose-dependent inhibitory effects at different concentrations, and the 9 compounds all showed relatively good IC50 values in the HepG2 cells.

### Example 8: Assessment of Activity of Modified Sequences P3464MW02, P3543MW02, and P3554MW02 in PCSK9 Humanized Mouse Model

In this example, the inhibitory activity of 3 modified sequences P3464MW02, P3543MW02, and P3554MW02 in a PCSK9 humanized mouse model was assessed by detecting the protein expression at different time points and by mRNA detection on day 21 of administration.

In this experiment, a total of 20 female PCSK9 humanized mice aged 6-8 weeks and 20 male PCSK9 humanized mice aged 6-8 weeks (Cyagen) were used. These mice were subcutaneously injected with P3464MW02, P3543MW02, and P3554MW02, as well as PPC2 (synthesized with reference to the patent CN104854242B) as a control, at a dose of 3 mg/kg. Meanwhile, the PBS control group was given the same volume of PBS. Blood was collected before administration, which was denoted as D0, and similarly, blood was collected on days 3, 7, 10, 14, 21, and 28 (denoted as D0, D3, D7, D10, D14, D21, and D28) after administration. The human PCSK9 protein in the serum was detected by the ELISA method for the serum of mice at different time points, and meanwhile, 3 mice in each group were randomly dissected for tissue mRNA detection on day 21 of administration. The obtained serum protein results and the residual mRNA expression levels are shown in Tables 16 and 17. The residual mRNA expression levels at different time points were normalized based on the protein expression on day 0 of administration.

The PCSK9 serum protein was detected according to the PCSK9 kit (human PCSK9 ELISA kit, Abcam, ab209884).

PCSK9 mRNA detection: The collected mouse liver tissue was firstly disrupted in a tissue disruptor and centrifuged after the disruption was completed, and a part of the supernatant was taken and transferred to a nucleic acid extractor. RNA extraction was performed using a nucleic acid extraction kit (a pre-loaded magnetic bead-based tissue RNA extraction kit KF(S), Foshan Onrew, KF643-TS1), and 500 ng of the RNA was taken according to the RNA concentration and reverse-transcribed using a reverse transcription kit (PrimeScriptRTMasterMix (Perfect Real Time), Takara, RR036A) to obtain cDNA. Finally, the residual expression level of mRNA was obtained by qPCR detection using the Taqman probe method.

TABLE 16
Inhibitory effects on protein at different time points of administration

| | Residual inhibitory effect (%) | | | | | | | | | | | |
|---|---|---|---|---|---|---|---|---|---|---|---|---|
| | D 3 | | D 7 | | D 10 | | D 14 | | D 21 | | D 28 | |
| Compound | Ave | STDEV | Ave | STDEV | Ave | STDEV | Ave | STDEV | Ave | STDEV | Ave | STDEV |
| PBS | 96.62 | 23.98 | 117.59 | 29.97 | 124.66 | 26.46 | 111.30 | 13.25 | 98.35 | 27.12 | 98.42 | 16.18 |
| PPC2 | 60.39 | 21.07 | 45.91 | 9.94 | 41.92 | 5.99 | 40.64 | 3.99 | 42.23 | 8.88 | 51.83 | 13.08 |
| P3464MW02 | 54.36 | 8.73 | 55.67 | 10.94 | 62.91 | 18.10 | 64.78 | 20.11 | 62.21 | 14.86 | 62.22 | 14.87 |
| P3543MW02 | 37.11 | 9.39 | 34.28 | 8.80 | 36.72 | 5.92 | 36.70 | 6.20 | 35.44 | 5.39 | 40.97 | 7.76 |
| P3554MW02 | 68.15 | 14.42 | 66.86 | 14.84 | 62.92 | 13.04 | 65.00 | 12.96 | 57.91 | 12.90 | 71.60 | 16.22 |

TABLE 17
Residual inhibitory effects of mRNAs in different
groups on day 21 of administration

Residual inhibitory effect

| Compound | Ave | STDEV |
|---|---|---|
| Model PBS | 100.00% | 98.39% |
| PPC2 | 62.34% | 63.48% |
| P3464MW02 | 60.31% | 54.01% |
| P3543MW02 | 41.85% | 37.66% |
| P3554MW02 | 53.67% | 46.32% |

It can be seen from the experiment that on day 28 of administration, P3543MW02 still exhibited greater than 50% inhibitory activity, which was significantly superior to that of the positive control.

### Example 9: Assessment of Activity of 6 Sequences Such as SP340MW02 in PCSK9 Humanized Mouse Model

In this example, the inhibitory activity of 6 modified sequences SP340MW02, SP341MW02, SP342MW02, SP345MW02, SP349MW02, and SP351MW02 in a PCSK9 humanized mouse model was assessed by detecting the protein expression at different time points and by mRNA detection on D21.

In this experiment, a total of 54 male PCSK9 humanized mice aged 6-8 weeks (Gempharmatech) were used. These mice were subcutaneously injected with SP340MW02, SP341MW02, SP342MW02, SP345MW02, SP349MW02, and SP351MW02, as well as P3543MW02 and PPC2 (synthesized with reference to the patent CN104854242B) as controls, at a dose of 5 mg/kg. Meanwhile, the PBS control group was given the same volume of PBS. Blood was collected before administration, which was denoted as D0, and similarly, blood was collected on days 3, 7, 10, 14, 21, 28, 35, 42, 49, 56, and 63 (denoted as D0, D3, D7, D10, D14, D21, D28, D35, D42, D49, D56, and D63) after administration. The human PCSK9 protein in the serum was detected by the ELISA method for the serum of mice at different time points, and meanwhile, 3 mice in each group were randomly dissected for tissue mRNA detection on day 21. The obtained serum protein results and the residual mRNA expression levels are shown in Tables 18 and 19. The residual mRNA expression levels at different time points were normalized based on the protein expression on D0.

The PCSK9 serum protein was detected according to the PCSK9 kit (human PCSK9 ELISA kit, Abcam, ab209884).

PCSK9 mRNA detection: The collected mouse liver tissue was firstly disrupted in a tissue disruptor and centrifuged after the disruption was completed, and a part of the supernatant was taken and transferred to a nucleic acid extractor. RNA extraction was performed using a nucleic acid extraction kit (a pre-loaded magnetic bead-based tissue RNA extraction kit KF(S), Foshan Onrew, KF643-TS1), and 500 ng of the RNA was taken according to the RNA concentration and reverse-transcribed using a reverse transcription kit (PrimeScript RT Master Mix (Perfect Real Time), Takara, RR036A) to obtain cDNA. Finally, the residual expression level of mRNA was obtained by qPCR detection using the Taqman probe method.

TABLE 18
Inhibitory effects on protein at different time points

| | D 3 | | D 7 | | D 10 | | D 14 | | D 21 |
|---|---|---|---|---|---|---|---|---|---|
| Compound | Ave | STDEV | Ave | STDEV | Ave | STDEV | Ave | STDEV | Ave |
| Model PBS | 125.85% | 22.33% | 123.10% | 15.66% | 92.08% | 13.69% | 88.91% | 11.83% | 117.55% |
| PPC2 | 51.72% | 14.55% | 30.01% | 6.39% | 11.16% | 4.29% | 13.24% | 8.64% | 38.25% |
| P3543MW02 | 25.61% | 12.23% | 18.45% | 5.87% | 9.60% | 1.98% | 9.60% | 1.98% | 18.36% |
| SP345MW02 | 60.34% | 13.60% | 34.01% | 9.20% | 6.18% | 3.88% | 10.76% | 3.33% | 15.36% |
| SP349MW02 | 80.18% | 20.36% | 72.54% | 12.09% | 40.66% | 8.97% | 49.57% | 14.09% | 52.08% |
| SP351MW02 | 46.84% | 8.19% | 29.62% | 7.57% | 10.48% | 1.09% | 11.39% | 12.11% | 14.70% |

| | D 21 | D 28 | | D 35 | | D 42 | |
|---|---|---|---|---|---|---|---|
| Compound | STDEV | Ave | STDEV | Ave | STDEV | Ave | STDEV |
| Model PBS | 31.39% | 152.77% | 76.86% | 118.13% | 15.47% | 127.24% | 18.54% |
| PPC2 | 12.90% | 38.83% | 8.14% | 58.24% | 21.78% | 54.59% | 7.40% |
| P3543MW02 | 6.73% | 23.55% | 4.02% | 35.80% | 4.96% | 31.48% | 21.34% |
| SP345MW02 | 7.34% | 15.07% | 8.77% | 29.82% | 4.86% | 32.01% | 6.35% |
| SP349MW02 | 17.77% | 70.72% | 9.42% | 81.84% | 8.89% | 88.88% | 11.17% |
| SP351MW02 | 2.98% | 27.70% | 0.90% | 34.75% | 4.58% | 48.28% | 1.10% |

TABLE 19
Residual inhibitory effects of mRNAs in different groups on D21

Residual inhibitory effect

| Compound | Ave | STDEV |
|---|---|---|
| Model PBS | 100.00% | 45.46% |
| PPC2 | 19.10% | 4.95% |
| P3543MW02 | 13.16% | 6.66% |
| SP345MW02 | 18.12% | 7.81% |
| SP349MW02 | 56.81% | 5.70% |
| SP351MW02 | 31.18% | 12.95% |

It can be seen from the experiment that on day 42 of administration, P3543MW02, SP340MW02, SP341MW02, and SP345MW02 still exhibited greater than 25% inhibitory activity, which were significantly superior to that of the positive control.

### Example 10: Assessment of Activity of 3 Sequences Such as Pt2888MW02 in PCSK9 Humanized Mouse Model

In this example, the inhibitory activity of 3 modified sequences Pt2888MW02, Pt3493MW02, and Pt3553MW02 in a PCSK9 humanized mouse model was assessed by detecting the protein expression at different time points and by mRNA detection on D21.

In this experiment, a total of 42 male PCSK9 humanized mice aged 6-8 weeks (Gempharmatech) were used. These mice were subcutaneously injected with 3 modified sequences Pt2888MW02, Pt3493MW02, and Pt3553MW02, as well as SP341MW02, PPC2 (synthesized with reference to the patent CN104854242B), and SP341MW02-2 (with reference to the patent US20110015252A1) as controls, at a dose of 5 mg/kg. Meanwhile, the PBS control group was given the same volume of PBS. Blood was collected before administration, which was denoted as D0, and similarly, blood was collected on days 7, 10, 14, 21, and 28 (denoted as D0, D3, D7, D10, D14, D21, and D28) after administration. The human PCSK9 protein in the serum was detected by the ELISA method for the serum of mice at different time points, and meanwhile, 3 mice in each group were randomly dissected for tissue mRNA detection on day 21. The obtained serum protein results are shown in Table 20. The residual mRNA expression levels at different time points were normalized based on the protein expression on D0.

The PCSK9 serum protein was detected according to the PCSK9 kit (human PCSK9 ELISA kit, Abcam, ab209884).

PCSK9 mRNA detection: The collected mouse liver tissue was firstly disrupted in a tissue disruptor and centrifuged after the disruption was completed, and a part of the supernatant was taken and transferred to a nucleic acid extractor. RNA extraction was performed using a nucleic acid extraction kit (a pre-loaded magnetic bead-based tissue RNA extraction kit KF(S), Foshan Onrew, KF643-TS1), and 500 ng of the RNA was taken according to the RNA concentration and reverse-transcribed using a reverse transcription kit (PrimeScript RT Master Mix (Perfect Real Time), Takara, RR036A) to obtain cDNA. Finally, the residual expression level of mRNA was obtained by qPCR detection using the Taqman probe method.

TABLE 20
Inhibitory effects on protein at different time points

| | D 7 | | D 10 | | D 14 | | D 21 | |
|---|---|---|---|---|---|---|---|---|
| Compound | Ave | SD | Ave | SD | Ave | SD | Ave | SD |
| PBS | 98.53% | 16.65% | 84.94% | 15.78% | 89.50% | 34.51% | 71.43% | 6.41% |
| PPC2 | 18.68% | 6.87% | 20.57% | 6.78% | 15.42% | 2.39% | 18.80% | 4.98% |
| SP341MW02 | 11.54% | 3.58% | 15.92% | 3.83% | 12.67% | 3.52% | 8.20% | 4.56% |
| SP341MW02-2 | 101.76% | 11.01% | 133.05% | 5.67% | 109.90% | 15.67% | 97.25% | 8.57% |
| Pt2888MW02 | 29.42% | 11.55% | 38.92% | 10.01% | 30.31% | 9.64% | 34.16% | 11.58% |
| Pt3493MW02 | 19.94% | 4.44% | 30.10% | 6.09% | 27.64% | 4.48% | 24.93% | 5.98% |
| Pt3553MW02 | 37.84% | 5.63% | 45.68% | 6.91% | 37.48% | 6.85% | 33.74% | 6.65% |

TABLE 21
Residual inhibitory effects of mRNAs in different groups on D21

Residual inhibitory effect

| Compound | Ave | STDEV |
|---|---|---|
| Model PBS | 100.00% | 14.24% |
| PPC2 5 mpk | 14.59% | 5.23% |
| SP341MW02 5 mpk | 3.49% | 1.13% |
| SP341MW02-2 5 mpk | 86.43% | 25.43% |
| Pt2888MW02 5 mpk | 24.66% | 9.90% |
| Pt3493MW02 5 mpk | 15.73% | 9.67% |
| Pt3553MW02 5 mpk | 34.25% | 21.54% |

### Example 11: IC50 Assay of SP341.1 and Other Sequences

In this example, the mRNA expression of a total of 22 compounds SP341.1MW02-SP341.11MW02 and Ps3543.1MW02-Ps3543.11 at different concentrations (starting from a final concentration of 50 nM, 10-fold gradient dilution, 6 concentration points) in HepG2 cells was detected by lipofection.

After the cells were washed once with PBS, the HepG2 cells that had grown to a nearly confluent state were digested with trypsin to prepare a cell suspension, and the cells were plated in the 12-well plate. The 10 μM siRNA test sample working solution was added to 90 μL of opti-MEM medium at 10 μL/well to prepare an siRNA test sample mixture. 3 μL of RNAiMAX was then added to 97 μL of opti-MEM medium to prepare an RNAiMAX mixture. After the mixture was prepared, the siRNA test sample mixture and the RNAiMAX mixture were mixed uniformly and incubated at room temperature for 15 min to prepare a transfection complex. After the incubation was completed, the transfection complex was transferred to the 12-well plate at 200 μL/well, and 800 μL of the cell suspension was added dropwise to each well. After the 12-well plate was placed in a cell incubator and cultured overnight for 48 h, the cells were collected and subjected to cellular RNA extraction using an RNA extraction kit. After the cell extraction was completed, the concentration was determined in NanodropOne.

The RNA was quantified to 500 ng according to the concentration determination result, and the RNA reverse transcription was performed using an RNA reverse transcription kit. The volume of RNA was specifically calculated as follows: V_{(RNA)}=500 ng/Conc._{(RNA)}. After the volume of RNA was determined, water was added to bring the volume to 8 μL. Subsequently, 2n μL of reverse transcriptase was added to each RNA, and reverse transcription was performed according to the procedures of 37° C. for 15 min and 85° C. for 5 min. After the reaction was completed, 40 μL of sterile enzyme-free water was added to each tube to obtain cDNA, which was subjected to qPCR detection by the Taqman method. The inhibitory effect was calculated according to the obtained results, and the specific results are shown in FIGS. 5 and 6.

It can be seen from this example that different compounds exhibited dose-dependent inhibitory effects at different concentrations, and meanwhile, compared to SP341MW02 and Ps3543MW02, SP341.8\SP341.9 MW02 and Ps3543.8\Ps3543.9MW02 exhibited an order of magnitude difference in IC50 by only changing 2-3 bases, and their IC50 values were significantly superior to that of the positive reference PPC2 in the prior art. This fully demonstrates that only a few base changes in the siRNA sequence can have a great impact on its inhibitory effect.

### Example 12: Off-Target Analysis of SP341MW02, Ps3543MW02, and SP345MW02 in HepG2

In this example, the off-target effects of a total of 3 compounds SP341MW02, Ps3543MW02, and SP345MW02 at different concentrations (100 nM and 1 nM) in HepG2 cells were detected by lipofection.

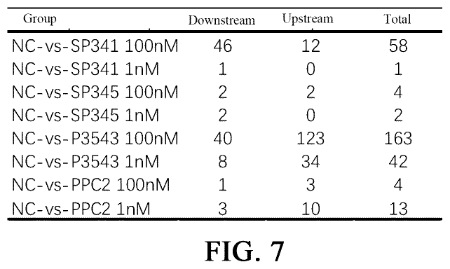

After the cells were washed once with PBS, the HepG2 cells that had grown to a nearly confluent state were digested with trypsin to prepare a cell suspension, and the cells were plated in the 12-well plate. The 10 μM siRNA test sample working solution was added to 90 μL of opti-MEM medium at 10 μL/well to prepare an siRNA test sample mixture. 3 μL of RNAiMAX was then added to 97 μL of opti-MEM medium to prepare an RNAiMAX mixture. After the mixture was prepared, the siRNA test sample mixture and the RNAiMAX mixture were mixed uniformly and incubated at room temperature for 15 min to prepare a transfection complex. After the incubation was completed, the transfection complex was transferred to the 12-well plate at 200 μL/well, and 800 μL of the cell suspension was added dropwise to each well. After the 12-well plate was placed in a cell incubator and cultured overnight for 48 h, the cells were lysed using Trizol, and BGI Genomics was entrusted to perform second-generation sequencing to detect the number of off-target genes. The genes with log 2 (Foldchange)≥1 and P value≤0.05 compared to the negative control group were set as differential genes, and the number of differential genes in each experimental group is shown in FIG. 7.

It can be seen from this example that compared to SP341MW02 and Ps3543MW02, SP345MW02 had fewer off-target genes than NC, and the number of off-target genes was similar to that of PPC2. It is believed that SP345MW02 has a lower off-target risk in HepG2 cells.

### Example 13: Off-Target Analysis of SP341MW02, Ps3543MW02, and SP345MW02 in Primary Human Hepatocytes

In this example, the off-target effects of a total of 3 compounds SP341MW02, Ps3543MW02, and SP345MW02 at different concentrations (500 nM, 100 nM, and 10 nM) in primary human hepatocytes were detected by free uptake.

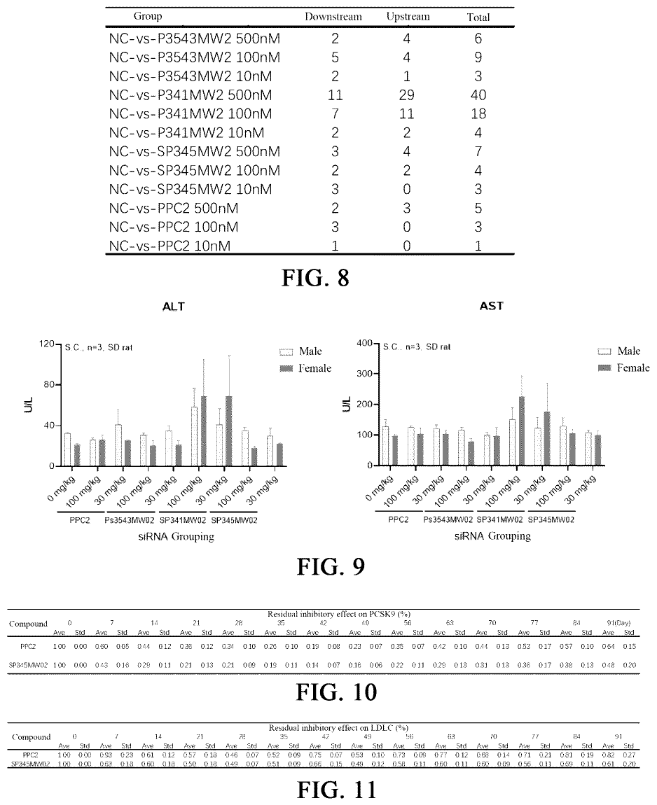

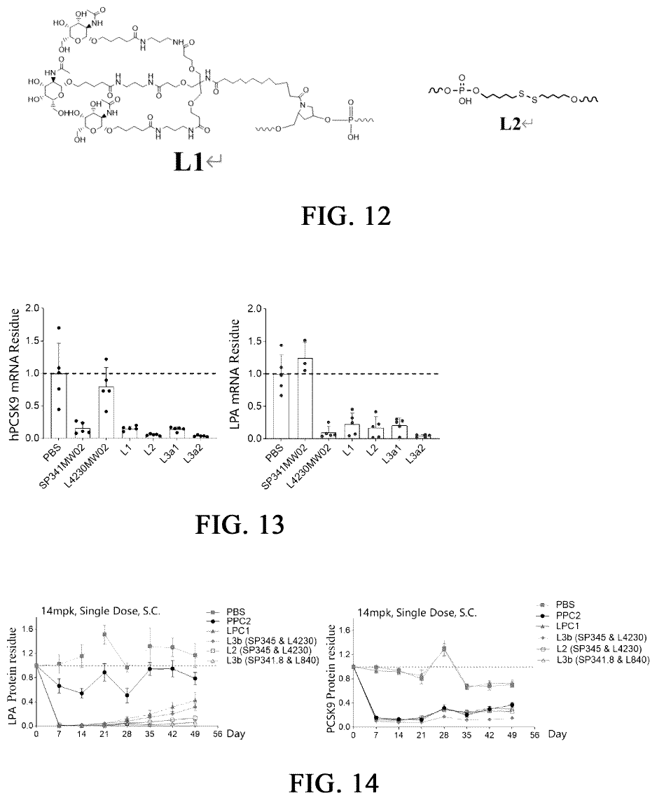

Cryopreserved primary human hepatocytes were rapidly thawed in a water bath. After counting, a cell suspension at a density of 5×10\hat{ }5 cells/mL was prepared using a primary cell adherent culture medium and added to a coated 12-well plate. After 4-6 h of adherence, non-adherent cells were washed away with PBS, and the medium was replaced by a maintenance medium containing a specific concentration of the compound. After 48 h of incubation, the cells were lysed using Trizol, and BGI Genomics was entrusted to perform second-generation sequencing to detect the number of off-target genes. The genes with log 2 (Foldchange)≥1 and P value≤0.05 compared to the negative control group were set as differential genes, and the number of differential genes in each experimental group is shown in FIG. 8.

It can be seen from this example that, consistent with the aforementioned conclusion, in the free uptake experiment in the primary human hepatocytes, the number of off-target genes of

SP345MW02 was also less than that of SP341MW02, and was substantially at the same level as that of PPC2. SP345MW02 also had a lower off-target risk in the primary human hepatocytes.

### Example 14: Toxicity Assessment of Ps3543MW02, SP341MW02, and SP345MW02 in Rats

In this example, the in vivo toxicity of the three compounds Ps3543MW02, SP341MW02, and SP345MW02 was assessed by detecting various toxicological indicators in rats after single administrations at high and low doses and after day 14 of administration.

In this experiment, a total of 60 SD rats aged 6-8 weeks and weighing 250-300 g (from Vital River) were used. Six rats (half male and half female) in each group were subcutaneously injected with Ps3543MW02, SP341MW02, SP345MW02, and PPC2 as a control (synthesized with reference to the patent CN104854242B) at a dose of 100 mg/kg or 30 mg/kg. The control group was given the same volume of PBS. Blood was collected before administration, which was denoted as D0. Cage-side observations were performed daily after administration, and samples were taken on day 14 (denoted as D14) for pathological indicator detection.

It was found by blood biochemical detection that the indicators such as ALB (albumin), GLO (globulin), A/G (albumin/globulin), and CRE were substantially similar among the groups; only in the SP341MW02 groups, ALT (alanine transaminase) and AST (aspartate transaminase) were slightly up-regulated as compared to those in the PBS groups, as shown in FIG. 9.

In addition, no significant difference was observed between each group and the PBS control group in blood routine examination, blood coagulation, viscera/body weight ratio, cage-side observation, and other detection items.

Therefore, it can be seen from this example that the three compounds Ps3543MW02, SP341MW02, and SP345MW02 exhibited very good safety at doses of 100 mpk and 30 mpk in rats. Still further, the toxicity risk of SP345 and Ps3543 was lower in the three groups.

### Example 15: Efficacy Assay of SP345MW02 in Non-Human Primates

In this example, the efficacy performance of the aforementioned SP345MW02 in non-human primates was investigated. Six male cynomolgus monkeys aged 3-4 years were selected, acclimatized, and then given SP345MW02 at 7 mg/kg or PPC2 at 7 mg/kg, with 3 animals in each group, and the administration volume was 1 mL/kg. Normal saline was added at an appropriate proportion to each test compound to prepare an injection, and the animals were administered by single-point subcutaneous injection in the morning of the day. The day of administration was defined as day 0. Blood was collected 7 days, 14 days, 21 days, 28 days, 35 days, 42 days, 49 days, 56 days, 63 days, and 70 days after the first administration, and serum was collected. The animals were fasted overnight before sampling, and serum Lp(a) and PCSK9 levels, as well as blood lipid levels, were detected. It can be seen (from FIGS. 10 and 11) that SP345MW02 achieved a greater decrease in PCSK9 protein and LDLC compared to PPC2.

### Example 16: Synthesis of Bivalent Molecules (L1\L2\L3a\L3b)

Oligonucleotide sequences were synthesized using the phosphoramidite technique in solid-phase synthesis. According to the scale, oligonucleotide sequences were synthesized using the phosphoramidite technique in solid-phase synthesis. According to the scale, oligonucleotide sequences were synthesized using the phosphoramidite technique in solid-phase synthesis. According to the scale, synthesis was performed using a YB-192 multichannel nucleic acid synthesizer and an HJ-12 nucleic acid synthesizer on a solid support made of controlled pore glass (CPG). All 2′-modified RNA phosphoramidite monomers and related synthesis reagents were purchased from the qualified supplier Chengdu HitGen. Other special starting materials used in the synthesis process included: L1 phosphoramidite monomer, L2-phosphoramidite monomer, NH2-C6 phosphoramidite monomer, L96 NHS, and the like.

All phosphoramidite monomers were dissolved in anhydrous acetonitrile (50 mM), and molecular sieves were added for drying. The synthesis was started according to a solid-phase synthesis cycle, with a monomer coupling time of 10-15 min. An iodine solution was used for oxidation to generate a phosphoester bond, a DDTT solution was used to generate a phosphorothioate bond, and acetic anhydride and an N-methylimidazole solution were used as capping reagents for unreacted active groups.

Complementary strands (SS strand and AS strand) with qualified purity were mixed in an equimolar ratio. The solution was incubated in a metal bath at 90° C. for 5 min and then slowly cooled to room temperature. Samples were collected and detected by SEC-HPLC, and the annealing purity of the double strands was calculated from the integral value of the ultraviolet absorption peak area at 260 nm.

TABLE 22
Linker structures

| Linker | Structure |
|---|---|
| LI | 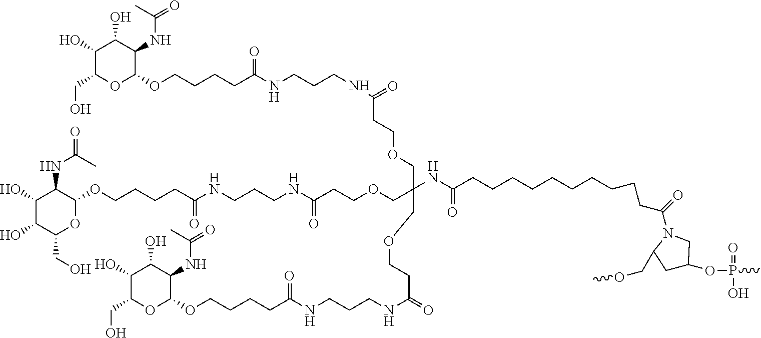 |
| L2 | 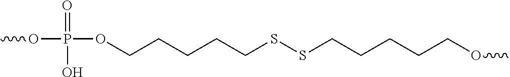 |
| L3a | Um Um Um or Um Um Um Um Um Um |
| L3b | s(dA)₃₋₆ s or s(dT)₃ s |

### Example 17: Efficacy Assay of Bivalent Molecules in PCSK9&LPA HDI Model

In order to assess the in vivo biological activity of different bivalent siRNA molecules in Example 16, based on previous studies, 4 pairs of dual-targeting siRNAs (with the sequence information shown in Table 23) for the PCSK9 and LPA genes were designed, and four linkers, L1, L2, L3a1, and L3a2, were used for linkage, respectively. Subsequently, the in vivo activity of these siRNA molecules was detected using a mouse HDI model. Firstly, a mouse model transiently transfected with the human LPA gene and PCSK9 gene was constructed to test the changes in the expression levels of the human LPA and PCSK9 genes in mice after the administration of the dual-target siRNAs. On day-3, NOD-SCID mice were intravenously injected with an equimolar amount of the siRNA solutions described above at 3 or 6 mg/Kg or a phosphate buffer (control group) with an injection volume of 10 mL/Kg. On day 0, a PCDNA3.1 plasmid containing human LPA and PCSK9 mRNA sequences was injected into the mice by high pressure injection (HDI) via the tail vein to construct a mouse model transiently transfected with the human LPA and PCSK9 genes. These mice were referred to as hLPA/hPCSK9-HDI mice. 24 h after the plasmid was injected, the hLPA/hPCSK9-HDI mice were sacrificed, and liver tissues were collected to extract RNA. The expression of human LPA and PCSK9 in the liver of the mice after modeling was detected by qPCR (as shown in FIG. 13). After tissue lysis, total RNA was extracted using a column extraction kit (Vazyme). The KanR gene on the PCDNA3.1 plasmid was selected as a housekeeping gene. The primer and probe sequences were as follows: F primer sequence (5′-3′): CGTTGGCTACCCGTGATATT (SEQ ID NO: 1195); R primer sequence (5′-3′): CTCGTCAA GAAGGCGATAGAAG (SEQ ID NO: 1196); and Probe sequence (5′-3′): CCGCTTCC TCGTGCTTTACGGTAT (SEQ ID NO: 1197).

TABLE 23

| siRNA | | | Second antisense |
|---|---|---|---|
| ID | Sense strand | First antisense strand | strand |
| SP341MW02 | CmsCmsAmAmCmUmUfUmU | AmsAfsAmCmAmGmGm | |
| | fCfUfAmGmAmCmCmUmGm | UmCmUmAmGmAmAfA | |
| | UmUmUm-L96 (SEQ ID | mAfGmUmUmGmGmsC | |
| | NO: 988) | msUm (SEQ ID NO: 1051) | |
| L4230MW02 | GmsGmsUmGmAmUmGfGm | UmsCfsGmAmUmAmAm | |
| | AfCfAfGmAmGmUmUmAmU | CmUmCmUmGmUmCfC | |
| | mCmGmAm-L96 (SEQ ID | mAfUmCmAmCmCmsU | |
| | NO: 1173) | msCm (SEQ ID NO: 1174) | |
| SP341MW02- | CmsCmsAmAmCmUmUfUmU | AmsAfsAmCmAmGmGm | UmsCfsGmAmUm |
| L1- | fCfUfAmGmAmCmCmUmGm | UmCmUmAmGmAmAfA | AmAmCmUmCmU |
| L4230MW02 | UmUmUm-L1- | mAfGmUmUmGmGmsC | mGmUmCfCmAfU |
| | GmsGmsUmGmAmUmGfGm | msUm (SEQ ID NO: 1051) | mCmAmCmCmsU |
| | AfCfAfGmAmGmUmUmAmU | | msCm (SEQ ID NO: |
| | mCmsGmsAm | | 1174) |
| SP341MW02- | L96- | AmsAfsAmCmAmGmGm | UmsCfsGmAmUm |
| L2- | CmsCmsAmAmCmUmUfUmU | UmCmUmAmGmAmAfA | AmAmCmUmCmU |
| L4230MW02 | fCfUfAmGmAmCmCmUmGm | mAfGmUmUmGmGmsC | mGmUmCfCmAfU |
| | UmUmUm-L2- | msUm (SEQ ID NO: 1051) | mCmAmCmCmsU |
| | GmsGmsUmGmAmUmGfGm | | msCm (SEQ ID NO: |
| | AfCfAfGmAmGmUmUmAmU | | 1174) |
| | mCmGmAm-L96 | | |
| SP341MW02- | L96- | AmsAfsAmCmAmGmGm | UmsCfsGmAmUm |
| L3a1- | CmsCmsAmAmCmUmUfUmU | UmCmUmAmGmAmAfA | AmAmCmUmCmU |
| L4230MW02 | fCfUfAmGmAmCmCmUmGm | mAfGmUmUmGmGmsC | mGmUmCfCmAfU |
| | UmsUmsUmUmUmUmGmsG | msUm(SEQ ID NO: 1051) | mCmAmCmCmsU |
| | msUmGmAmUmGfGmAfCfAf | | msCm (SEQ ID NO: |
| | GmAmGmUmUmAmUmCmG | | 1174) |
| | mAm-L96 (SEQ ID | | |
| | NO: 1176) | | |
| SP341MW02- | L96- | AmsAfsAmCmAmGmGm | UmsCfsGmAmUm |
| L3a2- | CmsCmsAmAmCmUmUfUmU | UmCmUmAmGmAmAfA | AmAmCmUmCmU |
| L4230MW02 | fCfUfAmGmAmCmCmUmGm | mAfGmUmUmGmGmsC | mGmUmCfCmAfU |
| | UmsUmsUmUmUmUmUmUm | msUm(SEQ ID NO: 1051) | mCmAmCmCmsU |
| | UmGmsGmsUmGmAmUmGf | | msCm (SEQ ID NO: |
| | GmAfCfAfGmAmGmUmUmA | | 1174) |
| | mUmCmGmAm-L96 (SEQ ID | | |
| | NO: 1177) | | |

The qPCR detection results (FIG. 13) show that the 4 pairs of dual-targeting siRNAs significantly knocked down the expression of the human LPA and PCSK9 genes in the hLPA/hPCSK9-HDI mice, where SP341MW02-L3a2-L4230MW02 exhibited the best inhibitory effect on the two target genes PCSK9 and LPA, which was superior to that of the corresponding single-targeting siRNA molecule at the same molar dose. It was found from further comparative analysis that the bivalent molecule conjugated to L3a2 had better inhibitory effects on the two target genes than the molecule conjugated to L3a1, which also indicates that the longer the interval between the front and back ends of the bivalent molecules, the smaller the mutual effect on efficacy.

### Example 18: Assessment of Activity of Bivalent Molecules in PCSK9 and LPA Double Humanized Mouse Model

In this example, the inhibitory activity of the sequences in Table 24 in a PCSK9 and LPA double humanized mouse model was assessed by detecting the protein expression at different time points.

In this experiment, a total of 30 male PCSK9 humanized mice aged 6-8 weeks (Biocytogen) were used. These mice were subcutaneously injected with bivalent siRNAs at a dose of 14 mg/kg and SP345MW02 and L4230MW02 as controls at a dose of 7 mg/kg. Meanwhile, the PBS control group was given the same volume of PBS. Blood was collected before administration, which was denoted as D0, and similarly, blood was collected on days 3, 7, 10, 14, 21, 28, 35, 42, and 49 (denoted as D0, D3, D7, D10, D14, D21, D28, D35, D42, and D49) after administration. The human PCSK9 protein and LPA protein in the serum were detected by an ELISA method for the serum of mice at different time points, and the residual expression levels of the proteins at different time points were normalized based on the protein expression on D0.

The sequences of the bivalent siRNAs used in this example are shown in Table 24. It should be noted that, considering that DNA is more stable in the circulatory system and the synthesis cost of DNA monomers is significantly lower than that of 2′-oxymethyl-modified RNA monomers, a L3b linker was designed based on the structure of L3a2.

TABLE 24

| | | First antisense | Second antisense |
|---|---|---|---|
| siRNA ID | Sense strand | strand | strand |
| SP345MW02 | CmsUmsUmUmUmCmUfAmGfAfC | AmsGfsCmAmAmA | |
| | fCmUmGmUmUmUmUmGmCmU | mAmCmAmGmGmU | |
| | m-L96 (SEQ ID NO: 991) | mCmUfAmGfAmAm | |
| | | AmAmGms UmsUm | |
| | | (SEQ ID NO: 1054) | |
| L4230MW02 | GmsGmsUmGmAmUmGfGmAfCf | UmsCfsGmAmUmA | |
| | AfGmAmGmUmUmAmUmCmGm | mAmCmUmCmUmG | |
| | Am-L96 (SEQ ID NO: 1173) | mUmCfCmAfUmCm | |
| | | AmCmCmsUmsCm | |
| | | (SEQ ID NO: 1174) | |
| SP345MW02- | L96- | AmsGfsCmAmAmA | UmsCfsGmAmUm |
| L2- | CmsUmsUmUmUmCmUfAmGfAfC | mAmCmAmGmGmU | AmAmCmUmCm |
| L4230MW02 | fCmUmGmUmUmUmUmGmCmsU | mCmUfAmGfAmAm | UmGmUmCfCmA |
| | ms-L2- | AmAmGmsUmsUm | fUmCmAmCmCm |
| | GmsGmsUmGmAmUmGfGmAfCf | (SEQ ID NO: 1054) | sUmsCm (SEQ ID |
| | AfGmAmGmUmUmAmUmCmGm | | NO: 1174) |
| | Am-L96 | | |
| SP345MW02- | L96- | AmsGfsCmAmAmA | UmsCfsGmAmUm |
| L3b- | CmsUmsUmUmUmCmUfAmGfAfC | mAmCmAmGmGmU | AmAmCmUmCm |
| L4230MW02 | fCmUmGmUmUmUmUmGmCmsU | mCmUfAmGfAmAm | UmGmUmCfCmA |
| | msdAdAdAdAdAdAsGmsGmUmG | AmAmGmsUmsUm | fUmCmAmCmCm |
| | mAmUmGfGmAfCfAfGmAmGmU | (SEQ ID NO: 1054) | sUmsCm (SEQ ID |
| | mUmAmUmCmGmAm-L96 (SEQ | | NO: 1174) |
| | ID NO: 1180) | | |
| SP341. | L96- | UmsAfsAmCmAmG | UmsGfsUmGmCm |
| 8MW02-L3b- | CmsCmAmAmCmUmUfUmUfCfUf | mGmUmCmUmAmG | CmUmCmGmAm |
| L840MW02 | AmGmAmCmCmUmGmUmUmsA | mAmAfAmAfGmUm | UmAmAmCfUmC |
| | msdAdAdAdAdAdAsGmsGmAmC | UmGmGmsUmsAm | fUmGmUmCmCm |
| | mAmGmAfGmUfUfAfUmCmGmA | (SEQ ID NO: 1156) | sAmsUm (SEQ ID |
| | mGmGmCmAmCmAm-L96 (SEQ | | NO: 1182) |
| | ID NO:1181) | | |

The PCSK9 serum protein was detected according to the PCSK9 kit (human PCSK9 ELISA kit, Abcam, ab209884). The LPA serum protein was detected according to the LPA kit (human LPA ELISA kit, Abcam, ab212165).

ELISA assay results (FIG. 14) show that these bivalent siRNAs significantly inhibited the expression of the LPA and PCSK9 genes in hLPA/hPCSK9 double-transgenic mice. SP345MW02-L3b-L4230MW02 exhibited the best inhibitory effect on the PCSK9 target gene, which was significantly superior to those of other bivalent molecules and the PPC2 positive reference, and its inhibitory effect on the LPA target gene was also superior to that of the LPC1 positive reference we selected (sense strand: CAGCCCCUUAUUGUUAUACG, and antisense strand: UCGUAUAACAAUAAGGGGCUG; for modified positive reference sequence, modified sense strand: NAG25-CmsAmsGmCmCmCmCmUmUfAfUfUmGmUmUmAmUmAmCmGms[invdA], and modified antisense strand: UmsCfsGmUfAmUfAmAmCmAmAmUfAmAfGmGfGmGf CmsUfsGm; synthesized with reference to the document Preclinical development and phase 1 trial of a novel siRNA targeting lipoprotein(a)). Moreover, the bivalent molecule SP341.8MW02-L3b-L840MW02 (i.e., after the sequences conjugated flanking the L3b linker was changed) in Table 24 still exhibited similar and significant inhibitory effects on the two target proteins PCSK9 and LPA compared to LPC1 and PPC2. This also indicates that the L3b linker is in line with our expectations and can relatively well maintain the original efficacy of the molecules conjugated flanking the linker in the bivalent siRNA.

### Example 19: In Vitro Stability Assessment of Bivalent Molecules

In this example, the in vitro stability of the aforementioned dual-targeting molecules SP345MW02-L2-L4230MW02 and SP345MW02-L3b-L4230MW02 in plasma and liver lysosomes was investigated.

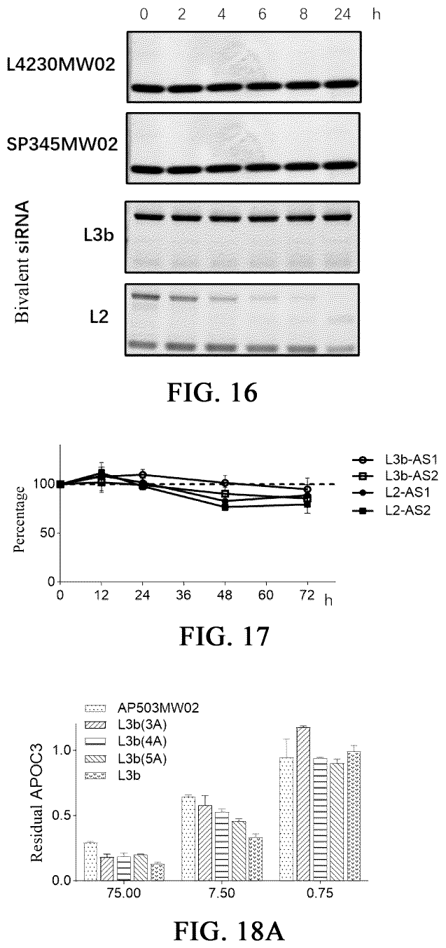

For the in vitro plasma stability detection, a dry powder of the bivalent siRNA molecule to be tested was first dissolved in normal saline to prepare a 20 μM working solution. Meanwhile, pure human plasma was diluted to a 90% plasma working solution with PBS. 12 μL of the siRNA working solution was uniformly mixed with 108 μL of the plasma working solution to prepare an siRNA plasma incubation solution, at a final concentration of 2 μM and with a total volume of 120 μL. The sample described above was uniformly mixed and then aliquoted into PCR tubes at 15 μL/tube for a total of 8 tubes. The tubes were incubated in a Bio-Rad T100 thermal cycler at 37° C., taken out at 0 h, 2 h, 4 h, 6 h, 8 h, and 24 h, immediately frozen in liquid nitrogen, and temporarily stored in a refrigerator at −80° C. After all samples were prepared, all of the aforementioned samples were diluted 5-fold with PBS, and 10 μL of the dilutions were separately mixed with a loading buffer (20 mM EDTA, 36 wt % glycerol, and 0.06 wt % bromophenol blue in water). Electrophoresis was performed for 60 min under a constant current of 80 mA using a non-denaturing 4%-20% gradient PAGE gel. The results are shown in FIG. 16. The results show that L3b and L4230MW02 as well as SP345MW02 exhibited similar stability in plasma, with substantially no significant degradation observed, while having greater plasma stability than the bivalent molecule conjugated to L2.

For the lysosome stability experiment, a citrate (100 nM) solution was first placed on a stirrer, and a sodium citrate (100 mM) solution was slowly added until the pH value reached 5.0 (=0.05). 8 μL of the compound working solution and 792 μL of a citrate buffer (with or without 0.3 mg/mL human liver lysosomes) were added to a DNA LoBind tube to achieve a final compound concentration of 2 μg/mL. Three replicates were set for each sample, and the incubation solution was incubated in a carbon dioxide incubator at 37° C. At 0 h, 12 h, 24 h, 48 h, and 72 h, 50 μL of the sample was taken out from the reaction solution. The reaction was terminated by adding 50 μL of ammonia water. The sample was then vortexed for 5 min and extracted by liquid-liquid extraction (LLE) and solid-phase extraction (SPE). The sample after liquid-liquid extraction and solid-phase extraction was evaporated to dryness under a heated nitrogen (40° C.) condition. The sample was then redissolved in 200 μL of the mobile phase and injected into an LC/MS/MS system for quantitative analysis. The specific results are shown in FIG. 17. The results show that the two AS strands of the bivalent molecules exhibited relatively good stability in liver lysosomes. In addition, consistent with the plasma stability results, L3b exhibited very better stability compared to L2.

### Example 20: Linker Optimization for Bivalent Molecules

In this example, the effects of linkers with different lengths and different base compositions on the conjugated 3′-end of SS strands and the efficacy of siRNAs were verified by free uptake experiments in primary hepatocytes of APOC3 humanized mice. The sequences of the bivalent siRNAs used in this example are shown in Table 25.

TABLE 25

| siRNA | | | Second antisense |
|---|---|---|---|
| ID | Sense strand | First antisense strand | strand |
| AP503MW02- | L96- | UmsUfsCmUmUmG | UmsCfsGmAmUm |
| L3b(3A)- | CmsCmsCmAmAmUmAfAmAfGfCf | mUmCmCmAmGm | AmAmCmUmCm |
| L4230MW02 | UmGmGmAmCmAmAmGmAmsAm | CmUmUfUmAfUm | UmGmUmCfCmAf |
| | sdAdAdAsGmsGmUmGmAmUmGf | UmGmGmGmsAms | UmCmAmCmCms |
| | GmAfCfAfGmAmGmUmUmAmUm | Gm (SEQ ID NO | UmsCm (SEQ ID |
| | CmGmAm-L96 (SEQ ID NO 1183) | 1184) | NO 1174) |
| AP503MW02- | L96- | UmsUfsCmUmUmG | UmsCfsGmAmUm |
| L3b(4A)- | CmsCmsCmAmAmUmAfAmAfGfCf | mUmCmCmAmGm | AmAmCmUmCm |
| L4230MW02 | UmGmGmAmCmAmAmGmAmsAm | CmUmUfUmAfUm | UmGmUmCfCmAf |
| | sdAdAdAdAsGmsGmUmGmAmUm | UmGmGmGmsAms | UmCmAmCmCms |
| | GfGmAfCfAfGmAmGmUmUmAm | Gm (SEQ ID NO | UmsCm (SEQ ID |
| | UmCmGmAm-L96 (SEQ ID NO | 1184) | NO 1174) |
| | 1185) | | |
| AP503MW02- | L96- | UmsUfsCmUmUmG | UmsCfsGmAmUm |
| L3b(5A)- | CmsCmsCmAmAmUmAfAmAfGfCf | mUmCmCmAmGm | AmAmCmUmCm |
| L4230MW02 | UmGmGmAmCmAmAmGmAmsAm | CmUmUfUmAfUm | UmGmUmCfCmAf |
| | sdAdAdAdAdAsGmsGmUmGmAm | UmGmGmGmsAms | UmCmAmCmCms |
| | UmGfGmAfCfAfGmAmGmUmUm | Gm (SEQ ID NO | UmsCm (SEQ ID |
| | AmUmCmGmAm-L96 (SEQ ID NO | 1184) | NO 1174) |
| | 1191) | | |
| AP503MW02- | L96- | UmsUfsCmUmUmG | UmsCfsGmAmUm |
| L3b- | CmsCmsCmAmAmUmAfAmAfGfCf | mUmCmCmAmGm | AmAmCmUmCm |
| L4230MW02 | UmGmGmAmCmAmAmGmAmsAm | CmUmUfUmAfUm | UmGmUmCfCmAf |
| | sdAdAdAdAdAdAsGmsGmUmGmA | UmGmGmGmsAms | UmCmAmCmCms |
| | mUmGfGmAfCfAfGmAmGmUmU | Gm (SEQ ID NO | UmsCm (SEQ ID |
| | mAmUmCmGmAm-L96 (SEQ ID | 1184) | NO 1174) |
| | NO 1186) | | |
| AP503MW02- | L96- | UmsUfsCmUmUmG | UmsCfsGmAmUm |
| L3b(3T)- | CmsCmsCmAmAmUmAfAmAfGfCf | mUmCmCmAmGm | AmAmCmUmCm |
| L4230MW02 | UmGmGmAmCmAmAmGmAmsAm | CmUmUfUmAfUm | UmGmUmCfCmAf |
| | sdTdTdTsGmsGmUmGmAmUmGfG | UmGmGmGmsAms | UmCmAmCmCms |
| | mAfCfAfGmAmGmUmUmAmUmC | Gm (SEQ ID NO | UmsCm (SEQ ID |
| | mGmAm-L96 (SEQ ID NO 1187) | 1184) | NO 1174) |
| AP503MW02- | L96- | UmsUfsCmUmUmG | UmsCfsGmAmUm |
| L3b(4T)- | CmsCmsCmAmAmUmAfAmAfGfCf | mUmCmCmAmGm | AmAmCmUmCm |
| L4230MW02 | UmGmGmAmCmAmAmGmAmsAm | CmUmUfUmAfUm | UmGmUmCfCmAf |
| | sdTdTdTdTsGmsGmsUmGmAmUm | UmGmGmGmsAms | UmCmAmCmCms |
| | GfGmAfCfAfGmAmGmUmUmAm | Gm (SEQ ID NO | UmsCm (SEQ ID |
| | UmCmGmAm-L96 (SEQ ID NO | 1184) | NO 1174) |
| | 1188) | | |
| AP503MW02- | L96- | UmsUfsCmUmUmG | UmsCfsGmAmUm |
| L3b(5T)- | CmsCmsCmAmAmUmAfAmAfGfCf | mUmCmCmAmGm | AmAmCmUmCm |
| L4230MW02 | UmGmGmAmCmAmAmGmAmsAm | CmUmUfUmAfUm | UmGmUmCfCmAf |
| | sdTdTdTdTdTsGmsGmUmGmAmU | UmGmGmGmsAms | UmCmAmCmCms |
| | mGfGmAfCfAfGmAmGmUmUmA | Gm (SEQ ID NO | UmsCm (SEQ ID |
| | mUmCmGmAm-L96 (SEQ ID NO | 1184) | NO 1174) |
| | 1189) | | |
| AP503MW02- | L96- | UmsUfsCmUmUmG | UmsCfsGmAmUm |
| L3b(6T)- | CmsCmsCmAmAmUmAfAmAfGfCf | mUmCmCmAmGm | AmAmCmUmCm |
| L4230MW02 | UmGmGmAmCmAmAmGmAmsAm | CmUmUfUmAfUm | UmGmUmCfCmAf |
| | sdTdTdTdTdTdTsGmsGmUmGmAm | UmGmGmGmsAms | UmCmAmCmCms |
| | UmGfGmAfCfAfGmAmGmUmUm | Gm (SEQ ID NO | UmsCm (SEQ ID |
| | AmUmCmGmAm-L96 (SEQ ID NO | 1184) | NO 1174) |
| | 1190) | | |
| AP503MW02 | CmsCmsCmAmAmUmAfAmAfGfCf | UmsUfsCmUmUmG | |
| | UmGmGmAmCmAmAmGmAmAm- | mUmCmCmAmGm | |
| | L96 (SEQ ID NO 1179) | CmUmUfUmAfUm | |
| | | UmGmGmGmsAms | |
| | | Gm (SEQ ID NO | |
| | | 1184) | |

Compared to the conventional siRNA, the bivalent molecule had greater steric hindrance at the 5′-position of the first inhibitor AS strand due to the conjugation of the linker and the second inhibitor to the 3′-end of the first inhibitor SS strand, so that its efficacy was easily affected. Therefore, for the bivalent molecules in Table 25, the effects of linkers formed by different types and numbers of bases on the efficacy of the bivalent molecules were assessed by assessing their inhibitory effects on hAPOC3 mRNA in primary hepatocytes of APOC3 humanized mice.

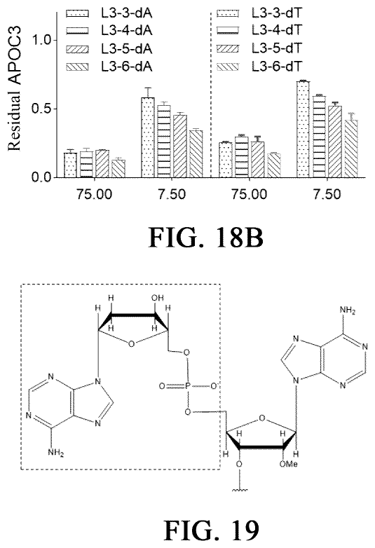

Primary cells freshly isolated from the liver of hAPOC3 mice were counted, and then a cell suspension at a density of 5×10\hat{ }5 cells/mL was prepared using a primary cell adherent culture medium and added to a coated 12-well plate. After 4-6 h of adherence, non-adherent cells were washed away with PBS, and the medium was replaced by a maintenance medium containing a specific concentration of the bivalent compound for culture. After 48 h of incubation, RNA was extracted, and the change in the mRNA level of hAPOC3 was detected. The primer sequences were as follows: F primer sequence (5′-3′): CTCAGCTTCATGCAGGGTTACAT; R primer sequence (5′-3′): CGCTGCTCAGTGCATCCTT; and Probe sequence (5′-3′): CACGCCACCAAGAC. The specific results are shown in FIGS. 18A-18B. From the results, it can be seen that when the linker in the bivalent molecule was 6 bases in length, the less the efficacy interference between two siRNAs with each other, the better the activity of the finally formed bivalent siRNA. In addition, when the length was the same, different base compositions also affected the efficacy of the final bivalent molecule. In FIG. 18B, the efficacy of the bivalent siRNA consisting of dA was significantly better than that of the bivalent molecule consisting of dT.

### Example 21: Detection of In Vivo Inhibitory Effects of Dual-Target Molecules on PCSK9 and LPA in NHP

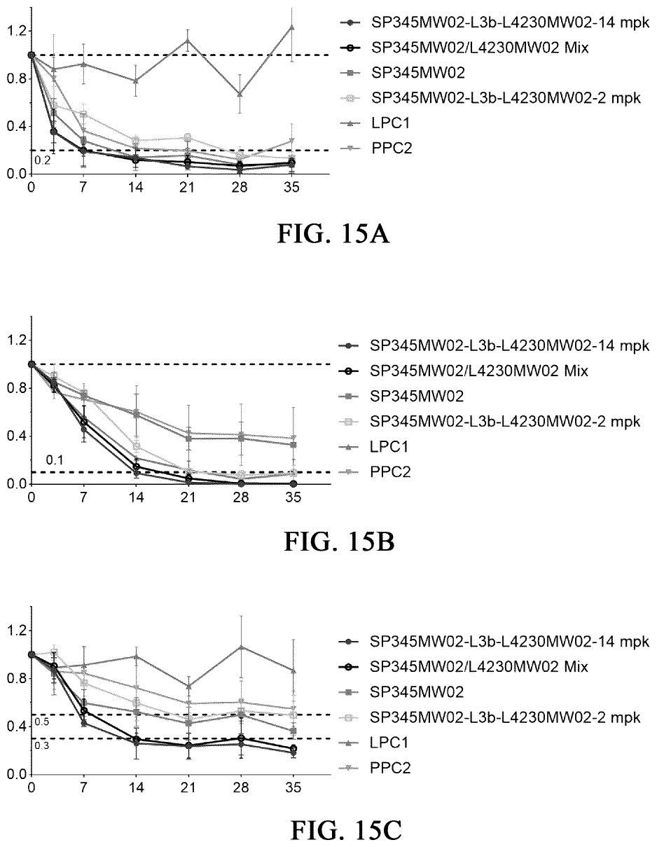

In this example, the efficacy performance of the aforementioned dual-targeting siRNAs at different doses and the corresponding combination strategy of PCSK9 siRNA and the corresponding positive reference was investigated in non-human primates. Eighteen male cynomolgus monkeys aged 3-4 years were selected, acclimatized, and then given LPC1, P345MW02, and PPC2, or a Mix of 7 mpk SP345MW02 and 7 mpk L4230MW02 at 7 mg/kg, and the dual-targeting siRNA molecule SP345MW02-L3b-L4230MW02 at 14 mg/kg or 2 mg/kg, respectively, with 3 animals in each group, and the administration volume was 1 mL/kg. Normal saline was added to each test compound at an appropriate proportion to prepare an injection, and the animals were administered by single-point subcutaneous injection in the morning of the day. The day of administration was defined as day 0. Blood was collected 3 days, 7 days, 14 days, and 28 days after the first administration, and serum was collected. The animals were fasted overnight before sampling, and serum Lp(a) and PCSK9 levels, as well as blood lipid levels were detected. It can be seen (from FIGS. 15A-15C) that the efficacy of the dual-targeting molecule was not affected and achieved a greater decrease in LDLC compared to SP345MW02 alone. Compared to the Mix of SP345MW02 and L4230MW02, the dual-targeting molecule had a similar ability to inhibit PCSK9, Lp(a), and LDLC, but its formulation form was simpler, so that the administration was more convenient. Compared to the PPC2 and LPC1 controls, the dual-targeting molecule had a stronger ability to inhibit PCSK9 and LPA proteins and a stronger ability to regulate LDLC.

### Example 22: Toxicity Experiment in Rats

In this example, the toxic reactions of the dual-target siRNA molecule of the present disclosure in rodents were evaluated by observing the clinical toxic reactions of rats administered different doses of the dual-target molecule SP345MW02-L3b-L4230MW02, detecting indicators such as body weight, hepatic and renal functions, blood routine examination, blood biochemistry, and blood lipids, and performing gross anatomy and histopathological detection.

SPF-grade SD rats aged 6-9 weeks and weighing 210-250 g were selected and divided into 3 groups (6 in each group) according to body weight, half being male and half being female. SP345MW02-L3b-L4230MW02 at 200 mpk or 60 mpk, or a 0.9% sodium chloride injection control, was injected subcutaneously. The drug amount was calculated according to the body weight, and a 60 mg/mL solution of each conjugate was prepared in 1×PBS (pH 7.4) and then injected.

After the single subcutaneous administration, the following indicators were detected:
cage-side observation twice daily;
detailed cage-side observation twice weekly;
body weight and food intake detection twice weekly;
detection of clinical pathological indicators such as blood biochemistry, hematology, and coagulation;
histopathological evaluation on the liver and kidney organs.

The results show that the rats could tolerate the two doses of the dual-targeting siRNA molecule SP345MW02-L3b-L4230MW02 selected in this experiment, and no significant toxic and side effects were observed. The safety of the dual-targeting molecule SP345MW02-L3b-L4230MW02 was good.

### Example 23: IC50 Assay in PHHs Under Free Uptake

In this example, the IC50 values of a total of 5 compounds, PPC2, LPC1, SP345MW02, L4230MW02, and SP345MW02-L3b-L4230MW02, under free uptake conditions in primary human hepatocytes were detected by the free uptake method.

The siRNA was diluted with nuclease-free water to prepare a 10-fold concentrate (e.g., if the final test concentration was 0.1 nM, 1 nM of the concentrate was prepared). One tube of primary human hepatocytes was thawed and transferred to 9 mL of human hepatocyte complete medium. The cell suspension was diluted to the desired final cell density of 6×10\hat{ }5 cells/mL with the complete medium. 10 μL of the prepared concentrate was added to each well of a 96-well plate coated in advance, and then 90 μL of the cell suspension was added. The cell culture plate was incubated in an incubator with 5% CO₂ at 37° C. for 48 h. The medium was removed, and the cell plate was collected for RNA extraction. The mRNA expression of PCSK9 and LPA was determined according to the aforementioned method, and IC50 was calculated.

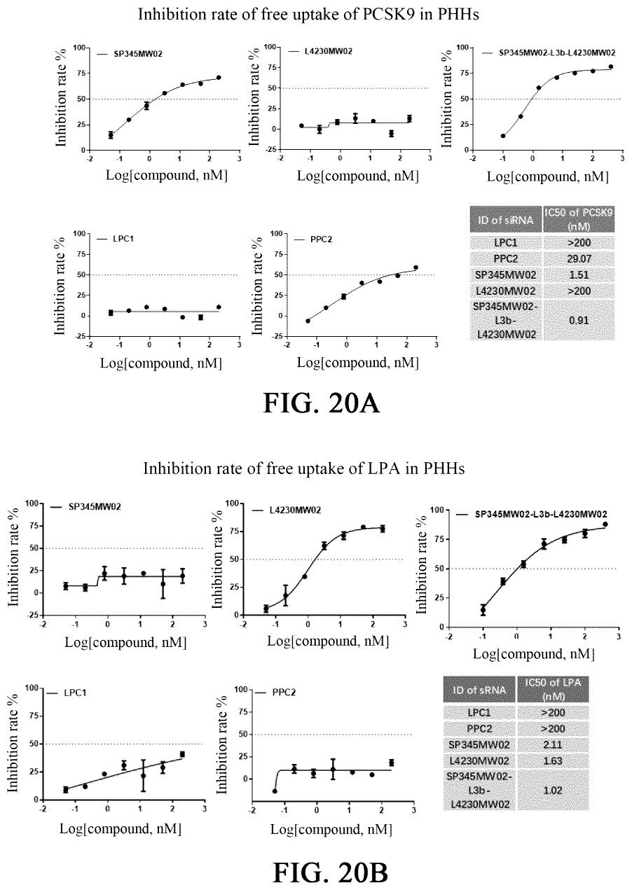

As shown in FIGS. 20A and 20B, it can be seen from this example that, consistent with the aforementioned conclusion, in the free uptake experiment of primary human hepatocytes, SP345MW02-L3b-L4230MW02 exhibited very good inhibitory efficiency against PCSK9 and LPA in PHHs, which was not only superior to those of the positive references PPC2 and LPC1 we selected, but also slightly superior to those of the single-target siRNA molecules SP345MW02 and L4230MW02, indicating that the bivalent siRNA molecule has the efficacy potential superior to the monovalent molecules.

1-65. (canceled)

66. An siRNA molecule that targets PCSK9, wherein the siRNA molecule comprises a sense strand comprising the nucleobase sequence of SEQ ID NO: 104 and an antisense strand comprising the nucleobase sequence of SEQ ID NO: 431.

67. The siRNA of claim 66, wherein the sense strand comprises the nucleic acid sequence of SEQ ID NO: 991.

68. The siRNA of claim 66, wherein the antisense strand comprises the nucleic acid sequence of SEQ ID NO: 1054.

69. The siRNA of claim 66, wherein the sense strand comprises the nucleic acid sequence of SEQ ID NO: 991 and the antisense strand comprises the nucleic acid sequence of SEQ ID NO: 1054.

70. A composition comprising a first siRNA molecule and a second siRNA molecule, wherein the first siRNA molecule targets PCSK9 and comprises a sense strand comprising the nucleobase sequence of SEQ ID NO: 104 and an antisense strand comprising the nucleobase sequence of SEQ ID NO: 431.

71. The composition of claim 70, wherein the sense strand comprises the nucleic acid sequence of SEQ ID NO: 991 and the antisense strand comprises the nucleic acid sequence of SEQ ID NO: 1054.

72. The composition of claim 70, wherein the second siRNA molecule targets Lp(a).

73. The composition of claim 72, wherein the second siRNA molecule comprises a sense strand comprising the nucleobase sequence of SEQ ID NO: 1171 and an antisense strand comprising the nucleobase sequence of SEQ ID NO: 1172.

74. The composition of claim 72, wherein the second siRNA molecule comprises a sense strand comprising the nucleic acid sequence of SEQ ID NO: 1173 and an antisense strand comprising the nucleic acid sequence of SEQ ID NO: 1174.

75. The composition of claim 73 wherein the sense strand of the first siRNA molecule and the sense strand of the second siRNA molecule are linked.

76. The composition of claim 75, wherein the sense strand of the first siRNA molecule and the sense strand of the second siRNA molecule are linked by a polynucleotide linker.

77. The composition of claim 76, wherein the polynucleotide linker comprises a plurality of deoxyadenosines (dA) or a plurality of deoxythymidines (dT).

78. The composition of claim 76, wherein the polynucleotide linker is sdAdAdAs, sdAdAdAdAs, sdAdAdAdAdAs, sdAdAdAdAdAdAs, sdTdTdTs, sdTdTdTdTs,
sdTdTdTdTdTs, or sdTdTdTdTdTdTs, wherein s is a phosphorothioate group.

79. The composition of claim 76, wherein the sense strand of the first siRNA molecule linked to the sense strand of the second siRNA molecule comprises the nucleic acid sequence of SEQ ID NO: 1180.

80. The composition of claim 70, further comprising a ligand targeting group conjugated to the sense strand.

81. The composition of claim 80, wherein the ligand targeting group is conjugated to the 5′ or the 3′ of the sense strand.

82. The composition of claim 80, wherein the ligand targeting group is an N-acetylgalactosamine.

83. The composition of claim 82, wherein the N-acetylgalactosamine is L96 having the formula of:

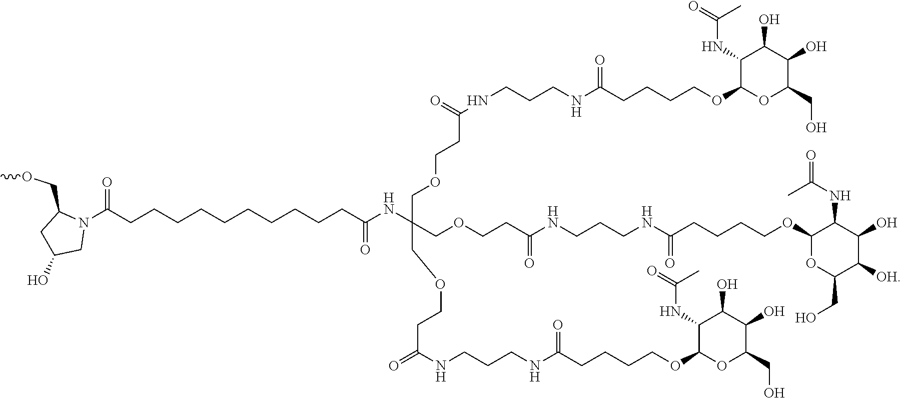

84. A composition comprising:
a first nucleic acid sequence comprising a sense strand comprising the nucleic acid sequence of SEQ ID NO: 1180 and a ligand targeting group conjugated to the 5′ and the 3′ of the sense strand;
a second nucleic acid sequence comprising an antisense strand comprising the nucleic sequence of SEQ ID NO: 1054; and
a third nucleic acid sequence comprising an antisense strand comprising the nucleic acid sequence of SEQ ID NO: 1174,
wherein the ligand targeting group is L96 having the formula of:

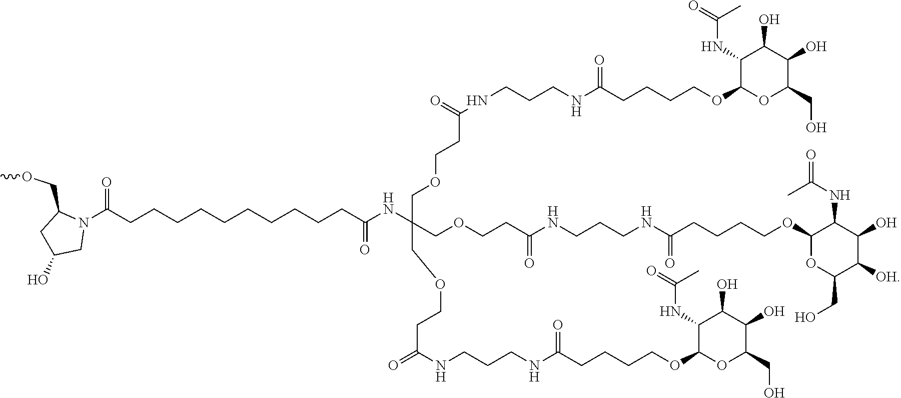

85. A pharmaceutical composition comprising the siRNA molecule of claim 66.

86. A pharmaceutical composition comprising the composition of claim 70.

87. A pharmaceutical composition comprising the composition of claim 72.

88. A pharmaceutical composition comprising the composition of claim 84.

89. A method of inhibiting the expression of PCSK9 in a subject in need thereof, the method comprising administering the pharmaceutical composition of claim 85 to the subject.

90. A method of inhibiting the expression of PCSK9 in a subject in need thereof, the method comprising administering the pharmaceutical composition of claim 86 to the subject.

91. A method of inhibiting the expression of PCSK9 and Lp(a) in a subject in need thereof, the method comprising administering the pharmaceutical composition of claim 87 to the subject.

92. A method of inhibiting the expression of PCSK9 and Lp(a) in a subject in need thereof, the method comprising administering the pharmaceutical composition of claim 88 to the subject.

93. A method of treating hypercholesterolemia in a subject in need thereof, the method comprising administering to the subject the pharmaceutical composition of claim 88.
